(19) United States
(12) Patent Application Publication
(10) Pub. No.: US 2022/0243164 A1
(43) Pub. Date: Aug. 4, 2022

(54) DEVICES, SYSTEMS, AND KITS FOR ELECTRO-MECHANICAL DELIVERY AND METHODS OF USE THEREOF

(71) Applicant: Kytopen Corporation, Cambridge, MA (US)

(72) Inventors: Paulo Andres GARCIA, Somerville, MA (US); Rameech MCCORMACK, Cambridge, MA (US); Jessica SIDO, Alton, IL (US); James HEMPHILL, Medford, MA (US); Harrison BRALOWER, Somerville, MA (US); Ross BEIGHLEY, Cambridge, MA (US); Cullen BUIE, Cambridge, MA (US); Bethany GRANT, Scituate, MA (US)

(21) Appl. No.: 17/624,535

(22) PCT Filed: Jul. 2, 2020
(86) PCT No.: PCT/US2020/040784

§ 371 Date: Jan. 3, 2022

Related U.S. Application Data
(60) Provisional application No. 62/869,958, filed on Jul. 2, 2019.

(30) Foreign Application Priority Data
Oct. 28, 2019 (US) PCT/US2019/058375

(51) Int. Cl. C12M 1/42; C12N 13/00; C12N 15/87; C12N 5/0783; C12N 5/0786

(52) U.S. Cl. CPC C12M 35/02; C12N 13/00; C12M 1/42; C12N 5/0646; C12N 5/0645; C12N 15/87

(57) ABSTRACT

Devices, systems, and kits for cell transfection are provided. A device includes a first electrode, a second electrode, and an electroporation zone therebetween where an electrical potential difference applied to the first and second electrodes generates an electric field in the electroporation zone sufficient to transfect at least a subset of the cells in the flow path. Methods of introducing a composition into at least a portion of a plurality of cells using the devices, systems, and kits of the invention are also provided.

### STATEMENT OF FEDERALLY SPONSORED RESEARCH

This invention was made with government support under Phase I SBIR Grant No. 1747096 and Phase II SBIR Grant. No 1853194 from the National Science Foundation (NSF). The government has certain rights in the invention.

### BACKGROUND OF THE INVENTION

Immunotherapy is currently at the cutting edge of both basic scientific research and pharmaceutically driven clinical application. This trend is in part due to the recent strides in targeted gene modification and the expanded use of CRISPR/Cas complex editing for therapeutic development. In order to identify genetic modifications of therapeutic interest, research organizations often have to screen thousands of genetic variants, which can include modification of an endogenous gene or insertion of an engineered gene. This drug discovery process is laborious, requiring significant manual labor within the laboratory, creating an industry-wide bottleneck due to the lack of adequate high-throughput technologies.

Biotech and pharmaceutical research and development activities have shifted to automating nearly all steps of the process. The workflows include liquid handling robots, powered by sophisticated laboratory management software, to enable high throughput discovery. However, transfection steps are limited to low throughput, poor efficiency technologies, and user-intensive systems that cannot be automated. Automated platforms for transfection not only have the potential to reduce process costs substantially, but also increase cell viability and the quantity of successfully engineered cells, all while reducing discovery time, which is critical in the competitive immunotherapy space.

A unique strength of transfection by way of electroporation is RNA delivery. Existing viral techniques to deliver DNA appear on par with transfection by way of electroporation, but there is a lack of GMP-quality non-retroviral RNA viruses. Therefore, companies with electroporation platforms have been the target of collaborations and acquisitions for the purpose of delivering mRNA into cells.

Current high-throughput gene transfer methods typically require the use of viral delivery (e.g., lentiviral vectors), in which viral particles infect a cell and transduce the genetic modification of interest. While a viral methodology can be applied to high-throughput automated systems, there are limitations in the production that extend timelines for research efforts: viral vectors have to be cloned, transfected into a viral production line, and then viral particles must be purified. This process can take research organizations months, significantly affecting their timelines for platform development while simultaneously increasing the cost of drug discovery. Additionally, the use of viral transduction for gene transfer is not amenable to genetic modification for all cell types, since some cells (such as specific immune cell subsets) are resistant to viral infection. Therefore, within the biotechnology industry there is an unmet need to have a high-throughput automated system for gene transfer that does not rely on viral delivery mechanisms.

### SUMMARY OF THE INVENTION

In one aspect, the invention features a device for electro-mechanical delivery of a composition into a plurality of cells suspended in a liquid (e.g., a liquid flowing through the device), the device including a first and second electrode and an electroporation zone. The first electrode includes a first inlet, a first outlet, and a first lumen including a minimum cross-sectional dimension, and the second electrode includes a second inlet, a second outlet, and a second lumen including a minimum cross-sectional dimension. The electroporation zone is disposed between the first outlet and the second inlet and has a minimum cross-sectional dimension that is greater than about 100 μm (e.g., from 100 μm to 10 mm, from 150 μm to 15 mm, from 200 μm to 10 mm, from 250 μm to 5 mm, from 500 μm to 10 mm, from 1 mm to 10 mm, from 1 mm to 50 mm, from 5 mm to 25 mm, or from 20 mm to 50 mm, e.g., about 0.5 mm, about 1.0 mm, about 1.5 mm, about 2 mm, about 5 mm, about 10 mm, about 15 mm, about 25 mm, or about 50 mm), wherein the electroporation zone has a substantially uniform transverse cross-sectional area. The first outlet, the electroporation zone, and the second inlet are in fluidic communication.

In another aspect, the invention features a device for electroporating a composition into a plurality of cells suspended in a liquid (e.g., a liquid flowing through the device), the device including a first and second electrode and an electroporation zone. The first electrode includes a first inlet, a first outlet, and a first lumen including a minimum cross-sectional dimension, and the second electrode includes a second inlet, a second outlet, and a second lumen including a minimum cross-sectional dimension. The electroporation zone is disposed between the first outlet and the second inlet and has a minimum cross-sectional dimension that is greater than about 100 μm (e.g., from 100 μm to 10 mm, from 150 μm to 15 mm, from 200 μm to 10 mm, from 250 μm to 5 mm, from 500 μm to 10 mm, from 1 mm to 10 mm, from 1 mm to 50 mm, from 5 mm to 25 mm, or from 20 mm to 50 mm, e.g., about 0.5 mm, about 1.0 mm, about 1.5 mm, about 2 mm, about 5 mm, about 10 mm, about 15 mm, about 25 mm, or about 50 mm), wherein the electroporation zone has a substantially uniform transverse cross-sectional area. The first outlet, the electroporation zone, and the second inlet are in fluidic communication. Transfection of cells can occur within the electroporation zone via an electro-mechanical delivery mechanism that is distinct from the delivery mechanism in static electroporation systems.

In some embodiments of either of the preceding aspects, a transverse cross-section of the electroporation zone is a shape selected from a group consisting of circular, disk, elliptical, regular polygon, irregular polygon, curvilinear shape, star, parallelogram, trapezoidal, and irregular shape (e.g., a shape having protrusions, such as protruding slots or grooves, irregular polygons, and/or curvilinear shapes). In some embodiments, the cross-section of the electroporation zone varies along the length (i.e., longitudinal axis or direction of flow) of the electroporation zone. In some embodiments, the shape is consistent along the length but varies in position relative to the central longitudinal axis along the length of the electroporation zone (e.g., the cross-sectional shape rotates about the central axis from one end of the electroporation zone to the other, such as a helix). In particular embodiments, the electroporation zone has a substantially circular transverse cross-section. In some embodiments, the electroporation zone has a transverse cross-sectional area of between about 7,850 μm² and about 2,000 mm² (e.g., between about 8,000 μm² and about 1 mm², between about 8,000 μm² and about 10 mm², between about 8,000 μm² and about 100 mm², between about 9,000 μm² and 5 mm², between about 1 mm² and about 10 mm², between about 1 mm² and about 100 mm², between about 3 mm² and about 20 mm², between about 10 mm² and about 50 mm², between about 25 mm² and about 75 mm², between about 50 mm² and about 100 mm², between about 75 mm² and about 200 mm², between about 100 mm² and about 350 mm², between about 150 mm² and about 500 mm², between about 300 mm² and about 750 mm², between about 500 mm² and about 1,000 mm², between about 750 mm² and about 1,500 mm², or between about 950 mm² and about 2,000 mm², e.g., about 8,000 μm², about 9,000 μm², about 1 mm², about 5 mm², about 10 mm², about 15 mm², about 20 mm², about 25 mm², about 50 mm², about 60 mm², about 75 mm², about 80 mm², about 100 mm², about 150 mm², about 200 mm², about 250 mm², about 300 mm², about 350 mm², about 400 mm², about 450 mm², about 500 mm², about 600 mm², about 700 mm², about 800 mm², about 900 mm², about 1,000 mm², about 1,100 mm², about 1,200 mm², about 1,300 mm², about 1,400 mm², about 1,500 mm², about 1,600 mm², about 1,700 mm², about 1,800 mm², about 1,900 mm², or about 2,000 mm²).

In some embodiments, the electroporation zone has a length of between 0.005 mm and 50 mm (e.g., between 0.005 mm and 0.05 mm, between 0.005 mm and 0.5 mm, between 0.005 mm and 25 mm, between 0.01 mm and 1 mm, between 0.05 mm and 5 mm, between 0.1 mm and 10 mm, between 0.1 mm and 50 mm, between 0.5 mm and 5 mm, between 0.5 mm and 25 mm, between 1 mm and 5 mm, between 1 mm and 10 mm, between 1 mm and 25 mm, between 3 mm and 15 mm, between 3 mm and 50 mm, between 10 mm and 20 mm, between 10 mm and 50 mm, between 15 mm and 25 mm, between 20 mm and 30 mm, between 25 mm and 40, or between 30 mm and 50 mm, e.g., about 0.005 mm, about 0.01 mm, about 0.05 mm, about 0.1 mm, about 0.5 mm, about 1.0 mm, about 1.5 mm, about 2 mm, about 3 mm, about 4 mm, about 5 mm, about 6 mm, about 7 mm, about 8 mm, about 9 mm, about 10 mm, about 15 mm, about 20 mm, about 25 mm, about 30 mm, about 35 mm, about 40 mm, about 45 mm, or about 50 mm). In some embodiments, the electroporation zone has a length of between 0.005 mm and 25 mm (e.g., between 0.005 mm and 0.05 mm, between 0.005 mm and 0.5 mm, between 0.01 mm and 1 mm, between 0.05 mm and 5 mm, between 0.1 mm and 10 mm, between 0.5 mm and 5 mm, between 0.5 mm and 10 mm, between 1 mm and 5 mm, between 1 mm and 10 mm, between 1 mm and 25 mm, between 3 mm and 10 mm, between 7 mm and 15 mm, between 10 mm and 20 mm, or between 15 mm and 25 mm, e.g., about 0.005 mm, about 0.01 mm, about 0.05 mm, about 0.1 mm, about 0.5 mm, about 1.0 mm, about 1.5 mm, about 2 mm, about 3 mm, about 4 mm, about 5 mm, about 6 mm, about 7 mm, about 8 mm, about 10 mm, about 12 mm, about 15 mm, about 18 mm, about 20 mm, about 23 mm, or about 25 mm).

In some embodiments, a lumen of any of the first electrode and/or the second electrode has a minimum cross-sectional dimension of between 0.01 mm and 500 mm (e.g., between 0.01 mm and 0.1 mm, between 0.01 mm and 0.5 mm, between 0.01 mm and 10 mm, between 0.05 mm and 5 mm, between 0.1 mm and 10 mm, between 0.5 mm and 5 mm, between 0.5 mm and 50 mm, between 1 mm and 5 mm, between 1 mm and 10 mm, between 1 mm and 25 mm, between 3 mm and 15 mm, between 3 mm and 50 mm, between 10 mm and 20 mm, between 10 mm and 100 mm, between 15 mm and 30 mm, between 20 mm and 40 mm, between 20 mm and 200 mm, between 30 mm and 50, between 30 mm and 300 mm, between 45 mm and 60 mm, between 50 mm and 100 mm, between 50 mm and 500 mm, between 75 mm and 150 mm, between 75 mm and 300 mm, between 100 mm and 200 mm, between 100 mm and 500 mm, between 150 mm and 300 mm, between 200 mm and 400 mm, between 300 mm and 450 mm, or between 350 mm and 500 mm, e.g., about 0.005 mm, about 0.01 mm, about 0.05 mm, about 0.1 mm, about 0.5 mm, about 1.0 mm, about 1.5 mm, about 2 mm, about 3 mm, about 4 mm, about 5 mm, about 6 mm, about 7 mm, about 8 mm, about 10 mm, about 15 mm, about 25 mm, about 30 mm, about 35 mm, about 40 mm, about 45 mm, about 50 mm, about 60 mm, about 70 mm, about 80 mm, about 90 mm, about 100 mm, about 150 mm, about 200 mm, about 250 mm, about 300 mm, about 350 mm, about 400 mm, about 450 mm, or about 500 mm).

In some embodiments, a ratio of the minimum cross-sectional dimension of a lumen of either of the first or second electrode to the minimum cross-sectional dimension of the electroporation zone is between 1:10 and 10:1 (e.g., between 1:10 and 1:5, between 1:10 and 1:2, between 1:10 and 1:1, between 1:10 and 2:1, between 1:10 and 5:1, between 1:5 and 1:2, between 1:5 and 1:1, between 1:5 and 2:1, between 1:5 and 5:1, between 1:2 and 2:3, between 1:2 and 1:1, between 1:2 and 2:1, between 1:2 and 6:1, between 2:3 and 2:1, between 2:3 and 4:1, between 1:1 and 2:1, between 1:1 and 3:1, between 1:1 and 10:1, between 3:2 and 3:1, between 3:2 and 6:1, between 2:1 and 3:1, between 2:1 and 5:1, between 5:2 and 5:1, between 3:1 and 4:1, between 7:2 and 5:1, between 7:2 and 10:1, between 4:1 and 8:1, between 5:1 and 10:1, or between 7:1 and 10:1, e.g., about 1:10, about 1:9, about 1:8, about 1:7, about 1:6, about 1:5, about 1:2, about 2:3, about 1:1, about 3:2, about 2:1, about 5:2, about 3:1, about 7:2, about 4:1, about 9:2, about 5:1, about 6:1, about 7:1, about 8:1, about 9:1, or about 10:1).

In some embodiments, a ratio of the minimum cross-sectional dimension of the electroporation zone to the length of the electroporation zone is between 1:100 and 100:1 (e.g., between 1:100 and 1:50, between 1:100 and 1:25, between 1:100 and 1:10, between 1:100 and 1:1, between 1:50 and 1:5, between 1:50 and 1:2, between 1:50 and 2:1, between 1:25 and 1:10, between 1:25 and 1:5, between 1:25 and 1:1, between 1:25 and 10:1, between 1:10 and 1:1, between 1:10 and 2:1, between 1:10 and 5:1, between 1:5 and 1:2, between 1:5 and 1:1, between 1:5 and 2:1, between 1:2 and 1:1, between 1:2 and 2:1, between 1:1 and 2:1, between 1:1 and 5:1, between 1:1 and 10:1, between 1:1 and 50:1, between 1:1 and 100:1, between 2:1 and 5:1, between 2:1 and 20:1, between 3:1 and 10:1, between 4:1 and 25:1, between 5:1 and 50:1, between 10:1 and 50:1, between 40:1 and 80:1, between 50:1 and 100:1, or between 75:1 and 90:1, e.g., about 1:100, about 1:75, about 1:50, about 1:25, about 1:10, about 1:5, about 1:2, about 1:1, about 3:2, about 2:1, about 5:2, about 3:1, about 7:2, about 4:1, about 5:1, about 10:1, about 20:1, about 30:1, about 40:1, about 50:1, about 60:1, about 70:1, about 80:1, about 90:1, or about 100:1).

In some embodiments, a ratio of a transverse cross-sectional area of a lumen of any of the first electrode and/or the second electrode to the transverse cross-sectional area of the electroporation zone is between 1:100 and 100:1 (e.g., between 1:100 and 1:50, between 1:100 and 1:25, between 1:100 and 1:10, between 1:100 and 1:1, between 1:50 and 1:5, between 1:50 and 1:2, between 1:50 and 2:1, between 1:25 and 1:10, between 1:25 and 1:5, between 1:25 and 1:1, between 1:25 and 10:1, between 1:10 and 1:1, between 1:10 and 2:1, between 1:10 and 5:1, between 1:5 and 1:2, between 1:5 and 1:1, between 1:5 and 2:1, between 1:2 and 1:1, between 1:2 and 2:1, between 1:1 and 2:1, between 1:1 and 5:1, between 1:1 and 10:1, between 1:1 and 50:1, between 1:1 and 100:1, between 2:1 and 5:1, between 2:1 and 20:1, between 3:1 and 10:1, between 4:1 and 25:1, between 5:1 and 50:1, between 10:1 and 50:1, between 40:1 and 80:1, between 50:1 and 100:1, or between 75:1 and 90:1, e.g., about 1:100, about 1:75, about 1:50, about 1:25, about 1:10, about 1:5, about 1:2, about 1:1, about 3:2, about 2:1, about 5:2, about 3:1, about 7:2, about 4:1, about 5:1, about 10:1, about 20:1, about 30:1, about 40:1, about 50:1, about 60:1, about 70:1, about 80:1, about 90:1, or about 100:1).

In some embodiments, the device further includes a first reservoir (e.g., a sample bag) in fluidic communication with the first inlet and/or a second reservoir (e.g., a collection bag, e.g., a recovery bag) in fluidic communication with the second outlet. Additionally, the device may include a third reservoir in fluidic communication with the first lumen or the second lumen. The third reservoir may contain one or more reagents for transfection (e.g., reagents having a composition selected for transfection), e.g., a payload suspension, such as a genetic composition, to be delivered to the cells. In some embodiments, the third reservoir is configured to contain buffer, buffer additive, and/or payload suspension comprising the composition in a non-liquid phase (e.g., solid salt) that can be diluted before or during the electroporation or electro-mechanical process. In some embodiments, either of the first electrode or the second electrode has an additional inlet or outlet for fluidic communication with the third reservoir.

In some embodiments, either of the first electrode or the second electrode can be porous or a conductive fluid (e.g., conductive liquid).

A device of any of the preceding embodiments may include a delivery source in fluidic communication with the first inlet. The delivery source can be configured to deliver the liquid and/or the plurality of cells in suspension through the first lumen to the second outlet. A delivery source can also be configured to deliver other components, such as a composition (e.g., genetic composition) to be introduced to the cells (e.g., as a transfection reagent reservoir) or additional buffer or buffer additive, using a fluid displacement mechanism (e.g., a single syringe pump or multiple syringe pumps) in fluidic connection with the delivery source and the first lumen.

In some embodiments, the delivery source is configured to deliver components prior to (e.g. upstream of) or within the electroporation zone. In some embodiments, the delivery source is configured such that the fluid displacement mechanism (e.g., a single syringe pump or multiple syringe pumps) is dedicated to one buffer, buffer additive, and/or payload suspension and is in fluidic communication with the plurality of cells in suspension.

In some embodiments, the device further includes one or more additional electroporation zones (e.g., one, two, three, four, six, eight, ten, 11, 12, 24, 27, 36, 48, 64, 96, 384, 1536, or more) additional electroporation zones, which can be configured in parallel, in series, or a combination thereof. The one or more additional electroporation zones can each have a substantially uniform transverse cross-sectional area.

In some embodiments of any of the aforementioned embodiments, the device can further include a housing configured to encase the first electrode, second electrode, and the electroporation zone. The housing may include a first electrical input operatively coupled to the first electrode and a second electrical input operatively coupled to the second electrode. In some embodiments, the housing further includes a thermal controller configured to increase the temperature of the device and/or of the liquid in which the plurality of cells is suspended, wherein the thermal controller is a heating element selected from a group consisting of a heating block, a liquid flow, a battery powered heater, and a thin-film heater. In some embodiments, the housing further includes a thermal controller configured to decrease the temperature of the device and/or of the liquid in which the plurality of cells is suspended, wherein the thermal controller is a cooling element selected from a group consisting of a liquid flow, an evaporative cooler, and a Peltier device. The housing can be integral or releasably connected to the device.

In another aspect, the invention includes a device for electro-mechanical delivery of a composition into a plurality of cells suspended in a liquid, wherein the device includes a first electrode including a first inlet, a first outlet, and a first lumen including a minimum cross-sectional dimension; a second electrode including a second inlet, a second outlet, and a second lumen including a minimum cross-sectional dimension; a third inlet and a third outlet, wherein the third inlet and the third outlet are in fluidic communication with the first lumen, wherein the third inlet and the third outlet intersect the first electrode between the first inlet and the first outlet; a fourth inlet and a fourth outlet, wherein the fourth inlet and fourth outlet are in fluidic communication with the second lumen, wherein the fourth inlet and fourth outlet intersect the second electrode between the second inlet and the second outlet; and an electroporation zone disposed between the first outlet and the second inlet, wherein the electroporation zone includes a minimum cross-sectional dimension greater than about 100 μm (e.g., from 100 μm to 10 mm, from 150 μm to 15 mm, from 200 μm to 10 mm, from 250 μm to 5 mm, from 500 μm to 10 mm, from 1 mm to 10 mm, from 1 mm to 50 mm, from 5 mm to 25 mm, or from 20 mm to 50 mm, e.g., about 0.5 mm, about 1.0 mm, about 1.5 mm, about 2 mm, about 5 mm, about 10 mm, about 15 mm, about 25 mm, or about 50 mm), wherein the electroporation zone has a substantially uniform cross-sectional area. The first outlet, the electroporation zone, and the second inlet are in fluidic communication. The transverse cross-section of the electroporation zone is a shape selected from a group consisting of circular, disk, elliptical, regular polygon, irregular polygon, curvilinear shape, star, parallelogram, trapezoidal, and irregular shape (e.g., a shape having protrusions, e.g., protruding slots or grooves, irregular polygons, and/or curvilinear shapes). In some embodiments, the cross-section of the electroporation zone varies along the length (i.e., longitudinal axis or direction of flow) of the electroporation zone). In some embodiments, the shape is consistent along the length but varies in position relative to the central longitudinal axis along the length of the electroporation zone (e.g., the cross-sectional shape rotates about the central axis from one end of the electroporation zone to the other, such as a helix). In particular embodiments, the electroporation zone has a substantially circular transverse cross-section. In some embodiments, the electroporation zone has a transverse cross-sectional area of between about 7850 μm² and about 2000 mm² (e.g., between about 8,000 μm² and about 1 mm², between about 8,000 μm² and about 10 mm², between about 8,000 μm² and about 100 mm², between about 9,000 μm² and 5 mm², between about 1 mm² and about 10 mm², between about 1 mm² and about 100 mm², between about 3 mm² and about 20 mm², between about 10 mm² and about 50 mm², between about 25 mm² and about 75 mm², between about 50 mm² and about 100 mm², between about 75 mm² and about 200 mm², between about 100 mm² and about 350 mm², between about 150 mm² and about 500 mm², between about 300 mm² and about 750 mm², between about 500 mm² and about 1,000 mm², between about 750 mm² and about 1,500 mm², or between about 950 mm² and about 2,000 mm², e.g., about 8,000 μm², about 9,000 μm², about 1 mm², about 5 mm², about 10 mm², about 15 mm², about 20 mm², about 25 mm², about 50 mm², about 60 mm², about 75 mm², about 80 mm², about 100 mm², about 150 mm², about 200 mm², about 250 mm², about 300 mm², about 350 mm², about 400 mm², about 450 mm², about 500 mm², about 600 mm², about 700 mm², about 800 mm², about 900 mm², about 1,000 mm², about 1,100 mm², about 1,200 mm², about 1,300 mm², about 1,400 mm², about 1,500 mm², about 1,600 mm², about 1,700 mm², about 1,800 mm², about 1,900 mm², or about 2,000 mm²).

In another aspect, the invention includes a device for transfecting a composition into a plurality of cells suspended in a liquid, wherein the device includes a first electrode including a first inlet, a first outlet, and a first lumen including a minimum cross-sectional dimension; a second electrode including a second inlet, a second outlet, and a second lumen including a minimum cross-sectional dimension; a third inlet and a third outlet, wherein the third inlet and the third outlet are in fluidic communication with the first lumen, wherein the third inlet and the third outlet intersect the first electrode between the first inlet and the first outlet; a fourth inlet and a fourth outlet, wherein the fourth inlet and fourth outlet are in fluidic communication with the second lumen, wherein the fourth inlet and fourth outlet intersect the second electrode between the second inlet and the second outlet; and an electroporation zone disposed between the first outlet and the second inlet, wherein the electroporation zone includes a minimum cross-sectional dimension greater than about 100 μm (e.g., from 100 μm to 10 mm, from 150 μm to 15 mm, from 200 μm to 10 mm, from 250 μm to 5 mm, from 500 μm to 10 mm, from 1 mm to 10 mm, from 1 mm to 50 mm, from 5 mm to 25 mm, or from 20 mm to 50 mm, e.g., about 0.5 mm, about 1.0 mm, about 1.5 mm, about 2 mm, about 5 mm, about 10 mm, about 15 mm, about 25 mm, or about 50 mm), wherein the electroporation zone has a substantially uniform cross-sectional area. The first outlet, the electroporation zone, and the second inlet are in fluidic communication. The transverse cross-section of the electroporation zone is a shape selected from a group consisting of circular, disk, elliptical, regular polygon, irregular polygon, curvilinear shape, star, parallelogram, trapezoidal, and irregular shape (e.g., a shape having protrusions, e.g., protruding slots or grooves, irregular polygons, and/or curvilinear shapes). In some embodiments, the cross-section of the electroporation zone varies along the length (i.e., longitudinal axis or direction of flow) of the electroporation zone). In some embodiments, the shape is consistent along the length but varies in position relative to the central longitudinal axis along the length of the electroporation zone (e.g., the cross-sectional shape rotates about the central axis from one end of the electroporation zone to the other, such as a helix). In particular embodiments, the electroporation zone has a substantially circular transverse cross-section. In some embodiments, the electroporation zone has a transverse cross-sectional area of between about 7850 μm² and about 2000 mm² (e.g., between about 8,000 μm² and about 1 mm², between about 8,000 μm² and about 10 mm², between about 8,000 μm² and about 100 mm², between about 9,000 μm² and 5 mm², between about 1 mm² and about 10 mm², between about 1 mm² and about 100 mm², between about 3 mm² and about 20 mm², between about 10 mm² and about 50 mm², between about 25 mm² and about 75 mm², between about 50 mm² and about 100 mm², between about 75 mm² and about 200 mm², between about 100 mm² and about 350 mm², between about 150 mm² and about 500 mm², between about 300 mm² and about 750 mm², between about 500 mm² and about 1,000 mm², between about 750 mm² and about 1,500 mm², or between about 950 mm² and about 2,000 mm², e.g., about 8,000 μm², about 9,000 μm², about 1 mm², about 5 mm², about 10 mm², about 15 mm², about 20 mm², about 25 mm², about 50 mm², about 60 mm², about 75 mm², about 80 mm², about 100 mm², about 150 mm², about 200 mm², about 250 mm², about 300 mm², about 350 mm², about 400 mm², about 450 mm², about 500 mm², about 600 mm², about 700 mm², about 800 mm², about 900 mm², about 1,000 mm², about 1,100 mm², about 1,200 mm², about 1,300 mm², about 1,400 mm², about 1,500 mm², about 1,600 mm², about 1,700 mm², about 1,800 mm², about 1,900 mm², or about 2,000 mm²).

In some embodiments of any of the preceding aspects, the electroporation zone has a minimum cross-sectional dimension of between 0.1 mm and 50 mm (e.g., between 0.1 mm and 0.5 mm, between 0.1 mm and 1 mm, between 0.1 mm and 5 mm, between 0.1 mm and 10 mm, between 0.5 mm and 5 mm, between 1 mm and 5 mm, between 1 mm and 10 mm, between 1 mm and 25 mm, between 3 mm and 15 mm, between 3 mm and 50 mm, between 10 mm and 20 mm, between 10 mm and 100 mm, between 15 mm and 30 mm, between 20 mm and 40 mm, between 20 mm and 200 mm, between 30 mm and 50, between 45 mm and 60 mm, between 50 mm and 100 mm, between 75 mm and 150 mm, between 100 mm and 200 mm, between 150 mm and 300 mm, between 200 mm and 400 mm, between 300 mm and 450 mm, or between 350 mm and 500 mm, e.g., about 0.1 mm, about 0.5 mm, about 1.0 mm, about 1.5 mm, about 2 mm, about 3 mm, about 4 mm, about 5 mm, about 6 mm, about 7 mm, about 8 mm, about 10 mm, about 15 mm, about 25 mm, about 30 mm, about 35 mm, about 40 mm, about 45 mm, or about 50 mm).

In some embodiments of any of the preceding aspects, the electroporation zone has a length of between 0.005 mm and 50 mm (e.g., between 0.005 mm and 0.05 mm, between 0.005 mm and 0.5 mm, between 0.005 mm and 25 mm, between 0.01 mm and 1 mm, between 0.05 mm and 5 mm, between 0.1 mm and 10 mm, between 0.1 mm and 50 mm, between 0.5 mm and 5 mm, between 0.5 mm and 25 mm, between 1 mm and 5 mm, between 1 mm and 10 mm, between 1 mm and 25 mm, between 3 mm and 15 mm, between 3 mm and 50 mm, between 10 mm and 20 mm, between 10 mm and 50 mm, between 15 mm and 25 mm, between 20 mm and 30 mm, between 25 mm and 40, or between 30 mm and 50 mm, e.g., about 0.005 mm, about 0.01 mm, about 0.05 mm, about 0.1 mm, about 0.5 mm, about 1.0 mm, about 1.5 mm, about 2 mm, about 3 mm, about 4 mm, about 5 mm, about 6 mm, about 7 mm, about 8 mm, about 9 mm, about 10 mm, about 15 mm, about 20 mm, about 25 mm, about 30 mm, about 35 mm, about 40 mm, about 45 mm, or about 50 mm). In some embodiments, the electroporation zone has a length of between 0.005 mm and 25 mm (e.g., between 0.005 mm and 0.05 mm, between 0.005 mm and 0.5 mm, between 0.01 mm and 1 mm, between 0.05 mm and 5 mm, between 0.1 mm and 10 mm, between 0.5 mm and 5 mm, between 0.5 mm and 10 mm, between 1 mm and 5 mm, between 1 mm and 10 mm, between 1 mm and 25 mm, between 3 mm and 10 mm, between 7 mm and 15 mm, between 10 mm and 20 mm, or between 15 mm and 25 mm, e.g., about 0.005 mm, about 0.01 mm, about 0.05 mm, about 0.1 mm, about 0.5 mm, about 1.0 mm, about 1.5 mm, about 2 mm, about 3 mm, about 4 mm, about 5 mm, about 6 mm, about 7 mm, about 8 mm, about 10 mm, about 12 mm, about 15 mm, about 18 mm, about 20 mm, about 23 mm, or about 25 mm).

In some embodiments of any of the preceding aspects, a lumen of any of the first electrode and/or the second electrode has a minimum cross-sectional dimension of between 0.01 mm and 500 mm (e.g., between 0.01 mm and 0.1 mm, between 0.01 mm and 0.5 mm, between 0.01 mm and 10 mm, between 0.05 mm and 5 mm, between 0.1 mm and 10 mm, between 0.5 mm and 5 mm, between 0.5 mm and 50 mm, between 1 mm and 5 mm, between 1 mm and 10 mm, between 1 mm and 25 mm, between 3 mm and 15 mm, between 3 mm and 50 mm, between 10 mm and 20 mm, between 10 mm and 100 mm, between 15 mm and 30 mm, between 20 mm and 40 mm, between 20 mm and 200 mm, between 30 mm and 50, between 30 mm and 300 mm, between 45 mm and 60 mm, between 50 mm and 100 mm, between 50 mm and 500 mm, between 75 mm and 150 mm, between 75 mm and 300 mm, between 100 mm and 200 mm, between 100 mm and 500 mm, between 150 mm and 300 mm, between 200 mm and 400 mm, between 300 mm and 450 mm, or between 350 mm and 500 mm, e.g., about 0.005 mm, about 0.01 mm, about 0.05 mm, about 0.1 mm, about 0.5 mm, about 1.0 mm, about 1.5 mm, about 2 mm, about 3 mm, about 4 mm, about 5 mm, about 6 mm, about 7 mm, about 8 mm, about 10 mm, about 15 mm, about 25 mm, about 30 mm, about 35 mm, about 40 mm, about 45 mm, about 50 mm, about 60 mm, about 70 mm, about 80 mm, about 90 mm, about 100 mm, about 150 mm, about 200 mm, about 250 mm, about 300 mm, about 350 mm, about 400 mm, about 450 mm, or about 500 mm). In some embodiments, a ratio of the minimum cross-sectional dimension of a lumen of any of the first electrode or the second electrode to the minimum cross-sectional dimension of the electroporation zone is between 1:10 and 10:1 (e.g., between 1:10 and 1:5, between 1:10 and 1:2, between 1:10 and 1:1, between 1:10 and 2:1, between 1:10 and 5:1, between 1:5 and 1:2, between 1:5 and 1:1, between 1:5 and 2:1, between 1:5 and 5:1, between 1:2 and 2:3, between 1:2 and 1:1, between 1:2 and 2:1, between 1:2 and 6:1, between 2:3 and 2:1, between 2:3 and 4:1, between 1:1 and 2:1, between 1:1 and 3:1, between 1:1 and 10:1, between 3:2 and 3:1, between 3:2 and 6:1, between 2:1 and 3:1, between 2:1 and 5:1, between 5:2 and 5:1, between 3:1 and 4:1, between 7:2 and 5:1, between 7:2 and 10:1, between 4:1 and 8:1, between 5:1 and 10:1, or between 7:1 and 10:1, e.g., about 1:10, about 1:5, about 1:2, about 2:3, about 1:1, about 3:2, about 2:1, about 5:2, about 3:1, about 7:2, about 4:1, about 9:2, about 5:1, about 6:1, about 7:1, about 8:1, about 9:1, or about 10:1).

In some embodiments of any of the preceding aspects, a ratio of the minimum cross-sectional dimension of the electroporation zone to the length of the electroporation zone is between 1:100 and 100:1 (e.g., between 1:100 and 1:50, between 1:100 and 1:25, between 1:100 and 1:10, between 1:100 and 1:1, between 1:50 and 1:5, between 1:50 and 1:2, between 1:50 and 2:1, between 1:25 and 1:10, between 1:25 and 1:5, between 1:25 and 1:1, between 1:25 and 10:1, between 1:10 and 1:1, between 1:10 and 2:1, between 1:10 and 5:1, between 1:5 and 1:2, between 1:5 and 1:1, between 1:5 and 2:1, between 1:2 and 1:1, between 1:2 and 2:1, between 1:1 and 2:1, between 1:1 and 5:1, between 1:1 and 10:1, between 1:1 and 50:1, between 1:1 and 100:1, between 2:1 and 5:1, between 2:1 and 20:1, between 3:1 and 10:1, between 4:1 and 25:1, between 5:1 and 50:1, between 10:1 and 50:1, between 40:1 and 80:1, between 50:1 and 100:1, or between 75:1 and 90:1, e.g., about 1:100, about 1:75, about 1:50, about 1:25, about 1:10, about 1:5, about 1:2, about 1:1, about 3:2, about 2:1, about 5:2, about 3:1, about 7:2, about 4:1, about 5:1, about 10:1, about 20:1, about 30:1, about 40:1, about 50:1, about 60:1, about 70:1, about 80:1, about 90:1, or about 100:1). In some embodiments, a ratio of a transverse cross-sectional area of a lumen of any of the first electrode and/or the second electrode to the transverse cross-sectional area of the electroporation zone is between 1:100 and 100:1 (e.g., between 1:100 and 1:50, between 1:100 and 1:25, between 1:100 and 1:10, between 1:100 and 1:1, between 1:50 and 1:5, between 1:50 and 1:2, between 1:50 and 2:1, between 1:25 and 1:10, between 1:25 and 1:5, between 1:25 and 1:1, between 1:25 and 10:1, between 1:10 and 1:1, between 1:10 and 2:1, between 1:10 and 5:1, between 1:5 and 1:2, between 1:5 and 1:1, between 1:5 and 2:1, between 1:2 and 1:1, between 1:2 and 2:1, between 1:1 and 2:1, between 1:1 and 5:1, between 1:1 and 10:1, between 1:1 and 50:1, between 1:1 and 100:1, between 2:1 and 5:1, between 2:1 and 20:1, between 3:1 and 10:1, between 4:1 and 25:1, between 5:1 and 50:1, between 10:1 and 50:1, between 40:1 and 80:1, between 50:1 and 100:1, or between 75:1 and 90:1, e.g., about 1:100, about 1:75, about 1:50, about 1:25, about 1:10, about 1:5, about 1:2, about 1:1, about 3:2, about 2:1, about 5:2, about 3:1, about 7:2, about 4:1, about 5:1, about 10:1, about 20:1, about 30:1, about 40:1, about 50:1, about 60:1, about 70:1, about 80:1, about 90:1, or about 100:1). Either the first or second electrode, or both, can be porous or a conductive fluid (e.g., liquid).

In some embodiments of any of the preceding aspects, the device further includes a first reservoir in fluidic communication with the first inlet. In some embodiments, the further includes a second reservoir in fluidic communication with the second outlet. In some embodiments, the device further includes a third reservoir in fluidic communication with the third inlet and the third outlet. In some embodiments, the device further includes a fourth reservoir in fluidic communication with the fourth inlet and the fourth outlet. In some embodiments, the device further includes a fifth reservoir in fluidic communication with a lumen of any of the first electrode or the second electrode, wherein any of the first electrode or the second electrode has at least one additional inlet for fluidic communication with the fifth reservoir. In some embodiments, the device further includes a fluid delivery source in fluidic communication with the first inlet, wherein the fluid delivery source is configured to deliver the liquid and/or the plurality of cells in suspension through the first lumen to the second outlet. In some embodiments, the device further includes a plurality of electroporation zones (e.g., arranged in series, in parallel, or a combination thereof). Each of the plurality of electroporation zones can have a substantially uniform transverse cross-sectional area.

In some embodiments of any of the preceding aspects, the device further includes a housing including a housing (e.g., a cartridge) configured to encase the first electrode, the second electrode, and the at least one electroporation zone of the device. The housing may include a first electrical input operatively coupled to the first electrode and a second electrical input operatively coupled to the second electrode. In some embodiments, the housing further includes a thermal controller configured to increase the temperature of the device and/or of the liquid in which the plurality of cells is suspended, wherein the thermal controller is a heating element selected from a group consisting of a heating block, a liquid flow, a battery powered heater, and a thin-film heater. In some embodiments, the housing further includes a thermal controller configured to decrease the temperature of the device and/or of the liquid in which the plurality of cells is suspended, wherein the thermal controller is a cooling element selected from a group consisting of a liquid flow, an evaporative cooler, and a Peltier device. In some embodiments, the housing is either integral to the device or releasably connected to the device. In some embodiments, the housing (e.g., cartridge) is configured for use with and/or insertion into an automated closed system that is used to deliver cell therapies to patients in a clinical or hospital setting.

In some embodiments, the housing further includes a cooling/heating area/enclosure for cell suspension and/or buffer storage during, before, and after electroporation of the specimen. In some embodiments, the system (e.g., device and housing) is externally powered.

In some embodiments, devices of the invention include a user interface or other alternative user interface that enables the user to select parameters such as flow rate, waveforms, applied potential, volume to transfect, time delay, cooling features, heating features, transfection status, progress and other parameters used to optimize the transfection protocol. In some embodiments, the user interface also contains pre-formulated parameter selections that enable the user to operate the system at standard conditions that have previously been validated by user or as recommended by the manufacturers. In some embodiments, the user interface may be connected to programming that allows for automated running of the system and/or running an algorithm to optimize transfection for a given sample of a known cell type. In some embodiments, the optimization algorithms have the ability to adjust transfection parameters independently or autonomously if the user selects this functionality. In some embodiments, the optimization algorithms allow for continuous adjustment of the parameters used in the transfection process that may depend on the cell type, conductivity of cell suspensions, volume of cell suspensions, viscosity, lifetime of the transfection cartridge(s), the physical state of the suspension, or the state of the transfection device(s).

In some embodiments, the optimization algorithms have the ability to perform predictive analysis based on known input cell-type parameters and to adjust transfection parameters accordingly. In some embodiments, the optimization algorithms adjust transfection parameters based on electrical signals within any of the devices of the invention. In some embodiments, the optimization algorithms adjust transfection parameters based on unique dimensionless input parameters. In some embodiments, the optimization algorithms have the ability to adjust transfection parameters based on unique multivariate combinations of parameters that are predictive of high viability results, high efficiency results, or matched viability and efficiency results. These unique multivariate combinations of parameters may be known relationships, such as Reynolds number, or can be new relationships, such as the relationship between shear rate and fluid velocity, channel diameter and fluid velocity, electric field and fluid velocity, etc. Generally speaking, there is a balance between the amount of electrical energy delivered via the applied electrical pulse and the amount mechanical energy delivered through fluid flow. Depending upon the desired outcome, e.g. high cell yield or high efficiency, an optimum combination of the electrical and mechanical effects exists. For a given electrical pulse condition, faster fluid flow decreases the amount of electrical energy exposed to the cell and tends to increase cell viability and cell yield. But this only occurs to a point, beyond which cell yield begins to decrease as the cells don't experience sufficient electrical energy for efficient payload delivery.

In another aspect, the invention includes a system for electro-mechanical delivery of a composition into a plurality of cells suspended in a liquid, wherein the system includes any of the aforementioned embodiments of the device.

In another aspect, the invention includes a system for electroporating a composition into a plurality of cells suspended in a liquid, wherein the system includes any of the aforementioned embodiments of the device (e.g., wherein the electroporating occurs via an electro-mechanical delivery mechanism).

In another aspect, the invention includes a system for electro-mechanical delivery of a composition into a plurality of cells suspended in a liquid, including a device and a source of electrical potential. The device includes a first electrode, a second electrode, and an electroporation zone. The first electrode includes a first inlet, a first outlet, and a first lumen including a minimum cross-sectional dimension; and the second electrode includes a second inlet, a second outlet, and a second lumen including a minimum cross-sectional dimension. The electroporation zone is disposed between the first outlet and the second inlet and has a minimum cross-sectional dimension greater than about 100 μm (e.g., from 100 μm to 10 mm, from 150 μm to 15 mm, from 200 μm to 10 mm, from 250 μm to 5 mm, from 500 μm to 10 mm, from 1 mm to 10 mm, from 1 mm to 50 mm, from 5 mm to 25 mm, or from 20 mm to 50 mm, e.g., about 0.5 mm, about 1.0 mm, about 1.5 mm, about 2 mm, about 5 mm, about 10 mm, about 15 mm, about 25 mm, or about 50 mm). The electroporation zone has a substantially uniform cross-sectional area. The first outlet, the electroporation zone, and the second inlet are in fluidic communication. The system further includes a source of electrical potential, wherein the first electrode and the second electrode of the device are releasably in operative contact with the source of electrical potential. In some embodiments, the device further includes a first reservoir in fluidic communication with the first inlet and/or a second reservoir in fluidic communication with the second outlet.

In another aspect, the invention includes a system for electroporating a composition into a plurality of cells suspended in a liquid, including a device and a source of electrical potential. The device includes a first electrode, a second electrode, and an electroporation zone. The first electrode includes a first inlet, a first outlet, and a first lumen including a minimum cross-sectional dimension; and the second electrode includes a second inlet, a second outlet, and a second lumen including a minimum cross-sectional dimension. The electroporation zone is disposed between the first outlet and the second inlet and has a minimum cross-sectional dimension greater than about 100 μm (e.g., from 100 μm to 10 mm, from 150 μm to 15 mm, from 200 μm to 10 mm, from 250 μm to 5 mm, from 500 μm to 10 mm, from 1 mm to 10 mm, from 1 mm to 50 mm, from 5 mm to 25 mm, or from 20 mm to 50 mm, e.g., about 0.5 mm, about 1.0 mm, about 1.5 mm, about 2 mm, about 5 mm, about 10 mm, about 15 mm, about 25 mm, or about 50 mm). The electroporation zone has a substantially uniform cross-sectional area. The first outlet, the electroporation zone, and the second inlet are in fluidic communication. The system further includes a source of electrical potential, wherein the first electrode and the second electrode of the device are releasably in operative contact with the source of electrical potential. In some embodiments, the device further includes a first reservoir in fluidic communication with the first inlet and/or a second reservoir in fluidic communication with the second outlet.

In some embodiments of either of the preceding aspects, the transverse cross-section of the electroporation zone is a shape selected from a group consisting of circular, disk, elliptical, regular polygon, irregular polygon, curvilinear shape, star, parallelogram, trapezoidal, and irregular. In some embodiments, the electroporation zone has a substantially circular transverse cross-section. In some embodiments, the electroporation zone has a minimum cross-sectional dimension of between 0.1 mm and 50 mm (e.g., between 0.1 mm and 0.5 mm, between 0.1 mm and 1 mm, between 0.1 mm and 5 mm, between 0.1 mm and 10 mm, between 0.5 mm and 5 mm, between 1 mm and 5 mm, between 1 mm and 10 mm, between 1 mm and 25 mm, between 3 mm and 15 mm, between 3 mm and 50 mm, between 10 mm and 20 mm, between 10 mm and 100 mm, between 15 mm and 30 mm, between 20 mm and 40 mm, between 20 mm and 200 mm, between 30 mm and 50, between 45 mm and 60 mm, between 50 mm and 100 mm, between 75 mm and 150 mm, between 100 mm and 200 mm, between 150 mm and 300 mm, between 200 mm and 400 mm, between 300 mm and 450 mm, or between 350 mm and 500 mm, e.g., about 0.1 mm, about 0.5 mm, about 1.0 mm, about 1.5 mm, about 2 mm, about 3 mm, about 4 mm, about 5 mm, about 6 mm, about 7 mm, about 8 mm, about 10 mm, about 15 mm, about 25 mm, about 30 mm, about 35 mm, about 40 mm, about 45 mm, or about 50 mm).

In some embodiments of any of the preceding systems of the invention, the electroporation zone has a transverse cross-sectional area of between about 7,850 μm² and about 2,000 mm² (e.g., between about 8,000 μm² and about 1 mm², between about 8,000 μm² and about 10 mm², between about 8,000 μm² and about 100 mm², between about 9,000 μm² and 5 mm², between about 1 mm² and about 10 mm², between about 1 mm² and about 100 mm², between about 3 mm² and about 20 mm², between about 10 mm² and about 50 mm², between about 25 mm² and about 75 mm², between about 50 mm² and about 100 mm², between about 75 mm² and about 200 mm², between about 100 mm² and about 350 mm², between about 150 mm² and about 500 mm², between about 300 mm² and about 750 mm², between about 500 mm² and about 1,000 mm², between about 750 mm² and about 1,500 mm², or between about 950 mm² and about 2,000 mm², e.g., about 8,000 μm², about 9,000 μm², about 1 mm², about 5 mm², about 10 mm², about 15 mm², about 20 mm², about 25 mm², about 50 mm², about 60 mm², about 75 mm², about 80 mm², about 100 mm², about 150 mm², about 200 mm², about 250 mm², about 300 mm², about 350 mm², about 400 mm², about 450 mm², about 500 mm², about 600 mm², about 700 mm², about 800 mm², about 900 mm², about 1,000 mm², about 1,100 mm², about 1,200 mm², about 1,300 mm², about 1,400 mm², about 1,500 mm², about 1,600 mm², about 1,700 mm², about 1,800 mm², about 1,900 mm², or about 2,000 mm²).

In some embodiments of any of the preceding systems, the electroporation zone has a length of between 0.005 mm and 50 mm (e.g., between 0.005 mm and 0.05 mm, between 0.005 mm and 0.5 mm, between 0.005 mm and 25 mm, between 0.01 mm and 1 mm, between 0.05 mm and 5 mm, between 0.1 mm and 10 mm, between 0.1 mm and 50 mm, between 0.5 mm and 5 mm, between 0.5 mm and 25 mm, between 1 mm and 5 mm, between 1 mm and 10 mm, between 1 mm and 25 mm, between 3 mm and 15 mm, between 3 mm and 50 mm, between 10 mm and 20 mm, between 10 mm and 50 mm, between 15 mm and 25 mm, between 20 mm and 30 mm, between 25 mm and 40, or between 30 mm and 50 mm, e.g., about 0.005 mm, about 0.01 mm, about 0.05 mm, about 0.1 mm, about 0.5 mm, about 1.0 mm, about 1.5 mm, about 2 mm, about 3 mm, about 4 mm, about 5 mm, about 6 mm, about 7 mm, about 8 mm, about 9 mm, about 10 mm, about 15 mm, about 20 mm, about 25 mm, about 30 mm, about 35 mm, about 40 mm, about 45 mm, or about 50 mm). In some embodiments of the systems, the length of the electroporation zone is between 0.005 mm and 25 mm (e.g., between 0.005 mm and 0.05 mm, between 0.005 mm and 0.5 mm, between 0.01 mm and 1 mm, between 0.05 mm and 5 mm, between 0.1 mm and 10 mm, between 0.5 mm and 5 mm, between 0.5 mm and 10 mm, between 1 mm and 5 mm, between 1 mm and 10 mm, between 1 mm and 25 mm, between 3 mm and 10 mm, between 7 mm and 15 mm, between 10 mm and 20 mm, or between 15 mm and 25 mm, e.g., about 0.005 mm, about 0.01 mm, about 0.05 mm, about 0.1 mm, about 0.5 mm, about 1.0 mm, about 1.5 mm, about 2 mm, about 3 mm, about 4 mm, about 5 mm, about 6 mm, about 7 mm, about 8 mm, about 10 mm, about 12 mm, about 15 mm, about 18 mm, about 20 mm, about 23 mm, or about 25 mm).

In some embodiments of any of the preceding systems, a lumen of any of the first electrode and/or the second electrode has a minimum cross-sectional dimension of between 0.01 mm and 500 mm (e.g., between 0.01 mm and 0.1 mm, between 0.01 mm and 0.5 mm, between 0.01 mm and 10 mm, between 0.05 mm and 5 mm, between 0.1 mm and 10 mm, between 0.5 mm and 5 mm, between 0.5 mm and 50 mm, between 1 mm and 5 mm, between 1 mm and 10 mm, between 1 mm and 25 mm, between 3 mm and 15 mm, between 3 mm and 50 mm, between 10 mm and 20 mm, between 10 mm and 100 mm, between 15 mm and 30 mm, between 20 mm and 40 mm, between 20 mm and 200 mm, between 30 mm and 50, between 30 mm and 300 mm, between 45 mm and 60 mm, between 50 mm and 100 mm, between 50 mm and 500 mm, between 75 mm and 150 mm, between 75 mm and 300 mm, between 100 mm and 200 mm, between 100 mm and 500 mm, between 150 mm and 300 mm, between 200 mm and 400 mm, between 300 mm and 450 mm, or between 350 mm and 500 mm, e.g., about 0.005 mm, about 0.01 mm, about 0.05 mm, about 0.1 mm, about 0.5 mm, about 1.0 mm, about 1.5 mm, about 2 mm, about 3 mm, about 4 mm, about 5 mm, about 6 mm, about 7 mm, about 8 mm, about 10 mm, about 15 mm, about 25 mm, about 30 mm, about 35 mm, about 40 mm, about 45 mm, about 50 mm, about 60 mm, about 70 mm, about 80 mm, about 90 mm, about 100 mm, about 150 mm, about 200 mm, about 250 mm, about 300 mm, about 350 mm, about 400 mm, about 450 mm, or about 500 mm).

In some embodiments of the systems of the invention, a ratio of the minimum cross-sectional dimension of a lumen of any of the first electrode or the second electrode to the minimum cross-sectional dimension of the electroporation zone is between 1:10 and 10:1 (e.g., between 1:10 and 1:5, between 1:10 and 1:2, between 1:10 and 1:1, between 1:10 and 2:1, between 1:10 and 5:1, between 1:5 and 1:2, between 1:5 and 1:1, between 1:5 and 2:1, between 1:5 and 5:1, between 1:2 and 2:3, between 1:2 and 1:1, between 1:2 and 2:1, between 1:2 and 6:1, between 2:3 and 2:1, between 2:3 and 4:1, between 1:1 and 2:1, between 1:1 and 3:1, between 1:1 and 10:1, between 3:2 and 3:1, between 3:2 and 6:1, between 2:1 and 3:1, between 2:1 and 5:1, between 5:2 and 5:1, between 3:1 and 4:1, between 7:2 and 5:1, between 7:2 and 10:1, between 4:1 and 8:1, between 5:1 and 10:1, or between 7:1 and 10:1, e.g., about 1:10, about 1:9, about 1:8, about 1:7, about 1:6, about 1:5, about 1:2, about 2:3, about 1:1, about 3:2, about 2:1, about 5:2, about 3:1, about 7:2, about 4:1, about 9:2, about 5:1, about 6:1, about 7:1, about 8:1, about 9:1, or about 10:1). In some embodiments, a ratio of the minimum cross-sectional dimension of the electroporation zone to the length of the electroporation zone is between 1:100 and 100:1 (e.g., between 1:100 and 1:50, between 1:100 and 1:25, between 1:100 and 1:10, between 1:100 and 1:1, between 1:50 and 1:5, between 1:50 and 1:2, between 1:50 and 2:1, between 1:25 and 1:10, between 1:25 and 1:5, between 1:25 and 1:1, between 1:25 and 10:1, between 1:10 and 1:1, between 1:10 and 2:1, between 1:10 and 5:1, between 1:5 and 1:2, between 1:5 and 1:1, between 1:5 and 2:1, between 1:2 and 1:1, between 1:2 and 2:1, between 1:1 and 2:1, between 1:1 and 5:1, between 1:1 and 10:1, between 1:1 and 50:1, between 1:1 and 100:1, between 2:1 and 5:1, between 2:1 and 20:1, between 3:1 and 10:1, between 4:1 and 25:1, between 5:1 and 50:1, between 10:1 and 50:1, between 40:1 and 80:1, between 50:1 and 100:1, or between 75:1 and 90:1, e.g., about 1:100, about 1:75, about 1:50, about 1:25, about 1:10, about 1:5, about 1:2, about 1:1, about 3:2, about 2:1, about 5:2, about 3:1, about 7:2, about 4:1, about 5:1, about 10:1, about 20:1, about 30:1, about 40:1, about 50:1, about 60:1, about 70:1, about 80:1, about 90:1, or about 100:1).

In some embodiments, a ratio of a transverse cross-sectional area of a lumen of any of the first electrode and/or the second electrode to the transverse cross-sectional area of the electroporation zone is between 1:100 and 100:1 (e.g., between 1:100 and 1:50, between 1:100 and 1:25, between 1:100 and 1:10, between 1:100 and 1:1, between 1:50 and 1:5, between 1:50 and 1:2, between 1:50 and 2:1, between 1:25 and 1:10, between 1:25 and 1:5, between 1:25 and 1:1, between 1:25 and 10:1, between 1:10 and 1:1, between 1:10 and 2:1, between 1:10 and 5:1, between 1:10 and 10:1, between 1:5 and 1:2, between 1:5 and 1:1, between 1:5 and 2:1, between 1:5 and 50:1, between 1:2 and 1:1, between 1:2 and 2:1, between 1:2 and 10:1, between 1:1 and 2:1, between 1:1 and 5:1, between 1:1 and 10:1, between 1:1 and 50:1, between 1:1 and 100:1, between 2:1 and 5:1, between 2:1 and 20:1, between 2:1 and 50:1, between 3:1 and 10:1, between 3:1 and 30:1, between 4:1 and 25:1, between 5:1 and 10:1, between 5:1 and 50:1, between 10:1 and 50:1, between 10:1 and 100:1, between 40:1 and 80:1, between 50:1 and 100:1, or between 75:1 and 90:1, e.g., about 1:100, about 1:75, about 1:50, about 1:25, about 1:10, about 1:5, about 1:2, about 1:1, about 3:2, about 2:1, about 5:2, about 3:1, about 7:2, about 4:1, about 5:1, about 6:1, about 7:1, about 8:1, about 9:1, about 10:1, about 15:1, about 20:1, about 25:1, about 30:1, about 40:1, about 50:1, about 60:1, about 70:1, about 80:1, about 90:1, or about 100:1). Either of the first electrode or the second electrode, or both, can be porous or a conductive fluid (e.g., liquid).

In some embodiments of any of the preceding systems, the system includes a third reservoir in fluidic communication with a lumen of any of the first electrode or the second electrode, wherein any of the first electrode or the second electrode has an additional inlet for fluidic communication with the third reservoir. In some embodiments, the system further includes a fluid delivery source in fluidic communication with the first inlet, wherein the fluid delivery source is configured to deliver the liquid and/or the plurality of cells in suspension through the first lumen to the second outlet.

In some embodiments, the system of the invention further includes a controller operatively coupled to the source of electrical potential to deliver voltage pulses to the first electrode and the second electrode, wherein the voltage pulses generate an electrical potential difference between the first electrode and the second electrode, thus producing an electric field in the electroporation zone. In some embodiments, the system includes a plurality of electroporation zones (e.g., as part of a plurality of any embodiment(s) of the devices provided herein). Each of the plurality of electroporation zones can have a substantially uniform or non-uniform transverse cross-sectional area.

In some embodiments, the system further includes an outer structure including a housing configured to encase the first electrode, the second electrode, and the at least one electroporation zone of the device (e.g., wherein the outer structure further includes a first electrical input operatively coupled to the first electrode and a second electrical input operatively coupled to the second electrode). The housing may include a thermal controller configured to increase the temperature of the device and/or of the liquid in which the plurality of cells is suspended. The thermal controller can be a heating element selected from a group consisting of a heating block, a liquid flow, a battery-powered heater, and a thin-film heater. Additionally or alternatively, the thermal controller can be configured to decrease the temperature of the device and/or of the liquid in which the plurality of cells is suspended, wherein the thermal controller is a cooling element selected from a group consisting of a liquid flow, an evaporative cooler, and a Peltier device.

In some embodiments of any of preceding the systems of the invention, the source of electrical potential is releasably connected to the first and second electrical inputs of the outer structure. The releasable connection between the first or second electrical inputs and the source of electrical potential can be selected from a group consisting of a clamp, a clip, a spring, a sheath, a wire brush, mechanical connection, inductive connection, or a combination thereof. The outer structure may be integral to, or releasably connected to, the device. In some embodiments, a housing (e.g., cartridge) is configured to energize a plurality of devices in parallel, in series, or offset in time, wherein the housing further includes a tray that accommodates a plurality of electroporation devices, wherein the tray is modified with two grid electrodes, wherein a first grid electrode is electrically isolated from a second grid electrode, wherein an exterior of the first electrode of each of the plurality of devices is releasably in operative contact with any of a first spring-loaded electrode, a first mechanically connected electrode, or a first inductively connected electrode, wherein an exterior of the second electrode of each of the plurality of devices is releasably in operative contact with any of a second spring-loaded electrode, a second mechanically connected electrode, or a second inductively coupled electrode, wherein each of the plurality of devices releasably enters the housing (e.g., cartridge) through an opening in the grid electrodes, wherein any of the first spring-loaded electrode, first mechanically connected electrode, or first inductively connected electrode of each device is in operative contact with the first grid electrode and any of the second spring-loaded electrode, second mechanically connected electrode, or second inductively connected electrode of each device is in operative contact with the second grid electrode, wherein the grid electrodes are connected to the source of electrical potential.

In some embodiments, the housing (e.g., cartridge) encapsulates one or more of the previously stated inventions or one or more electroporating devices used for continuous flow electroporation. In some embodiments, the housing (e.g., cartridge) is configured to allow use with and/or insertion into an automated closed system that delivers cell therapies to patients. In some embodiments, the housing further includes a cooling/heating area/enclosure for cell suspension and/or buffer storage during, before and after electroporation of the specimen. In some embodiments, the system (e.g., one or more devices and housing) is externally powered.

In some embodiments, the system also includes optimization algorithms that have the ability to adjust electroporation parameters independently or autonomously if the user selects this functionality.

These optimization algorithms allow for continuous adjustment of the parameters used in the electroporation process that may depend on the cell type, conductivity, volume of suspensions, viscosity, lifetime of the electroporating cartridge, the physical state of the suspension or the state of the electroporation device.

In some embodiments of any of the preceding aspects of the system, the source of electrical potential delivers voltage pulses to the grid electrodes, wherein the first grid electrode is energized at a particular applied voltage while the second grid electrode is energized at a particular applied voltage, wherein each of the plurality of devices is energized by the grid electrodes with an identical applied voltage pulse such that a magnitude of an electric field generated within each of the at least one electroporation zones of each device is substantially identical. In some embodiments, the source of electrical potential includes additional circuitry or programming configured to modulate the delivery of voltage pulses to the grid electrodes, wherein each of the plurality of devices may receive a different voltage from the grid electrodes, wherein a magnitude of an electric field generated within each of the at least one electroporation zones of each device is different.

In another aspect, the invention provides a system for electro-mechanical delivery of a composition into a plurality of cells suspended in a liquid, including: a device, including a first electrode including a first inlet, a first outlet, and a first lumen; a second electrode including a second inlet, a second outlet, and a second lumen; a third inlet and a third outlet, wherein the third inlet and the third outlet are in fluidic communication with the first lumen, wherein the third inlet and third outlet intersect the first electrode between the first inlet and the first outlet; a fourth inlet and a fourth outlet, wherein the fourth inlet and the fourth outlet are in fluidic communication with the second lumen, wherein the fourth inlet and fourth outlet intersect the second electrode between the second inlet and the second outlet; and an electroporation zone disposed between the first outlet and the second inlet, wherein the electroporation zone has a length of between 0.005 mm and 50 mm (e.g., between 0.005 mm and 0.05 mm, between 0.005 mm and 0.5 mm, between 0.005 mm and 25 mm, between 0.01 mm and 1 mm, between 0.05 mm and 5 mm, between 0.1 mm and 10 mm, between 0.1 mm and 50 mm, between 0.5 mm and 5 mm, between 0.5 mm and 25 mm, between 1 mm and 5 mm, between 1 mm and 10 mm, between 1 mm and 25 mm, between 3 mm and 15 mm, between 3 mm and 50 mm, between 10 mm and 20 mm, between 10 mm and 50 mm, between 15 mm and 25 mm, between 20 mm and 30 mm, between 25 mm and 40, or between 30 mm and 50 mm, e.g., about 0.005 mm, about 0.01 mm, about 0.05 mm, about 0.1 mm, about 0.5 mm, about 1.0 mm, about 1.5 mm, about 2 mm, about 3 mm, about 4 mm, about 5 mm, about 6 mm, about 7 mm, about 8 mm, about 9 mm, about 10 mm, about 15 mm, about 20 mm, about 25 mm, about 30 mm, about 35 mm, about 40 mm, about 45 mm, or about 50 mm) and includes a minimum cross-sectional dimension greater than about 100 μm (e.g., from 100 μm to 10 mm, from 150 μm to 15 mm, from 200 μm to 10 mm, from 250 μm to 5 mm, from 500 μm to 10 mm, from 1 mm to 10 mm, from 1 mm to 50 mm, from 5 mm to 25 mm, or from 20 mm to 50 mm, e.g., about 0.5 mm, about 1.0 mm, about 1.5 mm, about 2 mm, about 5 mm, about 10 mm, about 15 mm, about 25 mm, or about 50 mm), wherein a transverse cross-sectional area of the electroporation zone is substantially uniform; and wherein a ratio of a minimum cross-sectional dimension of the first lumen to the minimum cross-sectional dimension of the electroporation zone is between 1:10 and 10:1 (e.g., between 1:10 and 1:5, between 1:10 and 1:2, between 1:10 and 1:1, between 1:10 and 2:1, between 1:10 and 5:1, between 1:5 and 1:2, between 1:5 and 1:1, between 1:5 and 2:1, between 1:5 and 5:1, between 1:2 and 2:3, between 1:2 and 1:1, between 1:2 and 2:1, between 1:2 and 6:1, between 2:3 and 2:1, between 2:3 and 4:1, between 1:1 and 2:1, between 1:1 and 3:1, between 1:1 and 10:1, between 3:2 and 3:1, between 3:2 and 6:1, between 2:1 and 3:1, between 2:1 and 5:1, between 5:2 and 5:1, between 3:1 and 4:1, between 7:2 and 5:1, between 7:2 and 10:1, between 4:1 and 8:1, between 5:1 and 10:1, or between 7:1 and 10:1, e.g., about 1:10, about 1:5, about 1:2, about 2:3, about 1:1, about 3:2, about 2:1, about 5:2, about 3:1, about 7:2, about 4:1, about 9:2, about 5:1, about 6:1, about 7:1, about 8:1, about 9:1, or about 10:1), wherein a ratio of a minimum cross-sectional dimension of the second lumen to the minimum cross-sectional dimension of the electroporation zone is between 1:10 and 10:1 (e.g., between 1:10 and 1:5, between 1:10 and 1:2, between 1:10 and 1:1, between 1:10 and 2:1, between 1:10 and 5:1, between 1:5 and 1:2, between 1:5 and 1:1, between 1:5 and 2:1, between 1:5 and 5:1, between 1:2 and 2:3, between 1:2 and 1:1, between 1:2 and 2:1, between 1:2 and 6:1, between 2:3 and 2:1, between 2:3 and 4:1, between 1:1 and 2:1, between 1:1 and 3:1, between 1:1 and 10:1, between 3:2 and 3:1, between 3:2 and 6:1, between 2:1 and 3:1, between 2:1 and 5:1, between 5:2 and 5:1, between 3:1 and 4:1, between 7:2 and 5:1, between 7:2 and 10:1, between 4:1 and 8:1, between 5:1 and 10:1, or between 7:1 and 10:1, e.g., about 1:10, about 1:5, about 1:2, about 2:3, about 1:1, about 3:2, about 2:1, about 5:2, about 3:1, about 7:2, about 4:1, about 9:2, about 5:1, about 6:1, about 7:1, about 8:1, about 9:1, or about 10:1), and wherein the first outlet, the electroporation zone, and the second inlet are in fluidic communication; and a source of electrical potential, wherein the first and second electrodes of the device are releasably in operative contact with the source of electrical potential. The transverse cross-section of the electroporation zone is a closed shape selected from a group consisting of circular, disk, elliptical, regular polygon, irregular polygon, curvilinear shape, star, parallelogram, trapezoidal, and irregular. The electroporation zone can have a substantially circular transverse cross-section.

In another aspect, the invention provides a system for electroporating a composition into a plurality of cells suspended in a liquid, including: a device, including a first electrode including a first inlet, a first outlet, and a first lumen; a second electrode including a second inlet, a second outlet, and a second lumen; a third inlet and a third outlet, wherein the third inlet and the third outlet are in fluidic communication with the first lumen, wherein the third inlet and third outlet intersect the first electrode between the first inlet and the first outlet; a fourth inlet and a fourth outlet, wherein the fourth inlet and the fourth outlet are in fluidic communication with the second lumen, wherein the fourth inlet and fourth outlet intersect the second electrode between the second inlet and the second outlet; and an electroporation zone disposed between the first outlet and the second inlet, wherein the electroporation zone has a length of between 0.005 mm and 50 mm (e.g., between 0.005 mm and 0.05 mm, between 0.005 mm and 0.5 mm, between 0.005 mm and 25 mm, between 0.01 mm and 1 mm, between 0.05 mm and 5 mm, between 0.1 mm and 10 mm, between 0.1 mm and 50 mm, between 0.5 mm and 5 mm, between 0.5 mm and 25 mm, between 1 mm and 5 mm, between 1 mm and 10 mm, between 1 mm and 25 mm, between 3 mm and 15 mm, between 3 mm and 50 mm, between 10 mm and 20 mm, between 10 mm and 50 mm, between 15 mm and 25 mm, between 20 mm and 30 mm, between 25 mm and 40, or between 30 mm and 50 mm, e.g., about 0.005 mm, about 0.01 mm, about 0.05 mm, about 0.1 mm, about 0.5 mm, about 1.0 mm, about 1.5 mm, about 2 mm, about 3 mm, about 4 mm, about 5 mm, about 6 mm, about 7 mm, about 8 mm, about 9 mm, about 10 mm, about 15 mm, about 20 mm, about 25 mm, about 30 mm, about 35 mm, about 40 mm, about 45 mm, or about 50 mm) and includes a minimum cross-sectional dimension greater than about 100 μm (e.g., from 100 μm to 10 mm, from 150 μm to 15 mm, from 200 μm to 10 mm, from 250 μm to 5 mm, from 500 μm to 10 mm, from 1 mm to 10 mm, from 1 mm to 50 mm, from 5 mm to 25 mm, or from 20 mm to 50 mm, e.g., about 0.5 mm, about 1.0 mm, about 1.5 mm, about 2 mm, about 5 mm, about 10 mm, about 15 mm, about 25 mm, or about 50 mm), wherein a transverse cross-sectional area of the electroporation zone is substantially uniform; and wherein a ratio of a minimum cross-sectional dimension of the first lumen to the minimum cross-sectional dimension of the electroporation zone is between 1:10 and 10:1 (e.g., between 1:10 and 1:5, between 1:10 and 1:2, between 1:10 and 1:1, between 1:10 and 2:1, between 1:10 and 5:1, between 1:5 and 1:2, between 1:5 and 1:1, between 1:5 and 2:1, between 1:5 and 5:1, between 1:2 and 2:3, between 1:2 and 1:1, between 1:2 and 2:1, between 1:2 and 6:1, between 2:3 and 2:1, between 2:3 and 4:1, between 1:1 and 2:1, between 1:1 and 3:1, between 1:1 and 10:1, between 3:2 and 3:1, between 3:2 and 6:1, between 2:1 and 3:1, between 2:1 and 5:1, between 5:2 and 5:1, between 3:1 and 4:1, between 7:2 and 5:1, between 7:2 and 10:1, between 4:1 and 8:1, between 5:1 and 10:1, or between 7:1 and 10:1, e.g., about 1:10, about 1:5, about 1:2, about 2:3, about 1:1, about 3:2, about 2:1, about 5:2, about 3:1, about 7:2, about 4:1, about 9:2, about 5:1, about 6:1, about 7:1, about 8:1, about 9:1, or about 10:1), wherein a ratio of a minimum cross-sectional dimension of the second lumen to the minimum cross-sectional dimension of the electroporation zone is between 1:10 and 10:1 (e.g., between 1:10 and 1:5, between 1:10 and 1:2, between 1:10 and 1:1, between 1:10 and 2:1, between 1:10 and 5:1, between 1:5 and 1:2, between 1:5 and 1:1, between 1:5 and 2:1, between 1:5 and 5:1, between 1:2 and 2:3, between 1:2 and 1:1, between 1:2 and 2:1, between 1:2 and 6:1, between 2:3 and 2:1, between 2:3 and 4:1, between 1:1 and 2:1, between 1:1 and 3:1, between 1:1 and 10:1, between 3:2 and 3:1, between 3:2 and 6:1, between 2:1 and 3:1, between 2:1 and 5:1, between 5:2 and 5:1, between 3:1 and 4:1, between 7:2 and 5:1, between 7:2 and 10:1, between 4:1 and 8:1, between 5:1 and 10:1, or between 7:1 and 10:1, e.g., about 1:10, about 1:5, about 1:2, about 2:3, about 1:1, about 3:2, about 2:1, about 5:2, about 3:1, about 7:2, about 4:1, about 9:2, about 5:1, about 6:1, about 7:1, about 8:1, about 9:1, or about 10:1), and wherein the first outlet, the electroporation zone, and the second inlet are in fluidic communication; and a source of electrical potential, wherein the first and second electrodes of the device are releasably in operative contact with the source of electrical potential. The transverse cross-section of the electroporation zone is a closed shape selected from a group consisting of circular, disk, elliptical, regular polygon, irregular polygon, curvilinear shape, star, parallelogram, trapezoidal, and irregular. The electroporation zone can have a substantially circular transverse cross-section.

In some embodiments of either of the preceding aspects, the electroporation zone has a minimum cross-sectional dimension of between 0.1 mm and 50 mm (e.g., between 0.1 mm and 0.5 mm, between 0.1 mm and 1 mm, between 0.1 mm and 5 mm, between 0.1 mm and 10 mm, between 0.5 mm and 5 mm, between 1 mm and 5 mm, between 1 mm and 10 mm, between 1 mm and 25 mm, between 3 mm and 15 mm, between 3 mm and 50 mm, between 10 mm and 20 mm, between 10 mm and 100 mm, between 15 mm and 30 mm, between 20 mm and 40 mm, between 20 mm and 200 mm, between 30 mm and 50, between 45 mm and 60 mm, between 50 mm and 100 mm, between 75 mm and 150 mm, between 100 mm and 200 mm, between 150 mm and 300 mm, between 200 mm and 400 mm, between 300 mm and 450 mm, or between 350 mm and 500 mm, e.g., about 0.1 mm, about 0.5 mm, about 1.0 mm, about 1.5 mm, about 2 mm, about 3 mm, about 4 mm, about 5 mm, about 6 mm, about 7 mm, about 8 mm, about 10 mm, about 15 mm, about 25 mm, about 30 mm, about 35 mm, about 40 mm, about 45 mm, or about 50 mm).

In some embodiments, the electroporation zone has a transverse cross-sectional area of between about 7,850 μm² and about 2,000 mm² (e.g., between about 8,000 μm² and about 1 mm², between about 8,000 μm² and about 10 mm², between about 8,000 μm² and about 100 mm², between about 9,000 μm² and 5 mm², between about 1 mm² and about 10 mm², between about 1 mm² and about 100 mm², between about 3 mm² and about 20 mm², between about 10 mm² and about 50 mm², between about 25 mm² and about 75 mm², between about 50 mm² and about 100 mm², between about 75 mm² and about 200 mm², between about 100 mm² and about 350 mm², between about 150 mm² and about 500 mm², between about 300 mm² and about 750 mm², between about 500 mm² and about 1,000 mm², between about 750 mm² and about 1,500 mm², or between about 950 mm² and about 2,000 mm², e.g., about 8,000 μm², about 9,000 μm², about 1 mm², about 5 mm², about 10 mm², about 15 mm², about 20 mm², about 25 mm², about 50 mm², about 60 mm², about 75 mm², about 80 mm², about 100 mm², about 150 mm², about 200 mm², about 250 mm², about 300 mm², about 350 mm², about 400 mm², about 450 mm², about 500 mm², about 600 mm², about 700 mm², about 800 mm², about 900 mm², about 1,000 mm², about 1,100 mm², about 1,200 mm², about 1,300 mm², about 1,400 mm², about 1,500 mm², about 1,600 mm², about 1,700 mm², about 1,800 mm², about 1,900 mm², or about 2,000 mm²).

In some embodiments, the electroporation zone has a length of between 0.005 mm and 50 mm (e.g., between 0.005 mm and 0.05 mm, between 0.005 mm and 0.5 mm, between 0.005 mm and 25 mm, between 0.01 mm and 1 mm, between 0.05 mm and 5 mm, between 0.1 mm and 10 mm, between 0.1 mm and 50 mm, between 0.5 mm and 5 mm, between 0.5 mm and 25 mm, between 1 mm and 5 mm, between 1 mm and 10 mm, between 1 mm and 25 mm, between 3 mm and 15 mm, between 3 mm and 50 mm, between 10 mm and 20 mm, between 10 mm and 50 mm, between 15 mm and 25 mm, between 20 mm and 30 mm, between 25 mm and 40, or between 30 mm and 50 mm, e.g., about 0.005 mm, about 0.01 mm, about 0.05 mm, about 0.1 mm, about 0.5 mm, about 1.0 mm, about 1.5 mm, about 2 mm, about 3 mm, about 4 mm, about 5 mm, about 6 mm, about 7 mm, about 8 mm, about 9 mm, about 10 mm, about 15 mm, about 20 mm, about 25 mm, about 30 mm, about 35 mm, about 40 mm, about 45 mm, or about 50 mm). In some embodiments of the system of the invention, the length of the electroporation zone is between 0.005 mm and 25 mm (e.g., between 0.005 mm and 0.05 mm, between 0.005 mm and 0.5 mm, between 0.01 mm and 1 mm, between 0.05 mm and 5 mm, between 0.1 mm and 10 mm, between 0.5 mm and 5 mm, between 0.5 mm and 10 mm, between 1 mm and 5 mm, between 1 mm and 10 mm, between 1 mm and 25 mm, between 3 mm and 10 mm, between 7 mm and 15 mm, between 10 mm and 20 mm, or between 15 mm and 25 mm, e.g., about 0.005 mm, about 0.01 mm, about 0.05 mm, about 0.1 mm, about 0.5 mm, about 1.0 mm, about 1.5 mm, about 2 mm, about 3 mm, about 4 mm, about 5 mm, about 6 mm, about 7 mm, about 8 mm, about 10 mm, about 12 mm, about 15 mm, about 18 mm, about 20 mm, about 23 mm, or about 25 mm).

In some embodiments, the device or system further includes a plurality of fluid constraining cavities for cell transfection that are in fluidic communication with the at least one electroporation zone and in which the plurality of cells experience similar or different electric fields and similar or different flow velocities (e.g., displacement velocities). In some embodiments, the electrodes may be in any configuration (e.g., manifold) with multiple inlets and outlets that lead to a constriction and support cell electroporation in multiple non-conductive fluid constraining cavities. In some embodiments, each of the plurality of fluid constraining cavities for cell electroporation includes an individual housing (e.g., cartridge) and can operate independently. In some embodiments, each individual housing (e.g., cartridge) is configured to be exposed to the same or different operating parameters that control flow velocities (e.g., displacement velocities), electric field, or a combination of other parameters. In some embodiments, each housing is configured to be integrated into one or more controller systems. In other embodiments, the one or more controller systems are configured to accept one or more individual housings.

In some embodiments, the device or system further includes a plurality of fluid constraining cavities for cell electroporation that are in fluidic communication with the at least one electroporation zone and in which the plurality of cells experience similar or different electric fields and similar or different flow velocities (e.g., displacement velocities). In some embodiments, the electrodes may be in any configuration (e.g., manifold) with multiple inlets and outlets that lead to a constriction and support cell electroporation in multiple non-conductive fluid constraining cavities. In some embodiments, each of the plurality of fluid constraining cavities for cell electroporation includes an individual housing (e.g., cartridge) and can operate independently. In some embodiments, each individual housing (e.g., cartridge) is configured to be exposed to the same or different operating parameters that control flow velocities (e.g., displacement velocities), electric field, or a combination of other parameters. In some embodiments, each housing is configured to be integrated into one or more controller systems. In other embodiments, the one or more controller systems are configured to accept one or more individual housings.

In some embodiments, a lumen of any of the first electrode and/or the second electrode has a minimum cross-sectional dimension of between 0.01 mm and 500 mm (e.g., between 0.01 mm and 0.1 mm, between 0.01 mm and 0.5 mm, between 0.01 mm and 10 mm, between 0.05 mm and 5 mm, between 0.1 mm and 10 mm, between 0.5 mm and 5 mm, between 0.5 mm and 50 mm, between 1 mm and 5 mm, between 1 mm and 10 mm, between 1 mm and 25 mm, between 3 mm and 15 mm, between 3 mm and 50 mm, between 10 mm and 20 mm, between 10 mm and 100 mm, between 15 mm and 30 mm, between 20 mm and 40 mm, between 20 mm and 200 mm, between 30 mm and 50, between 30 mm and 300 mm, between 45 mm and 60 mm, between 50 mm and 100 mm, between 50 mm and 500 mm, between 75 mm and 150 mm, between 75 mm and 300 mm, between 100 mm and 200 mm, between 100 mm and 500 mm, between 150 mm and 300 mm, between 200 mm and 400 mm, between 300 mm and 450 mm, or between 350 mm and 500 mm, e.g., about 0.005 mm, about 0.01 mm, about 0.05 mm, about 0.1 mm, about 0.5 mm, about 1.0 mm, about 1.5 mm, about 2 mm, about 3 mm, about 4 mm, about 5 mm, about 6 mm, about 7 mm, about 8 mm, about 10 mm, about 15 mm, about 25 mm, about 30 mm, about 35 mm, about 40 mm, about 45 mm, about 50 mm, about 60 mm, about 70 mm, about 80 mm, about 90 mm, about 100 mm, about 150 mm, about 200 mm, about 250 mm, about 300 mm, about 350 mm, about 400 mm, about 450 mm, or about 500 mm).

In some embodiments, a ratio of the minimum cross-sectional dimension of a lumen of any of the first electrode or the second electrode to the minimum cross-sectional dimension of the electroporation zone is between 1:10 and 10:1 (e.g., between 1:10 and 1:5, between 1:10 and 1:2, between 1:10 and 1:1, between 1:10 and 2:1, between 1:10 and 5:1, between 1:5 and 1:2, between 1:5 and 1:1, between 1:5 and 2:1, between 1:5 and 5:1, between 1:2 and 2:3, between 1:2 and 1:1, between 1:2 and 2:1, between 1:2 and 6:1, between 2:3 and 2:1, between 2:3 and 4:1, between 1:1 and 2:1, between 1:1 and 3:1, between 1:1 and 10:1, between 3:2 and 3:1, between 3:2 and 6:1, between 2:1 and 3:1, between 2:1 and 5:1, between 5:2 and 5:1, between 3:1 and 4:1, between 7:2 and 5:1, between 7:2 and 10:1, between 4:1 and 8:1, between 5:1 and 10:1, or between 7:1 and 10:1, e.g., about 1:10, about 1:5, about 1:2, about 2:3, about 1:1, about 3:2, about 2:1, about 5:2, about 3:1, about 7:2, about 4:1, about 9:2, about 5:1, about 6:1, about 7:1, about 8:1, about 9:1, or about 10:1). In some embodiments, a ratio of the minimum cross-sectional dimension of the electroporation zone to the length of the electroporation zone is between 1:100 and 100:1 (e.g., between 1:100 and 1:50, between 1:100 and 1:25, between 1:100 and 1:10, between 1:100 and 1:1, between 1:50 and 1:5, between 1:50 and 1:2, between 1:50 and 2:1, between 1:25 and 1:10, between 1:25 and 1:5, between 1:25 and 1:1, between 1:25 and 10:1, between 1:10 and 1:1, between 1:10 and 2:1, between 1:10 and 5:1, between 1:10 and 10:1, between 1:5 and 1:2, between 1:5 and 1:1, between 1:5 and 2:1, between 1:5 and 50:1, between 1:2 and 1:1, between 1:2 and 2:1, between 1:2 and 10:1, between 1:1 and 2:1, between 1:1 and 5:1, between 1:1 and 10:1, between 1:1 and 50:1, between 1:1 and 100:1, between 2:1 and 5:1, between 2:1 and 20:1, between 2:1 and 50:1, between 3:1 and 10:1, between 3:1 and 30:1, between 4:1 and 25:1, between 5:1 and 10:1, between 5:1 and 50:1, between 10:1 and 50:1, between 10:1 and 100:1, between 40:1 and 80:1, between 50:1 and 100:1, or between 75:1 and 90:1, e.g., about 1:100, about 1:75, about 1:50, about 1:25, about 1:10, about 1:5, about 1:2, about 1:1, about 3:2, about 2:1, about 5:2, about 3:1, about 7:2, about 4:1, about 5:1, about 6:1, about 7:1, about 8:1, about 9:1, about 10:1, about 15:1, about 20:1, about 25:1, about 30:1, about 40:1, about 50:1, about 60:1, about 70:1, about 80:1, about 90:1, or about 100:1). In some embodiments, a ratio of a transverse cross-sectional area of a lumen of any of the first electrode and/or the second electrode to the transverse cross-sectional area of the electroporation zone is between 1:100 and 100:1 (e.g., between 1:100 and 1:50, between 1:100 and 1:25, between 1:100 and 1:10, between 1:100 and 1:1, between 1:50 and 1:5, between 1:50 and 1:2, between 1:50 and 2:1, between 1:25 and 1:10, between 1:25 and 1:5, between 1:25 and 1:1, between 1:25 and 10:1, between 1:10 and 1:1, between 1:10 and 2:1, between 1:10 and 5:1, between 1:10 and 10:1, between 1:5 and 1:2, between 1:5 and 1:1, between 1:5 and 2:1, between 1:5 and 50:1, between 1:2 and 1:1, between 1:2 and 2:1, between 1:2 and 10:1, between 1:1 and 2:1, between 1:1 and 5:1, between 1:1 and 10:1, between 1:1 and 50:1, between 1:1 and 100:1, between 2:1 and 5:1, between 2:1 and 20:1, between 2:1 and 50:1, between 3:1 and 10:1, between 3:1 and 30:1, between 4:1 and 25:1, between 5:1 and 10:1, between 5:1 and 50:1, between 10:1 and 50:1, between 10:1 and 100:1, between 40:1 and 80:1, between 50:1 and 100:1, or between 75:1 and 90:1, e.g., about 1:100, about 1:75, about 1:50, about 1:25, about 1:10, about 1:5, about 1:2, about 1:1, about 3:2, about 2:1, about 5:2, about 3:1, about 7:2, about 4:1, about 5:1, about 6:1, about 7:1, about 8:1, about 9:1, about 10:1, about 15:1, about 20:1, about 25:1, about 30:1, about 40:1, about 50:1, about 60:1, about 70:1, about 80:1, about 90:1, or about 100:1).

In some embodiments, the system further includes a first reservoir in fluidic communication with the first inlet, a second reservoir in fluidic communication with the second outlet, a third reservoir in fluidic communication with the third inlet and the third outlet, a fourth reservoir in fluidic communication with the fourth inlet and the fourth outlet, and/or a fifth reservoir in fluidic communication with a lumen of any of the first electrode or the second electrode, e.g., wherein any of the first electrode or the second electrode has at least one additional inlet for fluidic communication with the fifth reservoir. In some embodiments, the system further includes a fluid delivery source in fluidic communication with the first inlet, wherein the fluid delivery source is configured to deliver the liquid and/or the plurality of cells in suspension through the first lumen to the second outlet. In some embodiments, the device further includes a plurality of electroporation zones, e.g., wherein each of the plurality of electroporation zones has a substantially uniform or non-uniform transverse cross-sectional area.

The system of any of the previous aspects can additionally include a controller operatively coupled to the source of electrical potential to deliver voltage pulses to the first and second electrodes to generate an electrical potential difference between the first and second electrodes, thus producing an electric field in the electroporation zone.

In some embodiments, the system further includes an outer structure including a housing configured to encase the first electrode, the second electrode, and the at least one electroporation zone of the device. The system can further include a first electrical input operatively coupled to the first electrode and a second electrical input operatively coupled to the second electrode. The housing can further include a thermal controller configured to increase the temperature of the device and/or of the liquid in which the plurality of cells is suspended, wherein the thermal controller is a heating element selected from a group consisting of a heating block, a liquid flow, a battery-powered heater, and a thin-film heater. Additionally or alternatively, the housing can further include a thermal controller configured to decrease the temperature of the device and/or of the liquid in which the plurality of cells is suspended, wherein the thermal controller is a cooling element selected from a group consisting of a liquid flow, an evaporative cooler, and a Peltier device. In some embodiments, the source of electrical potential is releasably connected to the first and second electrical inputs of the outer structure, e.g., wherein the releasable connection between the first or second electrical inputs and the source of electrical potential is selected from a group consisting of a clamp, a clip, a spring, a sheath, a wire brush, mechanical connection, inductive connection, or a combination thereof. The outer structure and/or housing can be integral to, or releasably connected to, the device.

In some embodiments, the system further includes a plurality of devices, e.g., having a plurality of outer structures. In some embodiments, a housing is configured to energize a plurality of devices in parallel, in series, or offset in time, wherein the housing further includes a tray that accommodates a plurality of electroporation devices, wherein the tray is modified with two grid electrodes, wherein a first grid electrode is electrically isolated from a second grid electrode, wherein an exterior of the first electrode of each of the plurality of devices is releasably in operative contact with any of a first spring-loaded electrode, a first mechanically connected electrode, or a first inductively connected electrode, wherein an exterior of the second electrode of each of the plurality of devices is releasably in operative contact with any of a second spring-loaded electrode, a second mechanically connected electrode, or a second inductively coupled electrode, wherein each of the plurality of devices releasably enters the housing through an opening in the grid electrodes, wherein any of the first spring-loaded electrode, first mechanically connected electrode, or first inductively connected electrode of each device is in operative contact with the first grid electrode and any of the second spring-loaded electrode, second mechanically connected electrode, or second inductively connected electrode of each device is in operative contact with the second grid electrode, wherein the grid electrodes are connected to the source of electrical potential. In some embodiments, the source of electrical potential delivers voltage pulses to the grid electrodes, wherein the first grid electrode is energized at a particular applied voltage while the second grid electrode is energized at a particular applied voltage, wherein each of the plurality of devices is energized by the grid electrodes with an identical applied voltage pulse such that a magnitude of an electric field generated within each of the at least one electroporation zones of each device is substantially identical. In some embodiments, the source of electrical potential includes additional circuitry or programming configured to modulate the delivery of voltage pulses to the grid electrodes, wherein each of the plurality of devices may receive a different voltage from the grid electrodes, wherein a magnitude of an electric field generated within each of the at least one electroporation zones of each device may be different.

In another aspect, the invention provides a method of introducing a composition into a plurality of cells suspended in a flowing liquid using any of the devices or systems of the invention (e.g., by electro-mechanical delivery). In particular, methods of the invention include providing a device including a first electrode including a first outlet, a first inlet, and a first lumen including a minimum cross-sectional dimension; a second electrode including a second outlet, a second inlet, and a second lumen including a minimum cross-sectional dimension; and an electroporation zone disposed between the first outlet and the second inlet, wherein the electroporation zone includes a minimum cross-sectional dimension greater than about 100 μm (e.g., from 100 μm to 10 mm, from 150 μm to 15 mm, from 200 μm to 10 mm, from 250 μm to 5 mm, from 500 μm to 10 mm, from 1 mm to 10 mm, from 1 mm to 50 mm, from 5 mm to 25 mm, or from 20 mm to 50 mm, e.g., about 0.5 mm, about 1.0 mm, about 1.5 mm, about 2 mm, about 5 mm, about 10 mm, about 15 mm, about 25 mm, or about 50 mm), wherein the electroporation zone has a substantially uniform cross sectional area; and wherein the first outlet, the electroporation zone, and the second inlet are in fluidic communication; applying an electrical potential difference between the first and second electrodes, thereby producing an electric field in the electroporation zone; and passing the plurality of cells and the composition through the electroporation zone, thereby enhancing permeability of the plurality of cells and introducing the composition into the plurality of cells. In some embodiments, the passing the plurality of the cells includes applying a fluid-driven positive pressure. In some embodiments, none of the first lumen, second lumen, or electroporation zone has a minimum cross-sectional dimension that causes a cross-sectional dimension of any of the plurality of cells suspended in the liquid to be compressed temporarily. The electroporation can be substantially non-thermal reversible electroporation, substantially non-thermal irreversible electroporation, or substantially thermal irreversible electroporation. In some embodiments, a flow rate of a liquid and/or the plurality of cells in suspension delivered from a fluid delivery source from the first lumen to the electroporation zone is between 0.001 mL/min and 1,000 mL min (e.g., between 0.001 mL/min and 0.05 mL/min, between 0.001 mL/min and 0.1 mL/min, between 0.001 mL/min and 1 mL/min, between 0.05 mL/min and 0.5 mL/min, between 0.05 mL/min and 5 mL/min, between 0.1 mL/min and 1 mL/min, between 0.5 mL/min and 2 mL/min, between 1 mL/min and 5 mL/min, between 1 mL/min and 10 mL/min, between 1 mL/min and 100 mL/min, between 5 mL/min and 25 mL/min, between 5 mL/min and 150 mL/min, between 10 mL/min and 100 mL/min, between 15 mL/min and 150 mL/min, between 25 mL/min and 100 mL/min, between 25 mL/min and 200 mL/min, between 50 mL/min and 150 mL/min, between 50 mL/min and 250 mL/min, between 75 mL/min and 200 mL/min, between 75 mL/min and 350 mL/min, between 100 mL/min and 250 mL/min, between 100 mL/min and 400 mL/min, between 150 mL/min and 450 mL/min, between 200 mL/min and 500 mL/min, between 250 mL/min and 700 mL/min, between 300 mL/min and 1,000 mL/min, between 400 mL/min and 750 mL/min, between 500 mL/min and 1,000 mL/min, or between 750 mL/min and 1,000 mL/min, e.g., about 0.001 mL/min, about 0.01 mL/min, about 0.05 mL/min, about 0.1 mL/min, about 0.5 mL/min, about 1 mL/min, about 5 mL/min, about 10 mL/min, about 15 mL/min, about 20 mL/min, about 30 mL/min, about 40 mL/min, about 50 mL/min, about 60 mL/min, about 70 mL/min, about 80 mL/min, about 90 mL/min, about 100 mL/min, about 150 mL/min, about 200 mL/min, about 250 mL/min, about 300 mL/min, about 350 mL/min, about 400 mL/min, about 450 mL/min, about 500 mL/min, about 600 mL/min, about 700 mL/min, about 800 mL/min, about 900 mL/min, or about 1,000 mL/min), wherein the fluid delivery source is configured to deliver the liquid and/or the plurality of cells in suspension through the first lumen to the second outlet.

In some embodiments of any of the previous aspects, a Reynolds number of a liquid and/or the plurality of cells in suspension delivered from a fluid delivery source from the first lumen to the electroporation zone is between 0.04 and 2.43×10⁴ (e.g., between 10 and 2000, between 100 and 1600, between 100 and 1800, or between 183 and 1530), wherein the fluid delivery source is configured to deliver the liquid and/or the plurality of cells in suspension through the first lumen to the second outlet. In some embodiments, a maximum velocity of a liquid and/or the plurality of cells in suspension delivered from a fluid delivery source from the first lumen to the electroporation zone is between 5×10⁻⁵ m/s and 32.7 m/s (e.g., between 0.01 and 10 m/s, between 0.1 and 5 m/s, or between 0.26 and 2.08 m/s), wherein the fluid delivery source is configured to deliver the liquid and/or the plurality of cells in suspension through the first lumen to the second outlet. In some embodiments, shear rates of a liquid and/or the plurality of cells in suspension delivered from a fluid delivery source from the first lumen to the electroporation zone are between 0.1 1/s and 2×10⁶ 1/s (e.g., between 1/s and 100,000/s, between 10/s and 100,000/s, between 100/s and 100,000/s, between 1,000/s and 80,000/s, or between 2,600 and 54,000), wherein the fluid delivery source is configured to deliver the liquid and/or the plurality of cells in suspension through the first lumen to the second outlet. In some embodiments, a peak pressure of a liquid and/or the plurality of cells in suspension delivered from a fluid delivery source from the first lumen to the electroporation zone is between 1×10⁻³ Pa and 9.5×10⁴ Pa (e.g., between 0.1 Pa and 10,000 Pa, between 1 Pa and 5,000 Pa, between 100 Pa and 3000 Pa, or between 136 Pa and 1600 Pa), wherein the fluid delivery source is configured to deliver the liquid and/or the plurality of cells in suspension through the first lumen to the second outlet. In some embodiments, an average velocity of a liquid and/or the plurality of cells in suspension delivered from a fluid delivery source from the first lumen to the electroporation zone is between 1.5×10⁻⁵ m/s and 15.9 m/s (e.g., between 7.79×10⁻² m/s and 7.81×10⁻¹ m/s), wherein the fluid delivery source is configured to deliver the liquid and/or the plurality of cells in suspension through the first lumen to the second outlet. In some embodiments, a kinematic viscosity of a liquid and/or the plurality of cells in suspension delivered from a fluid delivery source from the first lumen to the electroporation zone is between 1×10⁻⁶ m²/s and 15×10⁻⁴ m²/s (e.g., between 1.3×10⁻⁶ m²/s and 5.6×10⁻⁴ m²/s), wherein the fluid delivery source is configured to deliver the liquid and/or the plurality of cells in suspension through the first lumen to the second outlet.

In some embodiments of any of the previous aspects, a residence time in the electroporation zone of any of the plurality of cells suspended in the liquid is between 0.5 ms and 50 ms (e.g., between 0.5 ms and 5 ms, between 1 ms and 10 ms, between 1 ms and 15 ms, between 5 ms and 15 ms, between 10 ms and 20 ms, between 15 ms and 25 ms, between 20 ms and 30 ms, between 25 ms and 35 ms, between 30 ms and 40 ms, between 35 ms and 45 ms, or between 40 ms and 50 ms, e.g., about 0.5 ms, about 0.6 ms, about 0.7 ms, about 0.8 ms, about 0.9 ms, about 1 ms, about 1.5 ms, about 2 ms, about 2.5 ms, about 3 ms, about 3.5 ms, about 4 ms, about 4.5 ms, about 5 ms, about 5.5 ms, about 6 ms, about 6.5 ms, about 7 ms, about 7.5 ms, about 8 ms, about 8.5 ms, about 9 ms, about 9.5 ms, about 10 ms, about 10.5 ms, about 11 ms, about 11.5 ms, about 12 ms, about 12.5 ms, about 13 ms, about 13.5 ms, about 14 ms, about 14.5 ms, about 15 ms, about 20 ms, about 25 ms, about 30 ms, about 35 ms, about 40 ms, about 45 ms, or about 50 ms). In some embodiments, the residence time is from 5-20 ms (e.g., from 6-18 ms, 8-15 ms, or 10-14 ms).

In some embodiments of any of the preceding aspects, the plurality of cells has from 0% to about 25% phenotypic change (e.g., from about 0% to about 2.5%, from about 1% to about 5%, from about 1% to about 10%, from about 5% to about 15%, from about 10% to about 20%, from about 15% to about 25%, or from about 20% to about 25%, e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, or about 25%) relative to a baseline measurement of cell phenotype upon exiting the second outlet of the device (e.g., within 48 hours after exiting the second outlet, e.g., within 24 hours after exiting the second outlet, e.g., between 1 minute and 24 hours, 5 minutes and 24 hours, 10 minutes and 24 hours, 30 minutes and 24 hours, 1 hour and 24 hours, or 2 hours and 24 hours after exiting the second outlet).

In some embodiments of any of the preceding aspects, the plurality of cells have no phenotypic change relative to a baseline measurement of cell phenotype upon exiting the second outlet of the device (e.g., within 48 hours after exiting the second outlet, e.g., within 24 hours after exiting the second outlet, e.g., between 1 minute and 24 hours, 5 minutes and 24 hours, 10 minutes and 24 hours, 30 minutes and 24 hours, 1 hour and 24 hours, or 2 hours and 24 hours after exiting the second outlet).

In some embodiments of any of the preceding aspects, the electric field is produced by voltage pulses, wherein the voltage pulses energize the first electrode at a particular applied voltage while the second electrode is energized at a particular applied voltage, thus applying an electrical potential difference between the first and second electrodes, wherein the voltage pulses each have an amplitude between −3 kV and 3 kV (e.g., between −3 kV and 1 kV, between −3 kV and −1.5 kV, between −2 kV and 2 kV, between −1.5 kV and 1.5 kV, between −1.5 kV and 2.5 kV, between −1 kV and 1 kV, between −1 kV and 2 kV, between −0.5 kV and 0.5 kV, between −0.5 kV and 1.5 kV, between −0.5 kV and 3 kV, between −0.01 kV and 2 kV, between 0 kV and 1 kV, between 0 kV and 2 kV, between 0 kV and 3 kV, between 0.01 kV and 0.1 kV, between 0.01 kV and 1 kV, between 0.02 kV and 0.2 kV, between 0.03 kV and 0.3 kV, between 0.04 kV and 0.4 kV, between 0.05 kV and 0.5 kV, between 0.05 kV and 1.5 kV, between 0.06 kV and 0.6 kV, between 0.07 kV and 0.7 kV, between 0.08 kV and 0.8 kV, between 0.09 kV and 0.9 kV, between 0.1 kV and 0.7 kV, between 0.1 kV and 1 kV, between 0.1 kV and 2 kV, between 0.1 kV and 3 kV, between 0.15 kV and 1.5 kV, between 0.2 and 0.6 kV, between 0.2 kV and 2 kV, between 0.25 kV and 2.5 kV, between 0.3 kV and 3 kV, between 0.5 kV and 1 kV, between 0.5 kV and 3 kV, between 0.6 kV and 1.5 kV, between 0.7 kV and 1.8 kV, between 0.8 kV and 2 kV, between 0.9 kV and 3 kV, between 1 kV and 2 kV, between 1.5 kV and 2.5 kV, or between 2 kV and 3 kV, e.g., about −3 kV, about −2.5 kV, about −2 kV, about −1.5 kV, about −1 kV, about −0.5 kV, about −0.01 kV, about 0 kV, about 0.01 kV, about 0.02 kV, about 0.03 kV, about 0.04 kV, about 0.05 kV, about 0.06 kV, about 0.07 kV, about 0.08 kV, about 0.09 kV, about 0.1 kV, about 0.2 kV, about 0.3 kV, about 0.4 kV, about 0.5 kV, about 0.6 kV, about 0.7 kV, about 0.8 kV, about 0.9 kV, about 1 kV, about 1.1 kV, about 1.2 kV, about 1.3 kV, about 1.4 kV, about 1.5 kV, about 1.6 kV, about 1.7 kV, about 1.8 kV, about 1.9 kV, about 2 kV, about 2.1 kV, about 2.2 kV, about 2.3 kV, about 2.4 kV, about 2.5 kV, about 2.6 kV, about 2.7 kV, about 2.8 kV, about 2.9 kV, or about 3 kV). In some embodiments, the first electrode is energized at a particular applied voltage while the second electrode is held at ground (e.g., 0 kV), thus applying an electrical potential difference between the first and second electrodes. In some embodiments, the voltage pulses have a duration of between 0.01 ms and 1,000 ms (e.g., between 0.01 ms and 0.1 ms, between 0.01 ms and 1 ms, between 0.01 ms and 10 ms, between 0.05 ms and 0.5 ms, between 0.05 ms and 1 ms, between 0.1 ms and 1 ms, between 0.1 ms and 5 ms, between 0.1 ms and 500 ms, between 0.5 ms and 2 ms, between 1 ms and 5 ms, between 1 ms and 10 ms, between 1 ms and 25 ms, between 1 ms and 100 ms, between 1 ms and 1,000 ms, between 5 ms and 25 ms, between 5 ms and 150 ms, between 10 ms and 100 ms, between 15 ms and 150 ms, between 25 ms and 100 ms, between 25 ms and 200 ms, between 50 ms and 150 ms, between 50 ms and 250 ms, between 75 ms and 200 ms, between 75 ms and 350 ms, between 100 ms and 250 ms, between 100 ms and 400 ms, between 150 ms and 450 ms, between 200 ms and 500 ms, between 250 ms and 700 ms, between 300 ms and 1,000 ms, between 400 ms and 750 ms, between 500 ms and 1,000 ms, or between 750 ms and 1,000 ms, e.g., about 0.01 ms, about 0.05 ms, about 0.1 ms, about 0.5 ms, about 1 ms, about 5 ms, about 10 ms, about 15 ms, about 20 ms, about 30 ms, about 40 ms, about 50 ms, about 60 ms, about 70 ms, about 80 ms, about 90 ms, about 100 ms, about 150 ms, about 200 ms, about 250 ms, about 300 ms, about 350 ms, about 400 ms, about 450 ms, about 500 ms, about 600 ms, about 700 ms, about 800 ms, about 900 ms, or about 1,000 ms). In some embodiments, the voltage pulses are applied to the first and second electrodes at a frequency of between 1 Hz and 50,000 Hz (e.g., between 1 Hz and 10 Hz, between 1 Hz and 100 Hz, between 1 Hz and 1,000 Hz, between 5 Hz and 20 Hz, between 5 Hz and 2,000 Hz, between 10 Hz and 50 Hz, between 10 Hz and 100 Hz, between 10 Hz and 1,000 Hz, between 10 Hz and 10,000 Hz, between 20 Hz and 50 Hz, between 20 Hz and 100 Hz, between 20 Hz and 2,000 Hz, between 20 Hz and 20,000 Hz, between 50 Hz and 500 Hz, between 50 Hz and 1,000 Hz, between 50 Hz and 50,000 Hz, between 100 Hz and 200 Hz, between 100 Hz and 500 Hz, between 100 Hz and 1,000 Hz, between 100 Hz and 10,000 Hz, between 100 Hz and 50,000 Hz, between 200 Hz and 400 Hz, between 200 Hz and 750 Hz, between 200 Hz and 2,000 Hz, between 500 Hz and 1,000 Hz, between 750 Hz and 1,500 Hz, between 750 Hz and 10,000 Hz, between 1,000 Hz and 2,000 Hz, between 1,000 Hz and 5,000 Hz, between 1,000 Hz and 10,000 Hz, between 1,000 Hz and 50,000 Hz, between 5,000 Hz and 10,000 Hz, between 5,000 Hz and 20,000 Hz, between 5,000 Hz and 50,000 Hz, between 10,000 Hz and 15,000 Hz, between 10,000 Hz and 25,000 Hz, between 10,000 Hz and 50,000 Hz, between 20,000 Hz and 30,000 Hz, or between 20,000 and 50,000 Hz, e.g., about 1 Hz, about 5 Hz, about 10 Hz, about 20 Hz, about 50 Hz, about 75 Hz, about 100 Hz, about 150 Hz, about 200 Hz, about 300 Hz, about 400 Hz, about 500 Hz, about 600 Hz, about 700 Hz, about 800 Hz, about 900 Hz, about 1,000 Hz, about 2,000 Hz, about 5,000 Hz, about 10,000 Hz, about 15,000 Hz, about 20,000 Hz, about 30,000 Hz, about 40,000 Hz, or about 50,000 Hz).

In some embodiments, a waveform of the voltage pulse is selected from a group consisting of DC, square, pulse, bipolar, sine, ramp, asymmetric bipolar, arbitrary, and any superposition or combinations thereof. In some embodiments, the electric field generated from the voltage pulses has a magnitude of between 1 V/cm and 50,000 V/cm (e.g., between 1 V/cm and 50 V/cm, between 1 V/cm and 500 V/cm, between 1 V/cm and 1,000 V/cm, between 1 V/cm and 20,000 V/cm, between 5 V/cm and 10,000 V/cm, between 25 V/cm and 200 V/cm, between 50 V/cm and 250 V/cm, between 50 V/cm and 500 V/cm, between 50 V/cm and 15,000 V/cm, between 100 V/cm and 1,000 V/cm, between 300 V/cm and 500 V/cm, between 500 V/cm and 10,000 V/cm, between 1000 V/cm and 25,000 V/cm, between 5,000 V/cm and 25,000 V/cm, between 10,000 V/cm and 20,000 V/cm, between 10,000 V/cm and 50,000 V/cm, e.g., about 1 V/cm, about 2 V/cm, about 3 V/cm, about 4 V/cm, about 5 V/cm, about 6 V/cm, about 7 V/cm, about 8 V/cm, about 9 V/cm, about 10 V/cm, about 20 V/cm, about 30 V/cm, about 40 V/cm, about 50 V/cm, about 60 V/cm, about 70 V/cm, about 80 V/cm, about 90 V/cm, about 100 V/cm, about 150 V/cm, about 200 V/cm, about 250 V/cm, about 300 V/cm, about 350 V/cm, about 400 V/cm, about 450 V/cm, about 500 V/cm, about 550 V/cm, about 600 V/cm, about 650 V/cm, about 700 V/cm, about 750 V/cm, about 800 V/cm, about 900 V/cm, about 1,000 V/cm, about 2,000 V/cm, about 3,000 V/cm, about 4,000 V/cm, about 5,000 V/cm, about 6,000 V/cm, about 7,000 V/cm, about 8,000 V/cm, about 9,000 V/cm, about 10,000 V/cm, about 15,000 V/cm, about 20,000 V/cm, about 25,000 V/cm, about 30,000 V/cm, about 35,000 V/cm, about 40,000 V/cm, about 45,000 V/cm, or about 50,000 V/cm).

In some embodiments, a duty cycle of the voltage pulses is between 0.001% and 100% (e.g., between 0.001% and 0.1%, between 0.001% and 10%, between 0.01% and 1%, between 0.01% to 100%, between 0.1% and 5%, between 0.1% and 99%, between 1% and 10%, between 1% and 97%, between 2.5% and 20%, between 5% and 25%, between 5% and 40%, between 10% and 25%, between 10% and 50%, between 10% and 95%, between 15% and 60%, between 15% and 85%, between 20% and 40%, between 30% and 50%, between 40% and 60%, between 40% and 75%, between 50% and 85%, between 50% and 100%, between 75% and 100%, or between 90% and 100%, e.g., about 0.001%, about 0.002%, about 0.003%, about 0.004%, about 0.005%, about 0.006%, about 0.007%, about 0.008%, about 0.009%, about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%).

In some embodiments, the liquid has a conductivity of between 0.001 mS/cm and 500 mS/cm (e.g., between 0.001 mS/cm and 0.05 mS/cm, between 0.001 mS/cm and 0.1 mS/cm, between 0.001 mS/cm and 1 mS/cm, between 0.05 mS/cm and 0.5 mS/cm, between 0.05 mS/cm and 5 mS/cm, between 0.1 mS/cm and 1 mS/cm, between 0.1 mS/cm and 100 mS/cm, between 0.5 mS/cm and 2 mS/cm, between 1 mS/cm and 5 mS/cm, between 1 mS/cm and 10 mS/cm, between 1 mS/cm and 100 mS/cm, between 1 mS/cm and 500 mS/cm, between 5 mS/cm and 25 mS/cm, between 5 mS/cm and 150 mS/cm, between 10 mS/cm and 100 mS/cm, between 10 mS/cm and 250 mS/cm, between 15 mS/cm and 150 mS/cm, between 25 mS/cm and 100 mS/cm, between 25 mS/cm and 200 mS/cm, between 50 mS/cm and 150 mS/cm, between 50 mS/cm and 250 mS/cm, between 50 mS/cm and 500 mS/cm, between 75 mS/cm and 200 mS/cm, between 75 mS/cm and 350 mS/cm, between 100 mS/cm and 250 mS/cm, between 100 mS/cm and 400 mS/cm, between 100 mS/cm and 500 mS/cm, between 150 mS/cm and 450 mS/cm, between 200 mS/cm and 500 mS/cm, between 300 mS/cm and 500 mS/cm, e.g., about 0.001 mS/cm, about 0.01 mS/cm, about 0.05 mS/cm, about 0.1 mS/cm, about 0.5 mS/cm, about 1 mS/cm, about 5 mS/cm, about 10 mS/cm, about 15 mS/cm, about 20 mS/cm, about 30 mS/cm, about 40 mS/cm, about 50 mS/cm, about 60 mS/cm, about 70 mS/cm, about 80 mS/cm, about 90 mS/cm, about 100 mS/cm, about 150 mS/cm, about 200 mS/cm, about 250 mS/cm, about 300 mS/cm, about 350 mS/cm, about 400 mS/cm, about 450 mS/cm, or about 500 mS/cm).

In some embodiments, a temperature of the plurality of cells suspended in the liquid is between 0° C. and 50° C. (between 0° C. and 5° C., between 2° C. and 15° C., between 3° C. and 30° C., between 4° C. and 10° C., between 4° C. and 25° C., between 5° C. and 30° C., between 7° C. and 35° C., between 10° C. and 25° C., between 10° C. and 40° C., between 15° C. and 50° C., between 20° C. and 40° C., between 25° and 50° C., or between 35° C. and 45° C., e.g., about 0° C., about 1° C., about 2° C., about 3° C., about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C. about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., or about 50° C.).

In some embodiments, the method further includes storing the plurality of cells suspended in the liquid in a recovery buffer after poration. In some embodiments, the cells have a viability after introduction of the composition of between 0.1% and 99.9% (e.g., between 0.1% and 5%, between 1% and 10%, between 2.5% and 20%, between 5% and 40%, between 10% and 30%, between 10% and 60%, between 10% and 90%, between 25% and 40%, between 25% and 85%, between 30% and 50%, between 30% and 80%, between 40% and 65%, between 50% and 75%, between 50% and 99.9%, between 60% and 80%, between 75% and 99.9%, or between 85% and 99.9%, e.g., about 0.1%, about 0.15%, about 0.2%, about 0.25%, about 0.3%, about 0.35%, about 0.4%, about 0.45%, about 0.5%, about 0.55%, about 0.6%, about 0.65%, about 0.7%, about 0.75%, about 0.8%, about 0.85%, about 0.9%, about 0.95%, about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or about 99.9%).

In some embodiments, the composition is introduced into a plurality of the cells at an efficiency of between 0.1% and 99.9% (e.g., between 0.1% and 5%, between 1% and 10%, between 2.5% and 20%, between 5% and 40%, between 10% and 30%, between 10% and 60%, between 10% and 90%, between 25% and 40%, between 25% and 85%, between 30% and 50%, between 30% and 80%, between 40% and 65%, between 50% and 75%, between 50% and 99.9%, between 60% and 80%, between 75% and 99.9%, or between 85% and 99.9%, e.g., about 0.1%, about 0.15%, about 0.2%, about 0.25%, about 0.3%, about 0.35%, about 0.4%, about 0.45%, about 0.5%, about 0.55%, about 0.6%, about 0.65%, about 0.7%, about 0.75%, about 0.8%, about 0.85%, about 0.9%, about 0.95%, about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or about 99.9%).

In some embodiments, any of the methods of the invention produces a cell recovery number of between 10⁴ cells and 10¹² cells (e.g., between 10⁴ cells and 10⁵ cells, between 10⁴ cells and 10⁶ cells, between 10⁴ cells and 10⁷ cells, between 5×10⁴ cells and 5×10⁵ cells, between 10⁵ cells and 10⁶ cells, between 10⁵ cells and 10⁷ cells, between 10⁵ cells and 10¹⁰ cells, between 2.5×10⁵ cells and 10⁶ cells, between 5×10⁵ cells and 5×10⁶ cells, between 10⁶ cells and 10⁷ cells, between 10⁶ cells and 10⁸ cells, between 10⁶ cells and 10¹² cells, between 5×10⁶ cells and 5×10⁷ cells, between 10⁷ cells and 10⁸ cells, between 10⁷ cells and 10⁹ cells, between 10⁷ cells and 10¹² cells, between 5×10⁷ cells and 5×10⁸ cells, between 10⁸ cells and 10⁹ cells, between 10⁸ cells and 10¹⁰ cells, between 10⁸ cells and 10¹² cells, between 5×10⁸ cells and 5×10⁹ cells, between 10⁹ cells and 10¹⁰ cells, between 10⁹ cells and 10¹¹ cells, between 10¹⁰ cells and 10¹¹ cells, between 10¹⁰ cells and 10¹² cells, or between 10¹¹ cells and 10¹² cells, e.g., about 10⁴ cells, about 2.5×10⁴ cells, about 5×10⁴ cells, about 10⁵ cells, about 2.5×10⁵ cells, about 5×10⁵ cells, about 10⁶ cells, about 2.5×10⁶ cells, about 5×10⁶ cells, about 10⁷ cells, about 2.5×10⁷ cells, about 5×10⁷ cells, about 10⁸ cells, about 2.5×10⁸ cells, about 5×10⁸ cells, about 10⁹ cells, about 2.5×10⁹ cells, about 5×10⁹ cells, about 10¹⁰ cells, about 5×10¹⁰ cells, about 10¹¹ cells, or about 10¹² cells).

In some embodiments, the method produces a cell recovery rate of between 0.1% and 100% (e.g., between 0.1% and 5%, between 1% and 10%, between 2.5% and 20%, between 5% and 40%, between 10% and 30%, between 10% and 60%, between 10% and 90%, between 25% and 40%, between 25% and 85%, between 30% and 50%, between 30% and 80%, between 40% and 65%, between 50% and 75%, between 50% and 100%, between 60% and 80%, between 75% and 100%, between 85% and 100%, e.g., about 0.1%, about 0.15%, about 0.2%, about 0.25%, about 0.3%, about 0.35%, about 0.4%, about 0.45%, about 0.5%, about 0.55%, about 0.6%, about 0.65%, about 0.7%, about 0.75%, about 0.8%, about 0.85%, about 0.9%, about 0.95%, about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%). In some embodiments, the method produces a live engineered cell yield (e.g., a recovery yield) of between 0.1% and 500% (e.g., between 0.1% and 5%, between 1% and 10%, between 2.5% and 20%, between 5% and 40%, between 10% and 30%, between 10% and 60%, between 10% and 90%, between 25% and 40%, between 25% and 85%, between 30% and 50%, between 30% and 80%, between 40% and 65%, between 50% and 75%, between 50% and 100%, between 60% and 80%, between 60% and 150%, between 75% and 100%, between 75% and 200%, between 85% and 150%, between 90% and 250%, between 100% and 200%, between 100% and 400%, between 150% and 300%, between 200% and 500%, or between 300% and 500%, e.g., about 0.1%, about 0.15%, about 0.2%, about 0.25%, about 0.3%, about 0.35%, about 0.4%, about 0.45%, about 0.5%, about 0.55%, about 0.6%, about 0.65%, about 0.7%, about 0.75%, about 0.8%, about 0.85%, about 0.9%, about 0.95%, about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, about 100%, about 150%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500%).

In some embodiments, the composition delivered to the plurality of cells (e.g., electro-mechanically delivered into the plurality of cells) includes at least one compound selected from the group consisting of therapeutic agents, vitamins, nanoparticles, charged molecules, uncharged molecules, engineered nucleases, DNA, RNA, CRISPR-Cas complex, transcription activator-like effector nucleases (TALENs), zinc-finger nucleases (ZFNs), homing nucleases, meganucleases (mns), megaTALs, enzymes, transposons, peptides, proteins, viruses, polymers, a ribonucleoprotein (RNP), and polysaccharides. In some embodiments, the composition has a concentration in the liquid of between 0.0001 μg/mL and 1,000 μg/mL (e.g., from about 0.0001 μg/mL to about 0.001 μg/mL, about 0.001 μg/mL to about 0.01 μg/mL, about 0.001 μg/mL to about 5 μg/mL, about 0.005 μg/mL to about 0.1 μg/mL, about 0.01 μg/mL to about 0.1 μg/mL, about 0.01 μg/mL to about 1 μg/mL, about 0.1 μg/mL to about 1 μg/mL, about 0.1 μg/mL to about 5 μg/mL, about 1 μg/mL to about 10 μg/mL, about 1 μg/mL to about 50 μg/mL, about 1 μg/mL to about 100 μg/mL, about 2.5 μg/mL to about 15 μg/mL, about 5 μg/mL to about 25 μg/mL, about 5 μg/mL to about 50 μg/mL, about 5 μg/mL to about 500 μg/mL, about 7.5 μg/mL to about 75 μg/mL, about 10 μg/mL to about 100 μg/mL, about 10 μg/mL to about 1,000 μg/mL, about 25 μg/mL to about 50 μg/mL, about 25 μg/mL to about 250 μg/mL, about 25 μg/mL to about 500 μg/mL, about 50 μg/mL to about 100 μg/mL, about 50 μg/mL to about 250 μg/mL, about 50 μg/mL to about 750 μg/mL, about 100 μg/mL to about 300 μg/mL, about 100 μg/mL to about 1,000 μg/mL, about 200 μg/mL to about 400 μg/mL, about 250 μg/mL to about 500 μg/mL, about 350 μg/mL to about 500 μg/mL, about 400 μg/mL to about 1,000 μg/mL, about 500 μg/mL to about 750 μg/mL, about 650 μg/mL to about 1,000 μg/mL, or about 800 μg/mL to about 1,000 μg/mL, e.g., about 0.0001 μg/mL, about 0.0005 μg/mL, about 0.001 μg/mL, about 0.005 μg/mL, about 0.01 μg/mL, about 0.02 μg/mL, about 0.03 μg/mL, about 0.04 μg/mL, about 0.05 μg/mL, about 0.06 μg/mL, about 0.07 μg/mL, about 0.08 μg/mL, about 0.09 μg/mL, about 0.1 μg/mL, about 0.2 μg/mL, about 0.3 μg/mL, about 0.4 μg/mL, about 0.5 μg/mL, about 0.6 μg/mL, about 0.7 μg/mL, about 0.8 μg/mL, about 0.9 μg/mL, about 1 μg/mL, about 1.5 μg/mL, about 2 μg/mL, about 2.5 μg/mL, about 3 μg/mL, about 3.5 μg/mL, about 4 μg/mL, about 4.5 μg/mL, about 5 μg/mL, about 5.5 μg/mL, about 6 μg/mL, about 6.5 μg/mL, about 7 μg/mL, about 7.5 μg/mL, about 8 μg/mL, about 8.5 μg/mL, about 9 μg/mL, about 9.5 μg/mL, about 10 μg/mL, about 15 μg/mL, about 20 μg/mL, about 25 μg/mL, about 30 μg/mL, about 35 μg/mL, about 40 μg/mL, about 45 μg/mL, about 50 μg/mL, about 55 μg/mL, about 60 μg/mL, about 65 μg/mL, about 70 μg/mL, about 75 μg/mL, about 80 μg/mL, about 85 μg/mL, about 90 μg/mL, about 95 μg/mL, about 100 μg/mL, about 200 μg/mL, about 250 μg/mL, about 300 μg/mL, about 350 μg/mL, about 400 μg/mL, about 450 μg/mL, about 500 μg/mL, about 550 μg/mL, about 600 μg/mL, about 650 μg/mL, about 700 μg/mL, about 750 μg/mL, about 800 μg/mL, about 850 μg/mL, about 900 μg/mL, about 950 μg/mL, or about 1,000 μg/mL).

In some embodiments of any of the preceding aspects, the plurality of cells suspended in the liquid includes eukaryotic cells (e.g., animal cells, e.g., human cells), prokaryotic cells (e.g., bacterial cells), plant cells, and/or synthetic cells. The cells can be primary cells (e.g., primary human cells), cells from a cell line (e.g., a human cell line), cells in suspension, adherent cells, stem cells, blood cells (e.g., peripheral blood mononuclear cells (PBMCs)), and/or immune cells (e.g., white blood cells (e.g., innate immune cells or adaptive immune cells)). In some embodiments, the cells (e.g., immune cells, e.g., T cells, B cell, natural killer cells, macrophages, monocytes, or antigen-presenting cells) are unstimulated cells, stimulated cells, or activated cells. In some embodiments, the cells are adaptive immune cells and/or innate immune cells. In some embodiments, the plurality of cells includes antigen presenting cells (APCs), monocytes, T-cells, B-cells, dendritic cells, macrophages, neutrophils, NK cells, Jurkat cells, THP-1 cells, human embryonic kidney (HEK-293) cells, Chinese hamster ovary (e.g., CHO-K1) cells, embryonic stem cells (ESCs), mesenchymal stem cells (MSCs), or hematopoietic stem cells (HSCs). In some embodiments, the cells can be primary human T-cells, primary human macrophages, primary human monocytes, primary human NK cells, or primary human induced pluripotent stem cells (iPSCs). In some embodiments of any of the methods described herein, the method further includes storing the plurality of cells suspended in the liquid in a recovery buffer after poration.

In another aspect, the invention provides a kit including any of the devices or systems described herein. For example, in one aspect, the invention provides a kit for electro-mechanically delivering a composition into a plurality of cells suspended in a liquid, wherein the kit includes a plurality of devices, each of the plurality of devices including: a first electrode including a first outlet, a first inlet, and a first lumen including a minimum cross-sectional dimension; a second electrode including a second outlet, a second inlet, and a second lumen including a minimum cross-sectional dimension; and an electroporation zone disposed between the first outlet and the second inlet, wherein the electroporation zone includes a minimum cross-sectional dimension greater than about 100 μm (e.g., from 100 μm to 10 mm, from 150 μm to 15 mm, from 200 μm to 10 mm, from 250 μm to 5 mm, from 500 μm to 10 mm, from 1 mm to 10 mm, from 1 mm to 50 mm, from 5 mm to 25 mm, or from 20 mm to 50 mm, e.g., about 0.5 mm, about 1.0 mm, about 1.5 mm, about 2 mm, about 5 mm, about 7 mm, about 10 mm, about 15 mm, about 25 mm, or about 50 mm), wherein the electroporation zone has a substantially uniform cross-sectional area, wherein the application of an electrical potential difference to the first and second electrodes produces an electric field in the electroporation zone; and a plurality of outer structures configured to encase the plurality of devices, wherein each of the plurality of outer structures includes: a housing configured to encase the first electrode, second electrode, and the electroporation zone of the at least one device; a first electrical input operatively coupled to the first electrode; and a second electrical input operatively coupled to the second electrode. In some embodiments, the plurality of outer structures is integral to the plurality of devices. In some embodiments, the plurality of outer structures is releasably connected to the plurality of devices. In some embodiments, the housing further includes a thermal controller configured to increase a temperature of the at least one device, wherein the thermal controller is a heating element selected from a group consisting of a heating block, a liquid flow, a battery-powered heater, and a thin-film heater. In some embodiments, the housing further includes a thermal controller configured to decrease a temperature of the at least one device, wherein the thermal controller is a cooling element selected from a group consisting of a liquid flow, an evaporative cooler, and a Peltier device.

In another aspect, the invention provides a kit for electroporating a composition into a plurality of cells suspended in a liquid, wherein the kit includes a plurality of devices, each of the plurality of devices including: a first electrode including a first outlet, a first inlet, and a first lumen including a minimum cross-sectional dimension; a second electrode including a second outlet, a second inlet, and a second lumen including a minimum cross-sectional dimension; and an electroporation zone disposed between the first outlet and the second inlet, wherein the electroporation zone includes a minimum cross-sectional dimension greater than about 100 μm (e.g., from 100 μm to 10 mm, from 150 μm to 15 mm, from 200 μm to 10 mm, from 250 μm to 5 mm, from 500 μm to 10 mm, from 1 mm to 10 mm, from 1 mm to 50 mm, from 5 mm to 25 mm, or from 20 mm to 50 mm, e.g., about 0.5 mm, about 1.0 mm, about 1.5 mm, about 2 mm, about 5 mm, about 7 mm, about 10 mm, about 15 mm, about 25 mm, or about 50 mm), wherein the electroporation zone has a substantially uniform cross-sectional area, wherein the application of an electrical potential difference to the first and second electrodes produces an electric field in the electroporation zone; and a plurality of outer structures configured to encase the plurality of devices, wherein each of the plurality of outer structures includes: a housing configured to encase the first electrode, second electrode, and the electroporation zone of the at least one device; a first electrical input operatively coupled to the first electrode; and a second electrical input operatively coupled to the second electrode. In some embodiments, the plurality of outer structures is integral to the plurality of devices. In some embodiments, the plurality of outer structures is releasably connected to the plurality of devices. In some embodiments, the housing further includes a thermal controller configured to increase a temperature of the at least one device, wherein the thermal controller is a heating element selected from a group consisting of a heating block, a liquid flow, a battery-powered heater, and a thin-film heater. In some embodiments, the housing further includes a thermal controller configured to decrease a temperature of the at least one device, wherein the thermal controller is a cooling element selected from a group consisting of a liquid flow, an evaporative cooler, and a Peltier device.

In another aspect, the invention provides a kit for electro-mechanically delivering a composition into a plurality of cells suspended in a liquid, including: a plurality of devices, each of the plurality of devices including a device of the aforementioned embodiments; and a plurality of outer structures configured to encase the plurality of devices, wherein each of the plurality of outer structures includes: a housing configured to encase the first electrode, second electrode, and the electroporation zone of the at least one device; a first electrical input operatively coupled to the first electrode; and a second electrical input operatively coupled to the second electrode. In some embodiments, the plurality of outer structures is integral to the plurality of devices. In some embodiments, the plurality of outer structures is releasably connected to the plurality of devices. In some embodiments, the housing further includes a thermal controller configured to increase the temperature of the at least one device, wherein the thermal controller is a heating element selected from a group consisting of a heating block, a liquid flow, a battery-powered heater, and a thin-film heater. In some embodiments, the housing further includes a thermal controller configured to decrease the temperature of the at least one device, wherein the thermal controller is a cooling element selected from a group consisting of a liquid flow, an evaporative cooler, and a Peltier device.

In another aspect, the invention provides a kit for electroporating a composition into plurality of cells suspended in a liquid, including: a plurality of devices, each of the plurality of devices including a device of the aforementioned embodiments; and a plurality of outer structures configured to encase the plurality of devices, wherein each of the plurality of outer structures includes: a housing configured to encase the first electrode, second electrode, and the electroporation zone of the at least one device; a first electrical input operatively coupled to the first electrode; and a second electrical input operatively coupled to the second electrode. In some embodiments, the plurality of outer structures is integral to the plurality of devices. In some embodiments, the plurality of outer structures is releasably connected to the plurality of devices. In some embodiments, the housing further includes a thermal controller configured to increase the temperature of the at least one device, wherein the thermal controller is a heating element selected from a group consisting of a heating block, a liquid flow, a battery-powered heater, and a thin-film heater. In some embodiments, the housing further includes a thermal controller configured to decrease the temperature of the at least one device, wherein the thermal controller is a cooling element selected from a group consisting of a liquid flow, an evaporative cooler, and a Peltier device.

In another aspect, the invention provides a device for electro-mechanically delivering a composition into plurality of cells suspended in a fluid, where the device includes: a first electrode having a first inlet and a first outlet, where a lumen of the first electrode defines an entry zone; a second electrode having a second inlet and a second outlet, where a lumen of the second electrode defines a recovery zone; and an electroporation zone, where the electroporation zone is fluidically connected to the first outlet of the first electrode and the second inlet of the second electrode, where the electroporation zone has a substantially uniform cross-section dimension, and where application of an electrical potential difference to the first and second electrodes produces an electric field in the electroporation zone. In the device, the plurality of cells suspended in the fluid are electroporated upon entering the electroporation zone.

In another aspect, the invention provides a device for electroporating a composition into plurality of cells suspended in a fluid, where the device includes: a first electrode having a first inlet and a first outlet, where a lumen of the first electrode defines an entry zone; a second electrode having a second inlet and a second outlet, where a lumen of the second electrode defines a recovery zone; and an electroporation zone, where the electroporation zone is fluidically connected to the first outlet of the first electrode and the second inlet of the second electrode, where the electroporation zone has a substantially uniform cross-section dimension, and where application of an electrical potential difference to the first and second electrodes produces an electric field in the electroporation zone. In the device, the plurality of cells suspended in the fluid are electroporated upon entering the electroporation zone.

In some embodiments of any of the preceding methods, the kit further includes one or more reservoirs, e.g., a first reservoir and a second reservoir, fluidically connected to a zone, e.g., the entry zone or recovery zone, of the device. For example, a first reservoir may be fluidically connected to the entry zone and a second reservoir may be fluidically connected to the recovery zone.

In certain embodiments, the cross-section of the electroporation zone is selected from the group consisting of cylindrical, ellipsoidal, polygonal, star, parallelogram, trapezoidal, and irregular.

In some cases, the cross-sectional dimension of the entry zone or the cross-sectional dimension of the recovery zone is between 0.01% to 100,000% of the cross-sectional dimension of the electroporation zone. For example, the cross-sectional dimension of the entry zone or the cross-sectional dimension of the recovery zone may be about 0.01% to about 1000% of the cross-sectional dimension of the electroporation zone, e.g., about 0.01% to about 1%, about 0.1% to about 10%, about 5% to about 25%, about 10% to about 50%, about 10% to about 1000%, about 25% to about 75%, about 25% to about 750%, or about 50% to about 1000% of the cross-sectional dimension of the electroporation zone. Alternatively, the cross-sectional dimension of the entry zone or the cross-sectional dimension of the recovery zone may be about 100% to about 100,000% of the of the cross-sectional dimension of the electroporation zone, e.g., about 100% to about 1000%, about 500% to about 5,000%, about 1,000% to about 10,000%, about 5,000% to about 25,000%, about 10,000% to about 50,000%, about 25,000% to about 75,000%, or about 50,000% to about 100,000% of the cross-sectional dimension of the electroporation zone.

In some embodiments, the cross-sectional dimension of the electroporation zone is between 0.005 mm and 50 mm. In some embodiments, the length of the electroporation zone is between 0.005 mm and 50 mm. In particular embodiments, the length of the electroporation zone is between 0.005 mm and 25 mm. In some embodiments, the cross-sectional dimension of any of the first electrode or the second electrode is between 0.1 mm to 500 mm. In particular embodiments, none of the entry zone, recovery zone, or electroporation zone reduce a cross-section dimension of any of the plurality of cells suspended in the fluid, e.g., cells can pass through the device without deformation.

In some embodiments, the plurality of cells has from 0% to about 25% phenotypic change relative to a baseline measurement of cell phenotype upon exiting the electroporation zone. In some embodiments, the plurality of cells has no phenotypic change upon exiting the electroporation zone.

In further embodiments, the device includes an outer structure having a housing configured to encase the first electrode, second electrode, and the electroporation zone of the device. In some embodiments, the outer structure is integral to the device. In certain embodiments, the outer structure is releasably connected to the device.

In another aspect, the invention provides a device for electro-mechanical delivery of a composition into a plurality of cells suspended in a fluid, where the device includes: a first electrode having a first inlet and a first outlet, where a lumen of the first electrode defines an entry zone; a second electrode having a second inlet and a second outlet, where a lumen of the second electrode defines a recovery zone; a third inlet and a third outlet, where the third inlet and third outlet intersect the first electrode between the first inlet and the first outlet; a fourth inlet and a fourth outlet, where the fourth inlet and fourth outlet intersect the second electrode between the second inlet and the second outlet; and an electroporation zone, where the electroporation zone is fluidically connected to the first outlet of the first electrode and the second inlet of the second electrode, where the electroporation zone has a substantially uniform cross-section dimension, and where application of an electrical potential difference to the first and second electrodes produces an electric field in the electroporation zone. In the device, the plurality of cells suspended in the fluid are transfected upon entering the electroporation zone.

In another aspect, the invention provides a device for electroporating a composition into a plurality of cells suspended in a fluid, where the device includes: a first electrode having a first inlet and a first outlet, where a lumen of the first electrode defines an entry zone; a second electrode having a second inlet and a second outlet, where a lumen of the second electrode defines a recovery zone; a third inlet and a third outlet, where the third inlet and third outlet intersect the first electrode between the first inlet and the first outlet; a fourth inlet and a fourth outlet, where the fourth inlet and fourth outlet intersect the second electrode between the second inlet and the second outlet; and an electroporation zone, where the electroporation zone is fluidically connected to the first outlet of the first electrode and the second inlet of the second electrode, where the electroporation zone has a substantially uniform cross-section dimension, and where application of an electrical potential difference to the first and second electrodes produces an electric field in the electroporation zone. In the device, the plurality of cells suspended in the fluid are transfected upon entering the electroporation zone.

In some embodiments of either of the preceding aspects, the device further includes one or more reservoir, e.g., a first reservoir and a second reservoir, fluidically connected to a zone, e.g., the entry zone or recovery zone, of the device. For example, a first reservoir may be fluidically connected to the entry zone and a second reservoir may be fluidically connected to the recovery zone. In particular embodiments, the device includes a third reservoir fluidically connected to the third inlet and the third outlet and a fourth reservoir fluidically connected to the fourth inlet and the fourth outlet.

In certain embodiments, the cross-section of the electroporation zone is selected from the group consisting of cylindrical, ellipsoidal, polygonal, star, parallelogram, trapezoidal, and irregular.

In some embodiments, the cross-sectional dimension of the entry zone or the cross-sectional dimension of the recovery zone is between 0.01% to 100,000% of the cross-sectional dimension of the electroporation zone. For example, the cross-sectional dimension of the entry zone or the cross-sectional dimension of the recovery zone may be about 0.01% to about 1,000% of the cross-sectional dimension of the electroporation zone, e.g., about 0.01% to about 1%, about 0.1% to about 10%, about 5% to about 25%, about 10% to about 50%, about 10% to about 1,000%, about 25% to about 75%, about 25% to about 750%, or about 50% to about 100% of the cross-sectional dimension of the electroporation zone. Alternatively, the cross-sectional dimension of the entry zone or the cross-sectional dimension of the recovery zone may be about 100% to about 100,000% of the of the cross-sectional dimension of the electroporation zone, e.g., about 100% to about 1000%, about 500% to about 5,000%, about 1,000% to about 10,000%, about 5,000% to about 25,000%, about 10,000% to about 50,000%, about 25,000% to about 75,000%, or about 50,000% to about 100,000% of the cross-sectional dimension of the electroporation zone.

In some embodiments, the cross-sectional dimension of the electroporation zone is between 0.005 mm and 50 mm. In some embodiments, the length of the electroporation zone is between 0.005 mm and 50 mm. In particular embodiments, the length of the electroporation zone is between 0.005 mm and 25 mm. In some embodiments, the cross-sectional dimension of any of the first electrode or the second electrode is between 0.1 mm to 500 mm. In particular embodiments, none of the entry zone, recovery zone, or electroporation zone reduce a cross-section dimension of any of the plurality of cells suspended in the fluid, e.g., cells can pass through the device without deformation.

In particular embodiments, the first and/or second electrodes is porous or a conductive fluid (e.g., liquid).

In some embodiments, the plurality of cells has from 0% to about 25% phenotypic change relative to a baseline measurement of cell phenotype upon exiting the electroporation zone. In some embodiments, the plurality of cells has no phenotypic change upon exiting the electroporation zone.

In further embodiments, the device includes an outer structure having a housing configured to encase the first electrode, second electrode, and the electroporation zone of the device. In some embodiments, the outer structure is integral to the device. In certain embodiments, the outer structure is releasably connected to the device.

In another aspect, the invention provides a system for electro-mechanical delivery of a composition into a plurality of cells suspended in a fluid, the system including a device that includes: a first electrode having a first inlet and a first outlet, where a lumen of the first electrode defines an entry zone; a second electrode having a second inlet and a second outlet, where a lumen of the second electrode defines a recovery zone; and an electroporation zone, where the electroporation zone is fluidically connected to the first outlet of the first electrode and the second inlet of the second electrode, where the electroporation zone has a substantially uniform cross-section dimension, and where application of an electrical potential difference to the first and second electrodes produces an electric field in the electroporation zone. The system further includes source of electrical potential, where the first and second electrodes of the device are releasably connected to the source of electrical potential. In the system, the plurality of cells suspended in the fluid are electroporated upon entering the electroporation zone.

In another aspect, the invention provides a system for electroporating a composition into a plurality of cells suspended in a fluid, the system including a device that includes: a first electrode having a first inlet and a first outlet, where a lumen of the first electrode defines an entry zone; a second electrode having a second inlet and a second outlet, where a lumen of the second electrode defines a recovery zone; and an electroporation zone, where the electroporation zone is fluidically connected to the first outlet of the first electrode and the second inlet of the second electrode, where the electroporation zone has a substantially uniform cross-section dimension, and where application of an electrical potential difference to the first and second electrodes produces an electric field in the electroporation zone. The system further includes source of electrical potential, where the first and second electrodes of the device are releasably connected to the source of electrical potential. In the system, the plurality of cells suspended in the fluid are electroporated upon entering the electroporation zone.

In some embodiments, the plurality of cells has from 0% to about 25% phenotypic change relative to a baseline measurement of cell phenotype upon exiting the electroporation zone. In some embodiments, the plurality of cells has no phenotypic change upon exiting the electroporation zone.

In further embodiments, the device includes an outer structure having a housing configured to encase the first electrode, second electrode, and the electroporation zone of the device. In some embodiments, the outer structure includes a first electrical input operatively coupled to the first electrode and a second electrical input operatively coupled to the second electrode. In some embodiments, the releasable connection between the first or second electrical inputs and the source of electrical potential is selected from the group consisting of a clamp, a clip, a spring, a sheath, a wire brush, mechanical connection, inductive connection, or a combination thereof.

In some embodiments, the outer structure is integral to the device. In certain embodiments, the outer structure is releasably connected to the device.

In some embodiments, any of the devices, systems, or methods of any of the previous aspects induces reversible or irreversible electroporation. In particular embodiments, the electroporation is substantially non-thermal reversible electroporation, substantially non-thermal irreversible electroporation, or substantially thermal irreversible electroporation.

In some embodiments, the releasable connection between the device and the source of electrical potential is selected from the group consisting of a clamp, a clip, a spring, a sheath, a wire brush, mechanical connection, inductive connection, or a combination thereof. In particular embodiments, the releasable connection between the device and the source of electrical potential is a spring.

In some embodiments, the device further includes one or more reservoir, e.g., a first reservoir and a second reservoir, fluidically connected to a zone, e.g., the entry zone or recovery zone, of a device. For example, a first reservoir may be fluidically connected to the entry zone and a second reservoir may be fluidically connected to the recovery zone.

In certain embodiments, the cross-section of the electroporation zone is selected from the group consisting of cylindrical, ellipsoidal, polygonal, star, parallelogram, trapezoidal, and irregular.

In some embodiments, the cross-sectional dimension of the entry zone or the cross-sectional dimension of the recovery zone is between 0.01% and 100,000% of the cross-sectional dimension of the electroporation zone. For example, the cross-sectional dimension of the entry zone or the cross-sectional dimension of the recovery zone may be about 0.01% to about 1000% of the cross-sectional dimension of the electroporation zone, e.g., about 0.01% to about 1%, about 0.1% to about 10%, about 5% to about 25%, about 10% to about 50%, about 10% to about 1,000%, about 25% to about 75%, about 25% to about 750%, or about 50% to about 100% of the cross-sectional dimension of the electroporation zone. Alternatively, the cross-sectional dimension of the entry zone or the cross-sectional dimension of the recovery zone may be about 100% to about 100,000% of the of the cross-sectional dimension of the electroporation zone, e.g., about 100% to about 1000%, about 500% to about 5,000%, about 1,000% to about 10,000%, about 5,000% to about 25,000%, about 10,000% to about 50,000%, about 25,000% to about 75,000%, or about 50,000% to about 100,000% of the cross-sectional dimension of the electroporation zone.

In some embodiments, the cross-sectional dimension of the electroporation zone is between 0.005 mm and 50 mm. In some embodiments, the length of the electroporation zone is between 0.005 mm and 50 mm. In particular embodiments, the length of the electroporation zone is between 0.005 mm and 25 mm. In some embodiments, the cross-sectional dimension of any of the first electrode or the second electrode is between 0.1 mm to 500 mm. In particular embodiments, none of the entry zone, recovery zone, or electroporation zone reduce a cross-section dimension of any of the plurality of cells suspended in the fluid, e.g., cells can pass through the device without deformation.

In further embodiments, the system includes a fluid delivery source fluidically connected to the entry zone, wherein the fluid delivery source is configured to deliver the plurality of cells suspended in the fluid through the entry zone to the recovery zone. In some embodiments, the delivery rate from the fluid delivery source is between 0.001 mL/min to 1,000 mL/min, e.g., 25 mL/min. In certain embodiments, the residence time of any of the plurality of cells suspended in the fluid is between 0.5 ms to 50 ms. In some embodiments, the conductivity of the fluid is between 0.001 mS/cm to 500 mS/cm, e.g., 1-20 mS/cm.

In further embodiments, the system includes a controller operatively coupled to the source of electrical potential to deliver voltage pulses to the first electrode and second electrodes to generate an electrical potential difference between the first and second electrodes. In some embodiments, the voltage pulses have an amplitude of −3 kV to 3 kV, e.g., 0.01 kV to 3 kV, e.g., 0.2-0.6 kV. In some cases, the duty cycle of the electroporation is between 0.001% to 100%, e.g., 10-95%. In some embodiments, the voltage pulses have a duration of between 0.01 ms to 1,000 ms, e.g., 1-10 ms. In certain embodiments, the voltage pulses are applied the first and second electrodes at a frequency between 1 Hz to 50,000 Hz, e.g., 100-500 Hz. The waveform of the voltage pulse may be DC, square, pulse, bipolar, sine, ramp, asymmetric bipolar, arbitrary, or any superposition or combination thereof. In particular embodiments, the electric field generated from the voltage pulses has a magnitude of between 1 V/cm to 50,000 V/cm, e.g., 100-1,000 V/cm.

In further embodiments, the system includes a housing (e.g., a housing structure) configured to house the electroporation device described herein. In further instances, the housing (e.g., housing structure) includes a thermal controller configured to increase or decrease the temperature of the housing or any component of the system thereof. In some embodiments, the thermal controller is a heating element, e.g., a heating block, liquid flow, battery powered heater, or a thin-film heater. In other embodiments, the thermal controller is a cooling element, e.g., liquid flow, evaporative cooler, or a thermoelectric, e.g., a Peltier, device.

In further embodiments, the system includes a plurality of cell porating devices, e.g., in series or in parallel. In particular embodiments, the system includes a plurality of outer structures for the plurality of cell porating devices.

In another aspect, the invention provides a system for electro-mechanical delivery of a composition into a plurality of cells suspended in a fluid, the system including a device that includes: a first electrode having a first inlet and a first outlet, where a lumen of the first electrode defines an entry zone; a second electrode having a second inlet and a second outlet, where a lumen of the second electrode defines a recovery zone; a third inlet and a third outlet, where the third inlet and third outlet intersect the first electrode between the first inlet and the first outlet; a fourth inlet and a fourth outlet, where the fourth inlet and fourth outlet intersect the second electrode between the second inlet and the second outlet; and an electroporation zone, where the electroporation zone is fluidically connected to the first outlet of the first electrode and the second inlet of the second electrode, where the electroporation zone has a substantially uniform cross-section dimension, and where application of an electrical potential difference to the first and second electrodes produces an electric field in the electroporation zone. In the device, the plurality of cells suspended in the fluid are transfected upon entering the electroporation zone. In some embodiments, the plurality of cells has from 0% to about 25% phenotypic change relative to a baseline measurement of cell phenotype upon exiting the electroporation zone. In some embodiments, the plurality of cells has no phenotypic change upon exiting the electroporation zone.

In another aspect, the invention provides a system for electroporating a composition into a plurality of cells suspended in a fluid, the system including a device that includes: a first electrode having a first inlet and a first outlet, where a lumen of the first electrode defines an entry zone; a second electrode having a second inlet and a second outlet, where a lumen of the second electrode defines a recovery zone; a third inlet and a third outlet, where the third inlet and third outlet intersect the first electrode between the first inlet and the first outlet; a fourth inlet and a fourth outlet, where the fourth inlet and fourth outlet intersect the second electrode between the second inlet and the second outlet; and an electroporation zone, where the electroporation zone is fluidically connected to the first outlet of the first electrode and the second inlet of the second electrode, where the electroporation zone has a substantially uniform cross-section dimension, and where application of an electrical potential difference to the first and second electrodes produces an electric field in the electroporation zone. In the device, the plurality of cells suspended in the fluid are electroporated upon entering the electroporation zone.

In some embodiments, the plurality of cells has from 0% to about 25% phenotypic change relative to a baseline measurement of cell phenotype upon exiting the electroporation zone. In some embodiments, the plurality of cells has no phenotypic change upon exiting the electroporation zone.

In further embodiments of any of the previous aspects, the device includes an outer structure having a housing (e.g., a housing structure) configured to encase the first electrode, second electrode, and the electroporation zone of the device. In some embodiments, the outer structure includes a first electrical input operatively coupled to the first electrode and a second electrical input operatively coupled to the second electrode. In some embodiments, the releasable connection between the first or second electrical inputs and the source of electrical potential is selected from the group consisting of a clamp, a clip, a spring, a sheath, a wire brush, mechanical connection, inductive connection, or a combination thereof.

In some embodiments, the outer structure is integral to the device. In certain embodiments, the outer structure is releasably connected to the device.

In some cases, the system induces reversible or irreversible electroporation. In particular embodiments, the electroporation is substantially non-thermal reversible electroporation, substantially non-thermal irreversible electroporation, or substantially thermal irreversible electroporation.

In some embodiments, the releasable connection between the device and the source of electrical potential is selected from the group consisting of a clamp, a clip, a spring, a sheath, a wire brush, mechanical connection, inductive connection, or a combination thereof. In particular embodiments, the releasable connection between the device and the source of electrical potential is a spring.

In some embodiments, the device further includes one or more reservoirs, e.g., a first reservoir and a second reservoir, fluidically connected to a zone, e.g., the entry zone or recovery zone, of a device. For example, a first reservoir may be fluidically connected to the entry zone and a second reservoir may be fluidically connected to the recovery zone.

In certain embodiments, the cross-section of the electroporation zone is selected from the group consisting of cylindrical, ellipsoidal, polygonal, star, parallelogram, trapezoidal, and irregular.

In some embodiments, the cross-sectional dimension of the entry zone or the cross-sectional dimension of the recovery zone is between 0.01% to 100,000% of the cross-sectional dimension of the electroporation zone. For example, the cross-sectional dimension of the entry zone or the cross-sectional dimension of the recovery zone may be about 0.01% to about 1000% of the cross-sectional dimension of the electroporation zone, e.g., about 0.01% to about 1%, about 0.1% to about 10%, about 5% to about 25%, about 10% to about 50%, about 10% to about 1,000%, about 25% to about 75%, about 25% to about 750%, or about 50% to about 100% of the cross-sectional dimension of the electroporation zone. Alternatively, the cross-sectional dimension of the entry zone or the cross-sectional dimension of the recovery zone may be about 100% to about 100,000% of the of the cross-sectional dimension of the electroporation zone, e.g., about 100% to about 1000%, about 500% to about 5,000%, about 1,000% to about 10,000%, about 5,000% to about 25,000%, about 10,000% to about 50,000%, about 25,000% to about 75,000%, or about 50,000% to about 100,000% of the cross-sectional dimension of the electroporation zone.

In some embodiments, the cross-sectional dimension of the electroporation zone is between 0.005 mm and 50 mm. In some embodiments, the length of the electroporation zone is between 0.005 mm and 50 mm. In particular embodiments, the length of the electroporation zone is between 0.005 mm and 25 mm. In some embodiments, the cross-sectional dimension of any of the first electrode or the second electrode is between 0.01 mm and 500 mm. In particular embodiments, none of the entry zone, recovery zone, or electroporation zone reduce a cross-section dimension of any of the plurality of cells suspended in the fluid, e.g., cells can pass through the device without deformation.

In further embodiments, the system includes a fluid delivery source fluidically connected to the entry zone, wherein the fluid delivery source is configured to deliver the plurality of cells suspended in the fluid through the entry zone to the recovery zone. In some embodiments, the delivery rate from the fluid delivery source is between 0.001 mL/min and 1,000 mL/min, e.g., 25 mL/min. In certain embodiments, the residence time of any of the plurality of cells suspended in the fluid is between 0.5 ms and 50 ms. In some embodiments, the conductivity of the fluid is between 0.001 mS/cm and 500 mS/cm, e.g., between 1 mS/cm and 20 mS/cm.

In further embodiments, the system includes a controller operatively coupled to the source of electrical potential to deliver voltage pulses to the first electrode and second electrodes to generate an electrical potential difference between the first and second electrodes. In some embodiments, the voltage pulses have an amplitude of −3 kV to 3 kV, e.g., 0.01 kV to 3 kV, e.g., 0.2-0.6 kV. In some cases, the duty cycle of the electroporation is between 0.001% to 100%, e.g., 10-95%. In some embodiments, the voltage pulses have a duration of between 0.01 ms to 1,000 ms, e.g., 1-10 ms. In certain embodiments, the voltage pulses are applied the first and second electrodes at a frequency between 1 Hz to 50,000 Hz, e.g., 100-500 Hz. The waveform of the voltage pulse may be DC, square, pulse, bipolar, sine, ramp, asymmetric bipolar, arbitrary, or any superposition or combination thereof. In particular embodiments, the electric field generated from the voltage pulses has a magnitude of between 1 V/cm and 50,000 V/cm, e.g., between 100 V/cm and 1,000 V/cm.

In further embodiments, the system includes a housing (e.g., a housing structure) configured to house the electroporation device described herein. In further instances, the housing structure includes a thermal controller configured to increase or decrease the temperature of the housing structure or any component of the system thereof. In some embodiments, the thermal controller is a heating element, e.g., a heating block, liquid flow, battery powered heater, or a thin-film heater. In other embodiments, the thermal controller is a cooling element, e.g., liquid flow, evaporative cooler, or a thermoelectric, e.g., a Peltier, device.

In further embodiments, the system includes a plurality of cell porating devices, e.g., in series or in parallel. In particular embodiments, the system includes a plurality of outer structures for the plurality of cell porating devices.

In another aspect, the invention provides methods of introducing a composition into at least a portion of a plurality of cells suspended in a fluid, the method including the steps of: a. providing a device including: a first electrode having a first inlet and a first outlet, where a lumen of the first electrode defines an entry zone; a second electrode having a second inlet and a second outlet, where a lumen of the second electrode defines a recovery zone; and an electroporation zone, wherein the electroporation zone is fluidically connected to the first outlet of the first electrode and the second inlet of the second electrode, and where application of an electrical potential difference to the first and second electrodes produces an electric field in the electroporation zone; b. energizing the first and second electrodes to produce an electrical potential difference between the first and second electrodes, thereby producing an electric field in the electroporation zone; and c. passing the plurality of cells suspended in the fluid with the composition through the electric field in the electroporation zone of the device. In the method, flow of the plurality of cells suspended in fluid with the composition through the electric field in the electroporation zone enhances temporary permeability of the plurality of cells, thereby introducing the composition into at least a portion of the plurality of cells.

In further embodiments, the method includes assessing the health of a portion of the plurality of cells suspended in the fluid. In certain embodiments, the assessing includes measuring the viability of the portion of the plurality of cells suspended in the fluid. In some embodiments, the assessing includes measuring the transfection efficiency of the portion of the plurality of cells suspended in the fluid. In some embodiments, the assessing includes measuring the cell recovery rate of the portion of the plurality of cells suspended in the fluid. In certain embodiments, the assessing includes flow cytometry analysis of cell surface marker expression.

In some embodiments, the plurality of cells has from 0% to about 25% phenotypic change relative to a baseline measurement of cell phenotype upon exiting the electroporation zone of the device. In some cases, the plurality of cells has no phenotypic change upon exiting the electroporation zone of the device.

In some embodiments, the method induces reversible or irreversible electroporation. In particular embodiments, the electroporation is substantially non-thermal reversible electroporation, substantially non-thermal irreversible electroporation, or substantially thermal irreversible electroporation.

In some embodiments, cells suspended in the fluid with the composition are passed through the electric field in the electroporation zone of the device by the application of a positive pressure, e.g., a pump, e.g., a syringe pump or peristaltic pump.

In certain embodiments, cells in the plurality of cells in the sample may be mammalian cells, eukaryotes, human cells, animal cells, plant cells, synthetic cells, primary cells, cell lines, suspension cells, adherent cells, unstimulated cells, stimulated cells, activated cells, immune cells, stem cells, blood cells, red blood cells, T cells, B cells, neutrophils, dendritic cells, antigen presenting cells (APCs), natural killer (NK) cells, monocytes, macrophages, or peripheral blood mononuclear cells (PBMCs), human embryonic kidney cells, e.g., HEK-293 cells, or Chinese hamster ovary (CHO) cells. In particular embodiments, the plurality of cells includes Jurkat cells. In particular embodiments, the plurality of cells includes primary human T-cells. In particular embodiments, the plurality of cells includes THP-1 cells. In particular embodiments, the plurality of cells includes primary human macrophages. In particular embodiments, the plurality of cells includes primary human monocytes. In particular embodiments, the plurality of cells includes natural killer (NK) cells. In particular embodiments, the plurality of cells includes Chinese hamster ovary cells. In particular embodiments, the plurality of cells includes human embryonic kidney cells. In particular embodiments, the plurality of cells includes B-cells. In particular embodiments, the plurality of cells includes primary human T-cells. In particular embodiments, the plurality of cells includes primary human monocytes. In particular embodiments, the plurality of cells includes primary human macrophages. In particular embodiments, the plurality of cells includes embryonic stem cells (ESCs), mesenchymal stem cells (MSCs), or hematopoietic stem cells (HSCs). In particular embodiments, the plurality of cells includes primary human induced pluripotent stem cells (iPSCs).

In some embodiments, the composition includes at least one compound selected from the group consisting of therapeutic agents, vitamins, nanoparticles, charged therapeutic agents, nanoparticles, charged molecules, e.g., ions in solution, uncharged molecules, nucleic acids, e.g., DNA or RNA, CRISPR-Cas complexes, proteins, polymers, ribonucleoproteins (RNPs), engineered nucleases, transcription activator-like effector nucleases (TALENs), zinc-finger nucleases (ZFNs), homing nucleases, meganucleases (MNs), megaTALs, enzymes, peptides, transposons, or polysaccharides, e.g., dextran, e.g., dextran sulfate. Compositions that can be delivered to cells in a suspension include nucleic acids (e.g., oligonucleotides, mRNA, or DNA), antibodies (or an antibody fragment, e.g., a bispecific fragment, a trispecific fragment, Fab, F(ab′)2, or a single-chain variable fragment (scFv)), amino acids, polypeptides (e.g., peptides or proteins), cells, bacteria, gene therapeutics, genome engineering therapeutics, epigenome engineering therapeutics, carbohydrates, chemical drugs, contrast agents, magnetic particles, polymer beads, metal nanoparticles, metal microparticles, quantum dots, antioxidants, antibiotic agents, hormones, nucleoproteins, polysaccharides, glycoproteins, lipoproteins, steroids, analgesics, local anesthetics, anti-inflammatory agents, anti-microbial agents, chemotherapeutic agents, exosomes, outer membrane vesicles, vaccines, viruses, bacteriophages, adjuvants, vitamins, minerals, organelles, and combinations thereof. In certain embodiments, the composition is a nucleic acid (e.g., an oligonucleotide, mRNA, or DNA). In certain embodiments, the composition is an antibody. In certain embodiments, the composition is a polypeptide (e.g., a peptide or a protein).

In certain embodiments, the composition has a concentration in the fluid of between 0.0001 μg/mL and 1,000 μg/mL (e.g., from about 0.0001 μg/mL to about 0.001 μg/mL, about 0.001 μg/mL to about 0.01 μg/mL, about 0.001 μg/mL to about 5 μg/mL, about 0.005 μg/mL to about 0.1 μg/mL, about 0.01 μg/mL to about 0.1 μg/mL, about 0.01 μg/mL to about 1 μg/mL, about 0.1 μg/mL to about 1 μg/mL, about 0.1 μg/mL to about 5 μg/mL, about 1 μg/mL to about 10 μg/mL, about 1 μg/mL to about 50 μg/mL, about 1 μg/mL to about 100 μg/mL, about 2.5 μg/mL to about 15 μg/mL, about 5 μg/mL to about 25 μg/mL, about 5 μg/mL to about 50 μg/mL, about 5 μg/mL to about 500 μg/mL, about 7.5 μg/mL to about 75 μg/mL, about 10 μg/mL to about 100 μg/mL, about 10 μg/mL to about 1,000 μg/mL, about 25 μg/mL to about 50 μg/mL, about 25 μg/mL to about 250 μg/mL, about 25 μg/mL to about 500 μg/mL, about 50 μg/mL to about 100 μg/mL, about 50 μg/mL to about 250 μg/mL, about 50 μg/mL to about 750 μg/mL, about 100 μg/mL to about 300 μg/mL, about 100 μg/mL to about 1,000 μg/mL, about 200 μg/mL to about 400 μg/mL, about 250 μg/mL to about 500 μg/mL, about 350 μg/mL to about 500 μg/mL, about 400 μg/mL to about 1,000 μg/mL, about 500 μg/mL to about 750 μg/mL, about 650 μg/mL to about 1,000 μg/mL, or about 800 μg/mL to about 1,000 μg/mL, e.g., about 0.0001 μg/mL, about 0.0005 μg/mL, about 0.001 μg/mL, about 0.005 μg/mL, about 0.01 μg/mL, about 0.02 μg/mL, about 0.03 μg/mL, about 0.04 μg/mL, about 0.05 μg/mL, about 0.06 μg/mL, about 0.07 μg/mL, about 0.08 μg/mL, about 0.09 μg/mL, about 0.1 μg/mL, about 0.2 μg/mL, about 0.3 μg/mL, about 0.4 μg/mL, about 0.5 μg/mL, about 0.6 μg/mL, about 0.7 μg/mL, about 0.8 μg/mL, about 0.9 μg/mL, about 1 μg/mL, about 1.5 μg/mL, about 2 μg/mL, about 2.5 μg/mL, about 3 μg/mL, about 3.5 μg/mL, about 4 μg/mL, about 4.5 μg/mL, about 5 μg/mL, about 5.5 μg/mL, about 6 μg/mL, about 6.5 μg/mL, about 7 μg/mL, about 7.5 μg/mL, about 8 μg/mL, about 8.5 μg/mL, about 9 μg/mL, about 9.5 μg/mL, about 10 μg/mL, about 15 μg/mL, about 20 μg/mL, about 25 μg/mL, about 30 μg/mL, about 35 μg/mL, about 40 μg/mL, about 45 μg/mL, about 50 μg/mL, about 55 μg/mL, about 60 μg/mL, about 65 μg/mL, about 70 μg/mL, about 75 μg/mL, about 80 μg/mL, about 85 μg/mL, about 90 μg/mL, about 95 μg/mL, about 100 μg/mL, about 200 μg/mL, about 250 μg/mL, about 300 μg/mL, about 350 μg/mL, about 400 μg/mL, about 450 μg/mL, about 500 μg/mL, about 550 μg/mL, about 600 μg/mL, about 650 μg/mL, about 700 μg/mL, about 750 μg/mL, about 800 μg/mL, about 850 μg/mL, about 900 μg/mL, about 950 μg/mL, or about 1,000 μg/mL).

In some embodiments, the device further includes one or more reservoirs, e.g., a first reservoir and a second reservoir, fluidically connected to a zone, e.g., the entry zone or recovery zone, of a device. For example, a first reservoir may be fluidically connected to the entry zone and a second reservoir may be fluidically connected to the recovery zone.

In some embodiments, the electroporation zone of the device has a uniform cross-sectional dimension. In other embodiments, the electroporation zone of the device has a non-uniform cross-sectional dimension. In further embodiments, the device further comprises a plurality of electroporation zones, where each of the plurality of electroporating zones, e.g., electroporation zone, may have a uniform cross-section or a non-uniform cross-section. In certain embodiments, the cross-section of the electroporation zone is selected from the group consisting of cylindrical, ellipsoidal, polygonal, star, parallelogram, trapezoidal, and irregular.

In some embodiments, the cross-sectional dimension of the entry zone or the cross-sectional dimension of the recovery zone is between 0.01% to 100,000% of the cross-sectional dimension of the electroporation zone. For example, the cross-sectional dimension of the entry zone or the cross-sectional dimension of the recovery zone may be about 0.01% to about 100% of the cross-sectional dimension of the electroporation zone, e.g., about 0.01% to about 1%, about 0.1% to about 10%, about 5% to about 25%, about 10% to about 50%, about 25% to about 75%, or about 50% to about 100% of the cross-sectional dimension of the electroporation zone. Alternatively, the cross-sectional dimension of the entry zone or the cross-sectional dimension of the recovery zone may be about 100% to about 100,000% of the of the cross-sectional dimension of the electroporation zone, e.g., about 100% to about 1000%, about 500% to about 5,000%, about 1,000% to about 10,000%, about 5,000% to about 25,000%, about 10,000% to about 50,000%, about 25,000% to about 75,000%, or about 50,000% to about 100,000% of the cross-sectional dimension of the electroporation zone.

In some embodiments, the cross-sectional dimension of the electroporation zone is between 0.005 mm and 50 mm. In some embodiments, the length of the electroporation zone is between 0.005 mm and 50 mm. In some embodiments, the length of the electroporation zone is between 0.005 mm and 25 mm. In some embodiments, the cross-sectional dimension of any of the first electrode or the second electrode is between 0.1 mm to 500 mm. In particular embodiments, none of the entry zone, recovery zone, or electroporation zone reduces a cross-section dimension of any of the plurality of cells suspended in the fluid, e.g., cells can pass through the device without deformation.

In further embodiments, the device includes an outer structure having a housing configured to encase the first electrode, second electrode, and the electroporation zone of the device. In some embodiments, the outer structure includes a first electrical input operatively coupled to the first electrode and a second electrical input operatively coupled to the second electrode. In some embodiments, the outer structure is integral to the device. In certain embodiments, the outer structure is releasably connected to the device.

In some embodiments, the delivery rate from the fluid delivery source is between 0.001 mL/min to 1,000 mL/min, e.g., 20-30 mL/min, e.g., 25 mL/min. In certain embodiments, the residence time of any of the plurality of cells suspended in the fluid is between 0.5 ms and 50 ms. In some embodiments, the conductivity of the fluid is between 0.001 mS/cm to 500 mS/cm, e.g., 1-20 mS/cm.

In further embodiments, the method includes a controller operatively coupled to the source of electrical potential to deliver voltage pulses to the first electrode and second electrodes to generate an electrical potential difference between the first and second electrodes. In some embodiments, the voltage pulses have an amplitude of −3 kV to 3 kV, e.g., 0.2-0.6 kV. In some cases, the duty cycle of the electroporation is between 0.001% and 100%, e.g., between 10% and 95%. In some embodiments, the voltage pulses have a duration of between 0.01 ms and 1,000 ms, e.g., between 1 ms and 10 ms. In certain embodiments, the voltage pulses are applied the first and second electrodes at a frequency between 1 Hz to 50,000 Hz, e.g., 100-500 Hz. The waveform of the voltage pulse may be DC, square, pulse, bipolar, sine, ramp, asymmetric bipolar, arbitrary, or any superposition or combination thereof. In particular embodiments, the electric field generated from the voltage pulses has a magnitude of between 1 V/cm and 50,000 V/cm, e.g., between 100 V/cm and 1,000 V/cm.

In further embodiments, the method includes a housing structure configured to house the electroporation device described herein. In further instances, the housing structure includes a thermal controller configured to increase or decrease the temperature of the housing or any component of the system thereof. In some embodiments, the thermal controller is a heating element, e.g., a heating block, liquid flow, battery powered heater, or a thin-film heater. In other embodiments, the thermal controller is a cooling element, e.g., liquid flow, evaporative cooler, or thermoelectric, e.g., Peltier device. In certain embodiments, the temperature of the plurality of cells suspended in the fluid is between 0° C. and 50° C.

In further embodiments, the device includes a plurality of cell porating devices, e.g., in series or in parallel. In particular embodiments, the device includes a plurality of outer structures for the plurality of devices.

In some embodiments, the method further includes storing the plurality of cells suspended in the fluid in a recovery buffer after poration. In certain embodiments, the electroporated cells have a viability after introduction of the composition between 0.1% and 99.9%, e.g., 25% and 85%. In other embodiments, the efficiency of the introduction of the composition into the cells is between 0.1 and 99.9%, e.g., between 25% and 85%. In certain embodiments, the cell recovery rate is between 0.1% and 100%. In particular embodiments, the cell recovery yield is between 0.1% and 500%. In some embodiments, the number of recovered cells (e.g., live cells) is between 10⁴ and 10¹².

In another aspect, the invention provides a kit for electro-mechanical delivery of a composition into a plurality of cells suspended in a fluid, the kit including a plurality of devices as described herein, a plurality of outer structures as described herein, and a transfection buffer.

In another aspect, the invention provides a kit for electroporation of a composition into a plurality of cells suspended in a fluid, the kit including a plurality of devices as described herein, a plurality of outer structures as described herein, and a transfection buffer.

In some embodiments of any of the preceding aspects, the outer structures are integral to the plurality of cell devices. In certain embodiments, the outer structures are releasably connected to the plurality of devices.

### BRIEF DESCRIPTION OF THE DRAWINGS

The application file contains at least one drawing executed in color. Copies of this patent application with color drawings will be provided by the Office upon request of the payment of the necessary fee.

FIGS. 1A-1C are schematics of an embodiment of a single electroporation device of the invention. FIG. 1A shows a schematic of the operation of the device of the invention. FIG. 1B shows a schematic of the components of the invention. FIG. 1C shows a photograph of the embodiment of the device of the invention shown in FIG. 1B.

FIGS. 2A-2B are example schematics of a housing for parallel delivery of electrical energy to embodiments of electroporation devices of the invention. FIG. 2A shows an isometric view of the housing with electrical grids concept to be used to energize 96 electroporation devices of the invention in parallel. FIG. 2B shown a zoomed in view of the interface of a single electroporation device of the invention and the housing with electrical grids using spring loaded electrodes to securely hold the first and second electrodes of each electroporation device against the electrical grids of the housing.

FIGS. 3A-3B are bar graphs of the optimization of fluid flow rate (mL/min) for the electroporation of Jurkat cells (1×10⁷ cells/mL) using devices of the invention. Recovering cells were cultured for 24 hours in RPMI with 10% FBS at 37° C. before flow cytometer analysis using the LSR II HTS (BD Bioscience). FIG. 3A shows the viability of Jurkat cells assessed using 7-AAD exclusion dye. FIG. 3B shows the transfection efficiency of the Jurkat cells assessed using GFP expression.

FIGS. 4A-4D are flow rate simulation illustrations along an active zone of a device. FIG. 4A is a 3D model representing a liquid volumetric flow rate of 10 mL per minute. FIG. 4C is a 3D model representing a liquid volumetric flow rate of 100 mL per minute. FIGS. 4B and 4D are 2D models corresponding to FIGS. 4A and 4C, respectively.

FIGS. 5A-5B are bar graphs for the optimization of the electric field in the electroporation zone of devices of the invention for the electroporation of Jurkat cells. Recovering cells were cultured for 24 hours in RPMI with 10% FBS at 37° C. before flow cytometer analysis using the LSR II HTS (BD Bioscience). FIG. 5A shows the viability of Jurkat cells assessed using 7-AAD exclusion dye. FIG. 5B shows the transfection efficiency of the Jurkat cells assessed using GFP expression.

FIGS. 6A-6B are bar graphs showing the effects of temperature on the transfection of Jurkat cells using devices of the invention. “RT” in the figures stands for room temperature. Recovering cells were cultured for 24 hours in RPMI with 10% FBS at 37° C. before flow cytometer analysis using the LSR II HTS (BD Bioscience). FIG. 6A shows the viability of Jurkat cells assessed using 7-AAD exclusion dye. FIG. 6B shows the transfection efficiency of the Jurkat cells assessed using GFP expression.

FIGS. 7A-7D are simulation illustrations showing electric field distributions along an active zone of a device of the invention. FIG. 7A shows an electric field distribution map of a device with an applied voltage of 225 V. FIG. 7B is a 2D model longitudinal cross-section of FIG. 7A. FIG. 7C shows an electric field distribution map of a device with an applied voltage of 275 V. FIG. 7D is a 2D model longitudinal cross-section of FIG. 7C.

FIGS. 8A-8D are simulation illustrations showing the effects of temperature distributions along an active zone of a device of the invention. FIG. 8A shows a temperature distribution map of the liquid in an active zone of the device at time=0 ms; FIG. 8B shows a temperature distribution map of the liquid in an active zone of the device at time=100 ms; FIG. 8C shows a temperature distribution map of the liquid in an active zone of the device at time=200 ms; and FIG. 8D shows a temperature distribution map of the liquid in an active zone of the device at time=300 ms.

FIGS. 9A-9B are bar graphs showing the optimization of the voltage pulse duration and number of pulses for the electroporation of Jurkat cells using devices of the invention. Recovering cells were cultured for 24 hours in RPMI with 10% FBS at 37° C. before flow cytometer analysis using the LSR II HTS (BD Bioscience). FIG. 8A shows the viability of Jurkat cells assessed using 7-AAD exclusion dye. FIG. 9B show the transfection efficiency of the Jurkat cells assessed using GFP expression.

FIGS. 10A-10B are bar graphs showing the optimization of sample volume for the electroporation of Jurkat cells using devices of the invention. Recovering cells were cultured for 24 hours in RPMI with 10% FBS at 37° C. before flow cytometer analysis using the LSR II HTS (BD Bioscience). FIG. 10A shows the viability of Jurkat cells assessed using 7-AAD exclusion dye. FIG. 10B shows the transfection efficiency of the Jurkat cells assessed using GFP expression.

FIGS. 11A-11B are bar graphs showing the optimization of the diameter of the electroporation zone for the electroporation of Jurkat cells using devices of the invention. Electroporations were performed at a fixed voltage with variable flow rates to substantially match total cell residence time across the different channel dimensions. Recovering cells were cultured for 24 hours in RPMI with 10% FBS at 37° C. before flow cytometer analysis using the LSR II HTS (BD Bioscience). FIG. 11A shows the viability of Jurkat cells assessed using 7-AAD exclusion dye. FIG. 11B shown the transfection efficiency of the Jurkat cells assessed using GFP expression.

FIGS. 12A-12L show bar graphs showing the effect of select voltage pulse waveforms for the electroporation of Jurkat cells using devices of the invention and exemplary waveform shapes. Recovering cells were cultured for 24 hours in RPMI with 10% FBS at 37° C. before flow cytometer analysis using the LSR II HTS (BD Bioscience). FIG. 12A shows the viability of Jurkat cells assessed using 7-AAD exclusion dye. FIG. 12B shows the transfection efficiency of the Jurkat cells assessed using GFP expression. FIG. 12C shows a direct current (DC) always on waveform. FIG. 12D shows a square wave waveform with a 50% duty cycle including an offset. FIG. 12E shows a 75% asymmetric ramp waveform. FIG. 12F shows a pulse waveform with a 95% duty cycle. FIG. 12G shows a square wave waveform with a 75% duty cycle including an offset. FIG. 12H shows a sine waveform. FIG. 12I shows a 25% asymmetric ramp waveform. FIG. 12J shows a square wave waveform with a 25% duty cycle including an offset. FIG. 12K shows a bipolar square wave waveform with no offset. FIG. 12L shows a symmetric ramp waveform.

FIGS. 13A-13B are bar graphs comparing the transfection efficiency and resulting cell viability for Jurkat cells using a device of the invention and a commercially available cell transfection instrument. Viability of Jurkat cells assessed using 7-AAD exclusion dye and transfection efficiency of the Jurkat cells assessed using GFP expression. FIG. 13A show results from transfection experiments performed using published parameters for Jurkat cell transfection (sample in a 100 μL tip; 3 pulse/10 ms/450 V/cm). FIG. 13B is a duplicated experiment of FIG. 13A which shows reproducibility in experiments performed using optimized parameters for the devices of the invention compared to published parameters for Jurkat cell transfection. FIG. 13C shows a workflow schematic of a Cas9 ribonucleoprotein arrayed library screen using a commercially available single strand sgRNA arrayed library to anneal the purified Cas9 protein to form an arrayed Cas9 ribonucleoprotein library. Using a device of the invention, the Cas9 ribonucleoprotein arrayed library screen will result in identification of gene targets for future immunotherapeutic research using plate-based analysis. Additionally, Cas9 ribonucleoprotein pooled library screening could be used to perform assays required to identify gene targets for future therapies.

FIGS. 14A-14B are bar graphs showing the viability and efficiency of the delivery of FITC dextran into primary human T-cells using devices of the invention, using variable molecular weight dextran polymers to assess any size restrictions for dextran delivery. Recovering cells were cultured for 24 hours in RPMI with 10% FBS at 37° C. before flow cytometer analysis using the LSR II HTS (BD Bioscience). FIG. 14A shows the viability of primary human T-cells assessed using 7-AAD exclusion dye. FIG. 14B shows the transfection efficiency of the primary human T-cells assessed using GFP expression.

FIGS. 15A-15B are bar graphs comparing transfection efficiency and viability in THP-1 monocytes using devices of the invention and a commercially available cell transfection instrument (NEON®) using published transfection protocols for THP-1 monocytes. Recovering cells were cultured for 24 hours in RPMI with 10% FBS at 37° C. before flow cytometer analysis using the LSR II HTS (BD Bioscience). FIG. 15A shows the viability of THP-1 monocytes assessed using 7-AAD exclusion dye. FIG. 15B shows the transfection efficiency of the THP-1 monocytes assessed using GFP expression.

FIGS. 16A-16B are bar graphs comparing the transfection efficiency and viability in primary human monocytes using devices of the invention and a commercially available cell transfection instrument using published transfection protocols for primary human monocytes. The primary human monocytes were isolated from peripheral blood using negative selection. Recovering cells were cultured for 24 hours in RPMI with 10% FBS at 37° C. before flow cytometer analysis using the LSR II HTS (BD Bioscience). FIG. 16A shows the viability of primary human monocytes assessed using 7-AAD exclusion dye. FIG. 16B shows the transfection efficiency of the primary human monocytes assessed using GFP expression.

FIGS. 17A-17B are bar graphs comparing the transfection efficiency and viability in the NK-92 cell line using devices of the invention and a commercially available cell transfection instrument using published transfection protocols for NK-92 cell line. After electroporation using devices of the invention or using a commercially available instrument, the cells were cultured for 24 hours in complete aMEM (aMEM with 25% serum, 0.2 mM inositol, 0.02 mM folic acid 0.1 mM mercaptoethanol) at 37° C. before flow cytometer analysis using the iQue (Intellicyt). FIG. 17A shows the viability assessed using 7-AAD exclusion dye. FIG. 17B shows the transfection efficiency assessed by GFP expression.

FIGS. 18A-18B are bar graphs comparing the transfection efficiency and viability in the NK-92MI cell line using devices of the invention and a commercially available cell transfection instrument using published transfection protocols for NK-92MI cell line. After electroporation the cells were cultured for 24 hours in complete aMEM (aMEM with 25% serum, 0.2 mM inositol, 0.02 mM folic acid 0.1 mM mercaptoethanol) at 37° C. before flow cytometer analysis using the iQue (Intellicyt). FIG. 18A shows the viability assessed using 7-AAD exclusion dye. FIG. 18B shows the transfection efficiency assessed by GFP expression.

FIGS. 19A-19F are bar graphs comparing T cells (FIGS. 19A-19C) with primary human monocytes (FIGS. 19D-19F) electroporated and transfected with SIRPalpha custom mRNA using devices of the invention compared to non-electroporated cells. Day 11 expanded T cell were transfected with 20 μg of SIRPalpha mRNA and assessed for over expression at 24 hours. Representative graphs for A) viability measured as 7-AAD negative cells, B) transfection efficiency measured as SIRPalpha positive cells, and C) SIRPalpha expression measured as mean fluorescent intensity (MFI). Monocytes isolated from PBMCs were transfected with 20 μg of SIRPalpha mRNA and assessed for over expression at 24 hours. Representative graphs for D) viability measured as 7-AAD negative cells, E) transfection efficiency measured as SIRPalpha positive cells, and F) SIRPalpha expression measured as mean fluorescent intensity (MFI). Graphs are Mean±SEM.

FIGS. 20A-20D are bar graphs showing delivery of GFP mRNA to human primary native T cells. FIG. 20A shows recovered cells, FIG. 20B shows naive T cell efficiency, FIG. 20C shows naive T cell viability, and FIG. 20D shows total yield. Naive T cell were transfected with 10 μg of commercial GFP mRNA and assessed for expression at 24 hours. Representative graphs for counts, viability, efficiency, and yield are shown. Graphs are Mean±SEM.

FIGS. 21A-21B are FACS plots showing that electroporation does not change the phenotype human primary naive T cells. FIG. 21A shows nontreated cells, and FIG. 21B shows electroporated cells. Naive T cell were transfected with 10 μg of commercial GFP mRNA and then stained for CD45RA and CD45RO at 24 hr, as shown in the dot plots. The CD45RA/CD45RO phenotypes are equivalent between nontreated and Flowfect™ electroporated naïve T cells.

FIG. 22 is a kinetic plot showing naive T cell expansion using a device of the invention compared to nontreated cells. Electroporation does not change the expansion of human primary naive T cells. Naive T cell were transfected with 10 μg of commercial GFP mRNA and then expanded with soluble CD3/CD28 activators. Cell counts were taken 1, 4, and 6 days after activation. The expansion rates are equivalent between nontreated and electroporated naïve T cells.

FIGS. 23A-23F show example embodiments of electroporation devices of the invention integrated into an electronic discharge device configured to energize and electroporate a plurality of cell samples simultaneously. FIG. 23A shows a top isometric view of an electronic discharge device. FIG. 23B shows side view of a device of the invention installed into an electronic discharge device showing how electrical contact is made in the system using pogo pin-style electrical contacts. FIG. 23C shows a side view of a full electronic discharge device. FIG. 23D shows a top isometric view of an alternate embodiment of an electronic discharge device. FIG. 23E shows a side view of a device of the invention installed into an electronic discharge device showing how electrical contact is made in the system using flexible spring-style electrical contacts. FIG. 23F shows an overhead view of an electronic discharge device configured to energize and electroporate a plurality of cell samples simultaneously.

FIGS. 24A-24B show embodiments of a temperature-controlled electroporation device of the invention using a thermal liquid for temperature control. FIG. 24A shows a schematic of the components of the temperature controlled electroporation device. FIG. 24B shows a side view of the temperature-controlled electroporation device showing the device in an external frame.

FIGS. 25A-25B show embodiments of a fluidic chip-based electroporation device of the invention configured to accept industry standard pipette tips for sample introduction. FIG. 25A shows an embodiment of a fluidic chip incorporating embedded electrodes and fluidic channels. FIG. 25B shows a schematic of the components of the fluidic chip-based electroporation device.

FIGS. 26A-26B show embodiments of a continuous flow electroporation device of the invention. FIG. 26A shows a cutaway schematic of the components of a continuous flow electroporation device. FIG. 26B shows an outside view with transparency to show the components of the continuous flow electroporation device.

FIGS. 27A-27F show the simulated electric field generated using computational modeling of an embodiment of a helical electrode. FIG. 27A shows the simulated electric field of a helical electrode shown along all three Cartesian axes. FIG. 27B shows the simulated electric field of a helical electrode shown from a cross-section along the Z-axis. FIGS. 27C-27F show the simulated electric field of a helical electrode along the X-Y axis shown from four different positions along the Z-axis.

FIGS. 28A-28C show embodiments of a two-part electroporation device of the invention configured for manufacturing scalability. FIG. 28A shows a top isometric 3D rendering of an embodiment of a two-part electroporation device of the invention. FIG. 28B shows a vertical cross-section of the embodiment of depicted in FIG. 28A showing how the two components mate. FIG. 28C shows an identical view of the embodiment depicted in FIG. 28B with dimensions (in mm) of the device overlaid.

FIGS. 29A-29B shows an embodiment of a two-part electroporation device of the invention that includes embedded electrodes with an interface for a liquid handling cannula. FIG. 29A shows a top isometric 3D rendering of an embodiment of a two-part electroporation device of the invention with embedded electrodes. FIG. 29B shows a vertical cross-section of the embodiment depicted in FIG. 29A showing the location of the embedded electrodes relative to the electroporation zone of the device of the invention.

FIG. 29C is a drawing of a multi-device approach that enhances throughput and parallelization of the present technology.

FIGS. 30A-30B show embodiments of an outer housing of the invention configured to house a plurality of devices of the invention, liquid handling components, controllers, and any electrical components. FIG. 30A shown an embodiment of an outer housing of the invention with a user interface.

FIG. 30B shows an embodiment of devices of the invention connected to a liquid dispensing manifold and a sample plate.

FIG. 31 shows a comparison between traditional (using a commercially available Lonza NUCLEOFECTOR 4D™ electroporation system, bottom) and adopted (using devices and systems of the invention, top) flow cytometry gating strategy for post-transfection analysis for cell count, viability, transfection efficiency, and detection of surface/intracellular markers.

FIGS. 32A-32B are bar graphs showing the viability and efficiency from the delivery of GFP-coding plasmid DNA into CHO-K1 cells using devices of the invention 24 hours after electroporation. FIG. 32A shows the viability of CHO-K1 cells. FIG. 32B shows the transfection efficiency of the CHO-K1 cells assessed using GFP expression.

FIGS. 33A-33D are bar graphs showing the viability and efficiency from the delivery of GFP-coding plasmid DNA into HEK-293T cells using devices of the invention 24 and 48 hours after electroporation. FIG. 33A shows the viability of HEK-293T cells 24 hours after electroporation. FIG. 33B shows the transfection efficiency of the HEK-293T cells assessed using GFP expression 24 hours after electroporation. FIG. 33C shows the viability of HEK-293T cells 48 hours after electroporation. FIG. 33D shows the transfection efficiency of the HEK-293T cells assessed using GFP expression 48 hours after electroporation.

FIGS. 34A-34B show the collected GFP fluorescence signals of Chinese Hamster Ovary (CHO-K1) cells before (FIG. 34A) and after (FIG. 34B) electroporation using devices and systems of the invention. The GFP fluorescence images were captured using an ECHO Revolve microscope equipped with a 10× objective.

FIGS. 35A-35B show the collected GFP fluorescence signals of HEK-293T cells before (FIG. 35A) and after (FIG. 35B) electroporation using devices and systems of the invention. The GFP fluorescence images were captured using an ECHO Revolve microscope equipped with a 10× objective.

FIGS. 36A-36D are bar graphs showing the post-electroporation total cell counts, viability, efficiency, and relative live positively transfected cells for delivery of 40 kD FITC dextran to primary human T-cells using a commercially available NEON® transfection system and devices of the invention. FIG. 36A shows total cell counts post electroporation. FIG. 36B shows viability of the primary human T-cells. FIG. 36C shows the efficiency of the delivery into primary human T-cells. FIG. 36D shows the relative live positively transfected cell population.

FIG. 37 is a bar graph showing a comparison between the NEON® transfection system and devices of the invention for the relative live positively transfected cell population after delivery of GFP plasmid to primary human T-cells.

FIGS. 38A-38D are bar graphs showing the recovery, viability, efficiency, and yield of the delivery of mRNA into primary human T-cells at 9 days of age. Electroporation was performed using two commercially available transfection systems (Lonza NUCLEOFECTOR 4D™ and Thermo Fisher NEON®) and devices of the invention. Either 1 million (10⁶ cells/mL) or 5 million (5×10⁶ cells/mL) were electroporated in 100 μL with 10 μg mRNA encoding EGFP. Analysis via flow cytometry was performed 24 hours post electroporation. Cell counts are normalized to 1 million cell inputs, and yield is normalized to the results collected using devices of the invention. FIG. 38A shows the recovery at both cell densities. FIG. 38B shows the viability at both cell densities. FIG. 38C shows the efficiency at both cell densities. FIG. 38D shows the yield at both cell densities.

FIGS. 39A-39D are line plots showing the recovery, viability, efficiency, and MFI of the delivery of Cas9 ribonucleoprotein complexes (RNPs) targeting CXCR3 in primary human T-cells using devices and systems of the invention. Cas9 RNPs were formulated with commercially available Cas9 protein and two commercial sources of sgRNA. Analysis via flow cytometry was performed 24-72 hours post-electroporation. FIG. 39A shows the cell recovery. FIG. 39B shows the viability. FIG. 39C shows the efficiency. FIG. 39D shows the total yield of target KO cells expanded out to 72 hours post-electroporation.

FIGS. 40A-40B are bar graphs showing the live cell counts for GFP expression from THP-1 cells and FITC labeled dextran delivery to NK-92MI cells for electroporation using a commercial NEON® transfection system and devices of the invention. FIG. 40A shows the live cell counts for GFP expression to THP-1 cells. FIG. 40B shows the live cell counts for FITC labeled dextran delivery to NK-92MI cells.

FIGS. 41A-41B are bar graphs showing a comparison of the resulting viability and efficiency of GFP mRNA delivery into THP-1 monocytes using a commercial NEON® transfection system and devices of the invention. FIG. 41A shows the viability of THP-1 monocytes assessed 24 hours after transfection.

FIG. 41B shows the transfection efficiency THP-1 monocytes assessed using GFP expression 24 hours after electroporation.

FIGS. 42A-42C are bar graphs showing the viability, efficiency, and yield of GFP mRNA delivery into THP-1 monocytes using devices of the invention with a control sample of non-electroporated cells. FIG. 42A shows the viability of the transfected cells assessed 24-72 hours post electroporation. FIG. 42B shows the efficiency of the uptake of GFP mRNA assessed 24-72 hours post electroporation. FIG. 42C shows the yield of the transfected cells assessed 24-72 hours post electroporation

FIGS. 43A-43B are bar graphs showing the viability and efficiency of the delivery of GFP mRNA delivery into LPS-activated THP-1 cells using devices of the invention. FIG. 43A shows the viability of LPS-activated THP-1 cells assessed 24 hours after transfection. FIG. 43B shows the transfection efficiency LPS-activated THP-1 cells assessed using GFP expression 24 hours after electroporation.

FIGS. 44A-44D are bar graphs showing the viability and efficiency of the delivery of 40 kD FITC dextran and GFP mRNA into primary peripheral blood monocytes using devices of the invention. FIG. 44A shows the viability of primary peripheral blood monocytes transfected with FITC dextran. FIG. 44B shows the transfection efficiency of the primary peripheral blood monocytes transfected with FITC dextran. FIG. 44C shows the viability of primary peripheral blood monocytes transfected with GFP mRNA. FIG. 44B shows the transfection efficiency of the primary peripheral blood monocytes transfected with GFP mRNA.

FIGS. 45A-45B are bar graphs showing the expression of CD80 and CD86 in primary peripheral blood monocytes that were transfected with GFP with LPS stimulation using devices of the invention. Expression of CD80 and CD86 was measured 24 hours and 96 hours after electroporation. FIG. 45A shows the expression of the activation marker CD80. FIG. 45B shows the expression of the lineage marker CD86.

FIGS. 46A-46C are bar graphs showing the macrophage phenotype, viability, and GFP expression of primary peripheral blood monocytes transfected with GFP mRNA using devices of the invention that differentiated into macrophages over 4-8 days. FIG. 46A shows macrophage phenotype assessed via flow cytometric analysis of FSC and SSC. FIG. 46B shows the viability of the transfected macrophages. FIG. 46C shows the percent GFP expression of the transfected macrophages.

FIG. 47A-47D are bar graphs showing the viability and efficiency of the delivery of 40 kD FITC dextran and GFP mRNA into peripheral blood differentiated macrophages using devices of the invention. FIG. 47A shows the viability of peripheral blood differentiated macrophages transfected with FITC dextran. FIG. 47B shows the transfection efficiency of peripheral blood differentiated macrophages transfected with FITC dextran. FIG. 47C shows the viability of peripheral blood differentiated macrophages transfected with GFP mRNA. FIG. 47D shows the transfection efficiency of peripheral blood differentiated macrophages transfected with GFP mRNA.

FIGS. 48A-48B are bar graphs showing the ability of peripheral blood differentiated macrophages to polarize into M1 and M2 macrophages after transfection with GFP mRNA using devices of the invention. FIG. 48A shows M1 polarized macrophages where M1 polarization with IFNg+LPS stimulation was indicated by elevated CD86 expression. FIG. 48B shows M2 polarized macrophages where M2 polarization, IL-4 stimulation, was indicated by CD206 expression.

FIGS. 49A-49C are bar graphs showing the viability, efficiency, and live cell count of primary human monocytes transfected with FITC dextran using a commercial NEON® transfection system and devices of the invention. FIG. 49A shows the viability of the primary human monocytes. FIG. 49B shows the efficiency of the delivery of FITC dextran into primary human monocytes. FIG. 49C shows the live cell count of the transfected primary human monocytes.

FIGS. 50A-50D are bar graphs comparing the recovery, viability, efficiency, and yield of DNA transfection into Jurkat cells of varying cell densities using single channel and continuous flow devices of the invention. FIG. 50A shows the recovery of the transfected Jurkat cells. FIG. 50B shows the viability of the transfected Jurkat cells. FIG. 50C shows the efficiency of the DNA transfection into Jurkat cells. FIG. 50D shows the yield of the transfected Jurkat cells.

FIGS. 51A-51B are bar graphs comparing the GFP and FITC yield of transfected Jurkat cells using single channel and continuous flow devices of the invention. FIG. 51A shows the GFP yield for transfected Jurkat cells. FIG. 51B shows the FITC yield for transfected Jurkat cells.

FIGS. 52A-52D are bar graphs showing the delivery of FITC dextran into of high cell density suspensions using continuous flow devices of the invention. Analysis via flow cytometry was performed 24 hours post electroporation. FIG. 52A shows the total recovered cell counts relative to 1 million cell inputs. FIG. 52B shows the viability of the transfected Jurkat cells. FIG. 52C shows the efficiency of the FITC dextran transfection into Jurkat cells. FIG. 52D shows the FITC yield of the transfected Jurkat cells.

FIG. 53A-53D are bar graphs showing the recovery, viability, efficiency, and yield of mRNA transfection into Jurkat cells at a cell number of 100 million cells using varying amounts of mRNA and varying cell concentrations in continuous flow devices of the invention. Analysis via flow cytometry was performed 24 hours post electroporation. FIG. 53A shows the number of recovered Jurkat cells at different concentrations of mRNA and cell concentrations. FIG. 53B shows the viability of the transfected Jurkat cells at different concentrations of mRNA and cell concentrations. FIG. 53C shows the efficiency of the mRNA transfection into Jurkat cells at different concentrations of mRNA and cell concentrations. FIG. 53D shows the yield of the transfected Jurkat cells at different concentrations of mRNA and cell concentrations.

FIG. 54 shows flow cytometric analysis of non-treated T-cells and electroporated T-cells comparing the commercial Lonza NUCLEOFECTOR 4D™ transfection system and the devices of the invention. The top panel shows the FSC/SSC total cell plots, and the bottom panel shows the viability staining. Dead cell populations are indicated with red arrows and red boxes. There is also a morphology shift of cells transfected with the Lonza NUCLEOFECTOR 4D™ at 24 h compared to the non-treated cells, indicating phenotypic changes occur during electroporation with the Lonza platform.

FIG. 55 shows a bar graph of the total cell yield from the electroporation of 50 million primary T cells with either FITC-dextran or EGFP mRNA using the commercial Lonza LV transfection system and a continuous flow device of the invention.

FIGS. 56A-56B are bar graphs showing the viability and efficiency of the delivery of FITC dextran into a suspension of 1 billion THP-1 cells using a continuous flow device of the invention for a period of up to 72 hours after electroporation. FIG. 56A shows the viability of the THP-1 cells. FIG. 56B shows the efficiency of the FITC dextran delivery into the THP-1 cells.

FIG. 57 is a bar graph showing the yield of live recoverable FITC dextran transfected cells starting from a suspension of 1 billion THP-1 cells using a continuous flow device of the invention. The yield was tracked for a period of up to 72 hours post electroporation culture and represents approximately 50% of the input number of cells. Analysis via flow cytometry was performed at 4 hours, 24 hours, 48 hours, and 72 hours post-electroporation.

FIGS. 58A-58D are bar graphs comparing the waveform shape and waveform voltage on the total cell counts, viability, efficiency, and yield of FITC dextran transfection into Jurkat cells using devices of the invention. FIG. 58A shows the number of recovered Jurkat cells at different waveform shapes and voltages. FIG. 58B shows the viability of the transfected Jurkat cells at different waveform shapes and voltages. FIG. 58C shows the efficiency of the FITC dextran transfection into Jurkat cells at different waveform shapes and voltages. FIG. 58D shows the yield of the transfected Jurkat cells at different waveform shapes and voltages.

FIGS. 59A-59D are bar graphs comparing the waveform maximum voltages and duty cycles on the total cell counts, viability, efficiency, and yield of FITC dextran transfection into primary T cells using devices of the invention. FIG. 59A shows the number of recovered primary T cells at different waveform maximum voltages and duty cycles. FIG. 59B shows the viability of the transfected primary T cells at different waveform maximum voltages and duty cycles. FIG. 59C shows the efficiency of the FITC dextran transfection into primary T cells at different waveform maximum voltages and duty cycles. FIG. 59D shows the yield of the transfected primary T cells at different waveform maximum voltages and duty cycles.

FIGS. 60A-60D are bar graphs comparing the waveform maximum voltages and duty cycles on the total cell counts, viability, efficiency, and yield of mRNA transfection into primary T cells using devices of the invention. FIG. 60A shows the number of recovered primary T cells at different waveform maximum voltages and duty cycles. FIG. 60B shows the viability of the transfected primary T cells at different waveform maximum voltages and duty cycles. FIG. 60C shows the efficiency of the mRNA transfection into primary T cells at different waveform maximum voltages and duty cycles. FIG. 60D shows the yield of the transfected primary T cells at different waveform maximum voltages and duty cycles.

FIG. 61 is a bar graph showing the efficiency of the delivery of CD3/CD28 Dynabeads into a suspension of 1 million primary human T cells using devices of the invention. Electroporation was performed with and without Dynabeads, with the Dynabead incorporation occurring for 5 minutes or overnight. Analysis via flow cytometry was performed 24 hours post electroporation.

FIGS. 62A-62B show an embodiment of an outer structure that is configured to encase the electrodes of devices of the invention. FIG. 62A shows the outer structure configured with a latch and a clamshell-type hinge to encase a device of the invention. FIG. 62B shows the outer structure of FIG. 62A with a device of the invention resting within the corresponding interior recesses of the outer structure.

FIGS. 63A-63B are bar graphs showing the viability and efficiency of the delivery of FITC dextran into THP-1 monocytes using devices of the invention, both with and without an outer structure covering the electrodes of the device. Analysis via flow cytometry was performed 24 hr post electroporation. FIG. 63A show the viability of the THP-1 monocytes. FIG. 63B shows the efficiency of the transfection of the THP-1 monocytes.

FIGS. 64A-64B are bar graphs showing the viability and efficiency of the delivery of FITC dextran into THP-1 monocytes using devices of the invention fabricated from different polymer resins. FIG. 64A shows the viability of the transfected THP-1 monocytes. FIG. 64B shows the efficiency of the transfection of the FITC dextran into the THP-1 monocytes.

FIGS. 65A-65B are bar graphs comparing the viability and efficiency of the delivery of both DNA and mRNA encoding GFP into Jurkat cells using devices of the invention operated manually or with an automated fluid handling platform. FIG. 65A shows the viability of the transfected Jurkat cells. FIG. 65B shows the efficiency of the transfection of DNA and mRNA encoding GFP into the Jurkat cells.

FIGS. 66A-66E are bar graphs and dot plots comparing the viability and efficiency of the delivery of multiple mRNAs encoding both GFP and mCherry into T cells in either parallel (same day) or series (2 days apart) using devices of the invention operated manually or with an automated fluid handling platform. FIG. 66A shows T cell viability 24 hours post electroporation of the delivery of multiple mRNAs encoding mCherry. FIG. 66B shows GFP efficiency 24 hours post electroporation. FIG. 66C shows mCherry efficiency 24 hours post electroporation. FIG. 66D shows dual GFP and mCherry efficiency 24 hours post electroporation. FIG. 66E shows the dot plots of both GFP (x-axis) and mCherry (y-axis) expression at 24 hours.

FIGS. 67A-67B are bar graphs demonstrating the efficiency of delivery for mRNA into peripheral blood mononuclear cells (PBMCs) using devices of the invention. These experiments were performed with a commercially sourced mRNA encoding GFP, followed by phenotype staining of surface receptors to identify specific cell populations. FIG. 67A shows efficiency in T cell subpopulations, and FIG. 67B shows efficiency in non-T cell populations from the PBMCs. Analysis via flow cytometry was performed 24 hours post electroporation.

FIG. 68 is a photograph of an embodiments of a system of the invention having a reservoir (a bag) in fluid communication with the first inlet and a reservoir (bag) in fluid communication with the second outlet.

FIG. 69A is a set of photomicrographs showing eGFP-mRNA expression using devices of the invention vs. non-treated controls. FIGS. 69B and 69C are bar graphs showing live cell percentages (FIG. 69B) and GFP+ cell percentages (FIG. 69C).

FIGS. 70A-70D are bar graphs showing total NK cell recovery (FIG. 70A), viability (FIG. 70B), transfection efficiency (FIG. 70C), and GFP+ cell yield (FIG. 70D).

FIGS. 71A and 71B are bar graphs showing a cross-platform comparison between a “no-electroporation” control (first bar to the left), the device of the invention (Kytopen; second bar to the left), and two conventional platforms—Thermo (Neon Transfection System) and Lonza 4D-Nucleofector (third and fourth bars, respectively). FIG. 71A shows % viability. FIG. 71B shows % transfection efficiency.

FIG. 71C is a heat map showing gene expression differences across the three platforms compared in FIGS. 71A and 71B. Dark shades correspond to larger degrees of gene expression difference relative to untreated cells.

FIGS. 72A-72D are heatmaps for relative cell count, viability (percent 7-AAD− cells), efficiency (percent live GFP+ cells), and yield cell counts (live GFP+ cell counts) per reaction at 24 hours after transfection, shown as a function of varying Vrms (y axis) and flow rate (x axis).

FIGS. 73A-73D are heatmaps for relative cell count, viability (percent 7-AAD− cells), efficiency (percent live GFP+ cells), and yield cell counts (live GFP+ cell counts) per reaction at 24 hours after transfection, shown as a function of varying Vrms (y axis) and flow rate (x axis).

FIG. 74 is a density plot showing live cell yield across varying applied energy (Vrms²/R)s and flow rates.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

The term “about,” as used herein, refers to +/−10% of a recited value.

The term “plurality,” as used herein, refers to more than one.

The term “substantially uniform,” as used herein, refers to +/−5% variance.

The term “minimum cross-sectional dimension,” as used herein, refers to a minimum length of a straight line that passes through the geometric center of a transverse cross-section of a lumen and intersects an inner wall of the lumen twice on the same plane of the transverse cross-section. The term “cross-sectional area,” unless otherwise specified, refers to the transverse cross-sectional area (e.g., along the plane perpendicular to the longitudinal axis or direction of flow).

The term “fluidically connected,” as used herein, refers to a direct connection between at least two device elements, e.g., an electroporation device, a reservoir, etc., that allows for fluid to move between such device elements without passing through an intervening element.

The term “fluidic communication,” as used herein, refers to an indirect connection between at least two device elements, e.g., an electroporation zone, a reservoir, etc., that allows for fluid to move between such device elements, e.g., through an intervening element, (e.g., through intervening tubing, an intervening channel, etc.). For example, in embodiments in which a fluid flows from a lumen of first electrode, through an electroporation zone, into a lumen of a second electrode, the first electrode is in fluidic communication with the second electrode.

The term “lumen,” as used herein, refers to an interior cavity of an electrode of the devices of the invention that allows for fluid to pass through. Part or all of a lumen of an electrode may be conductive or non-conductive. For example, a lumen of an electrode may encase a C-shaped conductive element that does not completely surround the perimeter of the lumen. In other embodiments, the electrode is substantially entirely composed of the conductive material that transmits current. When an electric potential difference is applied to a first and second electrode of the devices of the invention, an electric field that may be generated in a lumen of any one of the first or second electrodes is not high enough to cause cell electroporation to occur within the lumen.

The term “entry zone,” as used herein, comprises a lumen of a first electrode of the devices of the invention through which a fluid and a plurality of cells suspended in the fluid may pass prior to electroporation. An entry zone may further comprise an additional reservoir in fluidic communication with a lumen of a first electrode of the devices of the invention. When an electric potential difference is applied to a first and second electrode of the devices of the invention, the electric field that may be generated within an entry zone of the devices of the invention is not high enough to cause cell electroporation to occur.

The term “recovery zone,” as used herein, comprises a lumen of a second electrode of the devices of the invention through which a fluid and a plurality of cells suspended in the fluid may pass after electroporation. A recovery zone may further comprise an additional reservoir in fluidic communication with a lumen of a second electrode of the devices of the invention. When an electric potential difference is applied to a first and second electrode of the devices of the invention, the electric field that may be generated within a recovery zone of the devices of the invention is not high enough to cause cell electroporation to occur.

The term “electroporation zone,” as used herein, refers to a portion of a device that is disposed between, and in fluidic communication with, an outlet of an upstream electrode (e.g., a first outlet) and an inlet of a downstream electrode (e.g., a second inlet). The electric field is delivered to the fluid in the electroporation zone.

The term “transfection,” as used herein, refers to a process by which payloads can be introduced into cells utilizing means other than viral delivery methods, such as biological, chemical, electrical, mechanical, or physical methods.

The term “electroporation,” as used herein, refers to a process utilizing applied electric fields to create small pores in cell membranes through which payloads can be introduced into cells (e.g. as a method of transfection).

The term “electro-mechanical delivery,” as used herein, is a transfection process by which payloads can be introduced to cells utilizing any combination of applied electric fields and/or mechanical poration mechanisms. This delivery method has the potential to decrease and/or stabilize the overall electric field exposure of the cells in the electroporation zone, thereby enhancing cell viability and/or transfection efficiency, or both. The devices of the invention are configured to transfect cells via electro-mechanical delivery rather than by electroporation alone.

### DETAILED DESCRIPTION OF THE INVENTION

The present invention provides devices, systems, and methods for the transfection of cells, e.g., primary T cells, by electroporation at larger volumes, higher transfection efficiencies, higher throughputs, higher recovery rates, higher yields, and higher cell viabilities as compared with traditional cuvette based electroporation approaches or commercially available electroporation instruments. In particular, systems and methods are provided that can perform electroporation in a flow-through manner, a continuous manner, or using a plurality of electroporation devices of the invention to enhance throughput and cell numbers.

### Devices

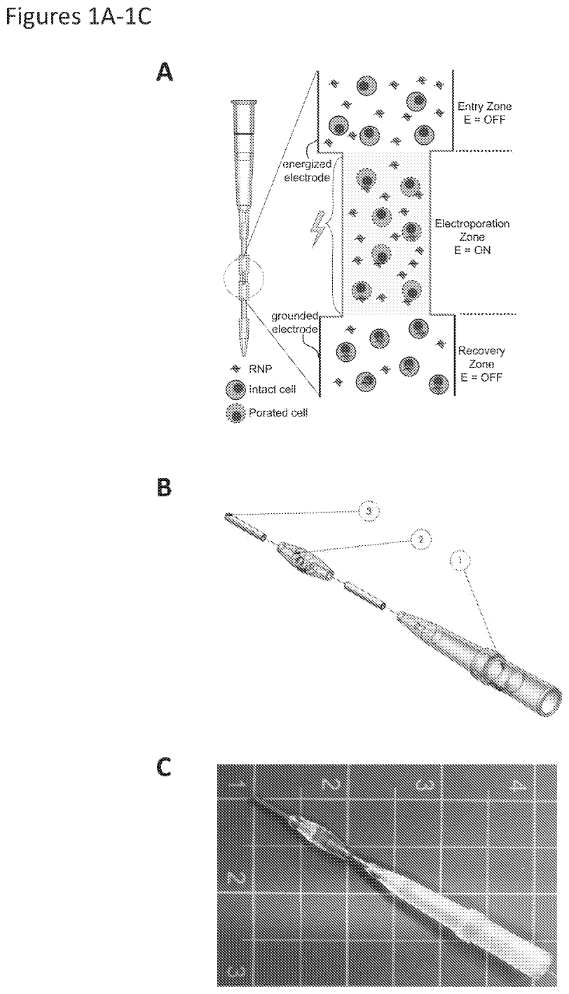

In general, devices of the present invention are configured to be flow through devices that may interface with existing liquid handling, pumps, or fluid transport apparatuses, such as conventional pipette tip robots or large-scale liquid handling systems, to provide continuous electroporation of cells suspended in a fluid. A device of the invention is configured for transfection of cells to occur within the electroporation zone via an electro-mechanical delivery mechanism that is distinct from the delivery mechanism in static electroporation systems. Devices of the invention typically feature three distinct regions: a first electrode having a first inlet and a first outlet, where a lumen of the first electrode defines an entry zone; a second electrode having a second inlet and a second outlet, where a lumen of the second electrode defines a recovery zone; and electroporation zone that is fluidically connected to the first outlet of the first electrode and the second inlet of the second electrode. An example of an embodiment of the device of the invention is shown in FIG. 1A, with the first electrode and second electrode fluidically connected by an electroporation zone therebetween. When an electrical potential difference is applied to the first and second electrodes, a localized electric field develops in the space between the two electrodes, e.g., the electroporation zone, and cells that are exposed to the electric field are electroporated. An individual device of the invention may include two electrodes, as shown in FIGS. 1A-1C; alternatively, individual devices of the invention may include three or more electrodes that define a plurality of electroporation zones, thus allowing for a plurality of electroporations on the cells suspended in a fluid. Devices of the invention may include a plurality of electroporation zones between the first and second electrodes, allowing for cells to experience different electric fields, e.g., developed by different geometries of each of the plurality of electroporation zones, while flowing in a single device or a plurality of devices.

In some cases, the first electrode and the second electrode may be electrically conductive wires, hollow cylinders, electrically conductive thin films, metal foams, mesh electrodes, liquid diffusible membranes, conductive liquids, or any combination thereof can be included in the device. The electrodes may be either aligned parallel with the axis of fluid flow of the device or may be aligned orthogonal to the axis of fluid flow of the device. For example, the first and second electrodes may be hollow cylindrical electrodes arranged in parallel with the axis of fluid flow within the device, such as the in the device of FIGS. 1A-1C, such that fluid flows through the electrodes. In an alternative example, the first and/or second electrodes may be made of a porous conductor, e.g., a metal mesh, with pores that are aligned to the axis of fluid flow of the device. In an alternative example, the first and/or second electrodes may be a conductive fluid, e.g., liquid. In some cases, the first and second electrodes may be configured as a helical, e.g., a double helix, made of a solid conductor, e.g., a wire, around the electroporation zone. In this configuration, the cross-sectional dimension of the electroporation zone remains substantially uniform but the first and second electrodes change in position along the length of the electroporation zone. The first and second electrodes are in fluid communication with the electroporation zone but the electric field generated when an electrical potential difference is applied to the electrodes rotates as the cells suspended in the fluid travel through the device of the invention. In certain embodiments, the first and second electrodes are embedded into the device of the invention and have active area disposed at or near the fluidic connections to the electroporation zone such that the fluid carrying the cells in suspension contacts a portion of the electrode, with the electric field generated in the electroporation zone.

When configured to be hollow cylindrical electrodes, the diameter of the electrode may be from about 0.1 mm to about 5 mm, e.g., from about 0.1 mm to about 1 mm, from about 0.5 mm to about 1.5 mm, from about 1 mm to about 2 mm, from about 1.5 mm to about 2.5 mm, about 2 mm to about 3 mm, from about 2.5 mm to about 3.5 mm, about 3 mm to about 4 mm, from about 3.5 mm to about 4.5 mm, or about 4 mm to about 5 mm, e.g., about 0.1 mm, about 0.2 mm, about 0.3 mm, about 0.4 mm, about 0.5 mm, about 0.6 mm, about 0.7 mm, about 0.8 mm, about 0.9 mm, about 1 mm, about 1.1 mm, about 1.2 mm, about 1.3 mm, about 1.4 mm, about 1.5 mm, about 1.6 mm, about 1.7 mm, about 1.8 mm, about 1.9 mm, about 2 mm, about 2.1 mm, about 2.2 mm, about 2.3 mm, about 2.4 mm, about 2.5 mm, about 2.6 mm, about 2.7 mm, about 2.8 mm, about 2.9 mm, about 3 mm, about 3.1 mm, about 3.2 mm, about 3.3 mm, about 3.4 mm, about 3.5 mm, about 3.6 mm, about 3.7 mm, about 3.8 mm, about 3.9 mm, about 4 mm, about 4.1 mm, about 4.2 mm, about 4.3 mm, about 4.4 mm, about 4.5 mm, about 4.6 mm, about 4.7 mm, about 4.8 mm, about 4.9 mm, or about 5 mm. An exemplary electrode outer diameter is 1.3 mm, corresponding to a 16 gauge electrode.

In some embodiments, when a device of the invention is configured to include hollow cylindrical electrodes, a lumen of an electrode, e.g., the first or second electrode, may include a zone, e.g., an entry zone or a recovery zone, that is not subject to the electric field of the electroporation zone. As is shown in FIG. 1A, the entry zone may be the lumen of the first electrode directly before an entrance to the electroporation zone where the cells in the suspension that are to be electroporated along with a composition to be delivered into the cells are located. The recovery zone may be the lumen of the second electrode directly after an exit to the electroporation zone where the cells that have had a composition delivered are moved to such that the pores in the cell membranes can close, thus ensuring that the delivered composition remains inside the cell. In this configuration, as cells pass through the lumen of the first electrode and towards the lumen of the second electrode, the first electrode is energized and the second electrode is held at ground, creating the localized electric field in the electroporation zone, thus electroporating the cells that pass through the device.

The electroporation zone fluidically connects the first and second electrodes of devices of the invention, and when the electrodes are energized, experiences a localized electric field therebetween. The cross-sectional shape of the electroporation zone may be of any suitable shape that allows cells to pass through the electroporation zone and the electric field within the electroporation zone. The cross-sectional shape may be, e.g., circular, ellipsoidal, or polygonal, e.g., square, rectangular, triangular, n-gon (e.g., a regular or irregular polygon having 4, 5, 6, 7, 8, 9, 10, or more sides), star, parallelogram, trapezoidal, or irregular, e.g., oval, or curvilinear shape. In some cases, the electroporation zone is a channel that has a substantially uniform cross-section dimension along its length, e.g., the electroporation zone may have a circular cross-section, where the diameter is constant from the fluidic connection to the entry zone to the fluidic connection of the recovery zone. In this configuration, the resulting electric field is more uniform, thus allowing for a more predictable electric field exposure of cells suspended in a fluid. Alternatively, the cross-sectional dimension of the electroporation zone may be varied along is length. For example, the cross-sectional dimension of the electroporation zone may either increase or decrease along its length, or may have more than one dimension change along its length, e.g., the cross-sectional dimension, e.g., the diameter, may increase or decrease by at least 1%, 5%, 10%, 20%, 30%, 40%, 50%, 60%, 70%, 80%, 90%, or 100%, or at most 1%, 5%, 10%, 20%, 30%, 40%, 50%, 60%, 70%, 80%, 90%, or 100%. In this configuration, the electroporation zone may have a truncated conical cross-section, with the diameter increasing from the top aperture to the bottom aperture or decreasing from the top aperture to the bottom aperture. In some cases, devices of the invention may include a plurality of electroporation zones fluidically connected in series, with each electroporation zone having either a uniform or non-uniform cross-section and each may have a different cross-section shape. As a non-limiting example, a device of the invention may include a plurality of serially-connected electroporation zones, each of the plurality of electroporation zones having a cylindrical cross-section of a different cross-sectional dimension, e.g., each has a different diameter.

In some embodiments, the cross-sectional dimension of the electroporation zone may be from about 0.005 mm to about 50 mm, e.g., about 0.005 mm to about 0.05 mm, about 0.01 mm to about 0.1 mm, about 0.05 mm to about 0.5 mm, about 0.1 mm to about 1 mm, about 0.5 mm to about 1 mm, from about 0.5 mm to about 2 mm, about 0.7 mm to about 1.5 mm, about 1 mm to about 5 mm, about 3 mm to about 7 mm, about 5 mm to about 10 mm, about 7 mm to about 12 mm, about 10 mm to about 15 mm, about 13 mm to about 18 mm, about 15 mm to about 20 mm, about 22 mm to about 30 mm about 25 mm to about 35 mm, about 30 mm to about 40 mm, about 35 mm to about 45 mm, or about 40 mm to about 50 mm, e.g., about 0.005 mm, about 0.006, about 0.007 mm, about 0.008 mm, about 0.009 mm, about 0.01 mm, about 0.02 mm, about 0.03 mm, about 0.04 mm, about 0.05 mm, about 0.06 mm, about 0.07 mm, about 0.08 mm, about 0.09 mm, about 0.1 mm, about 0.2 mm, about 0.3 mm, about 0.4 mm, about 0.5 mm, about 0.6 mm, about 0.7 mm, about 0.8 mm, about 0.9 mm, about 1 mm, about 2 mm, about 3 mm, about 4 mm, about 5 mm, about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, about 20 mm, about 21 mm, about 22 mm, about 23 mm, about 24 mm, about 25 mm, about 26 mm, about 27 mm, about 28 mm, about 29 mm, about 30 mm, about 31 mm, about 32 mm, about 33 mm, about 34 mm, about 35 mm, about 36 mm, about 37 mm, about 38 mm, about 39 mm, about 40 mm, about 41 mm, about 42 mm, about 43 mm, about 44 mm, about 45 mm, about 46 mm, about 47 mm, about 48 mm, about 49 mm, or about 50 mm. In general, the diameter of the electroporation zone is sized such that it does not have a constriction that contacts the cells to deform the cell membranes with the channel walls, e.g., poration of the cells is not induced by mechanical deformation due to cell squeezing—e.g., the cells can freely pass through the electroporation zone.

In some cases, the length of the electroporation zone may be from about 0.005 mm to about 50 mm, e.g., about 0.005 mm to about 0.05 mm, about 0.01 mm to about 0.1 mm, about 0.05 mm to about 0.5 mm, about 0.1 mm to about 1 mm, from about 0.5 mm to about 2 mm, about 1 mm to about 5 mm, about 3 mm to about 7 mm, about 4 mm to about 8 mm, about 5 mm to about 10 mm, about 7 mm to about 12 mm, about 10 mm to about 15 mm, about 13 mm to about 18 mm, about 15 mm to about 20 mm, about 22 mm to about 30 mm about 25 mm to about 35 mm, about 30 mm to about 40 mm, about 35 mm to about 45 mm, or about 40 mm to about 50 mm, e.g., about 0.005 mm, about 0.006, about 0.007 mm, about 0.008 mm, about 0.009 mm, about 0.01 mm, about 0.02 mm, about 0.03 mm, about 0.04 mm, about 0.05 mm, about 0.06 mm, about 0.07 mm, about 0.08 mm, about 0.09 mm, about 0.1 mm, about 0.2 mm, about 0.3 mm, about 0.4 mm, about 0.5 mm, about 0.6 mm, about 0.7 mm, about 0.8 mm, about 0.9 mm, about 1 mm, about 2 mm, about 3 mm, about 4 mm, about 5 mm, about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, about 20 mm, about 21 mm, about 22 mm, about 23 mm, about 24 mm, about 25 mm, about 26 mm, about 27 mm, about 28 mm, about 29 mm, about 30 mm, about 31 mm, about 32 mm, about 33 mm, about 34 mm, about 35 mm, about 36 mm, about 37 mm, about 38 mm, about 39 mm, about 40 mm, about 41 mm, about 42 mm, about 43 mm, about 44 mm, about 45 mm, about 46 mm, about 47 mm, about 48 mm, about 49 mm, or about 50 mm.

The cross-sectional dimension of the entry zone and/or the recovery zone may be independently substantially the same as the cross-sectional dimension of the electroporation zone. Alternatively, the entry zone and/or the recovery zone may be independently smaller or larger than the cross-sectional dimension of the electroporation zone. For example, when the cross-sectional dimension of the entry zone and/or the recovery zone is independently configured to be smaller than the cross-sectional dimension of the electroporation zone, the cross-sectional dimension of the entry zone and/or the recovery zone may be from about 0.01% to about 100% of the cross-sectional dimension of the electroporation zone, about 0.01% to about 1%, about 0.1% to about 10%, about 1% to about 5%, about 1% to about 10%, about 5% to about 25%, about 5% to about 10%, about 10% to about 25%, about 10% to about 50%, about 25% to about 75%, or about 50% to about 100%, e.g., about 0.01%, about 0.02%, about 0.03%, about 0.04%, about 0.05%, about 0.06%, about 0.07%, about 0.08%, about 0.09%, about 0.1%, about 0.15%, about 0.2%, about 0.25%, about 0.3%, about 0.35%, about 0.4%, about 0.45%, about 0.5%, about 0.55%, about 0.6%, about 0.65%, about 0.7%, about 0.75%, about 0.8%, about 0.85%, about 0.9%, about 0.95%, about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

Alternatively, when the cross-sectional dimension of the entry zone and/or the recovery zone is independently configured to be larger than the cross-sectional dimension of the electroporation zone, the cross-sectional dimension of the entry zone and/or the recovery zone may be from about 100% to about 100,000% of the cross-sectional dimension of the electroporation zone, e.g., about 100% to about 1000%, about 100% to about 250%, about 100% to about 500%, about 250% to about 750%, about 500% to about 1,000%, about 500% to about 5,000%, about 1,000% to about 10,000%, about 5,000% to about 25,000%, about 10,000% to about 50,000%, about 25,000% to about 75,000%, or about 50,000% to about 100,000%, e.g., about 100%, about 150%, about 175%, about 200%, about 225%, about 250%, about 300%, about 250%, about 400%, about 450%, about 500%, about 600%, about 700%, about 800%, about 900%, about 1,000%, about 2,000%, about 3,000%, about 4,000%, about 5,000%, about 6,000%, about 7,000%, about 8,000%, about 9,000%, about 10,000%, about 15,000%, about 20,000%, about 25,000%, about 30,000%, about 35,000%, about 40,000%, about 45,000%, about 50,000%, about 55,000%, about 60,000%, about 65,000%, about 70,000%, about 75,000%, about 80,000%, about 85,000%, about 90,000%, about 95,000%, or about 100,000%.

Devices of the invention may also include one or more reservoirs for fluid reagents, e.g., a buffer solution, or samples, e.g., a suspension of cells and a composition to be introduced to the cells. For example, devices of the invention may include a reservoir for the cells suspended in the fluid to flow in the first electrode into the electroporation zone and/or a reservoir for holding the cells that have been electroporated. Similarly, a reservoir for liquids to flow in additional components of a device, such as additional inlets that intersect the first or second electrodes, may be present. A single reservoir may also be connected to multiple devices of the invention, e.g., when the same liquid is to be introduced at two or more individual device of the invention configured to electroporate cells in parallel or in series. Alternatively, devices of the invention may be configured to mate with sources of the liquids, which may be external reservoirs such as vials, tubes, or pouches. Similarly, the device may be configured to mate with a separate component that houses the reservoirs. Reservoirs may be of any appropriate size, e.g., to hold 10 mL to 5000 mL, e.g., 10 mL to 3000 mL, 25 mL to 100 mL, 100 mL to 1000 mL, 40 mL to 300 mL, 1 mL to 100 mL, 10 mL to 500 mL, 250 mL to 750 mL, 250 mL to 1000 mL, or 1000 mL to 5000 mL. When multiple reservoirs are present, each reservoir may have the same or a different size.

In addition to the components discussed above, devices of the invention may include additional components. For example, the first and second electrodes of the devices of the invention may include one or more additional fluid inlets to allow for the introduction of non-sample fluids, e.g., buffer solutions, into the appropriate region of the device. For example, a recovery zone of a device of the invention may include an additional inlet and outlet to circulate a recovery buffer to aid in the closing of the pores opened in the cell membranes from the electroporation process.

### Systems and Kits

One or more electroporation devices of the invention may be combined with various external components, e.g., power supplies, pumps, reservoirs (e.g., bags), controllers, reagents, liquids, and/or samples in the form of a system. In some embodiments, a system of the invention includes a plurality of devices of the invention and a source of electrical potential that is releasably connected to the first and second electrodes of the device(s) of the invention. In this configuration, the device(s) of the invention are connected to the source of electrical potential, and the first electrode is energized and the second electrode is held at ground. This creates a localized electric field in the electroporation zone, thus electroporating the cells that pass through the device(s). Electroporation systems incorporating devices of the invention may induce either reversible or irreversible electroporation to the cells that pass through the device and system of the invention. For example, devices and systems of the invention may induce substantially non-thermal reversible electroporation, substantially non-thermal irreversible electroporation, or substantially thermal irreversible electroporation on the cells suspended in the fluid.

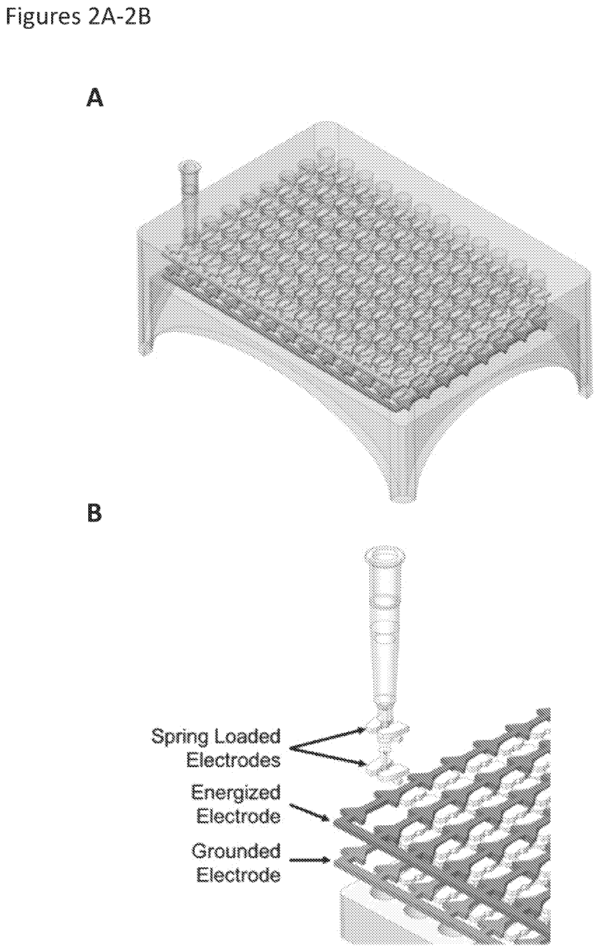

In some cases, the releasable connection to the first and second electrodes may include any practical electro-mechanical connection that can maintain consistent electrical contact between the source of electrical potential and the first and second electrodes. Example electrical connections include, but are not limited to clamps, clips, e.g., alligator clips, springs, e.g., a leaf spring, an external sheath or sleeve, wire brushes, flexible conductors, pogo pins, mechanical connections, inductive connections, or a combination thereof. Other types of electrical connections are known in the art. For example, a spring-type electrode can be integrated into a conductive platform such as that shown in FIGS. 2A-2B. In the embodiment shown in FIGS. 2A-2B, a device of the invention is inserted into a housing that incorporates two conducting grids electrically isolated from each other onto a base that contains individual openings for accepting devices of the invention. A device of the invention can be installed into an opening in the conducting grid such that the first and second electrodes of the device can contact the conducting grid. In particular, the conducting grid includes spring loaded electrodes, e.g., electrodes connected to a spring, such that when a device of the invention is installed into an opening of the conducting grid, the spring-loaded electrodes displace and compress the spring (which further provides a restoring force against the first and second electrodes of the device of the invention), thus ensuring electrical contact between the device of the invention and the source of electrical potential.

The source of electrical potential is configured to deliver an applied voltage to one or more electrode in order to provide an electrical potential difference between the electrodes and thus establish a uniform electric field in the electroporation zone. In some cases, such as in a two-electrode electroporation circuit, the applied voltage is delivered to a first electrode and the second electrode is held at ground. Without wishing to be bound by any particular theory, an applied voltage delivered to the electrode is delivered at a particular amplitude, a particular frequency, a particular pulse shape, a particular duration, a particular number of pulses applied, and a particular duty cycle. These parameters, coupled to the geometry of the electroporation zone, will deliver a particular electric field within the electroporation zone that will be experienced by the cells suspended in a fluid. The electrical parameters described herein may be optimized for a particular cell line and/or composition being delivered to a particular cell line. The application of the electrical potential to the electrodes of devices(s) of the invention may be initiated and/or controlled by a controller, e.g., a computer with programming, operatively coupled to the source of electrical potential.

Along with the electrical potential parameters described herein, the geometry of devices of the invention, e.g., the shape and dimensions of the cross-section of the electroporation zone, control the shape and intensity of the resulting electric field within the electroporation zone. Typically, a device with an electroporation zone that has a uniform cross section will exhibit a uniform electric field along its length. In order to modulate the resulting electric field in the electroporation zone, the electroporation zone may include a plurality of different cross-sectional dimensions and/or different cross-section shapes along its length. As a non-limiting example, a device of the invention may include a plurality of serially-connected electroporation zones, each of the plurality of electroporation zones having a circular cross-section of a different cross-sectional dimension, e.g., each has a different diameter. In this configuration, the different diameter circular cross-sections of the electroporation zone each act as an independent electroporation zone, and each will induce a different electric field at every change in dimension with an identical applied voltage, e.g., a constant DC voltage.

In some cases, devices of the invention may include a plurality of electroporation zones fluidically connected in series, with each electroporation zone having either a uniform or non-uniform cross-section and each may have a different cross-section shape. Alternatively, a system of the invention may include a plurality of devices of the invention in a parallel configuration, with each device operating independently of each other to increase the overall throughput of the electroporation.

In some cases, the amplitude of the applied voltage is from about −3 kV to 3 kV, e.g., about −3 kV to about −0.1 kV, about −2 kV to about −0.1 kV, about −1 kV to about −0.1 kV, about −0.1 kV to about −0.01 kV, 0.01 kV to about 3 kV, e.g., about 0.01 kV to about 0.1 kV, about 0.02 kV to about 0.2 kV, about 0.03 kV to about 0.3 kV, about 0.04 kV to about 0.4 kV, about 0.05 kV to about 0.5 kV, about 0.06 kV to about 0.6 kV, about 0.07 kV to about 0.7 kV, about 0.08 kV to about 0.8 kV, about 0.09 kV to about 0.9 kV, about 0.1 kV to about 1 kV, about 0.1 kV to about 2.0 kV, about 0.1 kV to about 3 kV, about 0.15 kV to about 1.5 kV, about 0.2 kV to about 2 kV, about 0.25 kV to about 2.5 kV, or about 0.3 kV to about 3 kV, e.g., about 0.01 to about 1 kV, about 0.1 kV to about 0.7 kV, or about 0.2 to about 0.6 kV, e.g., about 0.01 kV, about 0.02 kV, about 0.03 kV, about 0.04 kV, about 0.05 kV, about 0.06 kV, about 0.07 kV, about 0.08 kV, about 0.09 kV, about 0.1 kV, about 0.2 kV, about 0.3 kV, about 0.4 kV, about 0.5 kV, about 0.6 kV, about 0.7 kV, about 0.8 kV, about 0.9 kV, about 1 kV, about 1.1 kV, about 1.2 kV, about 1.3 kV, about 1.4 kV, about 1.5 kV, about 1.6 kV, about 1.7 kV, about 1.8 kV, about 1.9 kV, about 2 kV, about 2.1 kV, about 2.2 kV, about 2.3 kV, about 2.4 kV, about 2.5 kV, about 2.6 kV, about 2.7 kV, about 2.8 kV, about 2.9 kV, or about 3 kV.

In some cases, the frequency of the applied voltage is from about 1 Hz to about 50,000 Hz, e.g., from about 1 Hz to about 1,000 Hz, about 1 Hz to about 500 Hz, about 100 Hz to about 500 Hz, about 100 Hz to about 5,000 Hz, about 500 Hz to about 10,000 Hz, about 1000 Hz to about 25,000 Hz, or from about 5,000 Hz to about 50,000 Hz, e.g., from about 10 Hz to about 1000 Hz, about 10 Hz to about 500 Hz, about 500 Hz to about 750 Hz, or about 100 Hz to about 500 Hz, e.g., from about 1 Hz, about 2 Hz, about 3 Hz, about 4 Hz, about 5 Hz, about 6 Hz, about 7 Hz, about 8 Hz, about 9 Hz, about 10 Hz, about 20 Hz, about 30 Hz, about 40 Hz, about 50 Hz, about 60 Hz, about 70 Hz, about 80 Hz, about 90 Hz, about 100 Hz, about 110 Hz, about 120 Hz, about 130 Hz, about 140 Hz, about 150 Hz, about 160 Hz, about 170 Hz, about 180 Hz, about 190 Hz, about 200 Hz, about 210 Hz, about 220 Hz, about 230 Hz, about 240 Hz, about 250 Hz, about 260 Hz, about 270 Hz, about 270 Hz, about 280 Hz, about 290 Hz about 300 Hz, about 310 Hz, about 320 Hz, about 330 Hz, about 340 Hz, about 350 Hz, about 360 Hz, about 370 Hz, about 380 Hz, about 390 Hz, about 400 Hz, about 410 Hz, about 420 Hz, about 430 Hz, about 440 Hz, about 450 Hz, about 460 Hz, about 470 Hz, about 480 Hz, about 490 Hz, about 500 Hz, about 510 Hz, about 520 Hz, about 530 Hz, about 540 Hz, about 550 Hz, about 600 Hz, about 700 Hz, about 800 Hz, about 900 Hz, about 1,000 Hz, about 2,000 Hz, about 3,000 Hz, about 4,000 Hz, about 5,000 Hz, about 6,000 Hz, about 7,000 Hz, about 8,000 Hz, about 9,000 Hz, about 10,000 Hz, about 15,000 Hz, about 20,000 Hz, about 25,000 Hz, about 30,000 Hz, about 35,000 Hz, about 40,000 Hz, about 45,000 Hz, or about 50,000 Hz.

In some embodiments, the shape of the applied pulse, e.g., waveform, can be a square wave, pulse, a bipolar wave, a sine wave, a ramp, an asymmetric bipolar wave, or arbitrary. Other voltage waveforms are known in the art. The chosen waveform can be applied at any practical voltage pattern including, but not limited to, high voltage-low voltage, low voltage-high voltage, direct current (DC), alternating current (AC), unipolar, positive (+) polarity only, negative (−) polarity only, (+)/(−) polarity, (−)/(+) polarity, or any superposition or combination thereof. A skilled artisan can appreciate that these pulse parameters will depend on the cell line any electrical characteristics of the composition being delivered to the cell.

Applied voltage pulses can be delivered to the electroporation zone with durations from about 0.01 ms to about 1,000 ms, e.g., from about 0.01 ms to about 1 ms, about 0.1 ms to about 10 ms, about 0.1 ms to about 15 ms, about 1 ms to about 10 ms, about 1 ms to about 50 ms, about 10 ms to about 100 ms, about 25 ms to about 200 ms, about 50 ms to about 400 ms, about 100 ms to about 600 ms, about 300 ms to about 800 ms, or about 500 ms to about 1,000 ms, e.g., about 0.01 ms to 100 ms, about 0.1 ms to about 50 ms, or about 1 ms to about 10 ms, e.g., about 0.01 ms, about 0.02 ms, about 0.03 ms, about 0.04 ms, about 0.05 ms, about 0.06 ms, about 0.07 ms, about 0.08 ms, about 0.09 ms, about 0.1 ms, about 0.2 ms, about 0.3 ms, about 0.4 ms, about 0.5 ms, about 0.6 ms, about 0.7 ms, about 0.8 ms, about 0.9 ms, about 1 ms, about 2 ms, about 3 ms, about 4 ms, about 5 ms, about 6 ms, about 7 ms, about 8 ms, about 9 ms, about 10 ms, about 11 ms, about 12 ms, about 13 ms, about 14 ms, about 15 ms, about 20 ms, about 30 ms, about 40 ms, about 50 ms, about 60 ms, about 70 ms, about 80 ms, about 90 ms, about 100 ms, about 150 ms, about 200 ms, about 250 ms, about 300 ms, about 350 ms, about 400 ms, about 450 ms, about 500 ms, about 550 ms, about 600 ms, about 650 ms, about 700 ms, about 750 ms, about 800 ms, about 850 ms, about 900 ms, about 950 ms, or about 1,000 ms.

In some cases, the number of applied voltage pulses delivered can be from 0 to about 1000, or more, e.g., 1 or more, 2 or more, 3 or more, 4 or more, 5 or more, 6 or more, 7 or more, 8 or more, 9 or more, 10 or more, or 100 or more, e.g., 1-4, 2-5, 3-6, 4-7, 5-8, 6-9, 7-10, 8-11, 7-12, or 9-13, e.g., about 0.01 to about 1,000, e.g., from about 1 to about 10, about 1 to about 50, about 5 to about 10, about 5 to about 15, about 10 to about 100, about 25 to about 200, about 50 to about 400, about 100 to about 600, about 300 to about 800, or about 500 to about 1,000, e.g., about 1 to 100, about 1 to about 50, or about 1 to about 10, e.g., about 1, about 2, about 3, about 4, about 5, about 6, about 7, about 8, about 9, about 10, about 20, about 30, about 40, about 50, about 60, about 70, about 80, about 90, about 100, about 150, about 200, about 250, about 300, about 350, about 400, about 450, about 500, about 550, about 600, about 650, about 700, about 750, about 800, about 850, about 900, about 950, or about 1,000.

In some instances, the number of applied voltage pulses delivered can be from 1 to about 1,000,000. For example, in some instances, the number of applied voltage pulses delivered is from 1,000 to 1,000,000, e.g., from 1,000 to 10,000 (e.g., from 1,000 to 2,000, from 2,000 to 3,000, from 3,000 to 4,000, from 4,000 to 5,000, from 5,000 to 6,000, from 6,000 to 7,000, from 7,000 to 8,000, from 8,000 to 9,000, or from 9,000 to 10,000, e.g., about 1,000, about 2,000, about 3,000, about 4,000, about 5,000, about 6,000, about 7,000, about 8,000, about 9,000, or about 10,000), from 10,000 to 100,000 (e.g., from 10,000 to 20,000, from 20,000 to 30,000, from 30,000 to 40,000, from 40,000 to 50,000, from 50,000 to 60,000, from 60,000 to 70,000, from 70,000 to 80,000, from 80,000 to 90,000, or from 90,000 to 100,000, e.g., about 10,000, about 25,000, about 30,000, about 40,000, about 50,000, about 60,000, about 70,000, about 75,000, about 80,000, about 90,000, or about 100,000), or from 100,000 to 1,000,000 (e.g., from 100,000 to 200,000, from 200,000 to 300,000, from 300,000 to 400,000, from 400,000 to 500,000, from 500,000 to 600,000, from 600,000 to 700,000, from 700,000 to 800,000, from 800,000 to 900,000, or from 900,000 to 1,000,000, e.g., about 100,000, about 200,000, about 250,000, about 300,000, about 400,000, about 500,000, about 600,000, about 700,000, about 750,000, about 800,000, about 900,000, or about 1,000,000).

The pulses of applied voltage can, in some instances, be delivered at a duty cycle of about 0.001% to about 100%, e.g., from about 0.001% to about 0.1%, about 0.01% to about 1%, about 0.1% to about 5%, about 1% to about 10%, about 2.5% to about 20%, about 5% to about 40%, about 10% to about 60%, about 30% to about 80%, or about 50% to about 100%, e.g., about 0.01% to 100%, about 0.1% to about 99%, about 1% to about 97%, or about 10% to about 95%, e.g., about 0.001%, about 0.002%, about 0.003%, about 0.004%, about 0.005%, about 0.006%, about 0.007%, about 0.008%, about 0.009%, about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

Device(s) of the invention, when the electrodes are connected to the source of electrical potential and energized, generate a localized electric field in the electroporation zone that electroporate cells that pass through. In some cases, the electric field generated in the electroporation zone has a magnitude from about 2 V/cm to about 50,000 V/cm, e.g., about 2 V/cm to about 1,000 V/cm, about 100 V/cm to about 1,000 V/cm, about 100 V/cm to about 5,000 V/cm, about 500 V/cm to about 10,000 V/cm, about 1000 V/cm to about 25,000 V/cm, or from about 5,000 V/cm to about 50,000 V/cm, e.g., from about 2 V/cm to about 20,000 V/cm, about 5 V/cm to about 10,000 V/cm, or about 100 V/cm to about 1,000 V/cm, e.g., from about 2 V/cm, about 3 V/cm, about 4 V/cm, about 5 V/cm, about 6 V/cm, about 7 V/cm, about 8 V/cm, about 9 V/cm, about 10 V/cm, about 20 V/cm, about 30 V/cm, about 40 V/cm, about 50 V/cm, about 60 V/cm, about 70 V/cm, about 80 V/cm, about 90 V/cm, about 100 V/cm, about 200 V/cm, about 300 V/cm, about 400 V/cm, about 500 V/cm, about 600 V/cm, about 700 V/cm, about 800 V/cm, about 900 V/cm, about 1,000 V/cm, about 2,000 V/cm, about 3,000 V/cm, about 4,000 V/cm, about 5,000 V/cm, about 6,000 V/cm, about 7,000 V/cm, about 8,000 V/cm, about 9,000 V/cm, about 10,000 V/cm, about 15,000 V/cm, about 20,000 V/cm, about 25,000 V/cm, about 30,000 V/cm, about 35,000 V/cm, about 40,000 V/cm, about 45,000 V/cm, or about 50,000 V/cm.

Systems of the invention typically include a fluid delivery source that is configured to deliver the plurality of cells suspended in the fluid through the first electrode, e.g., the entry zone, to the second electrode, e.g., the recovery zone. Fluid delivery sources typically includes pumps, including, but not limited to, syringe pumps, micropumps, or peristaltic pumps. Alternatively, fluids can be delivered by the displacement of a working fluid against a reservoir of the fluid to be delivered or by air displacement. Other fluid delivery sources are known in the art. In some cases, the fluid delivery source is configured to flow cells suspended in a fluid by the application of a positive pressure. Without wishing to be bound by any particular theory, the flow rate at which cells in a suspension are flowed through devices of the invention and the specific geometry of the electroporation zone of devices of the invention will determine the residence time of the cells in the electric field in the electroporation zone.

In some instances, the volumetric flow rate of fluid delivered from a fluid delivery source has a volumetric flow rate of about 0.001 mL/min to about 1,000 mL/min, e.g., from about 0.001 mL/min to about 0.1 mL/min, about 0.01 mL/min to about 1 mL/min, about 0.1 mL/min to about 10 mL/min, about 1 mL/min to about 50 mL/min, about 10 mL/min to about 100 mL/min, about 25 mL/min to about 200 mL/min, about 50 mL/min to about 400 mL/min, about 100 mL/min to about 600 mL/min, about 300 mL/min to about 800 mL/min, or about 500 mL/min to about 1,000 mL/min, e.g., about 0.001 mL/min, about 0.002 mL/min, about 0.003 mL/min, about 0.004 mL/min, about 0.005 mL/min, about 0.006 mL/min, about 0.007 mL/min, about 0.008 mL/min, about 0.009 mL/min, about 0.01 mL/min, about 0.02 mL/min, about 0.03 mL/min, about 0.04 mL/min, about 0.05 mL/min, about 0.06 mL/min, about 0.07 mL/min, about 0.08 mL/min, about 0.09 mL/min, about 0.1 mL/min, about 0.2 mL/min, about 0.3 mL/min, about 0.4 mL/min, about 0.5 mL/min, about 0.6 mL/min, about 0.7 mL/min, about 0.8 mL/min, about 0.9 mL/min, about 1 mL/min, about 2 mL/min, about 3 mL/min, about 4 mL/min, about 5 mL/min, about 6 mL/min, about 7 mL/min, about 8 mL/min, about 9 mL/min, about 10 mL/min, about 15 mL/min, about 20 mL/min, about 25 mL/min, about 30 mL/min, about 35 mL/min, about 40 mL/min, about 45 mL/min, about 50 mL/min, about 55 mL/min, about 60 mL/min, about 65 mL/min, about 70 mL/min, about 75 mL/min, about 80 mL/min, about 85 mL/min, about 90 mL/min, about 95 mL/min, about 100 mL/min, about 150 mL/min, about 200 mL/min, about 250 mL/min, about 300 mL/min, about 350 mL/min, about 400 mL/min, about 450 mL/min, about 500 mL/min, about 550 mL/min, about 600 mL/min, about 650 mL/min, about 700 mL/min, about 750 mL/min, about 800 mL/min, about 850 mL/min, about 900 mL/min, about 950 mL/min, or about 1,000 mL/min. In particular embodiments, the flow rate is from 10 mL/min to about 100 mL/min, e.g., about 10 mL/min, 20 mL/min, 30 mL/min, 40 mL/min, 50 mL/min, 60 mL/min, 70 mL/min, 80 mL/min, 90 mL/min, or 100 mL/min.

In some instances, a Reynolds number of a liquid and/or the plurality of cells in suspension delivered from a fluid delivery source from the first lumen to the electroporation zone is between 0.04 and 2.43×10⁴, wherein the fluid delivery source is configured to deliver the liquid and/or the plurality of cells in suspension through the first lumen to the second outlet. In some instances, a maximum velocity of a liquid and/or the plurality of cells in suspension delivered from a fluid delivery source from the first lumen to the electroporation zone is between 5×10⁻⁵ m/s and 32.7 m/s, wherein the fluid delivery source is configured to deliver the liquid and/or the plurality of cells in suspension through the first lumen to the second outlet. In some instances, shear rates of a liquid and/or the plurality of cells in suspension delivered from a fluid delivery source from the first lumen to the electroporation zone are between 0.1 1/s and 2×10⁶ 1/s, wherein the fluid delivery source is configured to deliver the liquid and/or the plurality of cells in suspension through the first lumen to the second outlet. In some instances, a peak pressure of a liquid and/or the plurality of cells in suspension delivered from a fluid delivery source from the first lumen to the electroporation zone is between 1×10⁻³ Pa and 9.5×10⁴ Pa, wherein the fluid delivery source is configured to deliver the liquid and/or the plurality of cells in suspension through the first lumen to the second outlet. In some instances, an average velocity of a liquid and/or the plurality of cells in suspension delivered from a fluid delivery source from the first lumen to the electroporation zone is between 1.5×10⁻⁵ m/s and 15.9 m/s, wherein the fluid delivery source is configured to deliver the liquid and/or the plurality of cells in suspension through the first lumen to the second outlet. In some instances, a kinematic viscosity of a liquid and/or the plurality of cells in suspension delivered from a fluid delivery source from the first lumen to the electroporation zone is between 1×10⁻⁶ m²/s and 15×10⁻⁴ m²/s, wherein the fluid delivery source is configured to deliver the liquid and/or the plurality of cells in suspension through the first lumen to the second outlet.

The residence time of cells in the electroporation zone of devices of the invention may be from about 0.5 ms to about 50 ms, e.g., from about 0.5 ms to about 5 ms, about 1 ms to about 10 ms, about 5 ms to about 15 ms, about 10 ms to about 20 ms, about 15 ms to about 25 ms, about 20 ms to about 30 ms, about 25 ms to about 35 ms, about 30 ms to about 40 ms, about 35 ms to about 45 ms, or about 40 ms to about 50 ms, e.g., about 0.5 ms, about 0.6 ms, about 0.7 ms, about 0.8 ms, about 0.9 ms, about 1 ms, about 1.5 ms, about 2 ms, about 2.5 ms, about 3 ms, about 3.5 ms, about 4 ms, about 4.5 ms, about 5 ms, about 5.5 ms, about 6 ms, about 6.5 ms, about 7 ms, about 7.5 ms, about 8 ms, about 8.5 ms, about 9 ms, about 9.5 ms, about 10 ms, about 10.5 ms, about 11 ms, about 11.5 ms, about 12 ms, about 12.5 ms, about 13 ms, about 13.5 ms, about 14 ms, about 14.5 ms, about 15 ms, about 20 ms, about 25 ms, about 30 ms, about 35 ms, about 40 ms, about 45 ms, or about 50 ms. In some embodiments, the residence time is from 5-20 ms (e.g., from 6-18 ms, 8-15 ms, or 5-14 ms).

Systems of the invention typically feature a housing that contains and supports the device(s) of the invention and any necessary electrical connections, e.g., electrode connections. The housing may be configured to hold and energize a single device of the invention, or alternatively, may be configured to hold and simultaneously energize a plurality of devices of the invention. For example, in the embodiment of a system of the invention shown in FIGS. 2A-2B, the housing is configured as a rack that can accept and simultaneously energize 96 individual devices of the invention operating in parallel. The housing may include a thermal controller that is able to regulate the temperature of the devices of the invention or thermally regulate a component of the system, e.g., a fluid, e.g., a buffer or suspension containing cells, during electroporation. The thermal controller may be configured to heat the devices of the invention, or a component of a system thereof, cool the devices of the invention, or a component of a system thereof, or perform both operations. When configured to heat the devices of the invention, or a component of a system thereof, suitable thermal controllers include, but are not limited to, heating blocks or mantles, liquid heating, e.g., immersion or circulating fluid baths, battery operated heaters, or resistive heaters, e.g., thin film heaters, e.g., heat tape. When configured to cool the devices of the invention, or a component of a system thereof, suitable thermal controllers include, but are not limited to, liquid cooling, e.g., immersion or circulating fluid baths, evaporative coolers, or thermoelectric, e.g., Peltier coolers. For example, when implemented with liquid cooling, a device of the invention or a housing configured to hold devices of the invention may be in direct contact with tubing that circulates a chilled fluid or surrounded in a cooling jacket including tubing that circulates a chilled fluid. Other heating and cooling elements are known in the art.

In some embodiments, the housing (e.g., cartridge) is configured for use with and/or insertion into an automated closed system that is used to deliver cell therapies to patients in a clinical or hospital setting.

In some embodiments, the housing (e.g., cartridge) further includes a cooling/heating area/enclosure for cell suspension and/or buffer storage during, before and after electroporation of the specimen. In some embodiments, the system (e.g., device and housing) is externally powered.

In some embodiments, devices of the invention include a touchscreen user interface or other alternative user interface(s) that enables the user to select parameters such as flow rate, waveforms, applied potential, volume to transfect, time delay, cooling features, heating features, electroporation or transfection status, progress and other parameters used to optimize the electroporation or electro-mechanical protocol. In some embodiments, the user interface also contains pre-formulated parameter selections that enable the user to operate the system at specific parameters and conditions that have previously been validated by user or as recommended by the manufacturers. In some embodiments, the user interface may be connected to programming that allows for automated running of the system and/or running an algorithm to optimize electroporation for a given sample of a known cell type. In some embodiments, the optimization algorithms have the ability to adjust electro-mechanical parameters independently or autonomously if the user selects this functionality. In some embodiments, the optimization algorithms allow for continuous adjustment of the parameters used in the electroporation or electro-mechanical process that may depend on the cell type, conductivity of cell suspensions, volume of cell suspensions, viscosity, lifetime of the electroporating cartridge(s), the physical state of the suspension, or the state of the transfection device(s).

In some embodiments, the optimization algorithms have the ability to perform predictive analysis based on known input cell-type parameters and to adjust electro-mechanical parameters accordingly. In some embodiments, the optimization algorithms adjust electro-mechanical parameters based on electrical signals within any of the devices of the invention. In some embodiments, the optimization algorithms adjust electro-mechanical parameters based on detected flow parameters within any of the devices of the invention. In some embodiments, the optimization algorithms adjust electroporation parameters based on unique dimensionless input parameters. In some embodiments, the optimization algorithms have the ability to adjust electroporation parameters based on unique multivariate combinations of parameters that are predictive of high viability results, high efficiency results, or matched viability and efficiency results.

Systems of the invention may include one or more outer structures that are configured to cover the electrodes of one or more devices of the invention, e.g., to reduce end user exposure to live electrical connections. Typically, a device of the invention (e.g., a FLOWFECT™ device) will include one outer structure that covers its electrodes and electroporation zone. The outer structure may be a non-conductive material, e.g., a non-conductive polymer, that includes structural features for electro-mechanically engaging the parts of the device, e.g., the electrodes or electroporation zone. The outer structure may include one or more recesses, cutouts, or similar openings within the structure to accommodate the device. The outer structure may be configured to be a component that can be removed from the device. For example, the outer structure may include two separate components connected by a hinge, e.g., a living hinge, such that it can be folded over the device of the invention. Alternatively, the outer structure may be one or more separate pieces that can be connected together using suitable mating features to form a single structure. In these embodiments, the outer structure may be affixed to the device of the invention using any suitable fastener, e.g., snaps, latches, button, or clips, which may be integrated into the outer structure or externally connected to the outer structure. Other suitable fastener types are known in the art. In some embodiments, the outer structure includes one or more alignment features, e.g., pins, divots, grooves, or tabs, that ensure correct alignment of the one or more pieces of the outer structure. In some cases, the outer structure is configured to be permanently connected to the devices of the invention.

In some embodiments, the housing (e.g., cartridge, e.g., outer structure) encapsulates one or more of the previously stated inventions or one or more electroporating devices used for continuous flow electroporation. In some embodiments, the housing (e.g., cartridge) is configured to allow use with and/or insertion into an automated closed system that delivers cell therapies to patients. In some embodiments, the housing further includes a cooling/heating area/enclosure for cell suspension and/or buffer storage during, before and after electroporation of the specimen. In some embodiments, the system (e.g., one or more devices and housing) is externally powered.

In some embodiments, the system also includes optimization algorithms that have the ability to adjust electro-mechanical parameters independently or autonomously if the user selects this functionality. These optimization algorithms allow for continuous adjustment of the parameters used in the electroporation process that may depend on the cell type, conductivity, volume of suspensions, viscosity, lifetime of the electroporating cartridge, the physical state of the suspension or the state of the electroporation device.

In any of the embodiments of the outer structure described herein, the outer structure provides for electrical connection between an external source of electric potential and the electrodes of the devices of the invention. For example, the outer structure may include one or more electrical inputs for electrical connections, e.g., spades, banana plugs, or bayonet, e.g., BNC, connectors, that facilitate electrical connection between the source of electric potential and the electrodes of the devices of the invention inside the outer structure.

Devices and outer structures of the invention may be combined with additional external components, such as reagents, e.g., buffers, e.g., transfection or recovery buffers, and/or samples, in a kit. In some instances, a transfection buffer includes a composition appropriate for cell electroporation. In some instances, the transfection buffer includes a suitable concentration of one or more salts (e.g., potassium chloride, sodium chloride, potassium phosphate, potassium dihydrogen phosphate) or sugars (e.g., dextrose or myo-inositol), or any combination thereof, at a concentration from 0.1 to 200 mM (e.g., from 0.1 to 1.0 mM, from 1.0 mM to 10 mM, or from 10 mM to 100 mM).

### Buffers and Media

Devices and systems of the invention may be used with transfection buffer or with cell culture growth medium that contains additives to support transfection. Certain additives may be added to control the conductivity of transfection buffer and/or cell culture growth medium used, including KCl, MgCl₂, NaCl, glucose, Na₂HPO₄, NaH₂PO₄, Ca(NO₃)₂, mannitol, succinate, dextrose, hydroxyethyl piperazineethanesulfonic acid (HEPES), trehalose, CaCl₂), dimethyl sulfoxide (DMSO), K₂HPO₄, KH₂PO₄, ethylene-bis(oxyethylenenitrilo)tetraacetic acid (EGTA), KOH, NaOH, K₂SO₄, Na₂SO₄, histidine buffer, citrate buffer, phosphate-buffered saline (PBS), ATP-disodium salt, and NaHCO₃. Certain additives may be added to control the viscosity of transfection buffer and/or cell culture growth medium used, including Ficoll, dextran, polyethylene glycol (PEG), methylcellulose (MethoCel), collagen I, and Matrigel.

### Methods

The invention features methods of introducing a composition, e.g., transfection, into at least a portion of a plurality of cells suspended in a fluid, using the electroporation devices described herein. The methods described herein may be used to greatly increase the throughput of the delivery of compositions into cell types, often considered to be a bottleneck in the research fields of genetic engineering and therapeutic fields of gene-modified cell therapies. In particular, the methods described herein have significantly increased number of recovered cells, transfection efficiency and cell viability after transfection with applications to more cell types than typical methods of transfection, e.g., lentiviral transfection, or commercially available cell transfection instruments, e.g., the NEON® Transfection System (Thermo Fisher, Carlsbad, Calif.) or the 4D-NUCLEOFECTOR (Lonza, Switzerland).

A composition is introduced into at least a portion of a plurality of cells suspended in a fluid by passing the fluid with the suspended cells, also containing the composition to be introduced into the cells, through a device of the invention, e.g., an electroporation device, as described herein. The composition and the cells suspended in the fluid can be delivered through the device of the invention by the application of a positive pressure, e.g., from a pump connected to a source of fluid, e.g., a peristaltic pump, a digital pipette, or automated liquid handling source. The composition and the cells suspended in the fluid pass from the first electrode, e.g., including and entry zone, to an electroporation zone fluidically connected to the first electrode, and then to the recovery zone, which is fluidically connected to electroporation zone. As the composition and cells suspended in the fluid flow through the first electrode to the electroporation zone, a potential difference is applied to the first and second electrodes, producing and thus exposing the cells to an electric field in the electroporation zone. The exposure of the cells to the generated electric field enhances temporary permeability of the plurality of cells, thus introducing the composition into at least a portion of the plurality of cells.

In some instances of the methods, the phenotype of the cells may or may not be altered relative to a baseline measurement of cell phenotype upon exiting the electroporation zone of devices of the invention. In some cases, the phenotype of the cells is altered from 0% to about 25% relative to a baseline measurement of cell phenotype upon exiting the electroporation zone of devices of the invention, e.g., from about 0% to about 2.5%, from about 1% to about 5%, from about 1% to about 10%, from about 5% to about 15%, from about 10% to about 20%, from about 15% to about 25%, or from about 20% to about 25%, e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, or about 25%. In particular instances, the plurality of cells has no phenotypic change upon exiting the electroporation zone. For example, a baseline or control measurement to establish the cell phenotype may be the measurement of the expression of a cell surface marker on cells that have not been transfected using devices of the invention. A corresponding identical measurement of the expression of the same cell marker on cells that have been transfected using devices of the invention can be used to assess changes in cell phenotype. The cell phenotype is assessed via flow cytometry analysis of cell surface marker expression to ensure that the cell phenotype is minimally changed or unchanged after electroporation. Examples of the cell surface markers to evaluate include, but are not limited to, CD3, CD4, CD8, CD19, CD45RA, CD45RO, CD28, CD44, CD69, CD80, CD86, CD206, IL-2 receptor, CTLA4, OX40, PD-1, and TIM3. Cell morphology is assessed using bright field or fluorescent microscopy to confirm lack of phenotypic changes after electroporation.

In some instances, the after introduction of the composition into at least a portion of the plurality of cells, the plurality of cells are stored in a recovery buffer. The recovery buffer is configured to promote the final closing of the pores that were formed in the plurality of cells. Recovery buffers typically include cell culture media that may include other ingredients for cell nourishment and growth, e.g., serum, minerals, etc. A skilled artisan can appreciate that the choice of recovery buffer will depend on the cell type undergoing electroporation.

In some embodiments of the method described herein, the volume of fluid with the suspended cells (e.g., displacement volume) and the composition to be introduced to the cells that are flowed through the electroporation zone of devices of the invention may be from about 0.001 mL to about 2000 mL, about 0.001 mL to about 1000 mL, e.g., 0.001 mL to about 1000 mL, e.g., from about 0.001 mL to about 0.1 mL, about 0.01 mL to about 1 mL, about 0.01 mL to about 750 mL, about 0.01 mL to about 1500 mL, about 0.1 mL to about 5 mL, about 0.1 mL to 500 mL, about 0.1 mL to about 2000 mL, about 1 mL to about 10 mL, about 1 mL to about 1000 mL, about 2 mL to about 2000 mL, about 2.5 mL to about 20 mL, about 5 mL to about 40 mL, about 10 mL to about 60 mL, about 10 mL to 1000 mL, about 20 mL to about 2000 mL, about 30 mL to about 80 mL, about 50 mL to about 200 mL, about 100 mL to about 500 mL, or 250 mL to about 750 mL, about 500 mL to about 1000 mL, about 500 mL to 2000 mL, about 750 mL to 1500 mL, or about 1000 mL to 2000 mL, e.g., about 0.01 mL to 100 mL, about 0.1 mL to about 99 mL, about 1 mL to about 97 mL, or about 10 mL to about 95 mL, e.g., about 0.0025 mL to about 10 mL, about 0.01 mL to about 1 mL, or about 0.025 mL to about 0.1 mL, e.g., about 0.001 mL, about 0.0025 mL, about 0.005 mL, about 0.0075 mL, about 0.01 mL, about 0.025 mL, about 0.05 mL, about 0.075 mL, about 0.1 mL, about 0.25 mL, about 0.5 mL, about 0.75 mL, about 1 mL, about 2 mL, about 3 mL, about 4 mL, about 5 mL, about 6 mL, about 7 mL, about 8 mL, about 9 mL, about 10 mL, about 15 mL, about 20 mL, about 25 mL, about 30 mL, about 35 mL, about 40 mL, about 45 mL, about 50 mL, about 55 mL, about 60 mL, about 65 mL, about 70 mL, about 75 mL, about 80 mL, about 85 mL, about 90 mL, about 95 mL, about 100 mL, about 150 mL, about 200 mL, about 250 mL, about 300 mL, about 350 mL, about 400 mL, about 450 mL, about 500 mL, about 550 mL, about 600 mL, about 650 mL, about 700 mL, about 750 mL, about 800 mL, about 850 mL, about 900 mL, about 950 mL, about 1000 mL, about 1050 mL, about 1100 mL, about 1150 mL, about 1200 mL, about 1250 mL, about 1300 mL, about 1350 mL, about 1400 mL, about 1450 mL, about 1500 mL, about 1550 mL, about 1600 mL, about 1650 mL, about 1700 mL, about 1750 mL, about 1800 mL, about 1850 mL, about 1900 mL, about 1950 mL, or about 2000 mL.

In some embodiments, the volume of fluid that is flowed through the electroporation zone of devices of the invention (e.g., displacement volume), displacement rate, or other controlled parameters may or may not affect the transfection efficiency of a plurality of cells. In some embodiments, the devices of the invention are configured for use with an automated fluid handling platform that can process a plurality of cells in volumes less than 100 μL that can accumulate to volumes ranging from more than 100 μL to about 2000 mL. In some embodiments, the automated fluid handling platform is configured for use with one or more fluid delivery sources (e.g., pumps, e.g., syringe pumps, micropumps, or peristaltic pumps) that deliver the volume of fluid that is flowed through the electroporation zone of devices of the invention. In some embodiments, the volume of fluid that is flowed through the electroporation zone of devices of the invention can be delivered by the displacement of a working fluid against a reservoir of the fluid to be delivered or by air displacement. In some embodiments, the fluid delivery source is configured to flow cells suspended in a fluid by the application of a positive pressure.

In certain aspects, the electrical conductivity of the fluid where the cells are suspended can affect the electroporation of, and thus the delivery of a composition to, the cells in the suspension. The conductivity of the fluid with the suspended cells may be from about 0.001 mS to about 500 mS, e.g., from about 0.001 mS to about 0.1 mS, about 0.01 mS to about 1 mS, about 0.1 mS to about 10 mS, about 1 mS to about 50 mS, about 10 mS to about 100 mS, about 25 mS to about 200 mS, about 50 mS to about 400 mS, or about 100 mS to about 500 mS, e.g., about 0.01 mS to about 100 mS, about 0.1 mS to about 50 mS, or about 1 to 20 mS, e.g., about 0.001 mS, about 0.002 mS, about 0.003 mS, about 0.004 mS, about 0.005 mS, about 0.006 mS, about 0.007 mS, about 0.008 mS, about 0.009 mS, about 0.01 mS, about 0.02 mS, about 0.03 mS, about 0.04 mS, about 0.05 mS, about 0.06 mS, about 0.07 mS, about 0.08 mS, about 0.09 mS, about 0.1 mS, about 0.2 mS, about 0.3 mS, about 0.4 mS, about 0.5 mS, about 0.6 mS, about 0.7 mS, about 0.8 mS, about 0.9 mS, about 1 mS, about 2 mS, about 3 mS, about 4 mS, about 5 mS, about 6 mS, about 7 mS, about 8 mS, about 9 mS, about 10 mS, about 15 mS, about 20 mS, about 25 mS, about 30 mS, about 35 mS, about 40 mS, about 45 mS, about 50 mS, about 55 mS, about 60 mS, about 65 mS, about 70 mS, about 75 mS, about 80 mS, about 85 mS, about 90 mS, about 95 mS, about 100 mS, about 150 mS, about 200 mS, about 250 mS, about 300 mS, about 350 mS, about 400 mS, about 450 mS, or about 500 mS.

Methods of the invention can deliver compositions to a variety of cell types including, but not limited to, mammalian cells, eukaryotes, prokaryotes, synthetic cells, human cells, animal cells, plant cells, primary cells, cell lines, suspension cells, adherent cells, unstimulated cells, stimulated cells, or activated cells immune cells, stem cells (e.g., primary human induced pluripotent stem cells, e.g., iPSCs, embryonic stem cells, e.g., ESCs, mesenchymal stem cells, e.g., MSCs, or hematopoietic stem cells, e.g., HSCs), blood cells (e.g., red blood cells), T cells (e.g., primary human T cells), B cells, antigen presenting cells (APCs), natural killer (NK) cells (e.g., primary human NK cells), monocytes (e.g., primary human monocytes), macrophages (e.g., primary human macrophages), and peripheral blood mononuclear cells (PBMCs), neutrophils, dendritic cells, human embryonic kidney (e.g., HEK-293) cells, or Chinese hamster ovary (e.g., CHO-K1) cells. Typical cell numbers that can be electroporated may be from about 10⁴ cells to about 10¹² cells, (e.g., about 10⁴ cells to about 10⁵ cells, about 10⁴ cells to about 10⁶ cells, about 10⁴ cells to about 10⁷ cells, about 5×10⁴ cells to about 5×10⁵ cells, about 10⁵ cells to about 10⁶ cells, about 10⁵ cells to about 10⁷ cells, about 2.5×10⁵ cells to about 10⁶ cells, about 5×10⁵ cells to about 5×10⁶ cells, about 10⁶ cells to about 10⁷ cells, about 10⁶ cells to about 10⁸ cells, about 10⁶ cells to about 10¹² cells, about 5×10⁶ cells to about 5×10⁷ cells, about 10⁷ cells to about 10⁸ cells, about 10⁷ cells to about 10⁹ cells, about 10⁷ cells to about 10¹² cells, about 5×10⁷ cells to about 5×10⁸ cells, about 10⁸ cells to about 10⁹ cells, about 10⁸ cells to about 10¹⁰ cells, about 10⁸ cells to about 10¹² cells, about 5×10⁸ cells to about 5×10⁹ cells, about 10⁹ cells to about 10¹⁰ cells, about 10⁹ cells to about 10¹¹ cells, about 10¹⁰ cells to about 10¹¹ cells, about 10¹⁰ cells to about 10¹² cells, or about 10¹¹ cells to about 10¹² cells, e.g., about 10⁴ cells, about 2.5×10⁴ cells, about 5×10⁴ cells, about 10⁵ cells, about 2.5×10⁵ cells, about 5×10⁵ cells, about 10⁶ cells, about 2.5×10⁶ cells, about 5×10⁶ cells, about 10⁷ cells, about 2.5×10⁷ cells, about 5×10⁷ cells, about 10⁸ cells, about 2.5×10⁸ cells, about 5×10⁸ cells, about 10⁹ cells, about 2.5×10⁹ cells, about 5×10⁹ cells, about 10¹⁰ cells, about 5×10¹⁰ cells, about 10¹¹ cells, or about 10¹² cells).

Cell concentrations, i.e., number of cells per mL of fluid, for achieving cell poration numbers of about 10⁴ cells to about 10¹² cells typically ranges from about 10³ cells/mL to about 10¹¹ cells/mL, e.g., about 10³ cells/mL to about 10⁴ cells/mL, about 5×10³ cells/mL to about 5×10⁴ cells/mL, about 10⁵ cells/mL to about 10⁵ cells/mL, about 5×10⁵ cells/mL to about 5×10⁶ cells/mL, about 10⁶ cells/mL to about 10⁷ cells/mL, about 5×10⁶ cells/mL to about 5×10⁷ cells/mL, about 10⁷ cells/mL to about 10⁸ cells/mL, about 5×10⁷ cells/mL to about 5×10⁸ cells/mL, about 10⁸ cells/mL to about 10⁹ cells/mL, about 5×10⁸ cells/mL to about 5×10⁹ cells/mL, about 10⁹ cells/mL to about 10⁹ cells/mL, about 5×10⁹ cells/mL to about 5×10¹⁰ cells/mL, or about 10¹⁰ cells/mL to about 10¹¹ cells/mL, e.g., about 10³ cells/mL, about 5×10³ cells/mL, about 10⁴ cells/mL, about 5×10⁴ cells/mL, about 10⁵ cells/mL, about 5×10⁵ cells/mL, about 10⁶ cells/mL, about 5×10⁶ cells/mL, about 10⁷ cells/mL, about 5×10⁷ cells/mL, about 10⁸ cells/mL, about 5×10⁸ cells/mL, about 10⁹ cells/mL, about 5×10⁹ cells/mL, about 10¹⁰ cells/mL, about 5×10¹⁰ cells/mL, or about 10¹¹ cells/mL.

Methods of the invention described herein may deliver any composition to the cells suspended in the fluid. Compositions that can be delivered to the cells include, but are not limited to, therapeutic agents, vitamins, nanoparticles, charged molecules, e.g., ions in solution, uncharged molecules, nucleic acids, e.g., DNA or RNA, CRISPR-Cas complex, proteins, polymers, a ribonucleoprotein (RNP), engineered nucleases, transcription activator-like effector nucleases (TALENs), zinc-finger nucleases (ZFNs), homing nucleases, meganucleases (MNs), megaTALs, enzymes, peptides, transposons, or polysaccharides, e.g., dextran, e.g., dextran sulfate. Exemplary compositions that can be delivered to cells in a suspension include nucleic acids, oligonucleotides, antibodies (or an antibody fragment, e.g., a bispecific fragment, a trispecific fragment, Fab, F(ab′)2, or a single-chain variable fragment (scFv)), amino acids, peptides, proteins, gene therapeutics, genome engineering therapeutics, epigenome engineering therapeutics, carbohydrates, chemical drugs, contrast agents, magnetic particles, polymer beads, metal nanoparticles, metal microparticles, quantum dots, antioxidants, antibiotic agents, hormones, nucleoproteins, polysaccharides, glycoproteins, lipoproteins, steroids, anti-inflammatory agents, anti-microbial agents, chemotherapeutic agents, exosomes, outer membrane vesicles, vaccines, viruses, bacteriophages, adjuvants, minerals, and combinations thereof. A composition to be delivered may include a single compound, such as the compounds described herein. Alternatively, the composition to be delivered may include a plurality of compounds or components targeting different genes.

Typical concentrations of the composition in the fluid may be from about 0.0001 μg/mL to about 1000 μg/mL, (e.g., from about 0.0001 μg/mL to about 0.001 μg/mL, about 0.001 μg/mL to about 0.01 μg/mL, about 0.001 μg/mL to about 5 μg/mL, about 0.005 μg/mL to about 0.1 μg/mL, about 0.01 μg/mL to about 0.1 μg/mL, about 0.01 μg/mL to about 1 μg/mL, about 0.1 μg/mL to about 1 μg/mL, about 0.1 μg/mL to about 5 μg/mL, about 1 μg/mL to about 10 μg/mL, about 1 μg/mL to about 50 μg/mL, about 1 μg/mL to about 100 μg/mL, about 2.5 μg/mL to about 15 μg/mL, about 5 μg/mL to about 25 μg/mL, about 5 μg/mL to about 50 μg/mL, about 5 μg/mL to about 500 μg/mL, about 7.5 μg/mL to about 75 μg/mL, about 10 μg/mL to about 100 μg/mL, about 10 μg/mL to about 1,000 μg/mL, about 25 μg/mL to about 50 μg/mL, about 25 μg/mL to about 250 μg/mL, about 25 μg/mL to about 500 μg/mL, about 50 μg/mL to about 100 μg/mL, about 50 μg/mL to about 250 μg/mL, about 50 μg/mL to about 750 μg/mL, about 100 μg/mL to about 300 μg/mL, about 100 μg/mL to about 1,000 μg/mL, about 200 μg/mL to about 400 μg/mL, about 250 μg/mL to about 500 μg/mL, about 350 μg/mL to about 500 μg/mL, about 400 μg/mL to about 1,000 μg/mL, about 500 μg/mL to about 750 μg/mL, about 650 μg/mL to about 1,000 μg/mL, or about 800 μg/mL to about 1,000 μg/mL, e.g., about 0.0001 μg/mL, about 0.0005 μg/mL, about 0.001 μg/mL, about 0.005 μg/mL, about 0.01 μg/mL, about 0.02 μg/mL, about 0.03 μg/mL, about 0.04 μg/mL, about 0.05 μg/mL, about 0.06 μg/mL, about 0.07 μg/mL, about 0.08 μg/mL, about 0.09 μg/mL, about 0.1 μg/mL, about 0.2 μg/mL, about 0.3 μg/mL, about 0.4 μg/mL, about 0.5 μg/mL, about 0.6 μg/mL, about 0.7 μg/mL, about 0.8 μg/mL, about 0.9 μg/mL, about 1 μg/mL, about 1.5 μg/mL, about 2 μg/mL, about 2.5 μg/mL, about 3 μg/mL, about 3.5 μg/mL, about 4 μg/mL, about 4.5 μg/mL, about 5 μg/mL, about 5.5 μg/mL, about 6 μg/mL, about 6.5 μg/mL, about 7 μg/mL, about 7.5 μg/mL, about 8 μg/mL, about 8.5 μg/mL, about 9 μg/mL, about 9.5 μg/mL, about 10 μg/mL, about 15 μg/mL, about 20 μg/mL, about 25 μg/mL, about 30 μg/mL, about 35 μg/mL, about 40 μg/mL, about 45 μg/mL, about 50 μg/mL, about 55 μg/mL, about 60 μg/mL, about 65 μg/mL, about 70 μg/mL, about 75 μg/mL, about 80 μg/mL, about 85 μg/mL, about 90 μg/mL, about 95 μg/mL, about 100 μg/mL, about 200 μg/mL, about 250 μg/mL, about 300 μg/mL, about 350 μg/mL, about 400 μg/mL, about 450 μg/mL, about 500 μg/mL, about 550 μg/mL, about 600 μg/mL, about 650 μg/mL, about 700 μg/mL, about 750 μg/mL, about 800 μg/mL, about 850 μg/mL, about 900 μg/mL, about 950 μg/mL, or about 1,000 μg/mL).

In some cases, the temperature of the fluid with the suspended cells and the composition is controlled using a thermal controller that is incorporated into a housing that supports the device(s) of the invention. The temperature of the fluid is controlled to reduce the effects of Joule heating originating from the electric field generated in the electroporation zone, as too high a temperature may compromise cell viability post-electroporation. The temperature of the fluid may be from about 0° C. to about 50° C., e.g., from about 0° C. to about 10° C., about 1° C. to about 5° C., about 2° C. to about 15° C., about 3° C. to about 20° C., about 4° C. to about 25° C., about 5° C. to about 30° C., about 7° C. to about 35° C., about 9° C. to about 40° C., about 10° C. to about 43° C., about 15° C. to about 50° C., about 20° C. to about 40° C., about 25° C. to about 50° C., or about 35° C. to about 45° C., e.g., about 0° C., about 1° C., about 2° C., about 3° C., about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C. about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., or about 50° C.

Cells transfected using the methods of the invention are more efficiently transfected and have higher viability than using typical methods of transfection, e.g., lentiviral transfection, or commercially available cell transfection instruments, e.g., the NEON® Transfection System (Thermo Fisher, Carlsbad, Calif.) or 4D-NUCLEOFECTOR (Lonza, Switzerland). For example, the transfection efficiency, i.e., the efficiency of successfully delivering a composition to a cell, for the methods described herein, may be from about 0.1% to about 99.9%, e.g., from about 0.1% to about 5%, about 1% to about 10%, about 2.5% to about 20%, about 5% to about 40%, about 10% to about 60%, about 30% to about 80%, or about 50% to about 99.9%, e.g., from about 10% to about 90%, from about 25% to about 85%, e.g., about 0.1%, about 0.15%, about 0.2%, about 0.25%, about 0.3%, about 0.35%, about 0.4%, about 0.45%, about 0.5%, about 0.55%, about 0.6%, about 0.65%, about 0.7%, about 0.75%, about 0.8%, about 0.85%, about 0.9%, about 0.95%, about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 99.9%.

The cell viability, i.e., the number or percentage of cells that have survived electroporation, of the cells suspended in the fluid after having a composition introduced using methods of the invention described herein may be from about 0.1% to about 99.9%, e.g., from about 0.1% to about 5%, about 1% to about 10%, about 2.5% to about 20%, about 5% to about 40%, about 10% to about 60%, about 30% to about 80%, or about 50% to about 99.9%, e.g., from about 10% to about 90%, from about 25% to about 85%, e.g., about 0.1%, about 0.15%, about 0.2%, about 0.25%, about 0.3%, about 0.35%, about 0.4%, about 0.45%, about 0.5%, about 0.55%, about 0.6%, about 0.65%, about 0.7%, about 0.75%, about 0.8%, about 0.85%, about 0.9%, about 0.95%, about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 99.9%.

The number of recovered cells, i.e., the number of live cells collected after electroporation, may be from about 10⁴ cells to about 10¹² cells, e.g., about 10⁴ cells to about 10⁵ cells, about 10⁴ cells to about 10⁶ cells, about 10⁴ cells to about 10⁷ cells, about 5×10⁴ cells to about 5×10⁵ cells, about 10⁵ cells to about 10⁶ cells, about 10⁵ cells to about 10⁷ cells, about 2.5×10⁵ cells to about 10⁶ cells, about 5×10⁵ cells to about 5×10⁶ cells, about 10⁶ cells to about 10⁷ cells, about 10⁶ cells to about 10⁸ cells, about 10⁶ cells to about 10¹² cells, about 5×10⁶ cells to about 5×10⁷ cells, about 10⁷ cells to about 10⁸ cells, about 10⁷ cells to about 10⁹ cells, about 10⁷ cells to about 10¹² cells, about 5×10⁷ cells to about 5×10⁸ cells, about 10⁸ cells to about 10⁹ cells, about 10⁸ cells to about 10¹⁰ cells, about 10⁸ cells to about 10¹² cells, about 5×10⁸ cells to about 5×10⁹ cells, about 10⁹ cells to about 10¹⁰ cells, about 10⁹ cells to about 10¹¹ cells, about 10¹⁰ cells to about 10¹¹ cells, about 10¹⁰ cells to about 10¹² cells, or about 10¹¹ cells to about 10¹² cells, e.g., about 10⁴ cells, about 2.5×10⁴ cells, about 5×10⁴ cells, about 10⁵ cells, about 2.5×10⁵ cells, about 5×10⁵ cells, about 10⁶ cells, about 2.5×10⁶ cells, about 5×10⁶ cells, about 10⁷ cells, about 2.5×10⁷ cells, about 5×10⁷ cells, about 10⁸ cells, about 2.5×10⁸ cells, about 5×10⁸ cells, about 10⁹ cells, about 2.5×10⁹ cells, about 5×10⁹ cells, about 10¹⁰ cells, about 5×10¹⁰ cells, about 10¹¹ cells, or about 10¹² cells.

The recovery yield, i.e., the percentage of live engineered cells collected after electroporation, may be from about 0.1% to about 500%, e.g., from about 0.1% to about 5%, about 1% to about 10%, about 2.5% to about 20%, about 5% to about 40%, about 10% to about 60%, about 30% to about 80%, about 50% to about 99.9%, from about 75% to about 150%, from about 100% to about 200%, from about 150% to about 250%, from about 200% to about 300%, from about 250% to about 350%, from about 300% to about 400%, from about 350% to about 450%, or from about 400% to about 500%, e.g., about 0.1%, about 0.15%, about 0.2%, about 0.25%, about 0.3%, about 0.35%, about 0.4%, about 0.45%, about 0.5%, about 0.55%, about 0.6%, about 0.65%, about 0.7%, about 0.75%, about 0.8%, about 0.85%, about 0.9%, about 0.95%, about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99.9%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500%.

A skilled artisan will appreciate that optimal conditions may vary depending on cell type or other factors. For each new cell type, the following parameters can be adjusted as necessary: waveform, electric field, pulse duration, buffer exposure time, buffer temperatures, and post-electroporation conditions.

### EXAMPLES

### Example 1—Devices and Systems

A continuous flow electroporation device and related system were designed and fabricated to allow for a plurality of devices to be used in parallel to enhance or maximize the number of cell electroporation events occurring in a fixed time window, thereby enhancing or maximizing throughput of cell engineering and/or accelerating biological discovery. The electroporation device is configured to be compatible with current automated fluid handling systems, e.g., pipette tip-based dispensers, robotic fluid pumps, etc.

FIG. 1A shows a schematic of an exemplary embodiment of an electroporation device shown, in this configuration, as a pipette tip. FIG. 1A shows a close-up view of an active area of the device, including an electroporation zone. This device provides for continuous flow genetic manipulation of both eukaryotic and prokaryotic cells in a platform that can be easily automated through integration with liquid handling robots. In the device of FIGS. 1A-1C, the active area of the device includes three distinct zones: the entry zone, the electroporation zone, and the recovery zone. In the embodiment shown in FIGS. 1A-1C, a composition to be introduced into cells and the cells to be transfected are placed in the entry zone. The cells and composition are passed through the electroporation zone, and the transfected cells are dispensed into a buffer for storage in the recovery zone. Thus, the space between the entry and recovery zones is the electroporation zone, and all three zones are in fluid communication (e.g., fluidically connected), such that there is one flow path through the device.

In the embodiment shown in FIG. 1A, the entry zone and the recovery zone are fabricated from hollow electrodes made of a suitable material, e.g., stainless steel, with the entry zone electrode acting as the energized electrode and the recovery zone electrode acting as the grounded electrode, thus completing the circuit while allowing an electric field to develop between the two electrodes (in combination with the conductivity of the fluid carrying the cells and composition).

The electroporation devices of the invention have been designed to meet the requirements of injection and insert molding manufacturing techniques, both of which are scalable in nature, and are shown in FIGS. 1B and 1C. In FIGS. 1B and 1C, the device body integrates with the electroporation zone, which is located in between commercial stainless-steel electrodes, where the electric field is active. The electroporation zone geometry was modified to exhibit a substantially uniform cross-section, resulting in a more predictable electric field exposure during the residence time of the electroporation sample. Using current production methods, e.g., 3D printing, approximately 100 devices per day can be manufactured; this is scalable to over 10,000 devices a day using more robust large-scale production methods, e.g., injection and insert molding.

A housing can be configured to energize a plurality of electroporation devices, e.g., 96 electroporation devices in parallel in an industry standard 96-well pipette tip tray with grid electrodes, to energize all of the electroporation devices simultaneously with an identical applied voltage pulse such that the electric field within each electroporation device is identical. A single power supply can be used to deliver the electrical energy. Thus, a mechanism may be needed to distribute the power to each electroporation device. One method to implement this is shown in FIG. 2A, with an exploded view in FIG. 2B. This design features spring-loaded electrodes in which the 96 individual electroporation devices enter housing where the first and second electrodes of each electroporation device make physical contact with the electrical grids of the housing. The spring-loaded electrodes are each connected in parallel to the electrical grids of the housing, which in turn is connected to the power supply by a single set of leads. The housing is reusable so that once connected to the power supply it can facilitate genetic modification of up to 96 discrete samples simultaneously. The power supply may include additional circuitry or programming configured to modulate the pulse delivery so that each individual device of the invention, e.g., 96 individual devices, receives a different voltage or a different waveform.

### Example 2—Initial Development of Experimental Parameters for Optimal Transfection

Experiments have been conducted to study the physical and biological parameters influencing electroporation of the Jurkat immortalized T cell line using devices of the current invention. Using industry standard flow cytometry methods, both cell viability (measured by 7-AAD dye exclusion) and transfection efficiency (measured by GFP expression) of engineered Jurkat cells were assessed using our devices, both of which are common measures of electroporation success in the field of gene delivery.

Unless specified otherwise, experimental results shown below were generated by electroporating a population of Jurkat cells at a concentration of 1×10⁶ cells in 100 μL of buffer with 5 μg of plasmid (e.g., GFP expression plasmid). Electroporation experiments were performed at 100 Hz with square waveforms and a pulse duration of 9.5 ms. After 24-hour incubation, cells were stained with 7-AAD stain and analyzed via flow cytometry to measure viable cells and live GFP expressing cells. Experiments were performed in triplicate, with error bars representing the standard error of the mean (SEM). Table 1 below presents a summary of the parameters used for transfection using devices of the invention. Most of the parameters listed in Table 1 are independent of one another, with the notable exceptions falling within two distinct groups. The first group of dependent parameters are those which vary according to the physical channel dimensions and process parameters such as the Reynolds number, Velocity, Average Velocity, Shear Rates, and Peak Pressure. A skilled artisan will appreciate that the Reynolds number is a function of the Average Velocity, Kinematic Viscosity, and Channel Diameter. The Velocity (which can vary locally within the channel), Average Velocity, Shear Rates, and Peak Pressure are functions of the Flow Rate and Channel dimensions. The second group of dependent parameters are those related to the biological outcome of the transfection, including the Recovered cells, Cell Viability, Transfection Efficiency, and Yield from input cells. These parameters are dependent upon a host of factors including the cell type being transfected and the combination of the physical parameters indicated in Table 1.

TABLE 1

| | Minimum | Operating | Maximum |
|---|---|---|---|
| Parameter [Units] | Value | Value | Value |
| Samples in Parallel | 1, 4, 8, | 96 | 384, |
| | 12, 24, 48 | | 1536 |
| Samples in Series | 1 | 8 | 12 |
| Electrode Number | 1 | 2 | 3+ |
| Electrode Gauge | 6 | 16 | 34 |
| Channel Diameter [mm] | 0.005 | 0.5-1.0 | 50 |
| Channel Length [mm] | 0.005 | 4.0-8.0 | 50 |
| Flow Rate [mL/min] | 0.001 | 5-50 | 1,000 |
| Frequency [Hz] | 1 | 100-500 | 50,000 |
| Duty Cycle [%] | 0.001 | 10-95 | 100 |
| Pulse Number | 1 | 10 | 1,000,000 |
| Pulse Duration [ms] | 0.01 | 1-10 | 1,000 |
| Electric Field [V/cm] | 2.0 | 100-1,000 | 50,000 |
| Applied Voltage [V] | 10 | 200-600 | 3,000 |
| Electric Conductivity [mS/cm] | 0.001 | 1-20 | 500 |
| Sample Temperature [° C.] | 1.0 | 4.0-37 | 50 |
| Sample Volume [mL] | 0.001 | 0.025-0.10 | 2,000,000 |
| Cell Number | 1.0E4 | 21E5-10E6 | 100.0E10 |
| Recovered cells post EP | 1.0E4 | 1.0E6-10E6 | 100.0E10 |
| Cell Concentration [cells/mL] | 1.0E3 | 1.0E7 | 1.0E11 |
| Payload Concentration [g/mL] | 0.01 | 1-10 | 1,000 |
| Recovered cells [%] | 0.1 | 50 | 99.9 |
| Cell Viability [%] | 0.1 | 50 | 99.9 |
| Transfection Efficiency [%] | 0.1 | 50 | 99.9 |
| Yield from input cells [%] | 0.1 | 99.9 | 500 |
| Reynolds Number | 0.04 | 183-1530 | 2.43E4 |
| Velocity [m/s] * | 5E−5 | 0.26-2.08 | 32.7 |
| Shear Rates +5-9* | 0.1 | 2.6E3-5.4E4 | 2E6 |
| Peak Pressure [Pa] * | 1E−3 | 136-1600 | 9.5E4 |
| Average Velocity [m/s]* | 1.5E−5 | 7.79E−2-7.81E−1 | 15.9 |
| Kinematic Viscosity [m²/s]** | 1E−6 | 1.3E−6-5.6E−4 | 15E−4 |

| | |
|---|---|
| Waveform/Pulse Shape | Square, Pulse, Bipolar, Sine, Ramp, |
| | Asymmetric Bipolar, High |
| | Voltage-Low Voltage, Low |
| | Voltage-High Voltage, Direct Current |
| | (DC), Unipolar, (+) Polarity ONLY, |
| | (−) Polarity ONLY, (+)/(−) |
| | Polarity, (−)/(+) Polarity |
| Payload | Charged Molecules, Uncharged |
| | Molecules, DNA, RNA, CRISPR- |
| | Cas9, Proteins, Polymers, |
| | Ribonucleoprotein (RNP), Dextran |
| Electrode Material | 304 Stainless Steel, 316 Stainless |
| | Steel, Gold, Platinum, Carbon, |
| | Conductive liquid, Conductive Solution |

*Values calculated from simulation
**Values approximated from materials potentially used in the formulation of buffer

### Example 3—Transfection Data Using Devices of the Invention

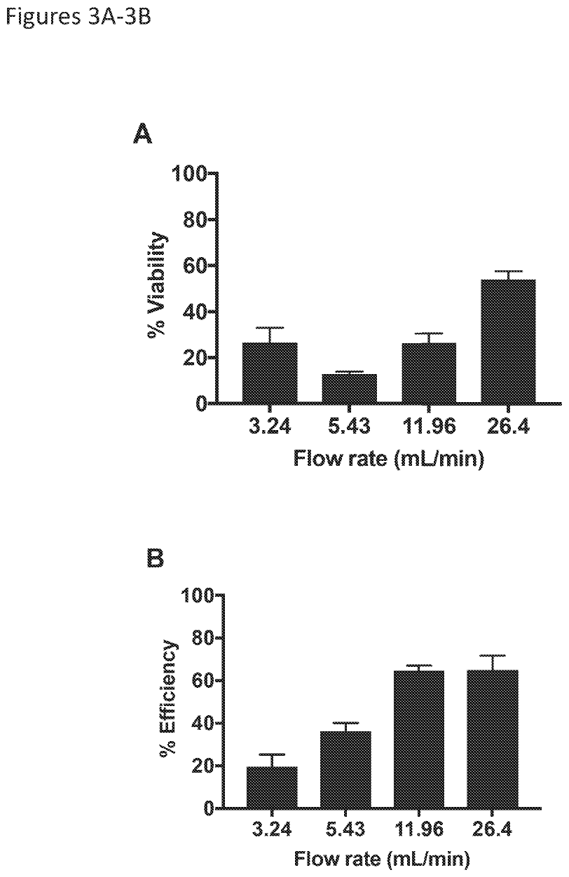

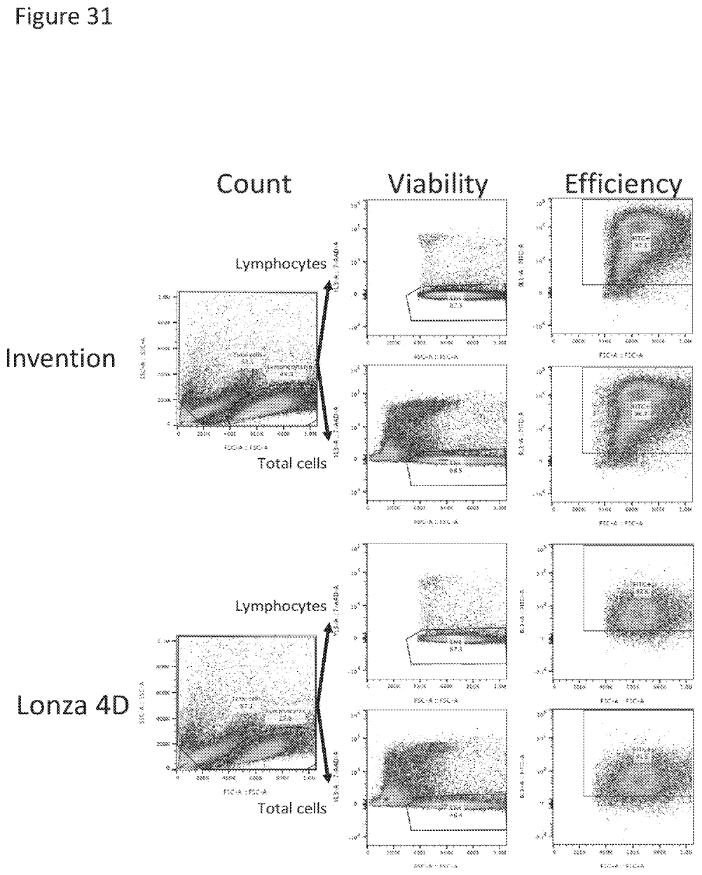

Devices of the invention show peak transfection performance when the flow rate is maximized through the electroporation channel (FIGS. 3A and 31B). The desired flow rate was achieved utilizing a controlled dispense rate pipette to increase both viability and efficiency, corresponding to a ˜6.5 ms residence time of the cell sample within the electric field. Peak cell viability of 54% was achieved, with transfection efficiency of 65%, demonstrating a significant advancement in the transfection of human immune cells with devices of the invention.

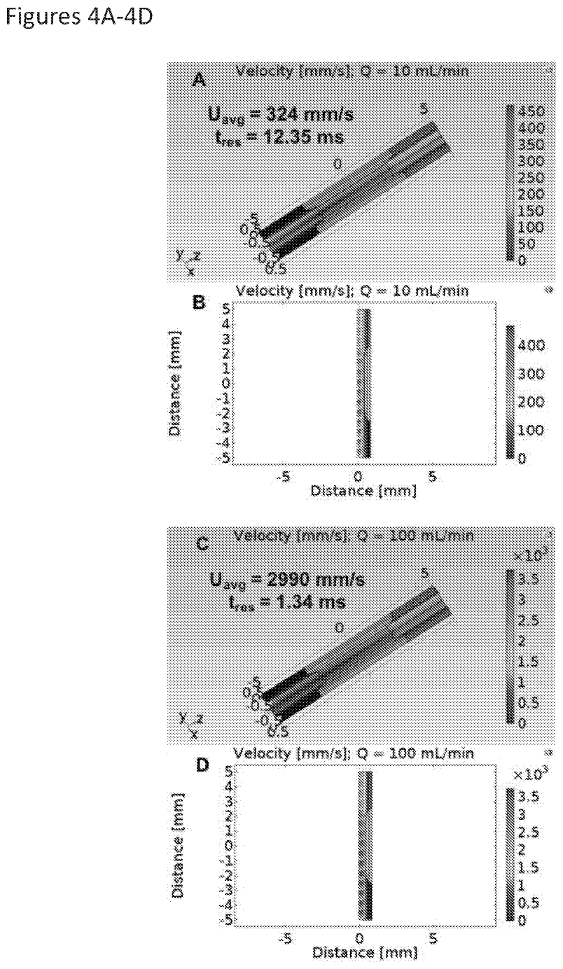

FIGS. 4A-4D illustrate flow rate simulation along an exemplary active zone of the device (i.e., from a first electrode lumen, through the electroporation zone, and into the second electrode lumen). In this embodiment, a medium contains flowing biological cells. From the simulated fluid flow at 10 mL/min and 100 mL/min, the average linear velocity of the samples going through the electroporation zone is determined. The lower flow rate of 10 mL/min results in an average linear velocity of 324 mm/s. The higher flow rate of 100 mL/min results in an average linear velocity of 2,990 mm/s. The two linear velocities can be correlated to estimated residence time (τᵣₑₛ) of 12.35 ms and 1.34 ms, respectively. These devices provided a flow rate of 16 mL per minute. Notably, for commercial systems to result in equivalent transfection efficiency, exposures of about 30 ms or longer are required under similar electric field exposure. This demonstrates that the combination of high flow rates and electric field result in improved delivery of genetic material into biological cells using devices of the present invention.

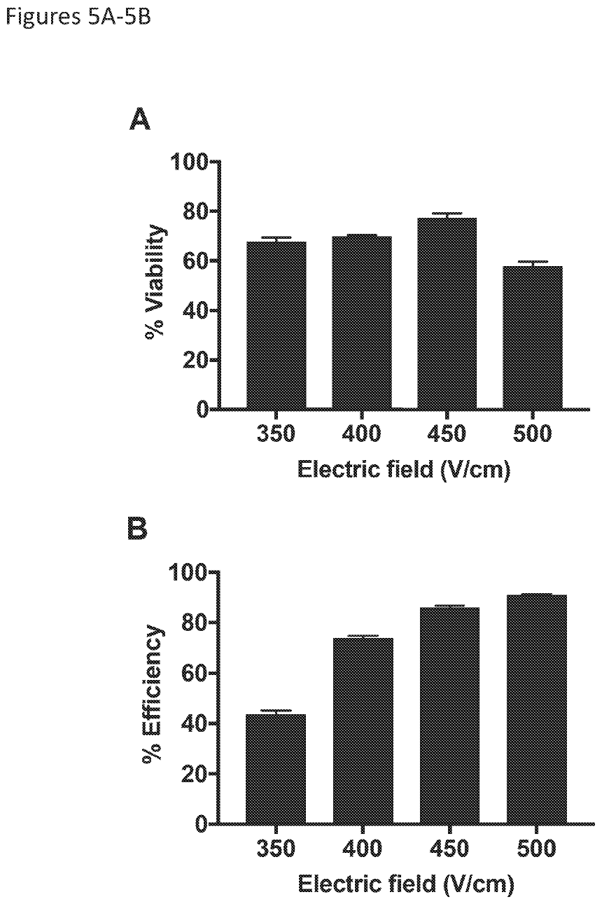

Transfection efficiency using devices of the invention is influenced by the electric field strength. FIGS. 5A and 5B show cell viability and transfection efficiency, respectively, that result from various electric field strengths. A transfection efficiency of 86% and a viability of 77% were achieved.

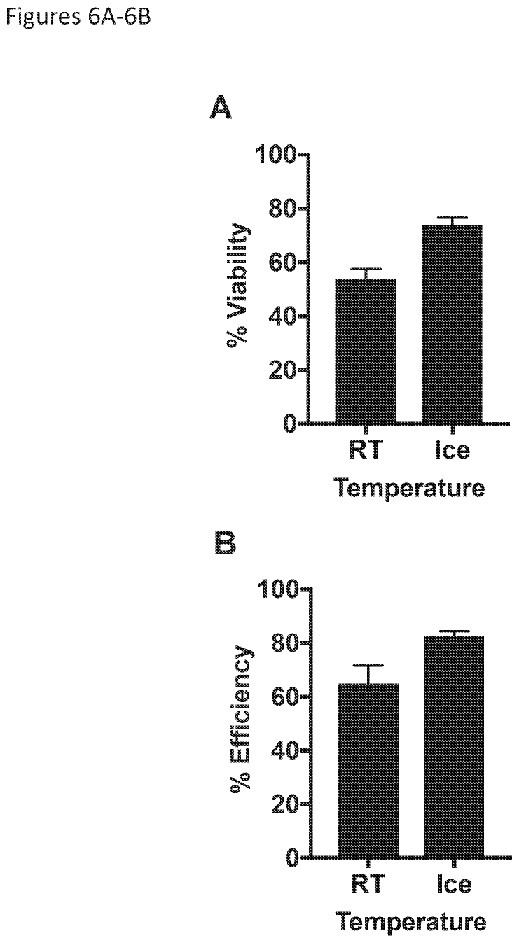

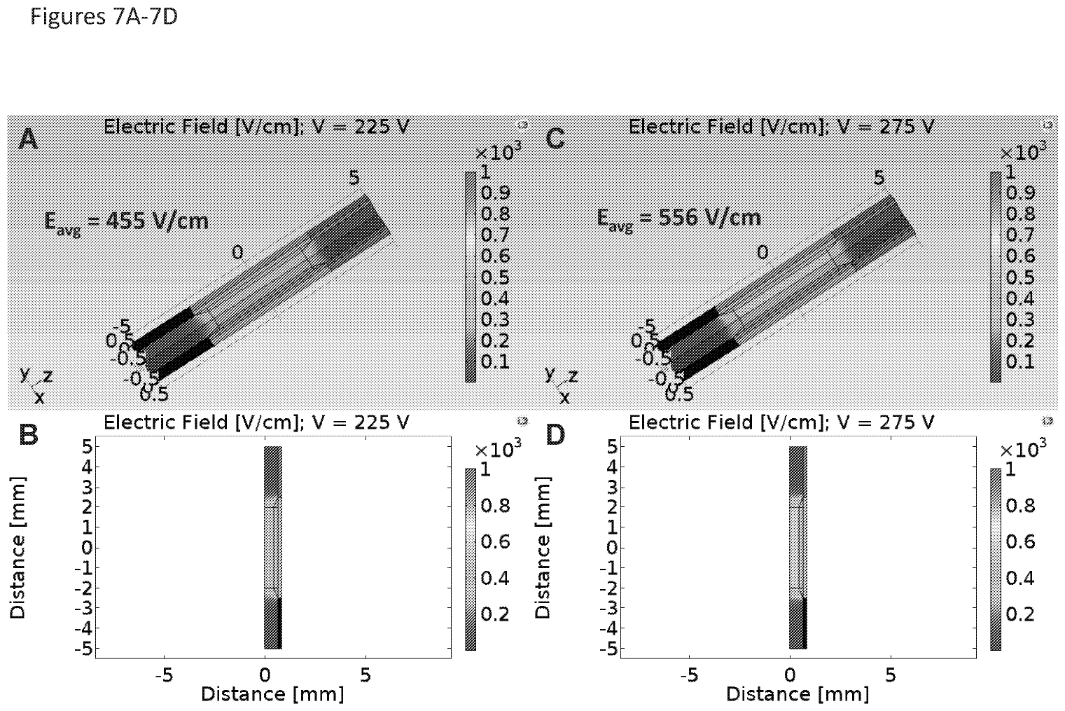

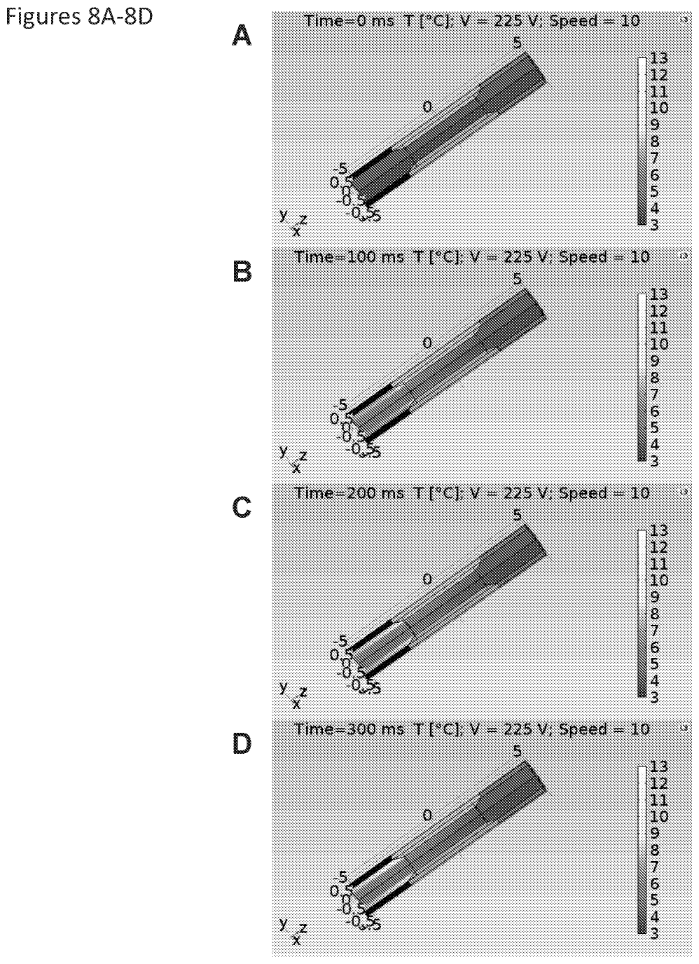

Devices of the invention showed ˜20% increases in both cell viability and transfection efficiency by chilling the sample on ice to minimize any potential deleterious thermal effects that may affect cell viability due to increased temperature during the electroporation (FIGS. 6A and 6B). Numerical modeling in COMSOL Multiphysics coupling the electric field, fluid flow, and thermal effects were also developed to better understand the impact of the sample temperature in device of the invention, using an applied voltage, in this model, of 225 V or 275 V. Results, shown in FIGS. 7A-7D, show a substantially uniform electric field in the electroporation zone. FIGS. 8A-8D show temperature distributions in the device over time.

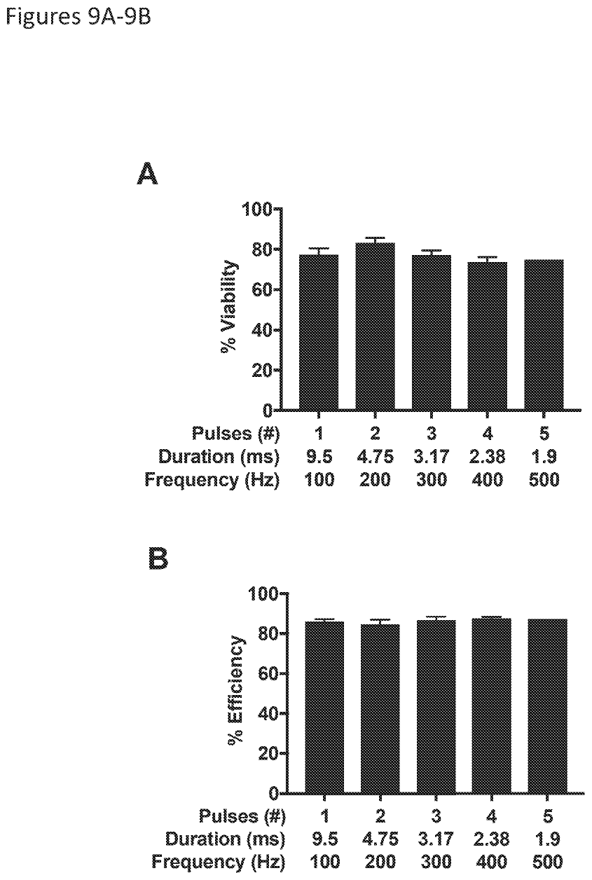

Electroporation using devices of the invention showed no significant changes in performance when electroporation was performed across a range of pulse durations with matched frequencies (FIGS. 9A and 9B). By varying the number of pulses within a 9.5 ms duration from 1 to 5, no significant changes were observed in either viability or efficiency, demonstrating the waveform flexibility for electroporation using devices of the invention. In this experiment, a peak cell viability of 83% was achieved, with a transfection efficiency of 88%.

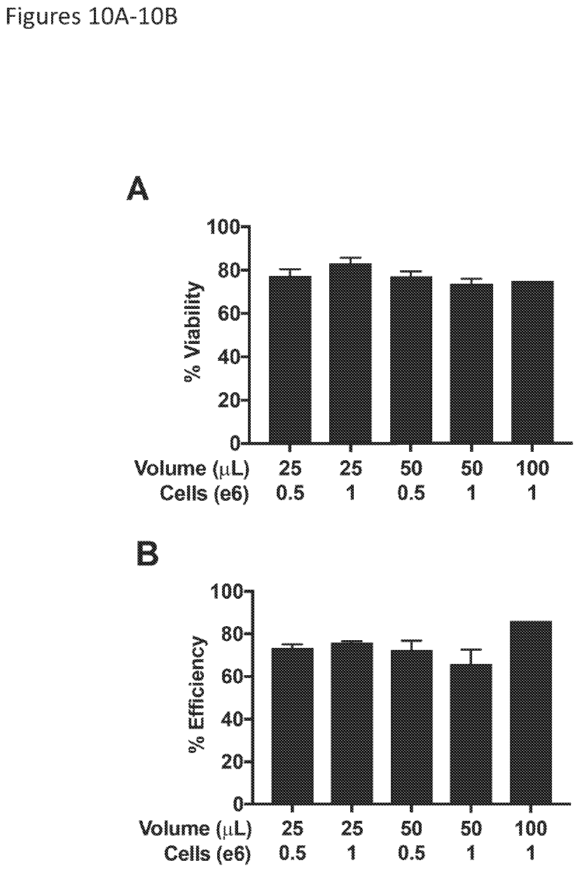

Electroporation using devices of the invention showed no significant changes in performance when electroporation was performed across a range of volumes and cell densities (FIGS. 10A and 10B). By varying the number of cells across a range of volumes from 25 to 100 μL, no significant changes were observed in either viability or efficiency, demonstrating the physical reaction flexibility for electroporation using devices of the invention. In this experiment, peak cell viability of 83% was achieved, with a transfection efficiency of 86%.

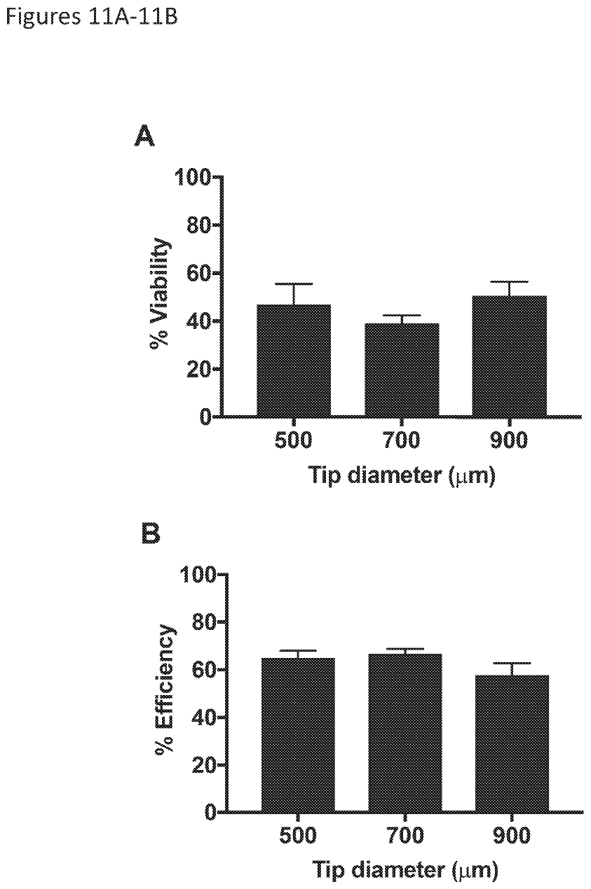

Electroporation using devices of the invention showed no significant changes in performance when electroporation was performed across a range of cross-sectional dimensions of the electroporation zone (FIGS. 11A and 11B). By varying the cross-sectional dimensions of the electroporation zone from 500 to 900 μm, similar viabilities were observed, with no significant changes in efficiency when the flow rates were modified to match total residence time within the electroporation zone, demonstrating the cross-sectional dimension flexibility for electroporation using devices of the invention. In this experiment, peak cell viability of 51% was achieved, with a transfection efficiency of 67%.

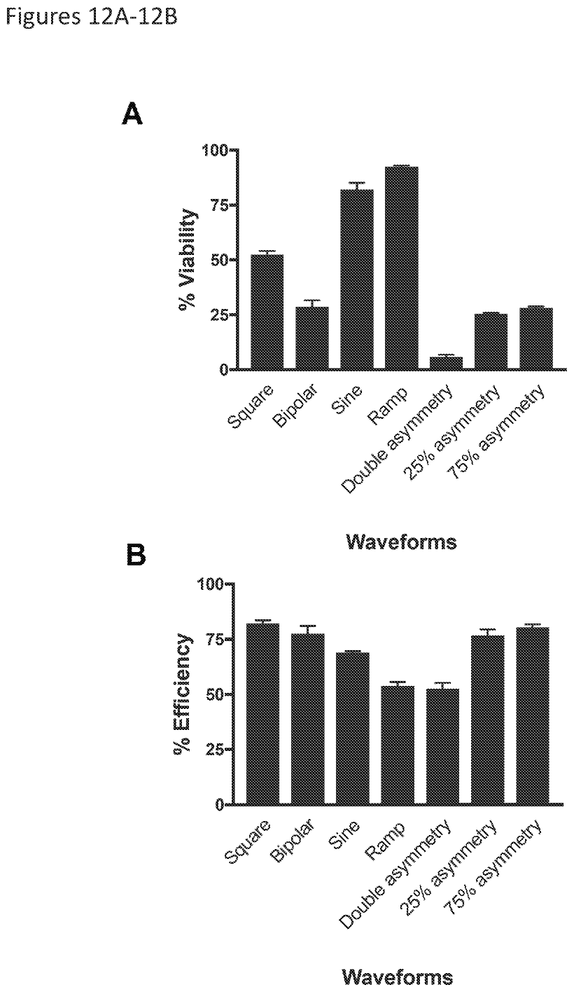

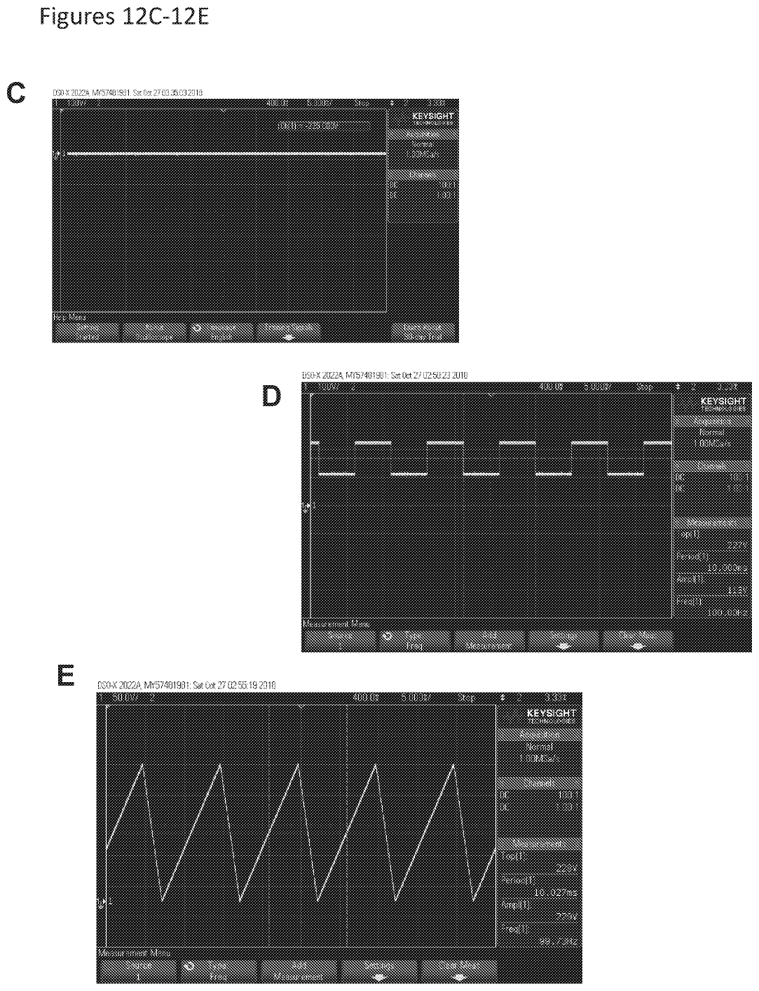

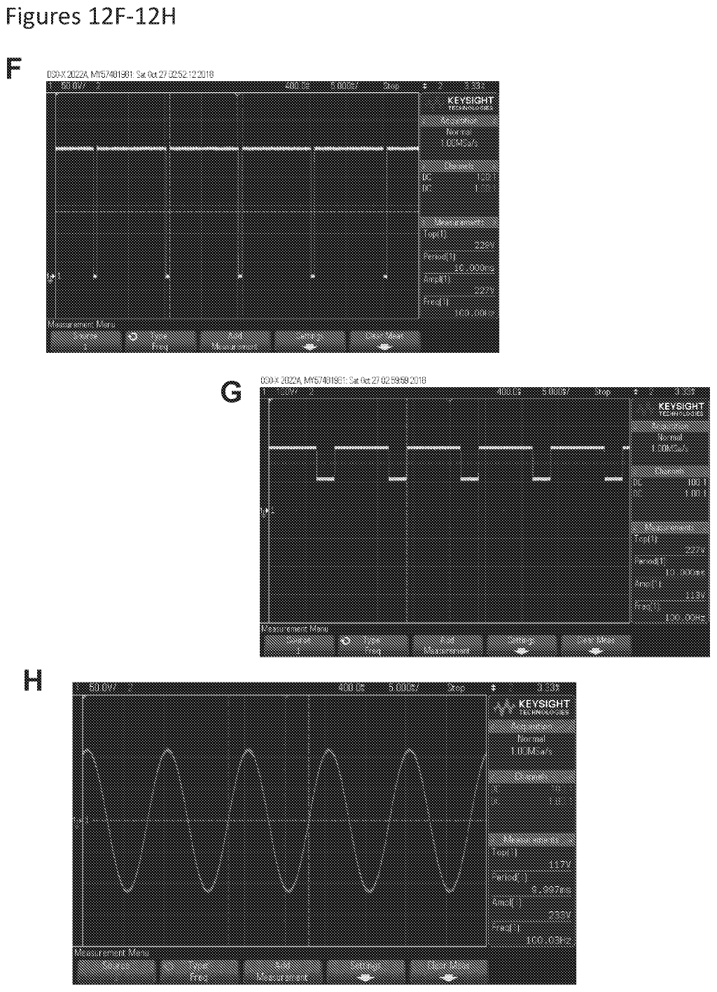

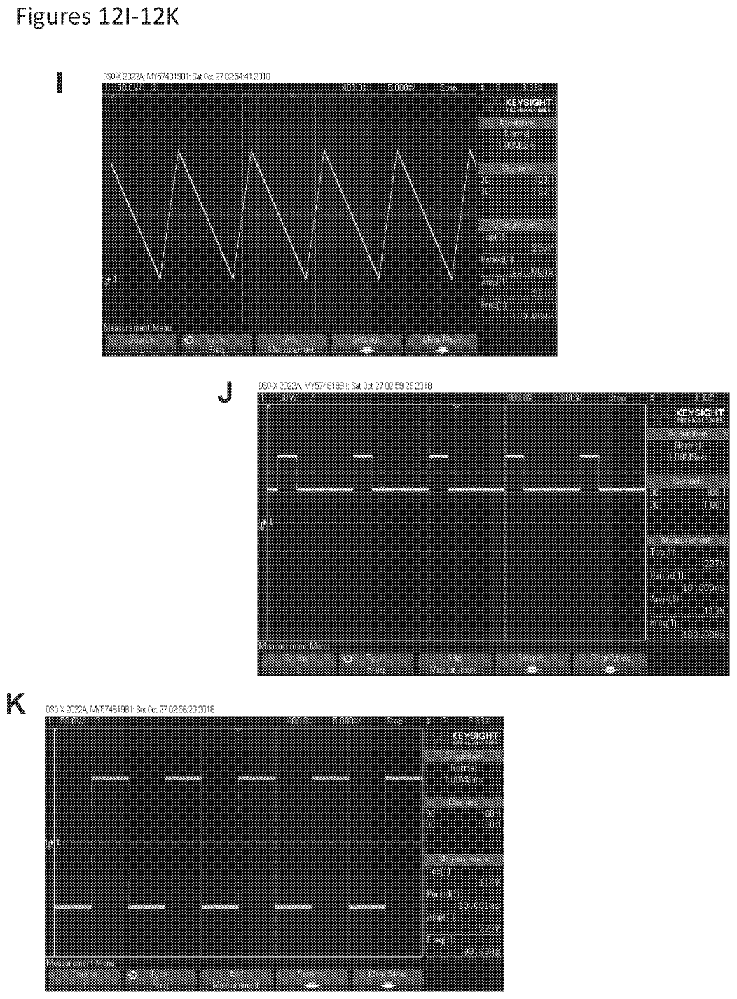

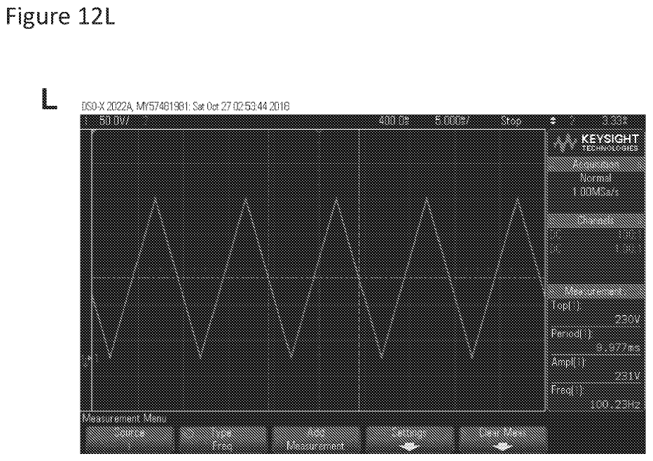

Viability and efficiency depended on the voltage pulse waveform shapes, as shown in FIGS. 12A and 12B. By changing the shape of the waveform, the time and strength of the electric current to which each Jurkat cell is exposed was adjusted, thereby altering the viability or efficiency. In this experiment, high cell viability was observed in combination with high transfection efficiency (above 50%) using square, sine, and ramp waveform shapes. Example waveforms useful for devices of the invention are shown in FIGS. 12C-12L.

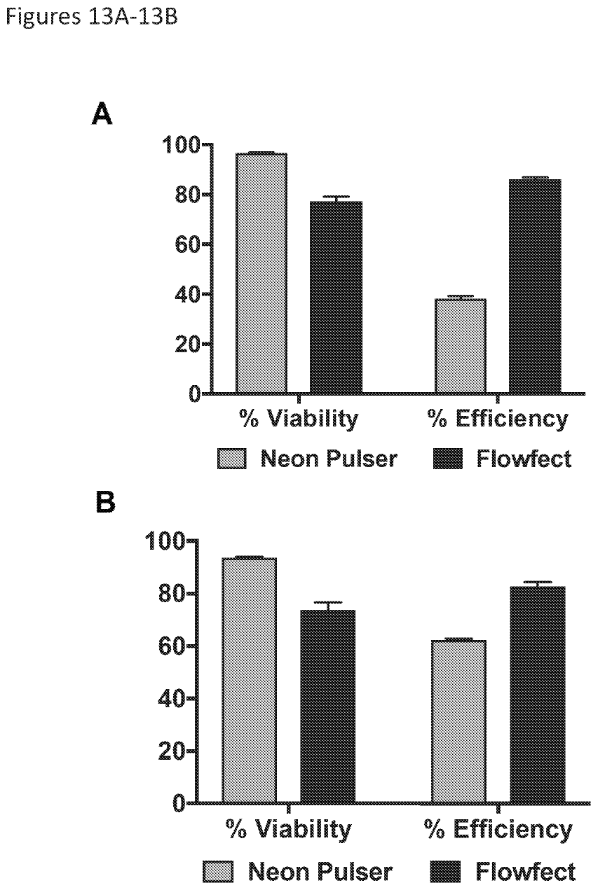

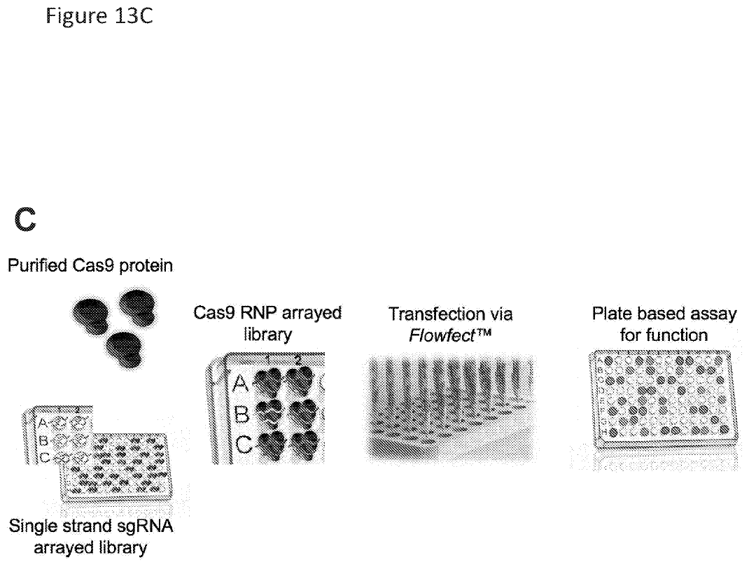

FIGS. 13A and 13B show viability and efficiency of the devices of the invention utilizing a flow rate of 10-25 mL per minute with an electric field of 400-700 V/cm under chilled conditions. All of the optimizations performed enable delivery of nucleic acids at a higher efficiency compared to the state-of-the-art commercially available NEON® Transfection System in multiple independent experiments (FIGS. 13A and 13B).

### Example 4—Applications of the Devices of the Invention to Genetic Engineering

The therapeutic application of primary human T-cells has shown significant advancement in the field of immuno-oncology by targeting the patient's immune system to be effective at fighting cancer. A number of technologies, including chimeric antigen receptors and engineered T-cell receptors, have shown clinical success in recent years. However, applications of genetically modifying the patient's immune system remains somewhat limited to treating blood cancers since the tumor microenvironment of solid tumors inhibit T-cell function at the tumor site. To overcome some of the biological challenges of tumor microenvironment suppression, there is a desire to further modify the T-cells to be more effective by knocking-out genes that express regulatory ligands on the T-cell surface. Identification of these genes is often achieved through CRISPR screens, in which Cas9 and guide RNA libraries are delivered into the T-cells to knock-out a wide range of endogenous genes to achieve functional enhancements against specific tumors. However, delivery of these libraries remains a hurdle for the identification of genes in “hard to transfect” cell types, such as primary T-cells and Natural Killer Cells. Typically, in these instances, the CRISPR libraries are delivered as lentiviral particles that will infect the cells and transduce the Cas9/guide RNA sequences into the cellular genome, which will then knock-out the gene of interest in a sequence-specific manner. These libraries are very laborious to produce, requiring cloning of viral expression plasmids and purification of the viral particles for delivery. Additionally, this methodology leaves the unwanted “baggage” of genetically incorporated Cas9/guide RNA sequences at random genomic insertion sites, which may interrupt other functional genes. The use of non-viral delivery for Cas9 ribonucleoprotein complexes is an attractive method to overcome these hurdles, enabling researchers to screen a large number of knock-outs in the absence of viral incorporation using a transient delivery of Cas9 protein complexed with the guide RNA molecules.

FIG. 13C is a flow chart of a method for delivering Cas9 ribonucleoprotein complexes to cells using devices of the invention. Delivery of Cas9 ribonucleoprotein complexes to cells with electroporation enables high-throughput analysis of targeted CRISPR knock-outs in a highly efficient manner, transforming the discovery process of novel gene targets for therapeutic application. Studies utilize a 200-1,000 gene subset or greater, e.g., 25,000, from commercially available cell surface receptor libraries to identify genes that inhibit the tumor microenvironment suppression of T-cell survival and persistence.

### Example 5—Electroporation of Human Cells

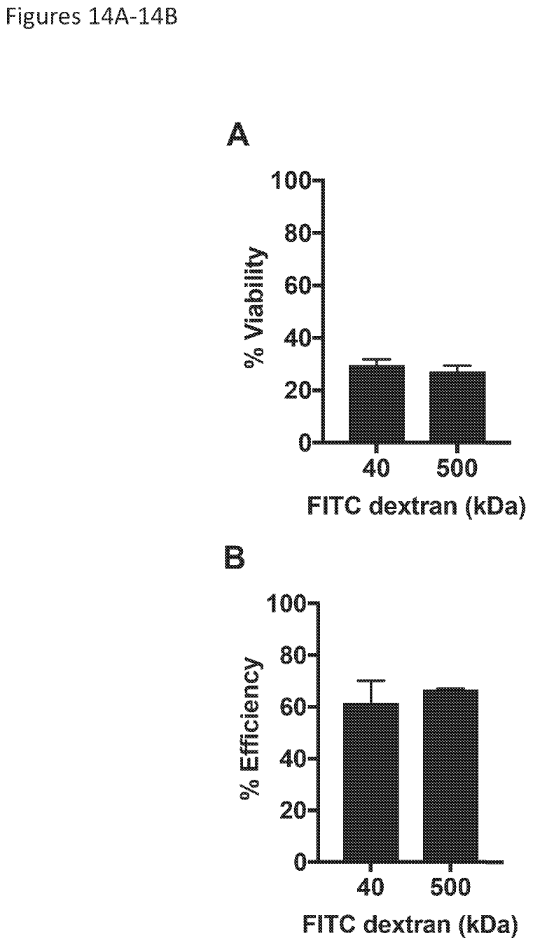

FIGS. 14A and 14B show viability and efficiency data for the electroporation of primary human T-cells using two different molecular weights of fluorescent dextran molecules at an electric field strength of 700 V/cm. In this experiment, a peak cell viability of 30% was achieved, with transfection efficiency of 67%, demonstrating a significant advancement in the transfection of primary human immune cells using devices of the invention.

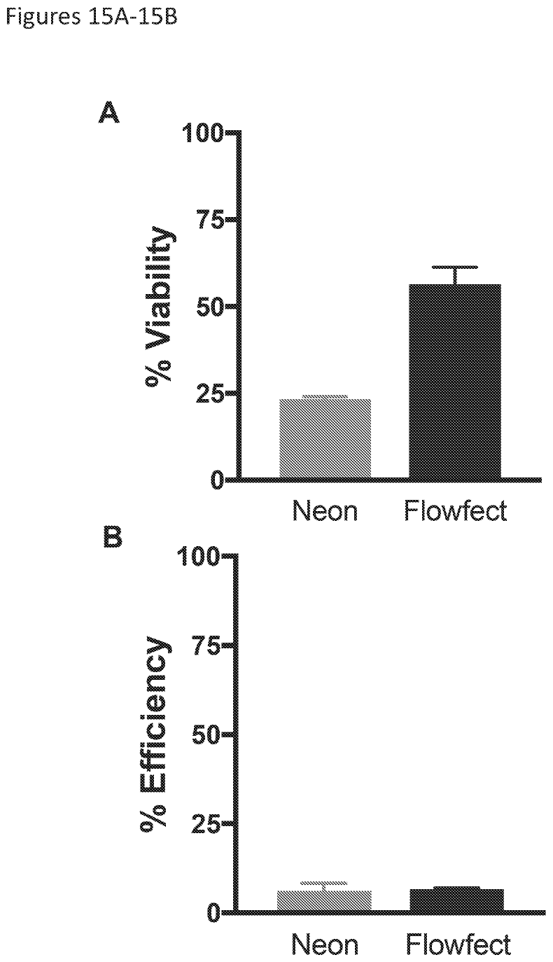

In a related experiment, electroporation using devices of the invention shows significantly increased performance compared to NEON® in the THP-1 monocyte cell line (ATCC number TIB-202) using published NEON® transfection system monocyte electroporation protocols (FIGS. 15A and 15B). In this experiment, increased cell viability of 56.4% was observed using devices of the invention, compared to 23.4% with the NEON® transfection system, while transfection efficiency was maintained at 6%.

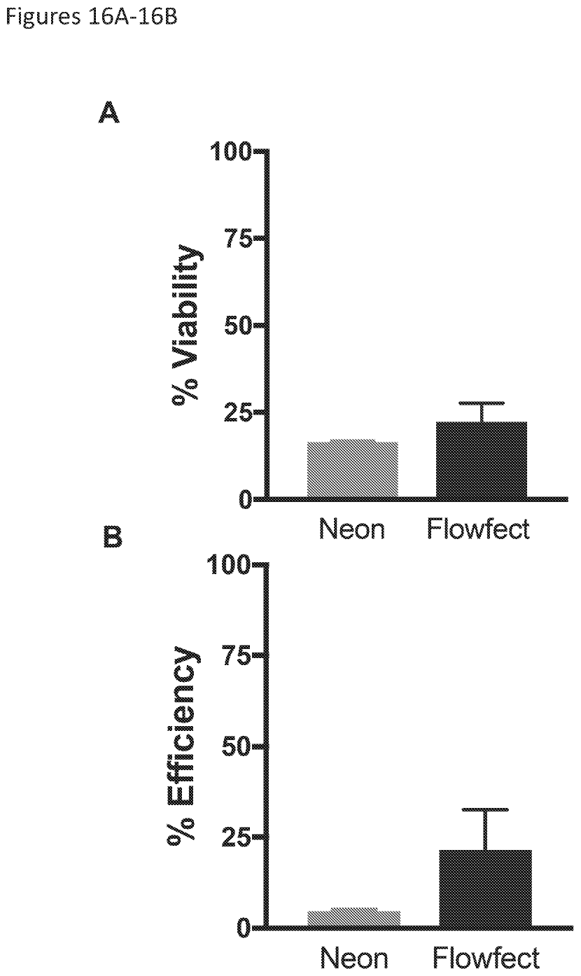

Electroporation using devices of the invention showed increased performance compared to NEON® transfection system in primary human monocytes using published NEON® transfect system monocyte electroporation protocols (FIGS. 16A and 16B). In this experiment, increased cell viability of 22.3% was observed using devices of the invention, compared to 16.6% observed with the NEON® transfection system, and increased transfection efficiency of 21.6% was observed using devices of the invention compared to 4.7% observed with the NEON® transfection system.

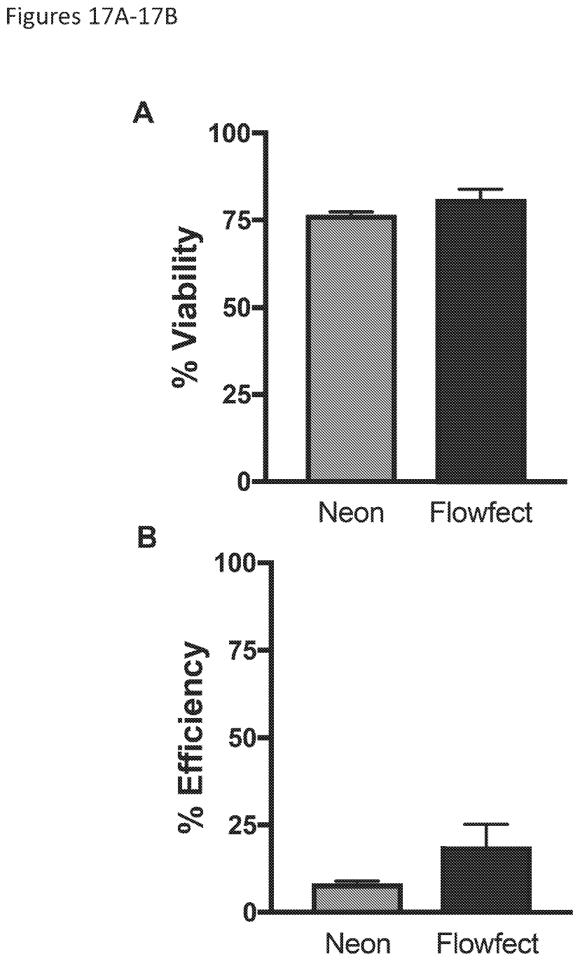

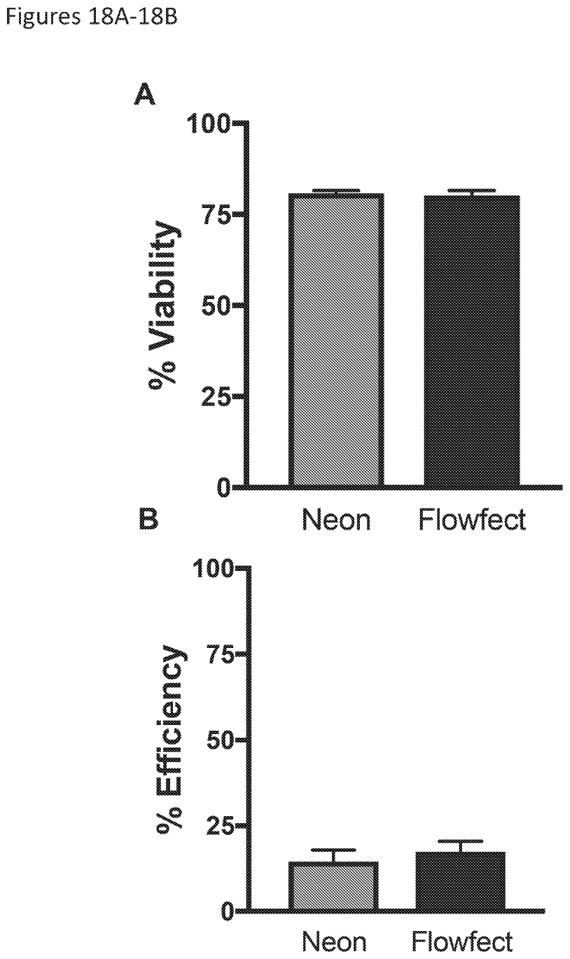

Electroporation using devices of the invention showed increased performance compared to NEON® transfection system in independent experiments and for the successful delivery of 40 kDa dextran molecules into Natural Killer Cell Lines of the NK-92 (ATCC) (FIGS. 17A and 17B) and NK-92MI (ATCC) (FIGS. 18A and 18B) lineages. These results confirm the ability of the devices of the invention to deliver molecules outside of the nucleic acid space with comparable cell viability and improved transfection efficiency to non-scalable commercially available platforms.

SIRPalpha mRNA Delivery to Primary Monocytes

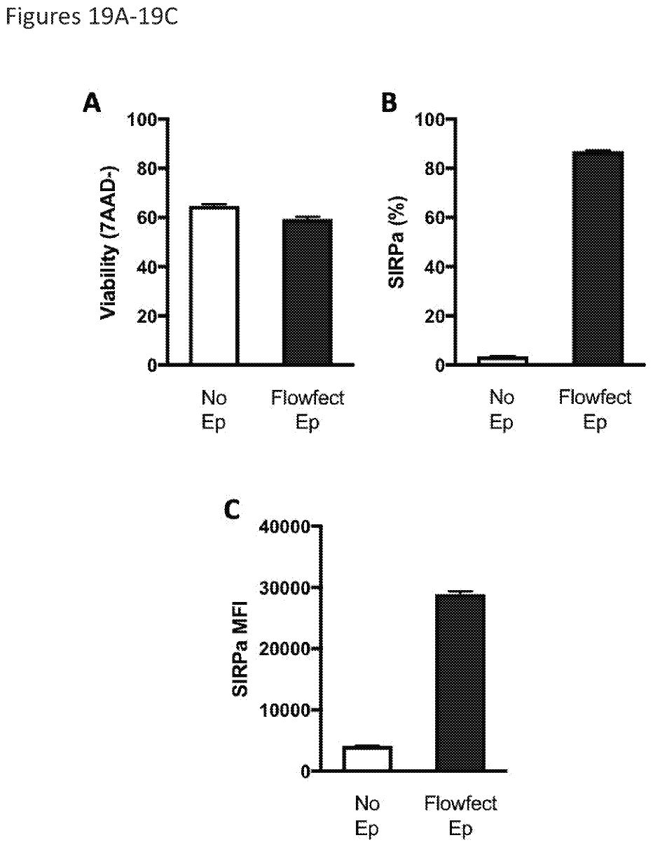

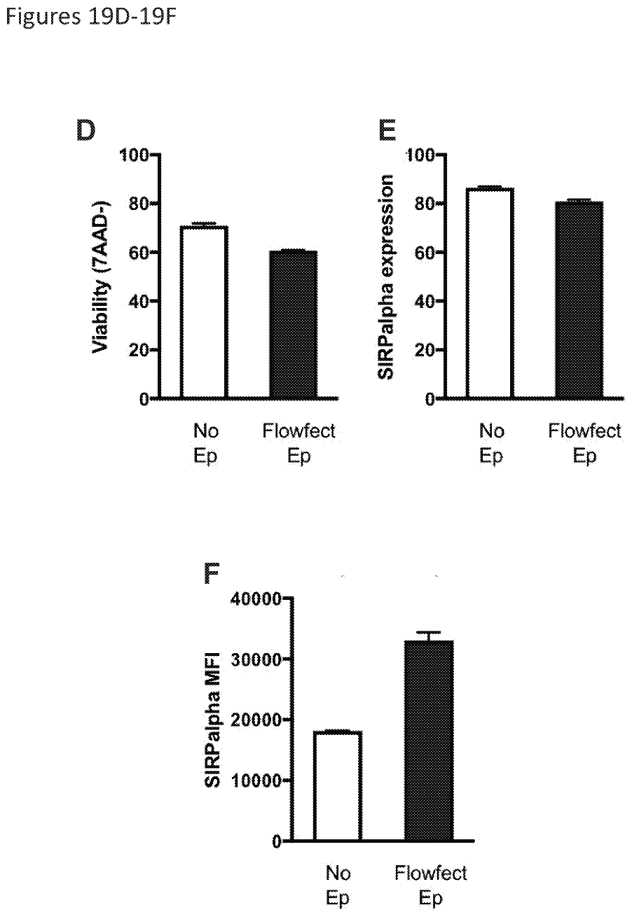

In another study, transient expression of SIRPalpha in primary human monocytes was achieved using devices of the invention (FIGS. 19A-19F). This delivery of a non-GFP mRNA in primary human monocytes further showcases the ability of the device of this transfection platform to function in this historically “hard-to-transfect” immune cell population. As a control for this overexpression demonstration, primary T cells were used, which are largely SIRPalpha negative (only 3.4% of live T cells were positive for the surface marker; FIG. 19B). After transfection, 86.9% of live T cells were positive for the SIRPalpha surface marker (FIG. 19B). In primary monocytes, which have a high baseline (86.5% positive for the surface marker (FIG. 19A)), mean fluorescence intensity (MFI) was quantified to determine if receptor expression density increased after transfection. A 1.8-fold increase over control cell baseline in SIRPalpha expression was observed 24 hours after delivery of mRNA (FIG. 19F).

CXCR4-Targeting Cas9-RNP Delivery to Primary Macrophages

eGFP labeled Cas9-RNP has also been successfully delivered to monocyte-derived human macrophages using devices of the invention. Delivery of the eGFP labeled Cas9-RNP to the nucleus was confirmed via microscopy and flow cytometry. eGFP expression was observed in up to 21.4% of differentiated macrophages 24 hours after transfection, which dropped to 5.1% within five days. While no gene editing was observed at the 24-hour time point, by 48 hours, a 13.9% KO efficiency was observed. Knock-out efficiency, as determined by flow cytometry, then increased to 16.5% by day five.

Naive T Cell Engineering with Delivery of mRNA

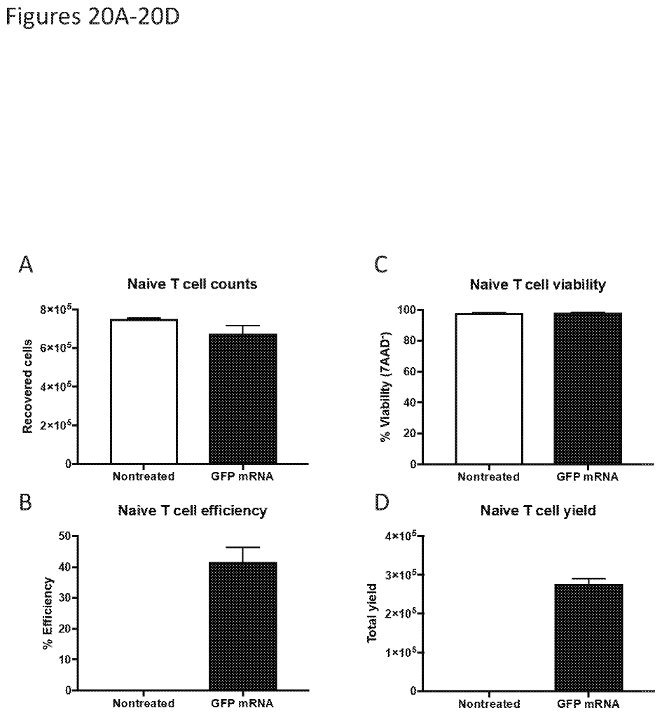

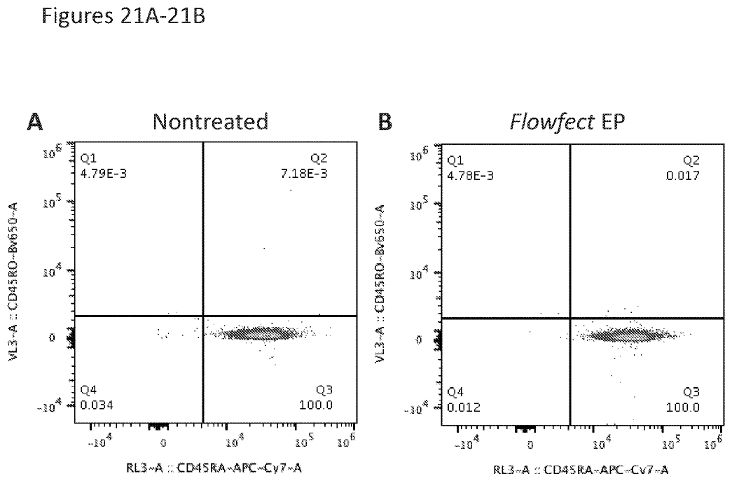

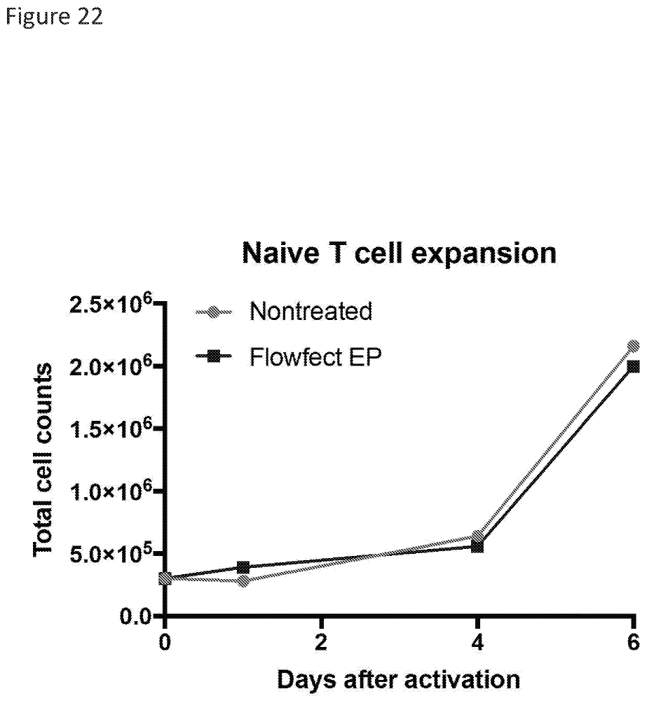

Isolated naïve T cells (CD45RA⁺/CD45RO⁻) were electroporated with mRNA encoding GFP using the device of the invention. After 24 hours, cells were analyzed for viability and efficiency metrics. The naïve cell counts and viabilities for electroporated cells were equivalent to nontreated cells, and ˜40% delivery efficiency was observed (FIGS. 20A-20D). Additionally, the cells were stained for naïve T cell markers CD45RA and CD45RO. This staining demonstrated there was no change in phenotype for the electroporated cells and that the cells retained their “naïve” CD45RA⁺/CD45RO⁻ state (FIGS. 21A and 21B). Lastly, the naïve T cells were expanded with CD3/CD28 activation reagents. In this experiment, the growth rates of electroporated cells were equivalent to the nontreated cells out to six days after activation (FIG. 22).

### Example 6—Devices for Energizing a Plurality of Devices of the Invention

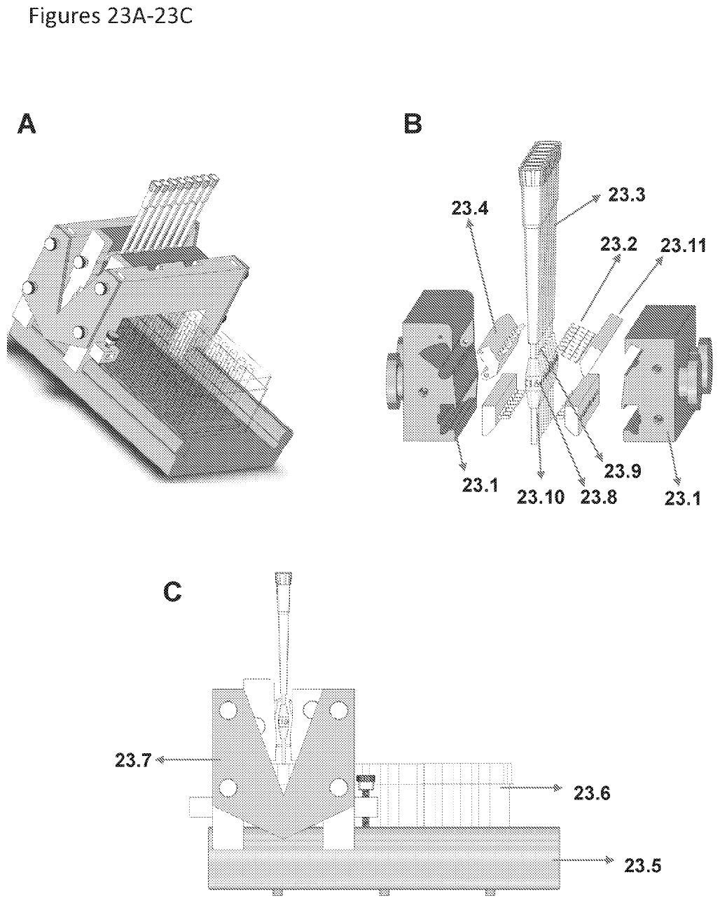

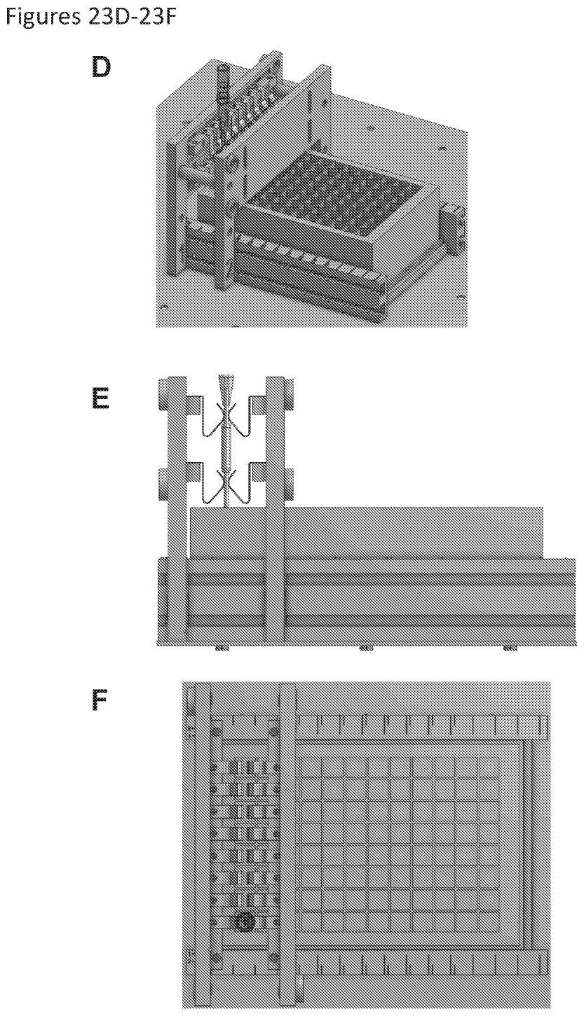

FIGS. 23A-23F show exemplary embodiments of electroporation devices of the invention integrated into an external device that can be further integrated into a liquid handling system for energizing the devices of the invention and complete the electroporation process on an automated liquid handling platform. The external device, called an electronics discharge machine (EDM) is used to energize the devices of the invention during the electroporation process. In the devices shown in FIGS. 23B, 23C and 23E, 23.1 are parallel beams that integrate with a support rails. These beams are interchangeable and allows for the change in electrical contact styles/mechanisms. In addition, the beam allows final positioning of the electrical contacts. 23.2 are mechanically retractable electrical contacts. The electrodes use a spring like mechanism to allow different regions of the device to slide throughout the EDM while maintaining contact with the body of the electroporation device. This element can be switched for other electrical contacts that are more flexible, e.g., leaf springs such as those shown in FIG. 23E or wire brush type electrodes. 23.3 is a reservoir of the electroporation device of the invention. 23.4 is a swinging support rail that allows for additional deflection of the electrode if needed. This rail feature uses a spring-like mechanism in order to rotate and allow more deflection of the electrical contact while the electroporation device is being placed into position by an operator or automated system, e.g., a robotic arm. 23.5 is a sliding rail that allows for linear translation of a sample holding plate, such as the sample plate shown in 23.6. 23.7 is an alignment system that provides for proper electroporation device positioning over the sample plate. The alignment system is used as a visual indicator when there are no automated alignment features, e.g., there are no robotic control applied to the EDM. With application of some form of linear translation device, the system has the ability to complete 1 or more samples in any array format. 23.8 is the electroporation zone of the devices of the invention and is fluidically connected to both entry zone 23.9 and recovery zone 23.10. 23.11 is a support rail that supports the mechanically retractable electrical contacts (23.2). The support rail 23.11 may be electrically conductive such that all the mechanically retractable electrical contacts (23.2) can be energized for a simultaneous electroporation experiment. Alternatively, the support rail 23.11 may be a non-conductive material that isolates the mechanically retractable electrical contacts (23.2) such that individual electroporation experiments may be performed.

When configured as an automated system, the sample of the specimen of interest is aspirated in another location on the liquid handling platform by the devices of the invention. The sample is then transported over to the EDM where the electrode contacts are suspended over the surface of the sample plate. The devices of the invention are then lowered into the device in order to establish contact with the electrode contacts of the EDM. The mechanism depicted in FIGS. 23A-23C uses a pogo pin connection to close the circuit while the embodiment of FIGS. 23D-23F uses flexible spring, e.g., leaf spring, electrodes to close the circuit. Alternative methods of connecting the circuits include the use of conductive fluids or electrolytes, conducting diaphragms that expanded to make contact, or other conductive flexible materials that have a sufficient spring constant to deflect during the insertion process. This enables the EDM to be amenable to the use of a variety of different sized devices of the invention. The system can be used to electroporate one or more samples independently or simultaneously depending on the experimental objectives. This technology can be scaled up to increase throughout. For example, the EDM can be used with a plurality of electroporation devices of the invention, or alternatively, with a single device of the invention in a single sample experiment or multi-sample experiment by the addition of two linear translation mechanisms.

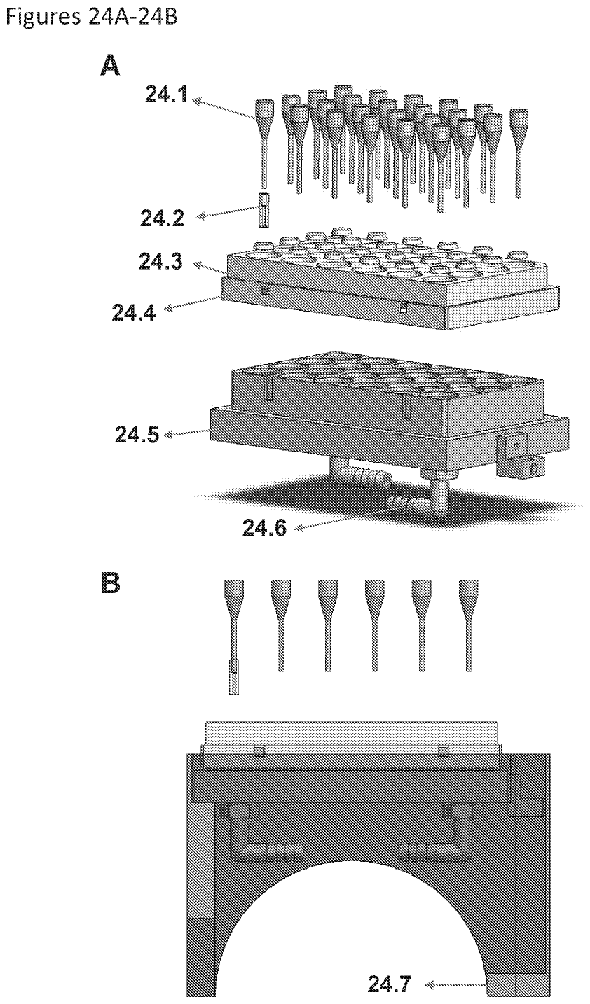

FIGS. 24A and 24B provide example embodiments of a housing configured to energize conductive devices of the invention in a temperature-controlled manner. In the device of FIG. 24A, 24.1 are hollow electrodes that are configured to be connected to a liquid handling manifold. The electrodes may further incorporate an interaction collar to reduce the stress on the electrode material induced by the friction generated by the connection to the liquid handling manifold. 24.2 is a connecting channel that is fluidically connected to the hollow electrodes and configured to amplify the electric field generated upon energizing the electrodes. The connecting channel further acts a barrier to confine the fluid flow in order to increase and control the electric pulse that the sample experiences. 24.3 is a conductive base electrode that connects to the connecting channel 24.2. 24.4 is a support base that is configured to hold hollow electrode 24.1, connecting channel 24.2, and conductive base electrode 24.3. 24.5 is a conductive base that both supports hollow electrode 24.1, connecting channel 24.2, conductive base electrode 24.3 and support base 24.4 and electrically connects to conductive base electrode 24.3 to complete the electroporation circuit. Conductive base 24.5 includes fluid connections 24.6 to flow heating or cooling fluid through the conductive base 24.5 to regulate the temperature of the electroporation process. In FIG. 24B, 24.7 is an outer frame that supports the other components.

In the device FIGS. 24A and 24B, as fluid flows from the hollow electrode 24.1, the conductivity of the sample fluid forms a closed circuit after interaction with the surface of the base electrodes 24.3. The base electrodes 24.3 can be of any shape that allows for a systematic and controllable electric field exposure that the cells experience which induced electroporation. The position of hollow electrodes 24.1 can be manipulated in the Z-coordinate from the support base 24.4 in order to limit the cells exposure to electric field. In this configuration, the base electrode 24.3 is raised from the bottom of the support base 24.4 to a position that sits above a specified volume collection limit. The electroporated cell will experience a finite electric field throughout the sample (except to close the electroporation circuit). This design reduces shear effects on the sample cells and increases the uniformity of the flow in the region where electroporation occurs. In addition, to create a stable electric field or to manipulate the electric field further, connecting channel 24.2 is added to the end of the hollow electrode 24.1, enabling the operator to amplify and control the electric pulse, and thus the electric field, experienced by the specimen. In addition, the electrode configuration in this system uses a non-parallel electrode configuration where the cannula is circular and parallel to the axis of the flowing specimens, but the base electrode's 24.3 surface is at some angle greater than 0 degrees with respect to the axis of the cannula. A variation of this design is the use of a suspended electrode that hovers over the well plate. As the sample flows across the surface the base electrode 24.3 and is electroporated, the sample falls into the well. In this configuration, the electrodes are not physically attached to the well plate.

### Example 7—Fluidic Chip-Based Electroporation Devices

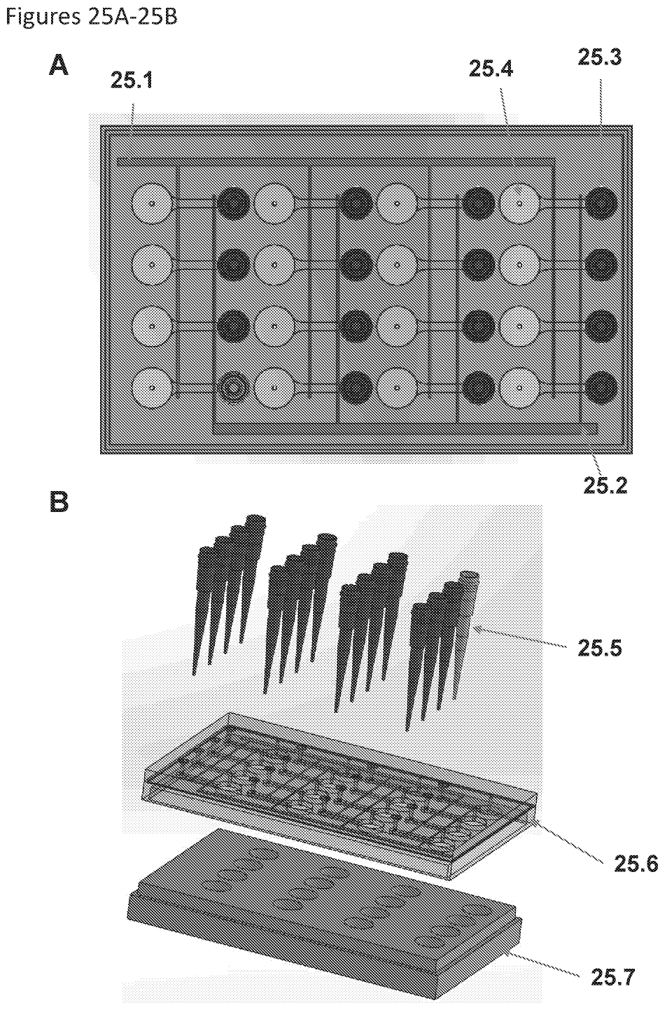

FIGS. 25A-25B show exemplary embodiments of a fluidic chip-based electroporation device that is configured to accept industry standard 1-5,000 μL conventional pipette tips to introduce samples to the device. In the device of FIGS. 25A, 25.1 and 25.2 are electrodes that are fluidically and electrically connected by an electroporation zone. 25.3 is a pipette tip insertion region fluidically connected to the electroporation zone and 25.4 is a collection reservoir. The electrodes 25.1 and 25.2 of the fluidic chip-based electroporation device are energized by an external power supply. In the exploded view of FIG. 25B, 25.5 are pipette tips, 25.6 is the fluidic chip-based electroporation device of FIGS. 25A and 25.7 show a collection plate to hold species after electroporation.

The pipette tips 25.5 hover over the surface of a fluidic chip-based electroporation device 25.6. The fluidic chip-based electroporation device includes two components: an electroporation plate that contains an encapsulated arrangement of electrodes and a cover plate that has embedded microfluidic channels that enable the user to modulate the pulse of the electric field that is delivered to the cells. The electroporation plate enables flow through electroporation of multiple samples simultaneously or individually if desired. After the electroporation of the specimen occurs in the electroporation plate the sample flows towards the bottom of the collection plate 25.7. This system uses industry standard liquid handling components, e.g., 1-5,000 μL pipette tips, facilitating integration into industry standard liquid handling manifolds.

### Example 8—Large Volume (Scalable) Continuous Flow Electroporation Device

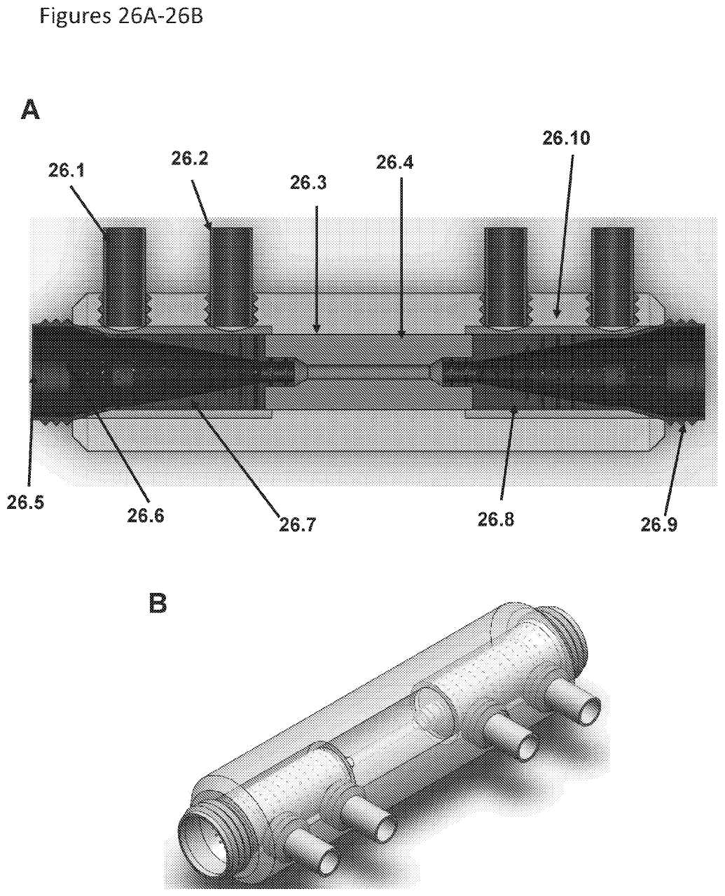

FIGS. 26A-26B show exemplary embodiments of a continuous flow electroporation devices designed for use with large volume cell manufacturing. In the embodiment shown in FIG. 26A, 26.1 and 26.2 are an inlet and outlet, respectively, for circulating a fluid, e.g., a buffer solution. 26.3 is an outer housing that holds the electroporation device. 26.4 is the electroporation zone and is fluidically connected to fluid inlet 26.5 and fluid outlet 26.9. After the inlet 26.5 and before the outlet 26.9 are cylindrical electrodes 26.7 and 26.8 that have pores 26.6 on their surface. 26.10 is a reservoir for holding a fluid, e.g., a growth media.

The cylindrical electrodes 26.7 and 26.8 in this embodiment are made of conductive porous material that allows the fluid to travel through its pores 26.6 into the cavity of the device. The pores 26.6 in the cylindrical electrode 26.7, 26.8 allow a buffer solution to stabilize the chemical reactions on the surface of the cylindrical electrodes 26.7, 26.8 and minimize the pH transition observed due to the application of an electrical potential during the electroporation process. The buffer introduced by the porous cylindrical electrodes 26.7, 26.8 allows for a change in the fluid flow to create a “lubricating” or sheath flow on the internal surface of the cylindrical electrodes 26.7, 26.8 or to induce other fluid dynamics elements to the electroporation process (such as rotation of the suspension with cells) as it is electroporated. The reduction of the pH transition reduces the negative effects of high variations in the pH of the suspended specimens used during electroporation. Cylindrical electrodes 26.7 and 26.8 complete the external circuit requirement and allow the system to be energized using an external power supply. In an alternative embodiment, the outlet 26.2 of the electroporation device can be used to remove a highly conductive buffer, e.g., a growth media or PBS, and inlet 26.1 can be used to introduce low electrical conductivity buffer to minimize heating of the liquid sample as it flows through the electroporation zone 26.4. This buffer exchange will result in a higher cell viability and higher transfection efficiency that ultimately will generate a greater number of successfully engineered cells. The low conductivity buffer can then be extracted in the outlet after the electroporation zone and supplemented with growth media upon contact with the inlet after the electroporation zone.

### Example 9—Modeling Electric Fields in a Novel Helical Electrode

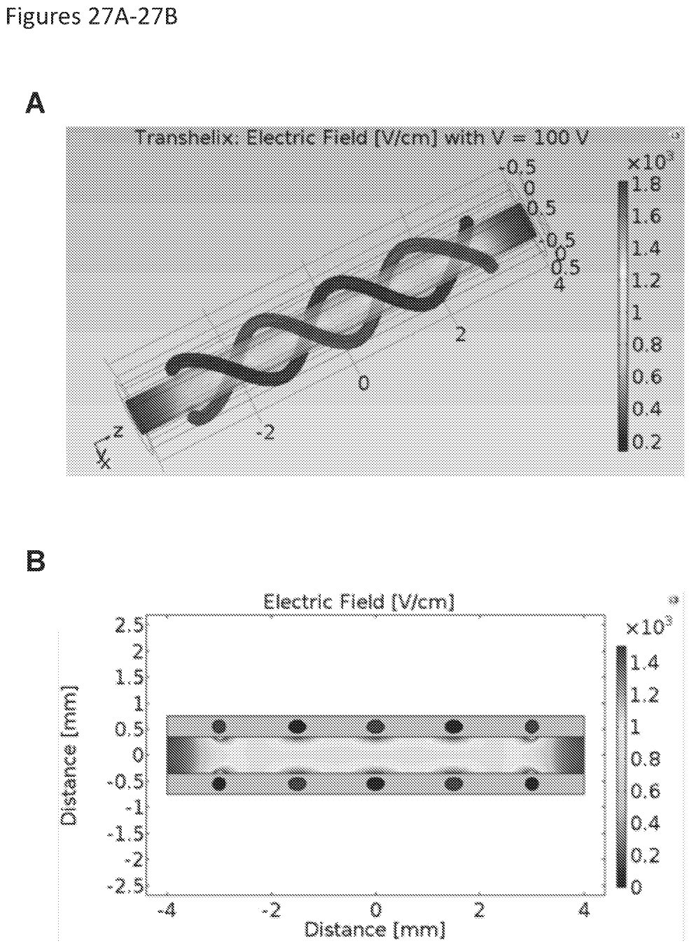

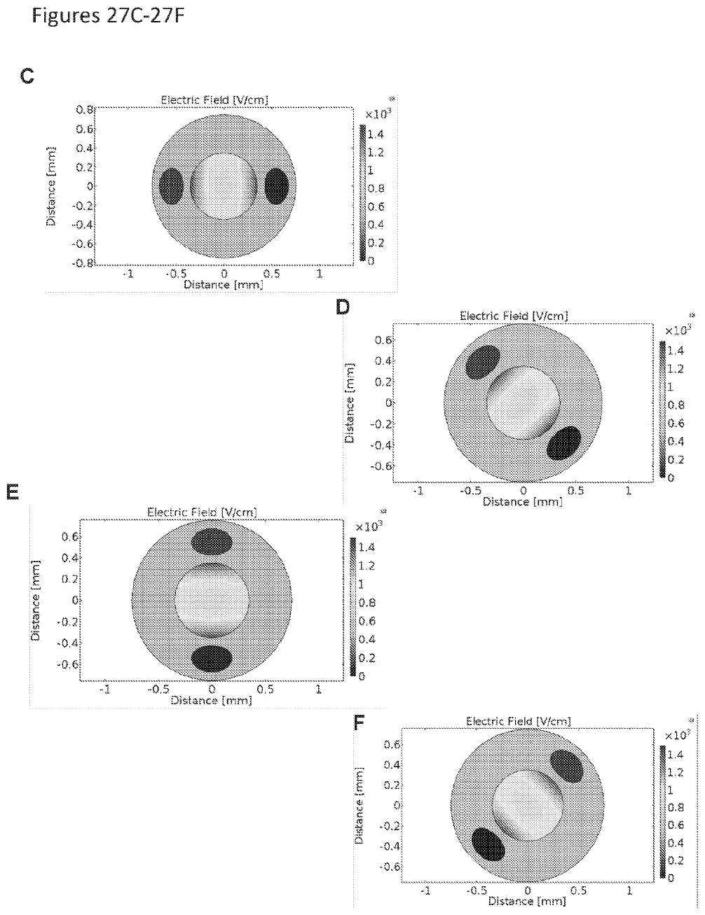

A Flowfect device with a particular electrode configuration to help increase the transformation/transfection efficiency of flowing cells has been designed and computationally modeled. FIG. 27A demonstrates the helical nature of the electrode configuration that is responsible for rotating the electric field as cells flow through the electroporation region. Without being bound by theory, this configuration allows a larger fraction of the cell surface to be electroporated and thereby requires lower electric fields to achieve equivalent effects. FIGS. 27B-27F show the cross-sectional area of the electroporation region, viewed from different axes. The energized and grounded electrodes are perpendicular to the flow direction as opposed to in the parallel direction, e.g., as in FIGS. 1A-1C. This design allows for lower sample volume and reduced applied voltage, which is desirable, e.g., in such applications as primary human cell (e.g., immune cell or stem cell) electroporation, in which cell number is limited. In another embodiment, the helical electrodes are not in fluid contact with the electroporation zone; the use of high-frequency pulses may induce an electric field inside of the electroporation zone (e.g., through an intermediate medium) to deliver composition into cells.

### Example 10—Two-Part Devices of the Invention for Manufacturing Scalability

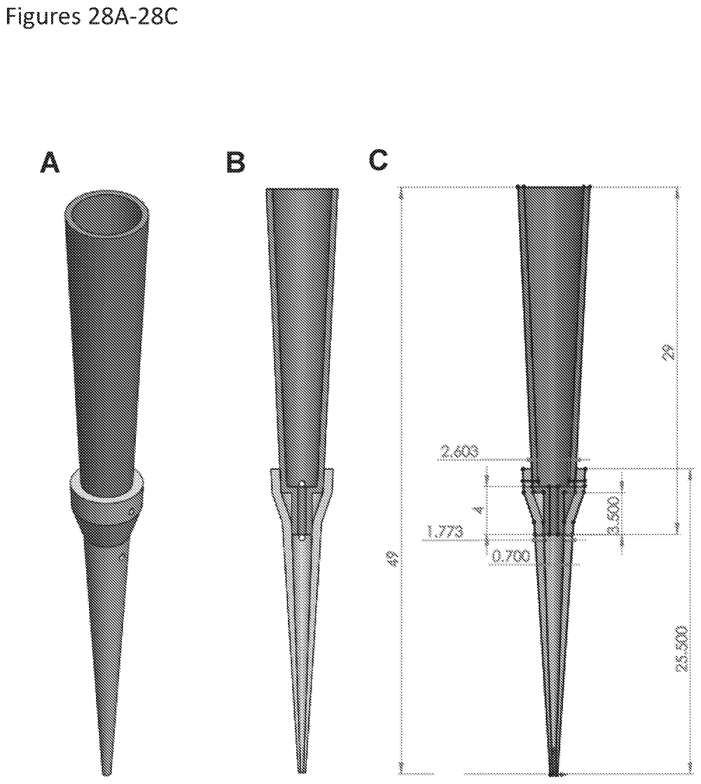

FIGS. 28A-28C show an embodiment of a device of the invention that is configured to be manufactured in two separate components that mate together to form a complete device that is capable for being used with commercially available liquid handling systems. In this configuration, the insert molded electrodes, shown as small dots near the junction of the two components in FIGS. 28A-28B will then be welded together via established industrial processes (e.g., spin welding, sonic, e.g., ultrasonic, thermal welding, e.g., a hot plate, or laser). In this design, the fluid flow of a sample, e.g., a cell-DNA sample, through the device is decoupled from the electric field exposure required for electroporation.

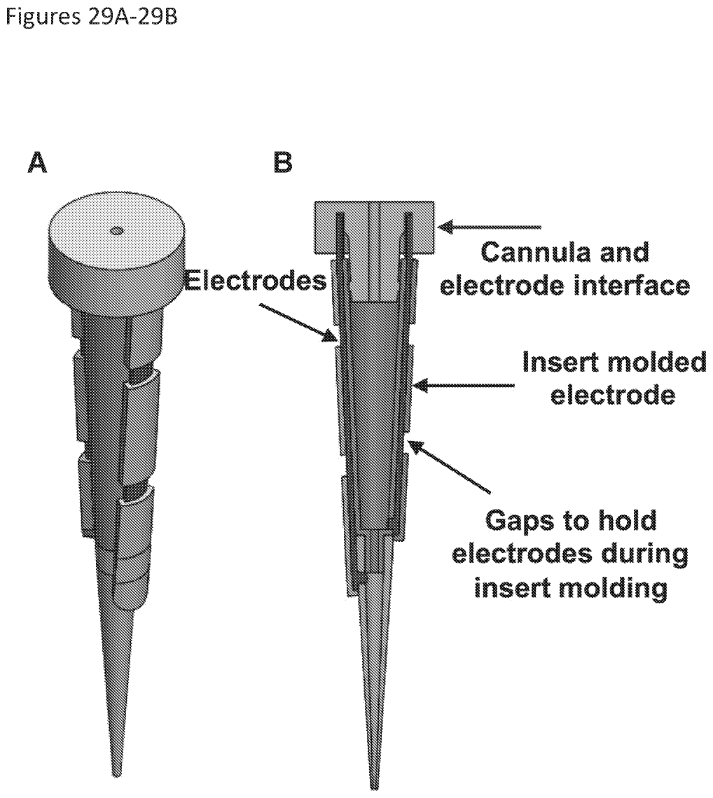

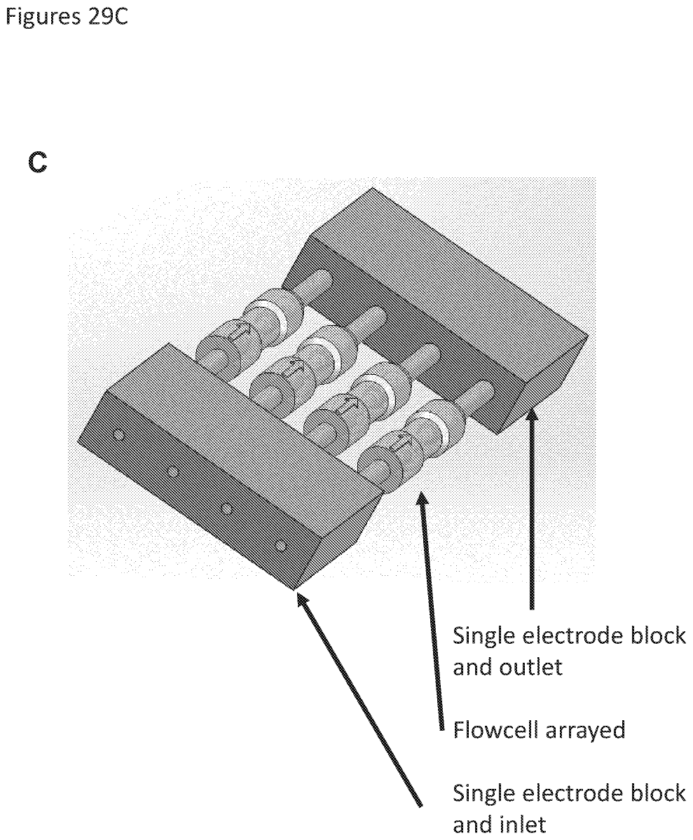

FIGS. 29A and 29B show the device depicted in FIGS. 28A-28C, e.g., identical internal dimensions, with 4 mm distance between insert molded electrodes above and below a 700 μm diameter electroporation zone. The difference between this embodiment of the device of the invention and the embodiment shown in FIGS. 28A-28C is that in this concept the fluid flow control is coupled with the electric field exposure. Specifically, the cannula (shown at the top of the device of FIGS. 29A-29B) is the interface between the liquid handling system and the electroporation device of the invention. Once the electroporation device of the invention interlocks into the cannula, the embedded electrodes (shown in red in the device of FIGS. 29A and 29B) will be in electrical connection with the power supply for voltage pulse delivery. In the embodiment shown in FIGS. 29A-29B, a single cannula is shown, but can be scaled up in a system of the invention to include a plurality of electroporation devices of the invention, e.g., a system containing 4 (FIG. 29C), 8, 12, 24, 36, 64, 96, or 384 electroporation devices of the invention configured to electroporate cells suspended in a fluid in parallel. FIG. 29C illustrates such a method/device that can increase the number of simultaneous or sequential transfections that occur using either a plurality of cartridges or a plural of controllers. In particular, it incorporates the multiple inlets to modify cells in combination/separately using multiple fluid constraining boundaries/constriction that are exposed similar/different electrical signals and similar/different displacement velocities. Alternatively, cavities for cell modification are independent cartridges that are integrated into one/multitude of controller systems or the controller system can accept one/multitude of cartridges.

### Example 11—Examples of Housing and Interfaces

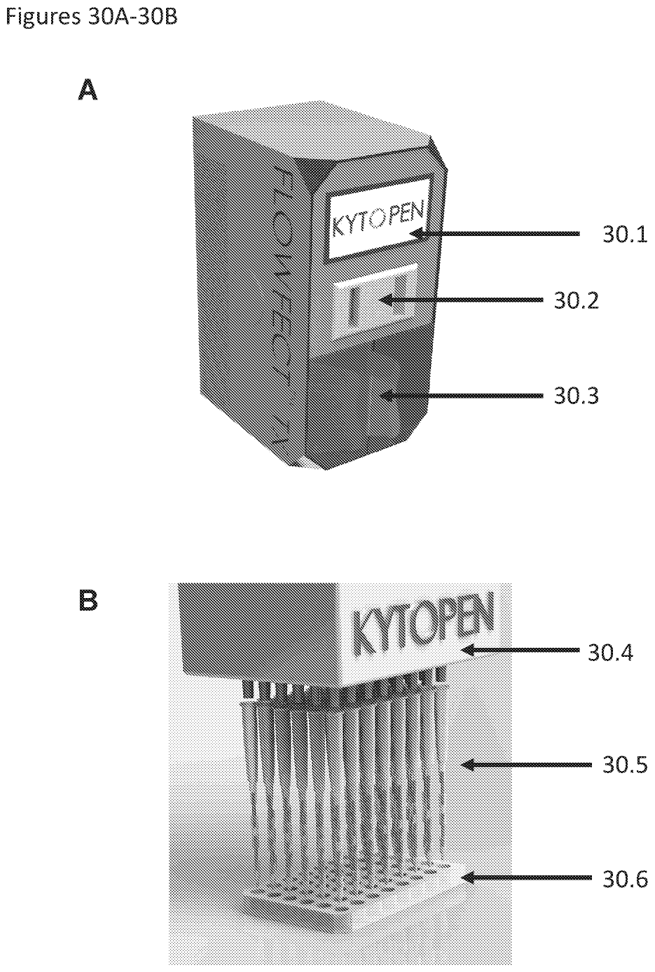

FIGS. 30A and 30B provide exemplary embodiments of devices of the invention showing an outer housing including a user interface (FIG. 30A) and a plurality of devices of the invention fluidically connected to a liquid dispensing manifold and a sample plate (FIG. 30B).

FIG. 30A is an embodiment of the continuous flow transfection/transformation system. The 3D model shows a standalone electroporation system that contains a touchscreen user interface (30.1) or another alternative user interface(s) that enables the user to select parameters such as flow rate, waveforms, applied potential, volume to electroporate, time delay, cooling features, heating features, electroporation status, progress and other parameters used to optimize the electroporation protocol. The interface also contains pre-formulated parameter selections that enable the user to operate the system at standard conditions that have previously been validated by user or as recommended by the manufacturers. The interface may be connected to programming that allows for automated running of the system and/or running an algorithm to optimize electroporation for a given sample of a known cell type. The device also contains a cartridge (30.2) that encapsulates one or more of the previously stated inventions or another electroporating devices used for continuous flow electroporation. The device also contains a cooling/heating area/enclosure (30.3) for cell/buffer storage during, before and after electroporation of the specimen. The system is externally powered. The system also contains, algorithms that have the ability to adjust parameters independently/autonomously if the user selects this functionality. This allows for continuous adjustment of the parameters used in the electroporation process that may depend on the cell type, conductivity, volume of suspensions, viscosity, lifetime of the electroporating cartridge, the physical state of the suspension or the state of the electroporation device.

FIG. 30B shows an array of electroporating devices previously described in the document. 30.4 is the liquid handling manifold that transport the invention across the liquid handling platform and enable the device to aspirate fluid. 30.5 is the device shown in FIGS. 1A-1C. 30.6 is a well plate used to store sample before, during, and/or after the specimen transfer.

### Example 12—Gating Strategies for Flow Cytometry to Optimize Electroporation Parameters

FIG. 31 provides an example comparing two gating strategies. Historically, developers of electroporation technology have used a canonical “lymphocyte” pre-gate, which ignores cells that are not within the “lymphocyte” population, such as those with an altered morphology or undergoing apoptosis. As shown in FIG. 31, this artificially increases the viability metrics by selecting a specific subpopulation of cells for analysis. A “total cell” pre-gating is a more accurate depiction of the experimental outcomes from electroporation. Therefore, the reported viabilities shown in the table below may appear lower than expected in the field, but the data have been processed to focus on performance metrics which depict the impact of the electroporation devices of the invention on all input cells. In FIG. 31, FSC stands for Forward Scatter and SSC is Side Scatter, indicating how cell morphology data is collected during the flow cytometry analysis.

Using the gating strategy described herein, performance data for Jurkat cells, activated primary human T-cells, THP-1 monocytes, primary human monocytes, and differentiated primary human macrophages are shown below in Table 2. In Table 2, Yield represents the ratio of the numbers of cells that are viable and expressing the payload of interest to the input number of cells that entered the process. For example, Yield of 0.5× means that one half of the input cells are viable and express the desired payload at the time of analysis. For perspective, a cell therapy product is administered to a patient if the yield with viral delivery is greater than approximately 0.1× at the time of harvest.

TABLE 2
Representative performance metrics achieved
with devices of the invention in different primary
cells and cell lines with a wide variety of payloads.

| Input | | Peak performance metrics | | |
|---|---|---|---|---|
| Cell type | Payload | Viability | Efficiency | Yield |
| Jurkat cell line | dextran | 75-80% | 55-60% | 0.3X |
| | pDNA | 70-75% | 55-60% | 0.2X |
| | mRNA | 75-80% | 90-95% | 0.6X |
| Primary human | dextran | 75-80% | 85-90% | 0.5X |
| T-cells (activated) | mRNA | 75-80% | 90-95% | 0.6X |
| THP-1 | dextran | 65-70% | 85-90% | 0.5X ‡ |
| Primary human | dextran | 45-50% | 85-90% | 0.3X ‡ |
| monocytes | mRNA | 55-60% | 80-85% | 0.4X ‡ |
| Primary human | dextran | 70-75% | 70-75% | 0.4X ‡ |
| macrophages | mRNA | 45-50% | 75-80% | 0.2X ‡ |
| (differentiated) | | | | |

‡ Represents yield based on non-treated no-electroporation control counts

### Example 13—Electroporation into Chinese Hamster Ovary (CHO-K1) Cells and Human Embryonic Kidney (HEK-293T) Cells

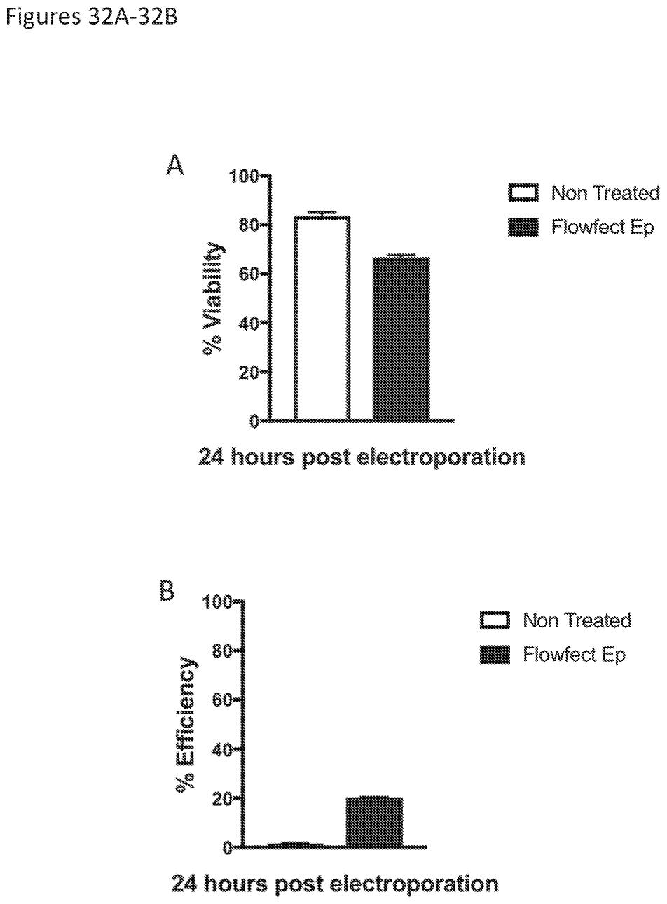

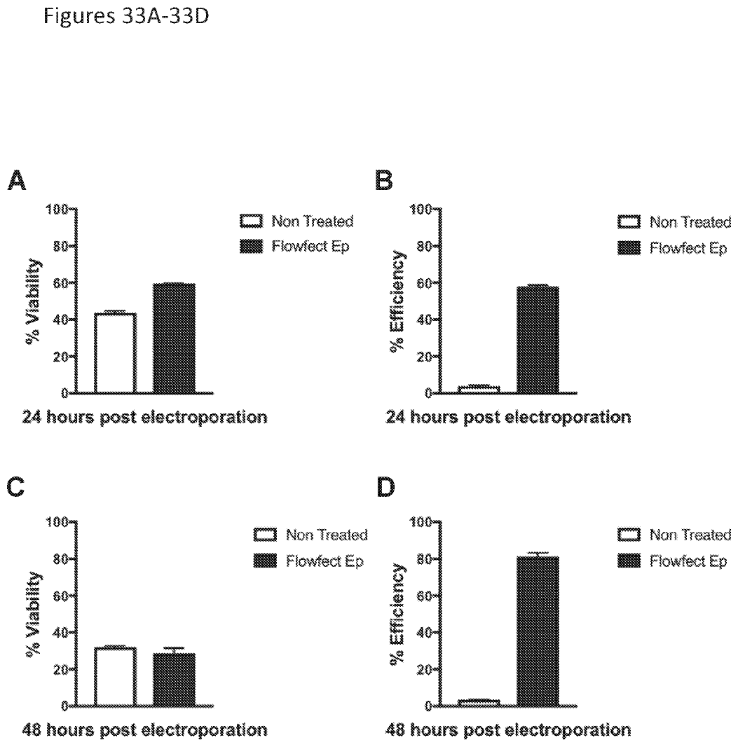

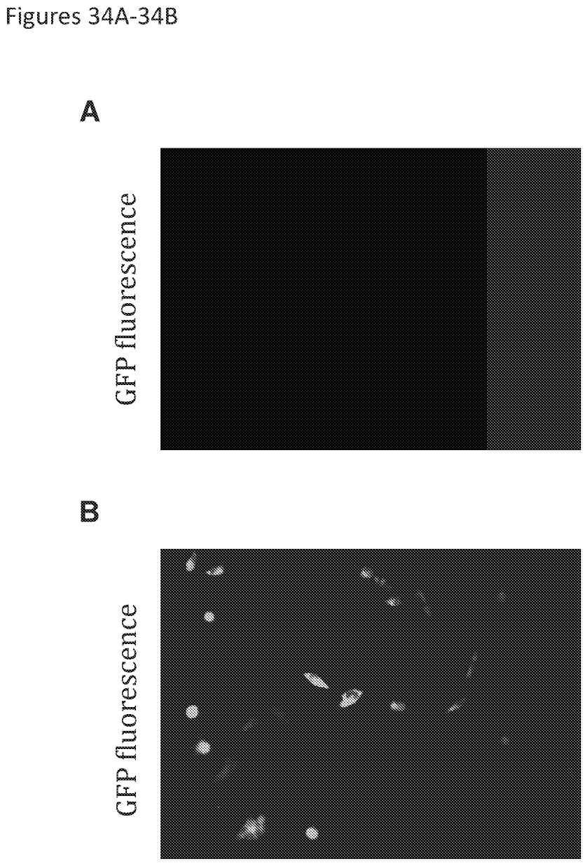

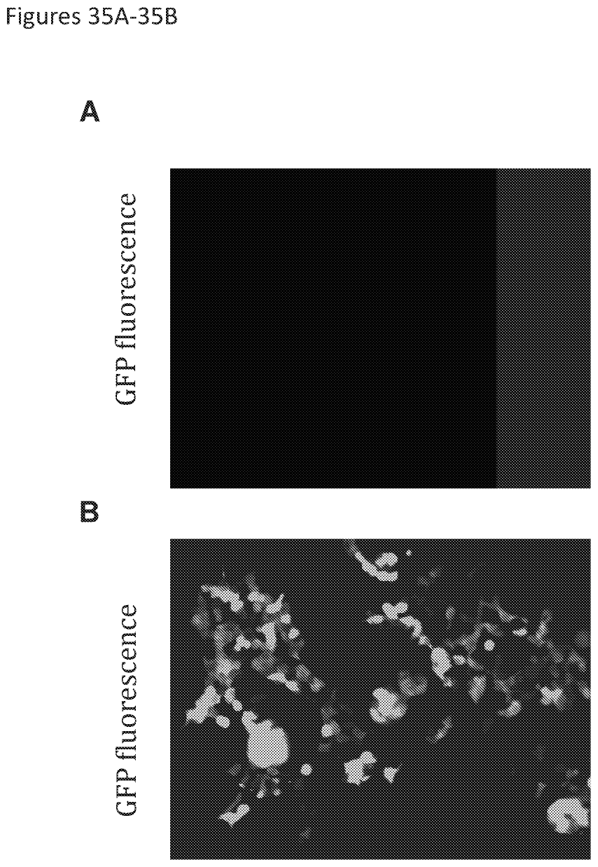

Electroporation of the CHO-K1 (Chinese hamster ovary cells) and HEK-293T (human embryonic kidney cells) cell lines has been conducted. Devices of the invention can be used for electroporation of adherent cells that have been lifted and resuspended in an electroporation buffer. CHO-K1 (FIGS. 32A and 32B) and HEK-293T (FIGS. 33A-33D) cells can be successfully transfected with GFP plasmid DNA using devices of the invention. Peak transfection efficiency in HEK-293T cells was observed after a 48 hours culture, post electroporation. Without being bound by theory, the reduced cell viability may be due to lifting the adherent cells and placing them in suspension for analysis via flow cytometer, whereas microscopy methods showed healthy GFP+ cells with normal morphology (FIGS. 34A, 34B, 35A, and 35B).

### Example 14—Transfection of Primary T-Cells

Studies in primary T-cells have been conducted. Fluorescent reporters that have been primarily utilized for analysis of electroporation efficiency include fluorescent small molecules (e.g., FITC-labeled dextran), genes expressed from plasmid DNA (e.g., GFP), and genes expressed from mRNA (e.g., GFP). Delivery and expression of these reporters is determined using flow cytometry, in which the live cells are pre-gated using the gating strategy as described herein to determine fluorescent detection on a single-cell basis. These assays demonstrate intercellular detection of the fluorescent reporter, and in some cases, direct nuclear delivery. Due to the gentle nature of electroporations performed with devices of the invention, higher cells counts are achieved after transfection compared to commercial systems, e.g., the Lonza 4D-NUCLEOFECTOR system or NEON® transfection system (Thermo Fisher, Carlsbad, Calif.).

a. Expanded T-Cell Demonstrations

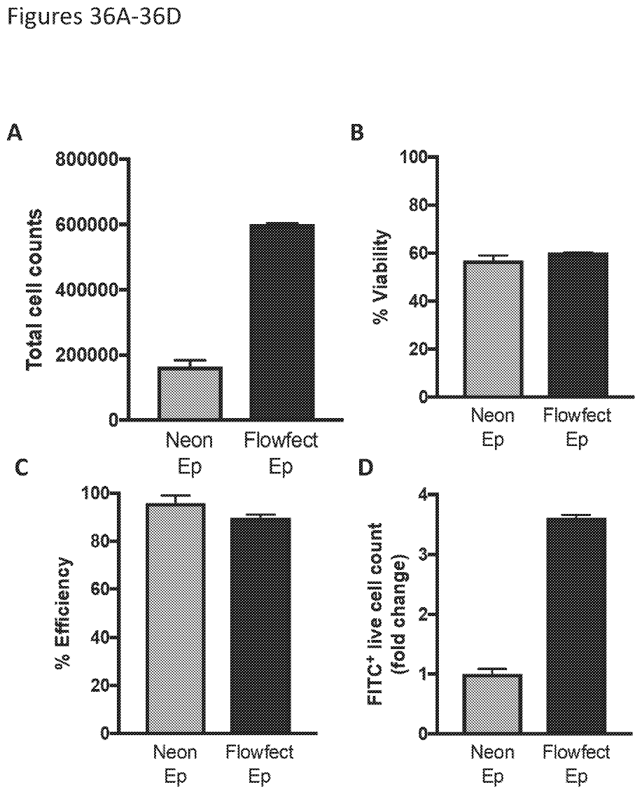

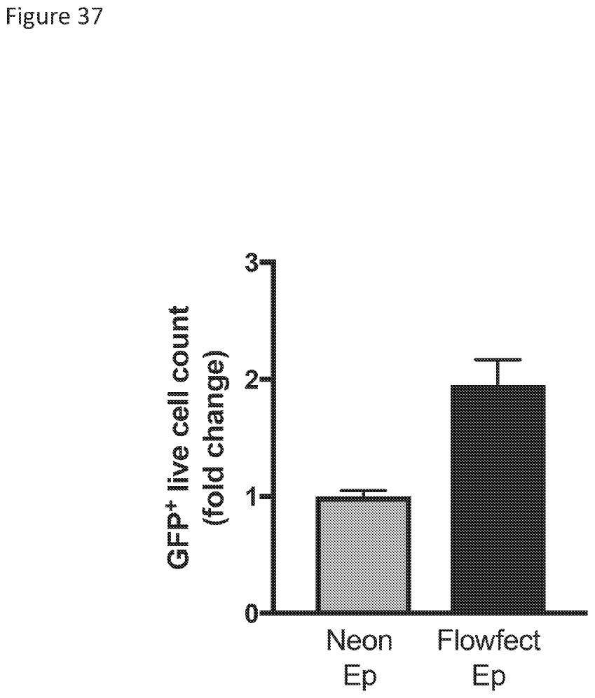

Transfection using devices of the invention to deliver fluorescently labeled (FITC) dextran molecules (40 kDa) into primary human T-cells (starting at cell density of 10⁶ cells/experimental condition) was performed, and analysis of four metrics against a commercially available bench-top electroporation device (e.g., a Thermo Fisher NEON® transfection system) was conducted: total cell count (post EP), cell viability, transfection efficiency, and total number of live transfected cells. Results are shown in FIGS. 36A-36D. In addition to the data shown in FIGS. 36A-36D using fluorescently labeled molecules, delivery of plasmid DNA encoding GFP (3.5 kB) into primary human T-cells (at a cell density of 10⁶ cells/experimental condition) was tested using devices of the invention. These experiments again demonstrated superiority to the NEON® transfection system, shown as the total number of GFP expressing T-cells after a 24 h incubation depicted in FIG. 37. Importantly, expression of GFP from DNA plasmid also demonstrated effective delivery of genetic information (i.e., nucleic acids) into the nucleus, where DNA is transcribed into RNA prior to translation into the final GFP protein.

b. Delivery of mRNA with Platform Comparison

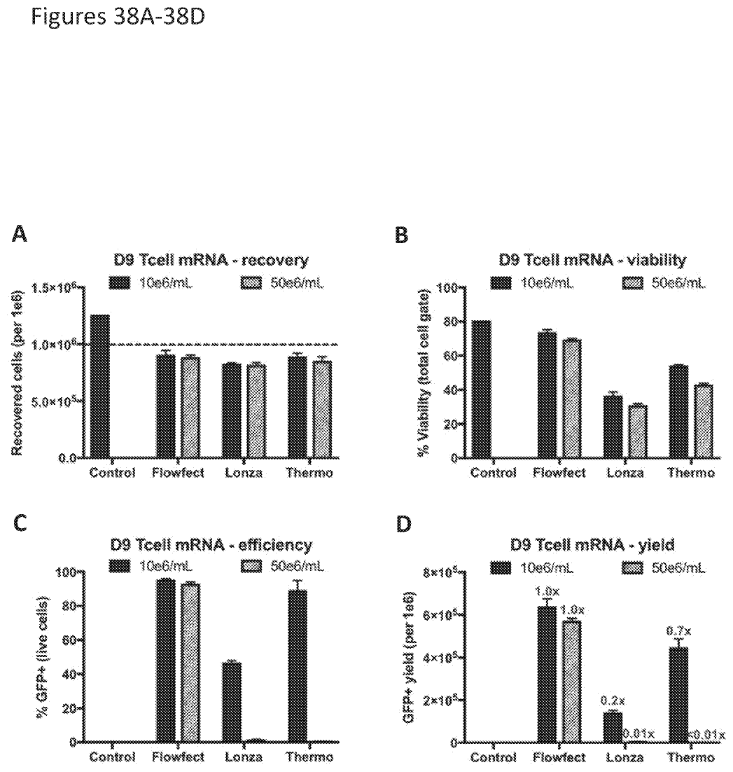

Delivery of mRNA to cells was also demonstrated using devices of the invention. These experiments were performed with a commercially sourced mRNA at two operating cell densities. The experiments were then completed on two commercially available systems (Lonza 4D-NUCLEOFECTOR and Thermo Fisher NEON® Transfection System) and the devices of the invention for comparison as shown in FIGS. 38A-38D). The devices of the invention outcompeted the commercially available systems in terms of viability, efficiency, and yield. In addition, the performance of the devices of the invention was independent of cell concentration, unlike the commercially available systems, as indicated by the experimental results shown in FIGS. 38A-38D.

### Example 15—Delivery of a Non-Transient Payload

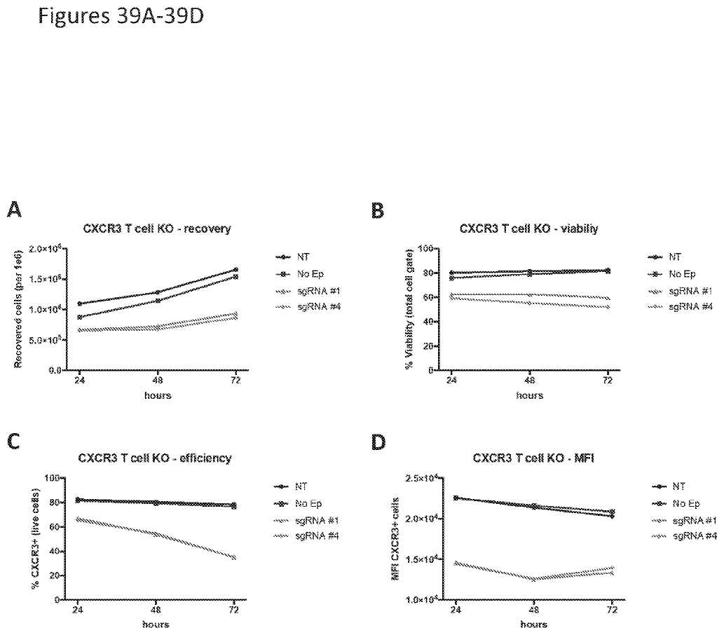

Each of the payloads described in Examples 13 and 14 are transient upon delivery. To demonstrate delivery of reagents stable genome modification (i.e., CRISPR gene knock-out), experiments were performed with Cas9 ribonucleoprotein complexes (RNPs) for CRISPR knock-out in primary cells. As is shown in FIGS. 39A-39D, knock-out of an endogenous gene in primary T-cells as confirmed through surface receptor staining on a single-cell basis was successful using devices of the invention and confirmed using flow cytometry. Devices of the invention may also be used for simultaneous CRISPR integration of an exogenous gene to demonstrate stable genomic integration through electroporation of Cas9 RNPs.

### Example 16—Monocyte (THP-1) and Natural Killer (NK-92M1) Cell Line Transfection

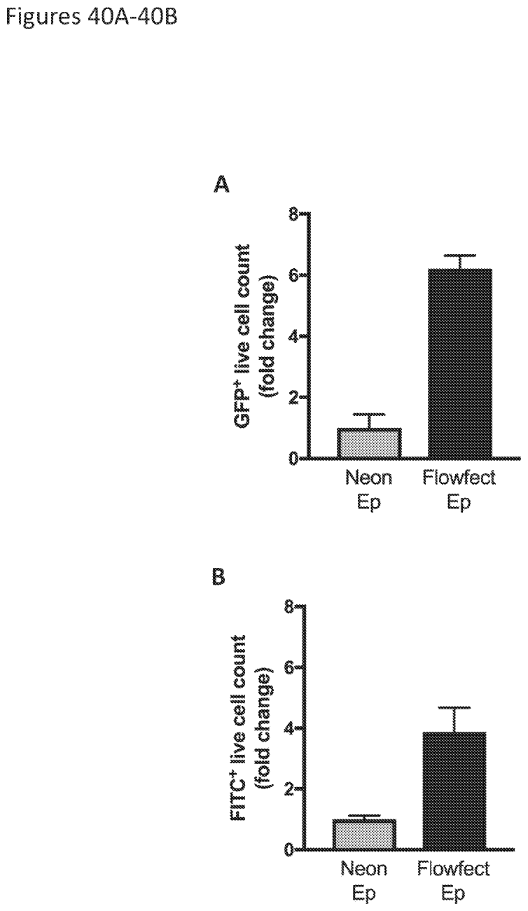

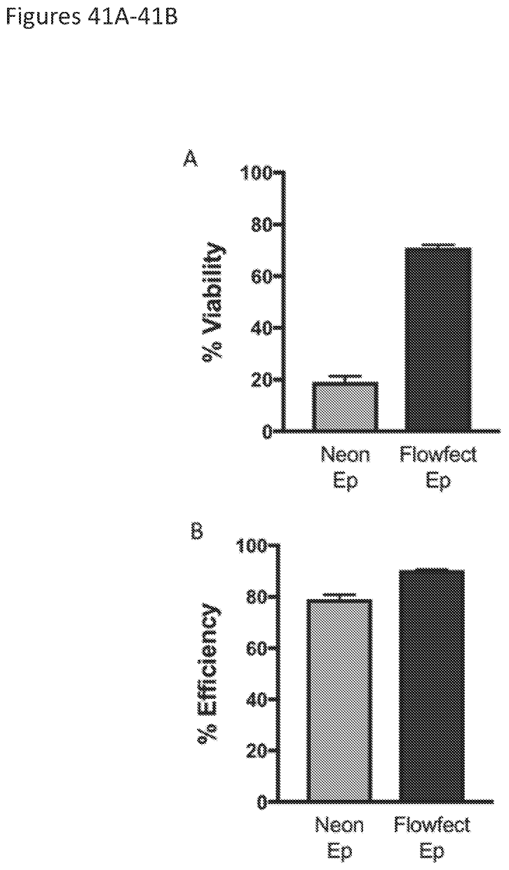

FIGS. 40A and 40B show bar graphs comparing the delivery of GFP plasmid and FITC labeled dextran to THP-1 and NK-92MI cells, respectively, using devices of the invention and a commercial NEON® transfection system. As is seen in FIGS. 40A and 40B, electroporation using devices of the invention consistently outperforms the NEON® for producing viable transfected cells of either type with either payload. As an additional comparative example, FIGS. 41A and 41B show increased cell viability and transfection efficiency in samples containing THP-1 monocytes, where GFP mRNA was delivered using devices of the invention compared to the NEON® transfection system.

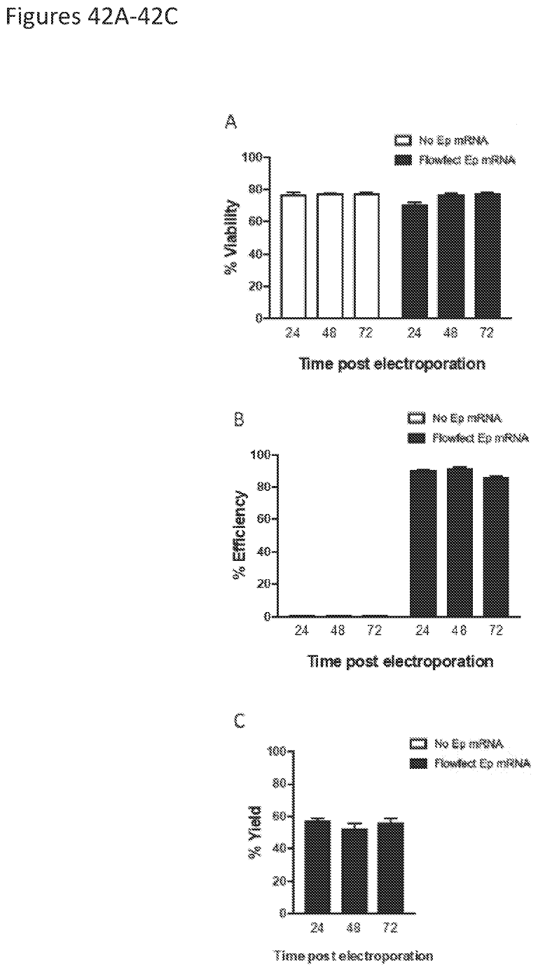

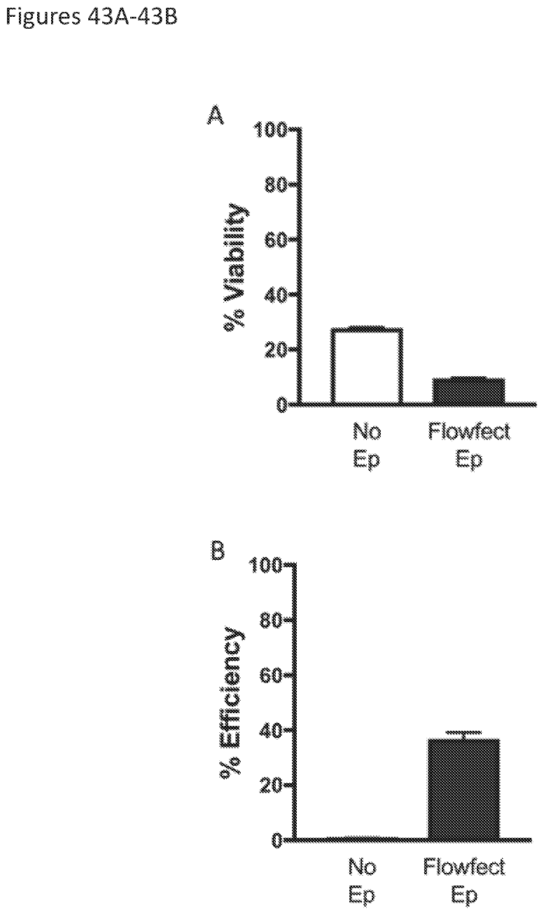

THP-1, an immortalized monocyte cell line, was further used for comparison studies with both monocytes and macrophages. Activation of THP-1 cells with LPS (lipopolysaccharide) endotoxin induces macrophage-like THP1-Mac immortalized cells. As shown in FIGS. 42A-42C and FIGS. 43A and 43B, both THP-1 (FIGS. 42A-42C) and THP1-Mac (FIGS. 43A and 43B) cells were successfully transfected with GFP mRNA using devices of the invention.

### Example 17—Primary Monocyte and Differentiated Macrophages Transfection

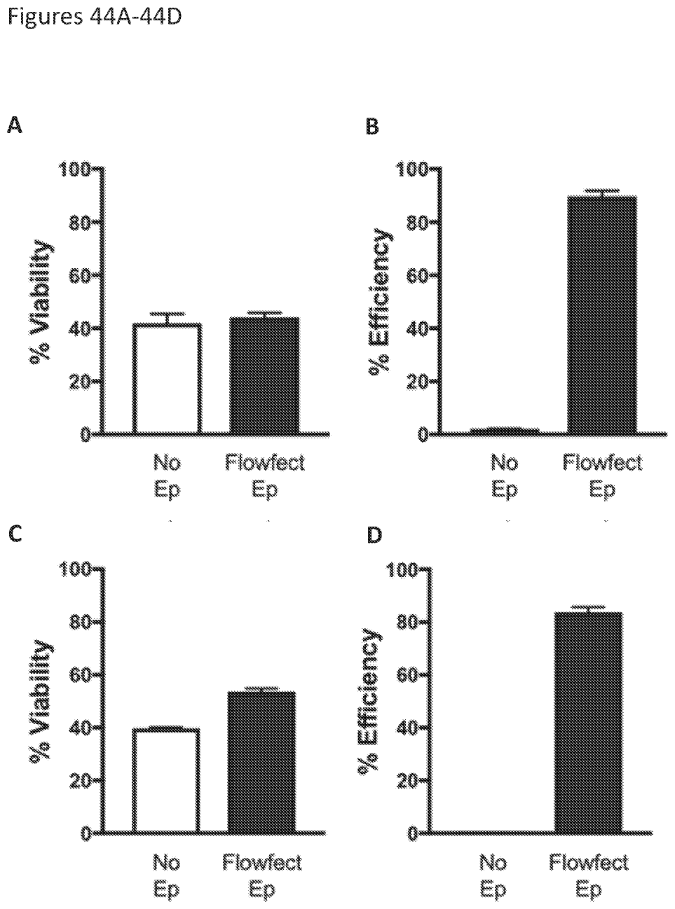

Primary human monocyte cells, a notoriously challenging cell type to transfect through conventional means, have been successfully transfected using devices of the invention. As is shown FIGS. 44A-44D, primary human monocytes, isolated from peripheral blood, were successfully transfected with FITC labeled dextran molecules and GFP mRNA using devices of the invention.

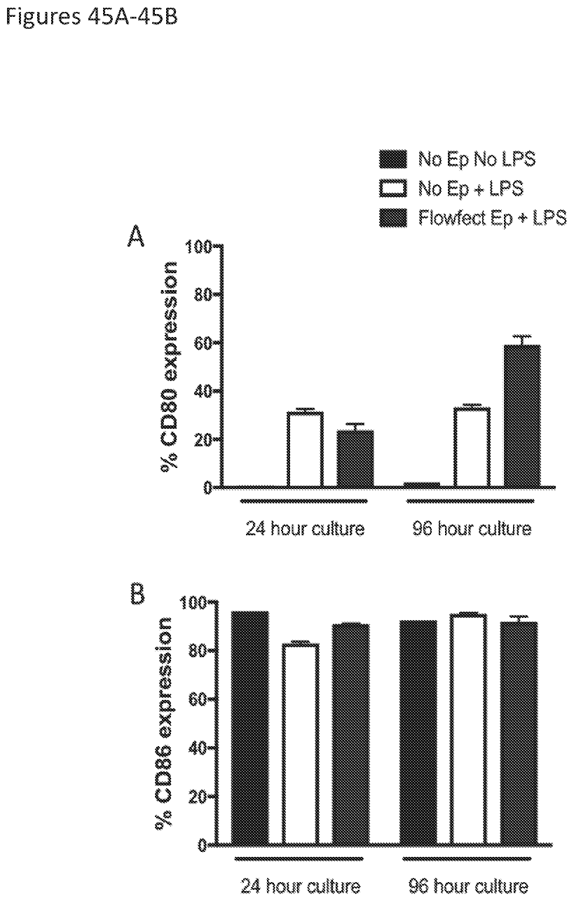

FIGS. 45A and 45B show the expression of specific markers in primary peripheral blood monocytes transfected with GFP mRNA using devices of the invention. As is shown in FIGS. 45A and 45B, the ability of CD86+ monocytes (gated on viable GFP+ cells) to become activated (represented here as CD80 expression) after LPS stimulation was maintained out to 96 hours, indicating that electroporation does not negatively impact expression of activation marker CD80 (FIG. 45A) or lineage marker CD86 (FIG. 45B).

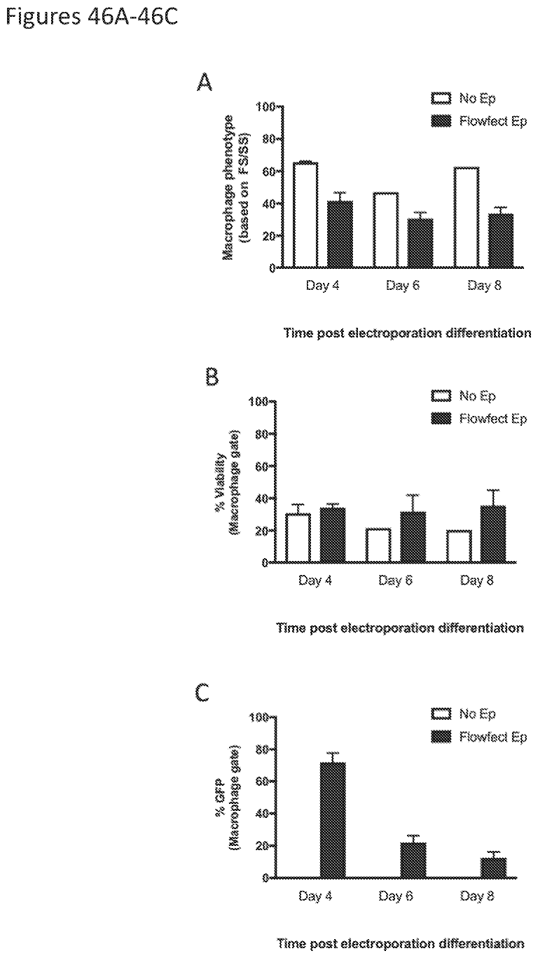

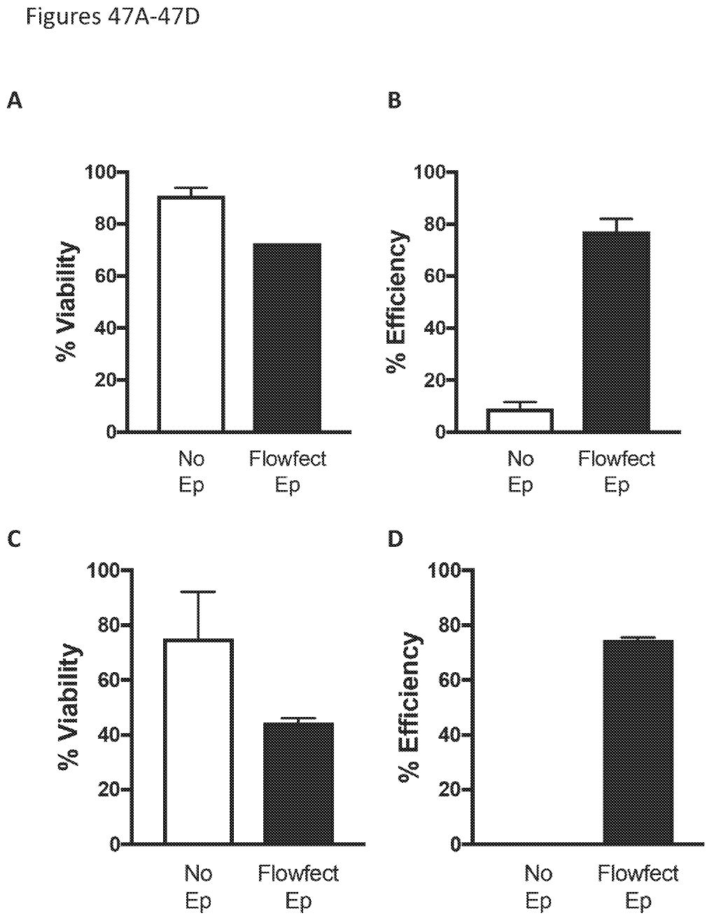

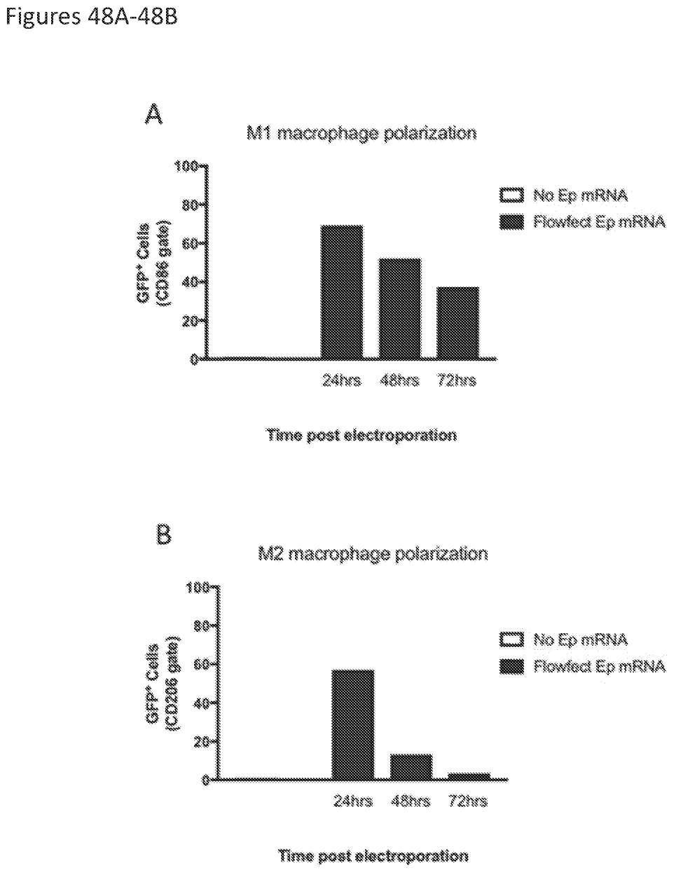

Further, primary monocytes electroporated using devices of the invention retained the ability to differentiate into macrophages, as shown in FIGS. 46A-46C, which indicates that the cells retain their function after electroporation. As shown in FIGS. 47A-47D, differentiated human macrophages were successfully transfected with FITC labeled dextran molecules (FIGS. 47A-47B) and GFP mRNA (FIGS. 47C-47D) using devices of the invention. Macrophages electroporated using devices of the invention polarized into M1 or M2 phenotypes (as shown in FIGS. 48A-48B), suggesting that cell health and function are retained after electroporation using devices of the invention. Electroporated macrophages were polarized into M1 (FIG. 48A) or M2 (FIG. 48B) phenotypes and retain GFP mRNA expression out to 72 hours post electroporation using devices of the invention.

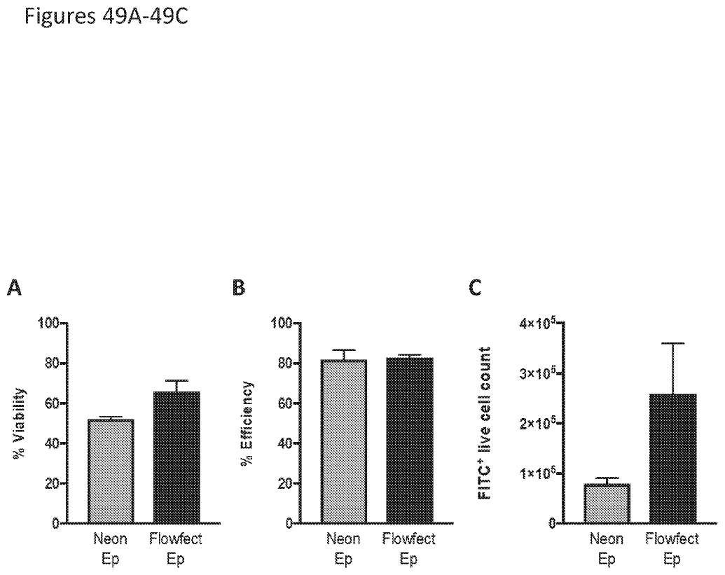

Devices of the invention can outperform commercial transfection system for the electroporation of primary monocytes. As shown in FIGS. 49A-49C, delivery of FITC labeled dextran into primary monocytes using devices of the invention exceeds the performance of the NEON® transfection system for primary human cells, with a marked increase in the total number of output live cells that are successfully transfected.

### Example 18—Continuous Flow Devices of the Invention: Large Volume and High Cell Number Cell Manufacturing

Devices of the invention can be used for the electroporation of large volumes and high cell number suspensions in a truly continuous flow manner. Existing technologies, such as the Lonza 4D-NUCLEOFECTOR™ LV Unit and the Maxcyte Scalable Transfection Systems (STX, VLX, or GT) rely on fluid flow to load the samples into their NUCLEOCUVETTE™ cartridge or processing assembly, respectively. However, during electrical pulse delivery, the cell and payload suspensions are stationary. Commercially available electroporation systems treat static or stationary cell suspensions, which is a critical difference from the devices of the invention. Devices of the invention allow for continuous flow of the cell and payload suspension during the exposure to the electric fields. Specifically, rapidly flowing cells are exposed to sufficient electric field to disrupt the cell membrane and internalize the genetic payload of interest but are immediately dispensed into their growth media for cell recovery. Additionally, any heat that is generated during the electroporation process is dissipated due to convective heat transfer that is facilitated by the flowing samples directly into recovery media. This study expands significantly on the data generated, both in cell type and in scale of the electroporations.

a. Initial Demonstration in Jurkat Cells

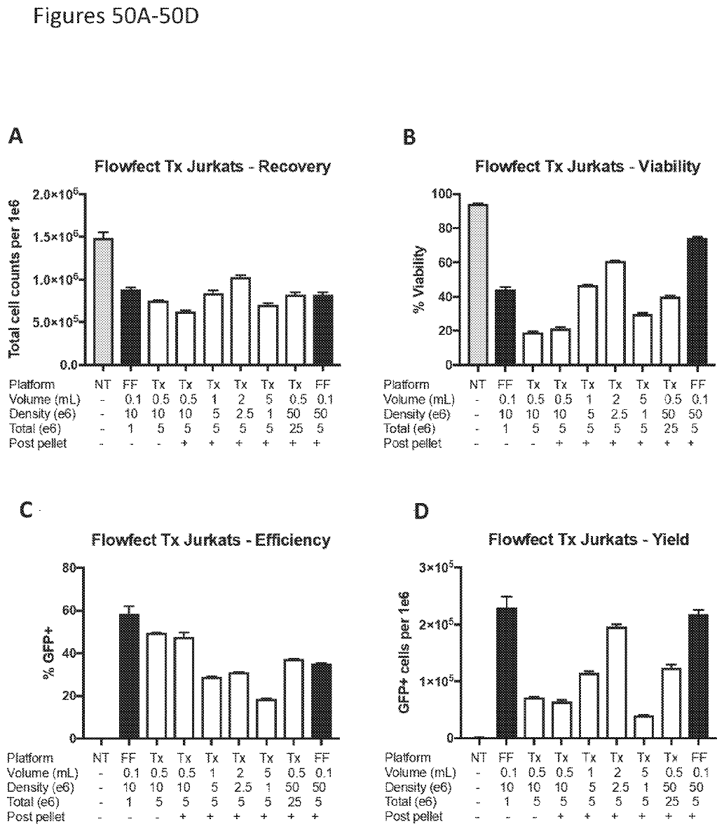

A range of cell densities and electroporation volumes were used to demonstrate the scalability of a continuous flow platform relative to a single device platform using devices of the invention. In these experiments, it is demonstrated that the scalable platform of the invention operates across a wide range of Jurkat cell densities, shown in FIGS. 50A-50D.

b. Comparability Studies Between Platforms of the Invention

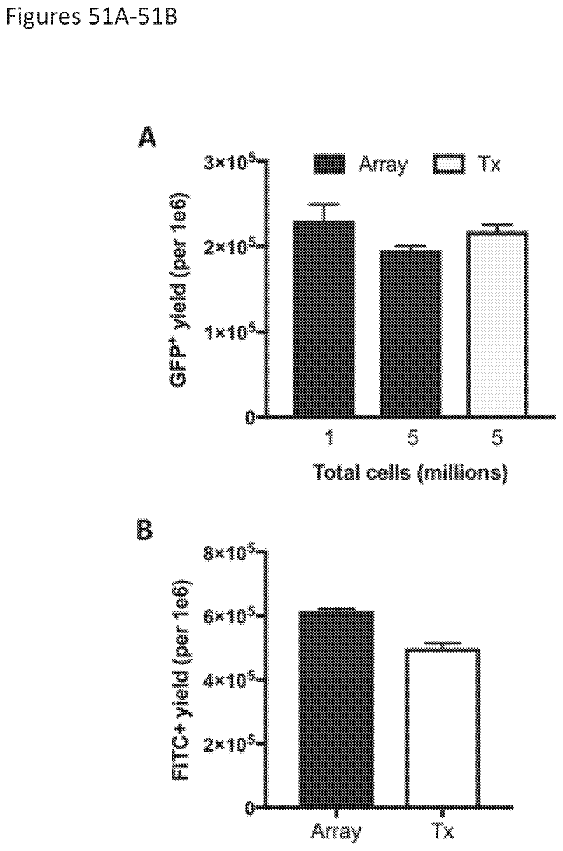

Follow-up experiments were performed to compare the electroporation performance of the devices of the invention and the continuous flow electroporation platform of the invention using the same delivery conditions for both Jurkat and primary T cells. In these comparative experiments, 5 million cells were processed through the continuous flow platform, showing comparable results to the single channel devices of the invention for Jurkat cells and primary T cells, as shown in FIGS. 51A and 51B.

c. Increased Scale of T Cell Electroporation

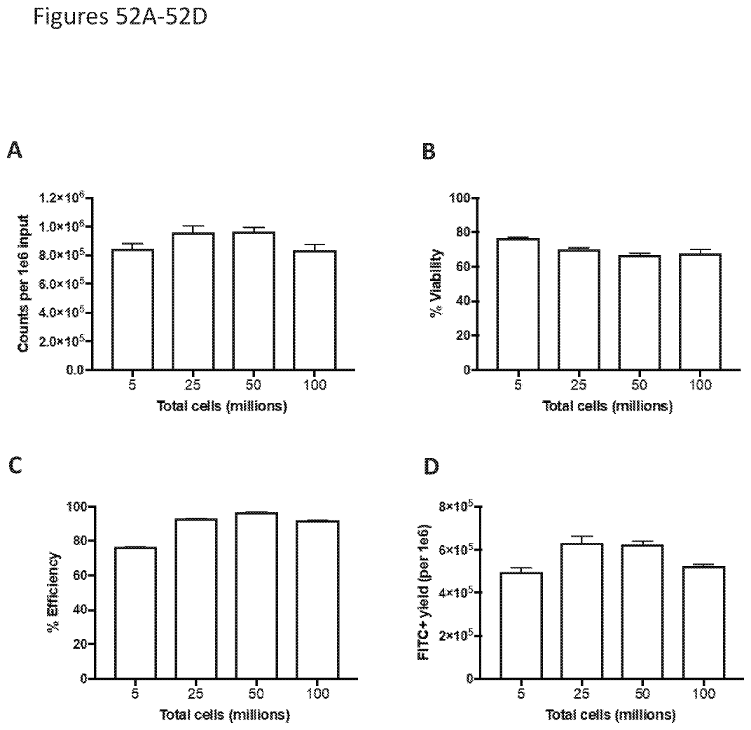

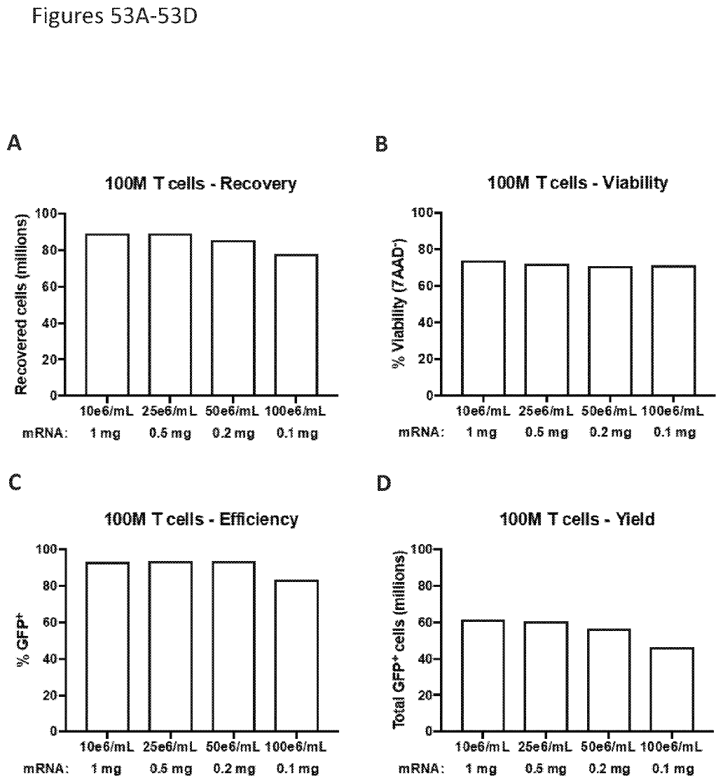

To test whether the electroporation was dependent on cell density, the electroporation experiments described in FIGS. 51A and 51B were expanded to cell suspensions containing up to 100 million primary T cells. In the first experiment, increasing numbers of T cells were processed at the same cell density, increasing the scale from 5 million (as shown in FIG. 51B) up to 100 million T cells (as shown in FIGS. 52A-52D), without a loss in yield. Desired cell density was then assessed, showing that T cells can be processed through the scalable platform of the invention at up to 100×10⁶ cells/mL, as shown in FIGS. 53A-53D. Importantly, the processing of 100 million T cells was successful with 5-fold lower mRNA quantities compared to T cells processed at the lowest cell density, demonstrating a potential cost of goods savings for payloads delivered at high cell densities. The total processing time for the 100 million T cells in this experiment ranged from 2.4 to 24 seconds.

d. Comparability Study with the Lonza Large Volume (LV) System

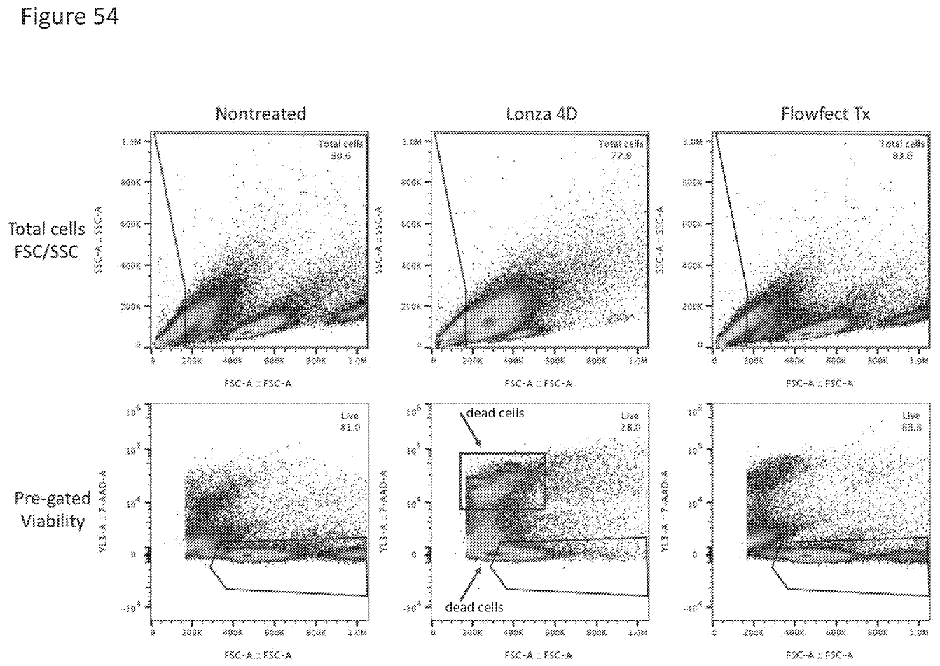

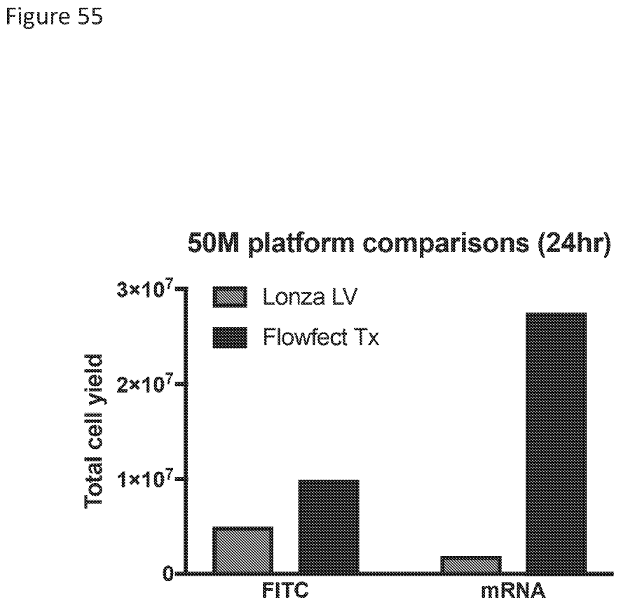

We performed a comparison of the scalable platform of the invention to the Lonza 4D LV system using primary T cells with both FITC-dextran and EGFP mRNA payloads. The experiments were performed with 50 million T cells. At 24 hours, cell staining revealed that the morphology and phenotype of the Lonza treated cells differed significantly from non-treated cells (shown in the flow cytometry plots of FIG. 54). Additionally, there were significant dead cell populations observed with the Lonza LV treated cells. These outcomes did not occur in the T cells electroporated with the continuous flow platform of the invention, indicating that the continuous flow platform of the invention maintained the T cell morphology through the electroporation process. As is shown in FIG. 55, the total cell yield using the continuous flow platform of the invention is higher than the Lonza 4D LV system, independent of the payload being delivered, e.g., FITC labeled dextran or GFP mRNA.

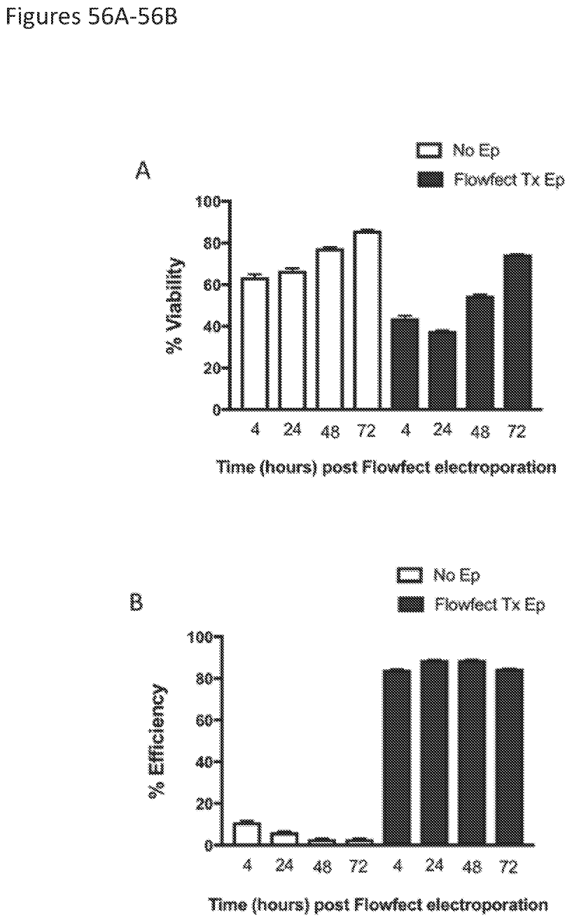

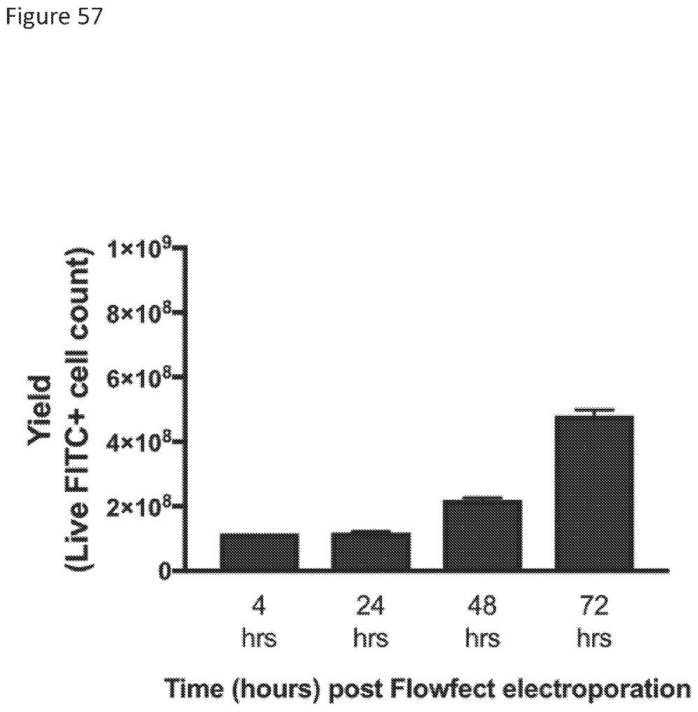

The continuous flow platform of the invention has shown successful electroporation of payloads into very high density, e.g., 1 billion-cell, suspensions. As shown in FIGS. 56A and 56B, 1 billion THP-1 cells in a volume of 10 mL (concentration of 100×10⁶ cells/mL) were successfully transfected with 40 kDa FITC labeled dextran molecules using the continuous flow platform of the invention. FIG. 57 shows the yield, represented as the live FITC cell count, for the experiment shown in FIGS. 56A and 56B, measured up to 72 hours post-electroporation. At this time point, the number of FITC positive cells was approximately 500 million, resulting from an input cell count of 1 billion, indicating the ability of the continuous flow platform of the invention to deliver 1 out of every 2 input cells as modified cell products at 72 hours.

### Example 19—Pulsed Waveforms, DC Voltage, High Voltage—Low Voltage Combination, and Combinations Thereof

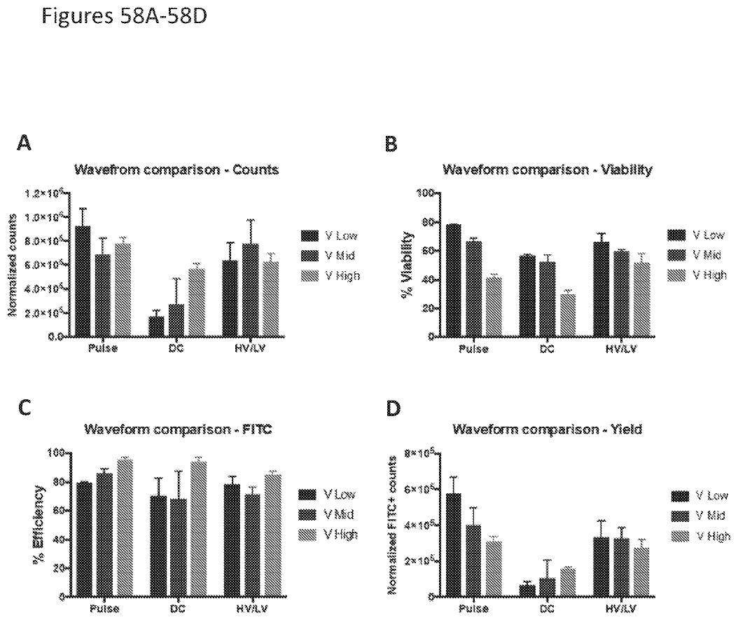

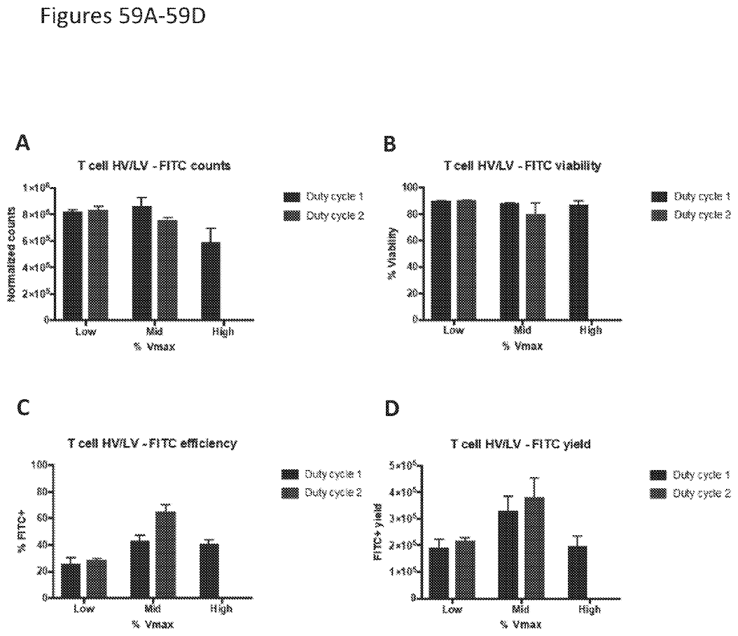

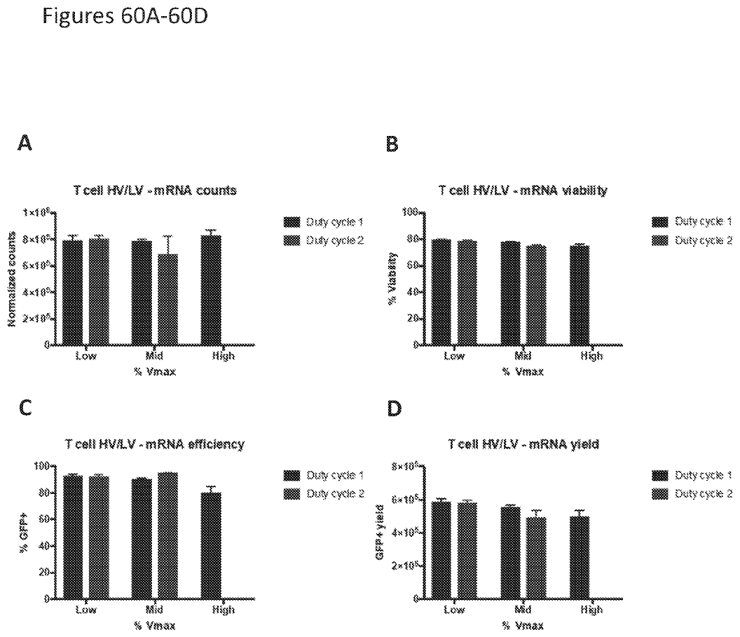

Devices of the invention were tested with both pulse and direct current (DC) power sources, as shown in FIGS. 58A-58D. At the higher voltages tested, both power supplies showed similar delivery efficiency of FITC-dextran in Jurkat cells. Additionally, initial electroporations with high voltage and low voltage combinations were tested for the same system. As shown in FIGS. 59A-59D, we have analyzed the use of modified waveforms for enhancement of electroporation using devices of the invention with high voltage and low voltage combinations for optimization of primary human T cell delivery, initially with FITC-dextran. The experiment of FIGS. 59A-59D was repeated for the delivery of a commercially available mRNA payload encoding eGFP fluorescent reporter protein, shown in FIGS. 60A-60D.

### Example 20—Dynabead Electroporation

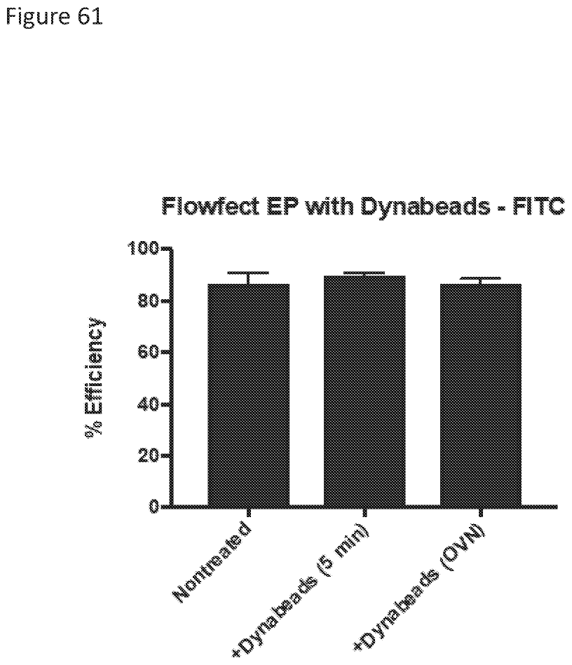

To demonstrate the compatibility of devices of the invention with certain T cell expansion protocols, T cells that had been expanded with CD3/CD28 Dynabeads were electroporated using devices of the invention. Electroporation of Dynabead-expanded samples was performed with immediate bead addition (5 min prior to electroporation) to the suspension of 1 million primary human T cells or after an overnight (OVN) treatment, with both time periods demonstrating equivalent efficiency results when the magnetic beads were present to when the beads were not present (FIG. 61).

### Example 21—Outer Structure for Energizing Devices of the Invention

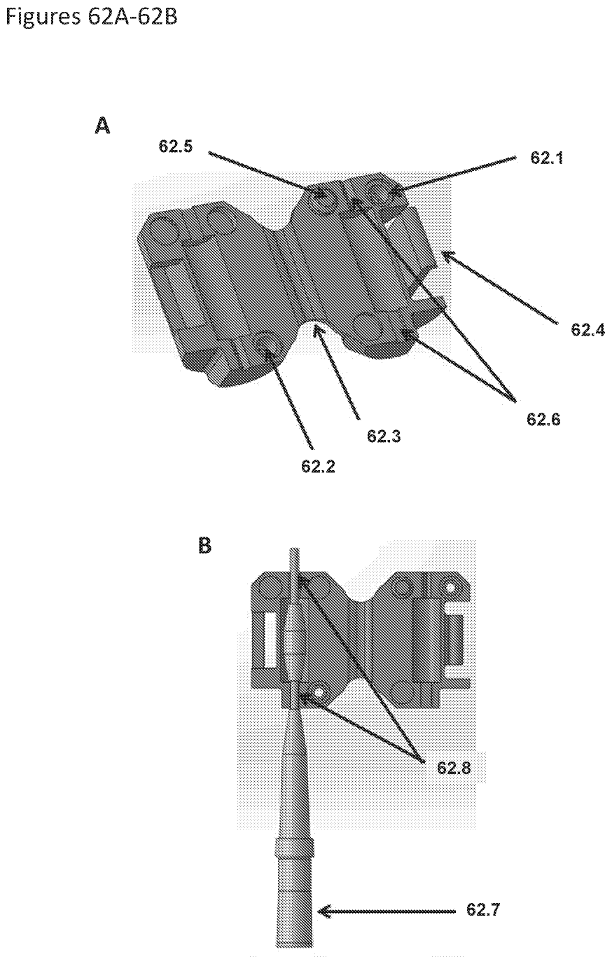

The invention provides an outer structure that fits over and secures to devices of the invention, designed to enhance the ease of use, the efficiency, and the safety during electroporation with the devices of the invention. The outer structure is made from non-conductive polymers on the outer surfaces that shields the users from high voltage exposures and minimize the risk of electrical shock to the user during the electroporation workflow. The outer structure accommodates the current design of the devices of the invention and can be modified to accept future designs variation of the devices of the invention. The outer structure accepts the electrical signal supplied from a power supply or high voltage amplifier and redistributes the signal to the electrodes of the devices of the invention by encapsulating the device within the outer structure. The encapsulation of the electrode of the devices of the invention creates a safer work environment for the user of the devices by minimizing the high voltage surfaces that are exposed. The outer structure also makes it easier to repeatedly do experiments without removal of electrical connections. An embodiment of an outer structure of the invention featuring a clamshell-style hinge and clasp is shown in FIGS. 62A and 62B. In FIG. 62A, 62.1 is a positive/negative electrode through hole for connections to the power supply.

62.2 is a second positive/negative electrode through hole for connections to the power supply. 62.3 is the clamshell-style hinge. For example, the hinge may be a living hinge, thus enabling the outer structure to close onto itself and engage the locking mechanism. This enclosure mechanism allows the outer structure to encase the electrodes of the device of the invention, ensuring electrical contact between both devices. 62.4 is a latch or other mechanical fastener used to ensure enclosure of the outer structure during electroporation. This design also enables the outer structure to be reusable by making the latching mechanism temporarily engaged. 62.5 is an alignment pin that ensures the outer structures folds with the correct alignment to minimize any offsets that would distort the electrode connections between the outer structure and the devices of the invention. 62.6 are recesses for the electrodes of the device of the invention. 62.7 and 62.8 are the body of a device of the invention and the first and second electrodes defining the electroporation zone of the device of the invention, respectively.

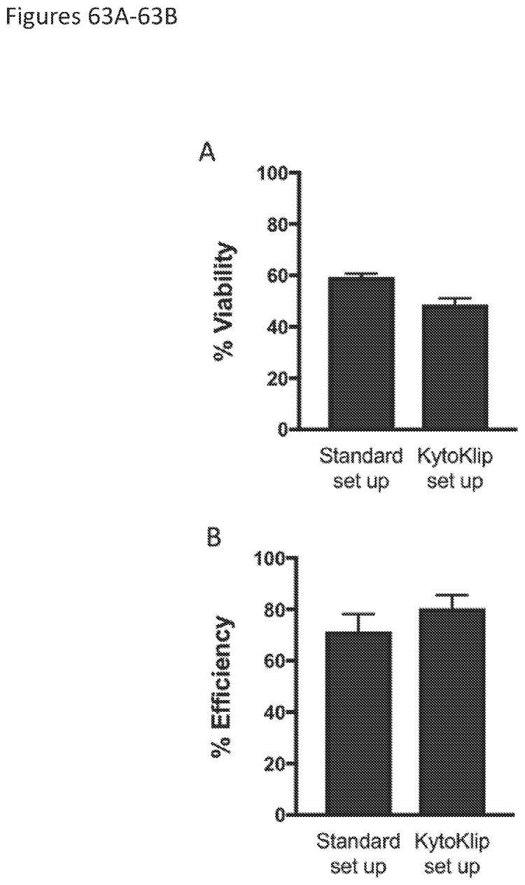

In use, the outer structure connected to the devices of the invention showed no significant loss in transfection efficiency or viability when performing electroporation using devices of the invention without the outer structure. As shown in FIGS. 63A-63B, the viability and efficiency of THP-1 monocytes transfected with FITC labeled dextran was approximately the same using devices of the invention with or without the outer structure over the electrodes of the device.

### Example 22—Manufacturing Material for Disposable Devices

Devices of the invention are constructed from resin formulations produced and sold by Formlabs (Somerville, Mass. USA). In particular, devices of the invention are fabricated from either the “Clear resin” or the Formlabs' marketed “Durable resin”. The major difference between the Durable and Clear resins is the mechanical properties. The Clear resin is more brittle in terms of mechanical behavior and the Durable resin has a greater ductility to the extent that the mechanical performance is more similar to that of polypropylene, the material from which conventional pipette tips are manufactured.

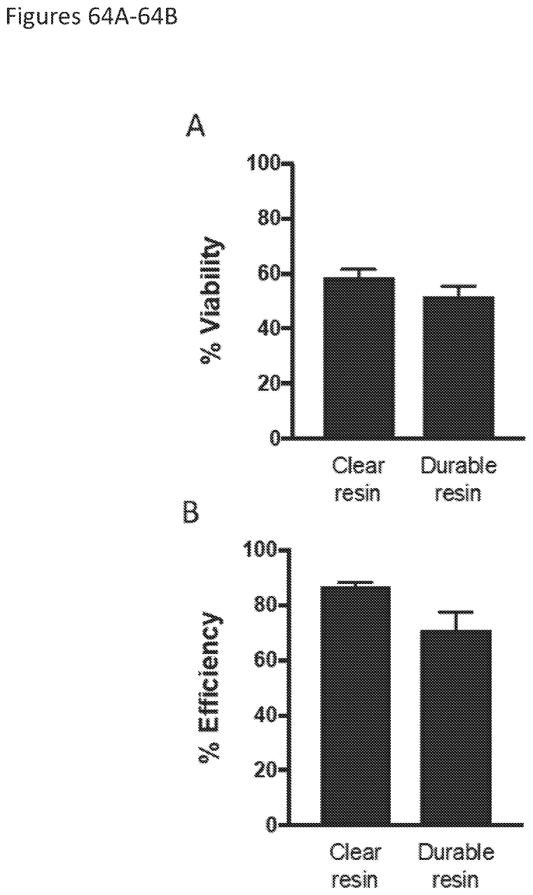

Devices of the invention are 3D printed using stereolithography technology for prototyping purposes. For large scale processing, such as injection molding, device of the invention will be fabricated from other resins, such as the Durable resin which closely simulates polypropylene's mechanical properties. To examine whether the resin material impacts electroporation, FIGS. 64A and 64B show the delivery of FITC labeled dextran into THP-1 monocytes using devices of the invention fabricated from the Formlabs' Clear resin and Durable resins. The choice of material resulted in no significant change in performance of the devices of the invention.

### Example 23—Automated Transfection vs. Manual (Electronic) Sample Driving

Devices of the invention have enabled rapid, high throughput, and automated engineering of human cells. Applications of this technology are widespread, ranging from fundamental research in cell physiology to the discovery of new targets for cellular therapies. The applications in cell therapies alone can contribute to a growing multi-billion dollar industry. The current state of the art in genetic manipulation at the research scale is manually intensive and difficult to incorporate with automated liquid handling systems. Devices of the invention can be readily incorporated into a diverse array of liquid handling platforms. This integration will allow researchers in academia and industry to quickly explore a wide array of questions related to genetics. The devices of the invention have the potential to facilitate research-scale cell engineering thousands of times faster than the current state of the art, leading to life changing discoveries in healthcare and the fundamental biological sciences.

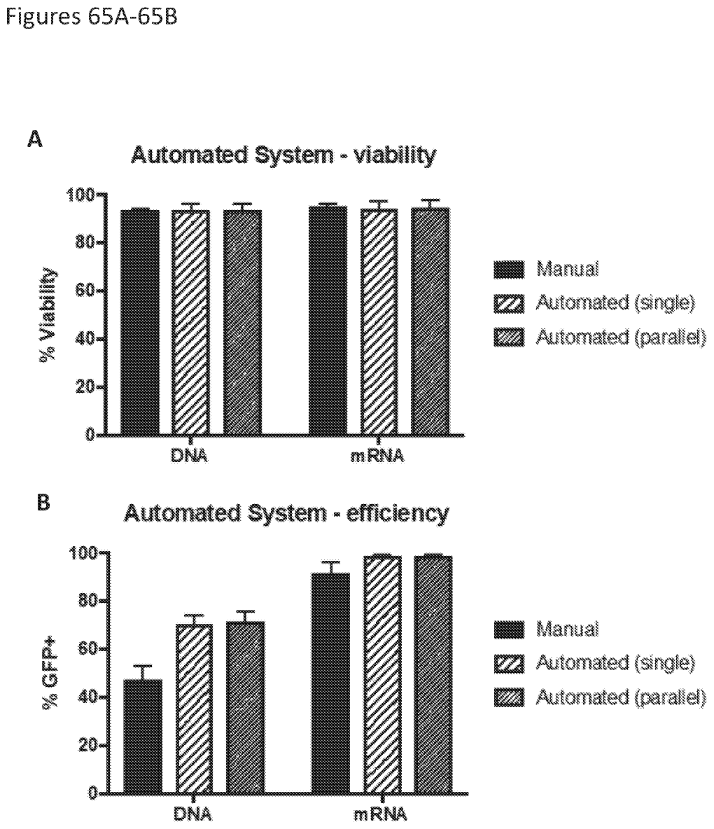

The experiments on T-cells described herein were originally conducted with single-use devices of the invention. With the automated system incorporating devices of the invention, transfection can be streamlined and configured in a high-throughput manner. Eight independently controlled syringes were programmed to drive the cell suspension into single use devices of the invention. 100 μL samples were aspirated above the electroporation zone of each device and were energized during active dispensing into the recovery growth media. Three automated methods of transfection that used air-displacement (manual electronic pipette) or fluid-displacement (automated system) to drive the samples were compared. The resulting viability remained at high levels (>90%) when using the lymphocyte gate methodology for the 3 systems evaluated (shown in FIGS. 65A and 65B). However, when looking at transfection efficiency, it is clear that the automated system, which employs fluid displacement technology to precisely control flow rate, is superior to the manual.

### Example 24—Co-Delivery of mRNA Reagent into Primary T Cells

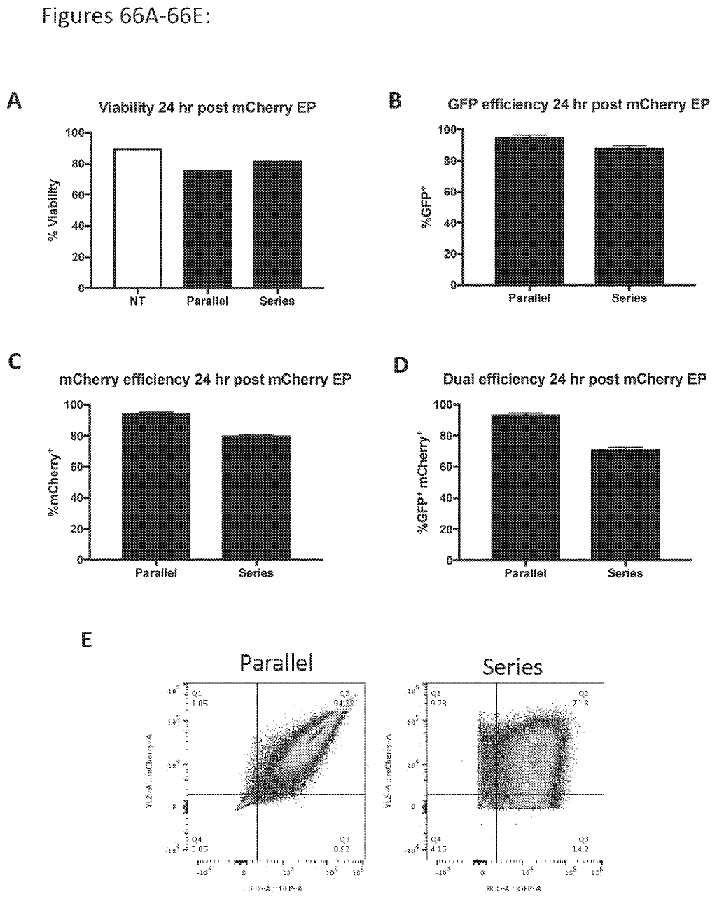

Co-deliver two mRNA types into T cells was evaluated using devices of the invention. These experiments were performed with two commercially sourced mRNAs encoding either GFP or mCherry. The experiments were completed either in parallel (same day) or in series (two days apart). The devices of the invention were successfully able to deliver both mRNAs as demonstrated by the GFP and mCherry expression observed in FIGS. 66A-66E.

### Example 25—Transfections of Mixed Population Peripheral Blood Mononuclear Cells

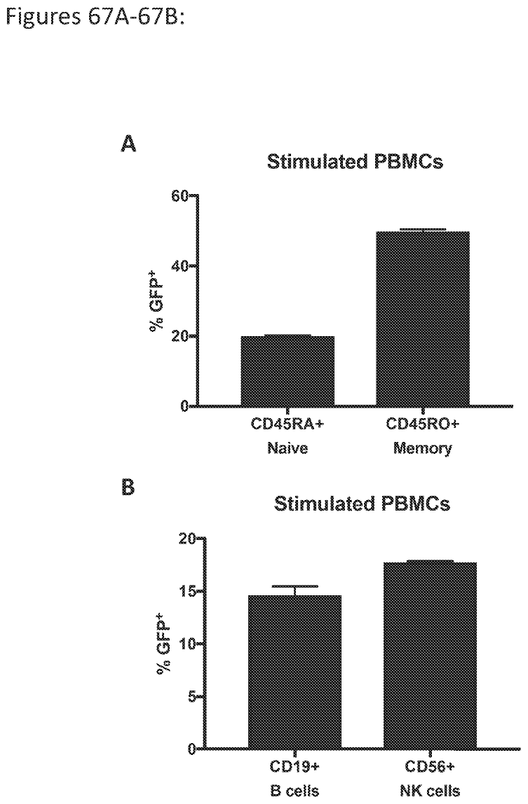

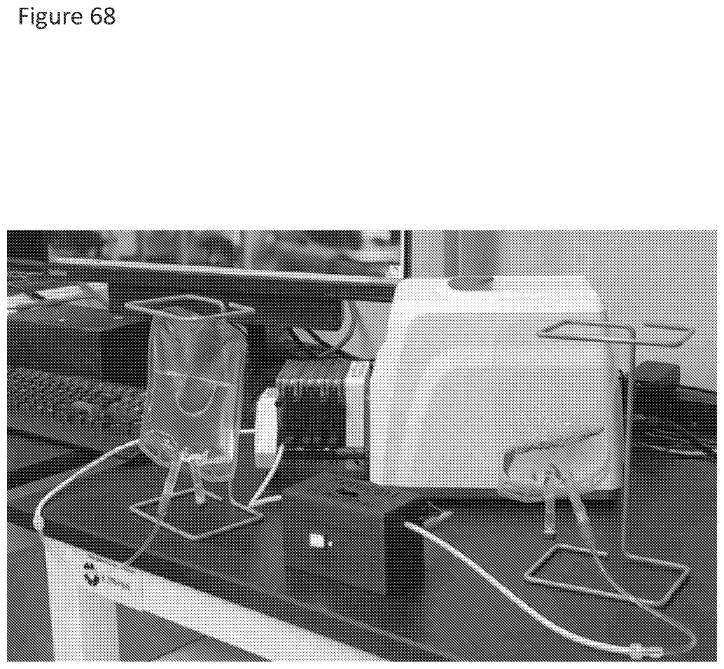

mRNA delivery into primary human mixed cell populations (i.e., PBMCs) was also demonstrated using devices of the invention. These experiments were performed with a commercially sourced mRNA encoding GFP, followed by phenotype staining of surface receptors to identify specific cell populations. Delivery of mRNA to both naïve (CD45RA+) and memory (CD45RO+) T cells was achieved, as shown in FIG. 67A. Additionally, delivery of mRNA to B cells (CD19+) and natural killer NK cells (CD56+) from the mixed population was achieved, as shown in FIG. 67B.

### Example 26—mRNA Transfection of Primary Adherent iPSCs

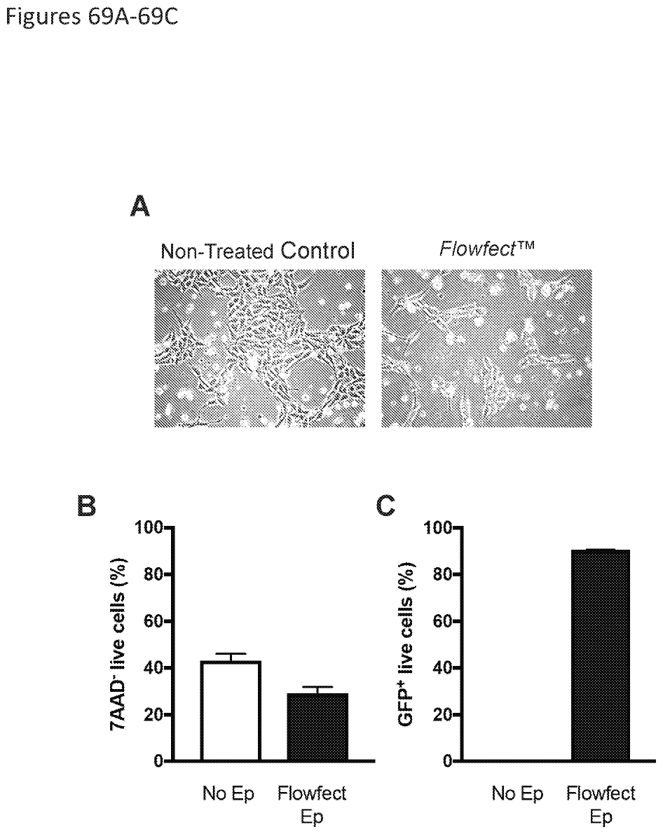

Induced pluripotent stem cells (iPSCs) were transected with eGFP-mRNA, in suspension, using a device of the invention (FLOWFECT™). Cells were assessed 24 hours after transfection for indication of positive transfection using florescent microscopy. Images are depicted as an overlay image of GFP and brightfield to capture adherence, cell morphology, and expression of eGFP-mRNA (representative images shown at 10× magnification; FIG. 69A). Cells were also assessed at 96 hours after transfection via flow cytometer for the proportion of viable (7AAD⁻) and positively transfected (GFP⁺7AAD⁻) cells (representative data shown as Mean±SEM; FIGS. 69B and 69C).

### Example 27—mRNA Transfection of Primary Human Natural Killer Cells

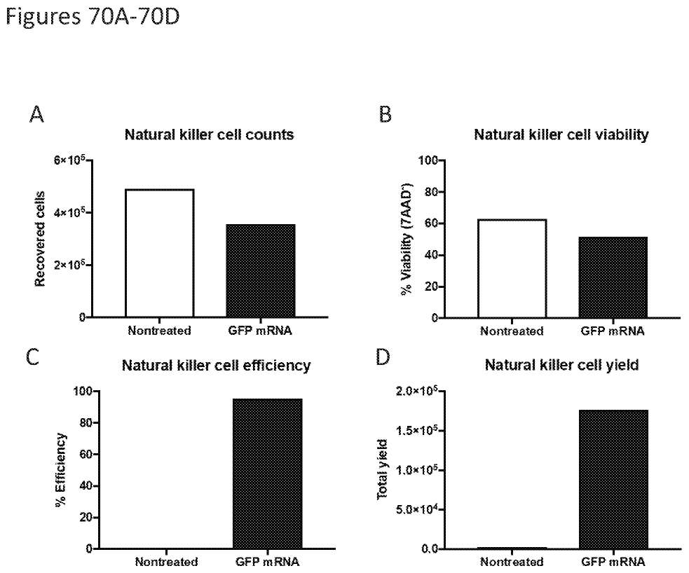

Isolated NK cells (CD56⁺) were electroporated with mRNA encoding GFP. After 24 hours, the cells were analyzed for viability and efficiency. The NK counts and viabilities are shown in FIGS. 70A-70B. The devices of the invention were successfully able to deliver mRNAs, as demonstrated by the ˜95% GFP expression observed in FIG. 70C. The total yield of live GFP⁺ cells compared to live nontreated cells at 24 hours was ˜57%, as shown in FIG. 70D.

### Example 28—Cross Platform Comparison

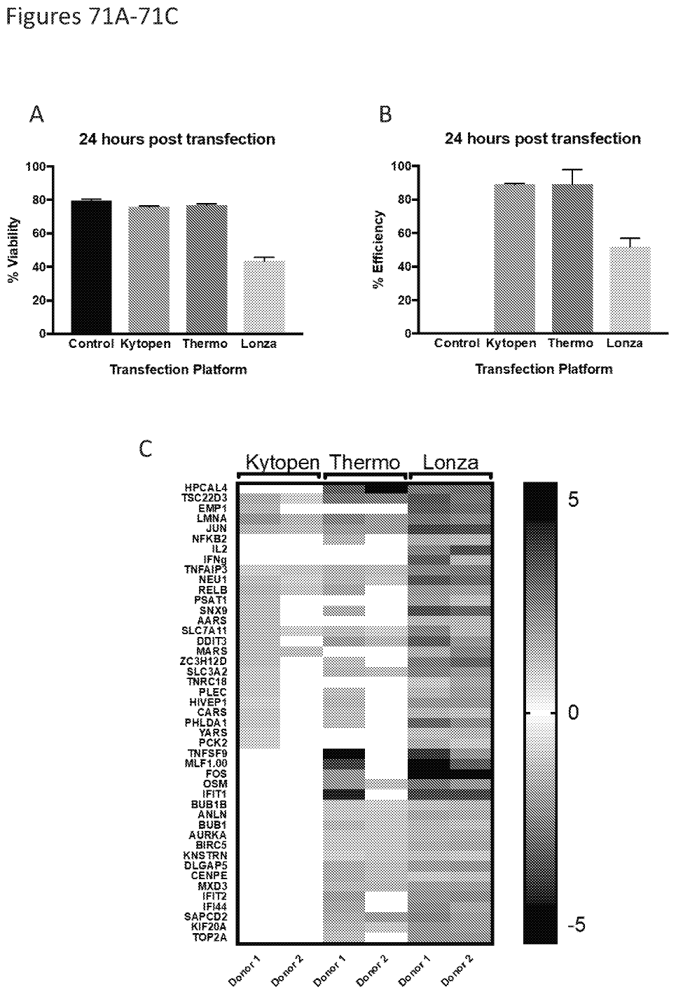

Human primary T cells were transfected with a reporter molecule using a device of the invention, ThermoFisher's Neon transfection platform (2100V/1 pulse/20 ms), or Lonza 4D-Nucleofector (Program: EO115) using the manufacturer recommending settings as listed. Shown in FIGS. 71A and 71B are the viability and transfection efficiency 24 hours after transfection. Using the same the settings human primary T cells were processed without payload and assessed for gene dysregulation six hours after processing (FIG. 71C).

### Example 29—Voltage—Flow-Rate Profiles

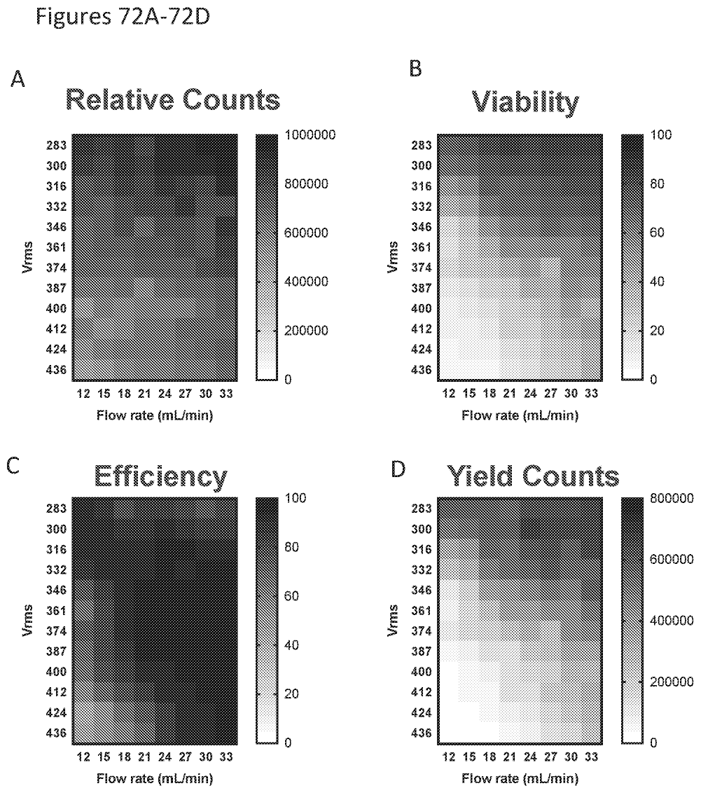

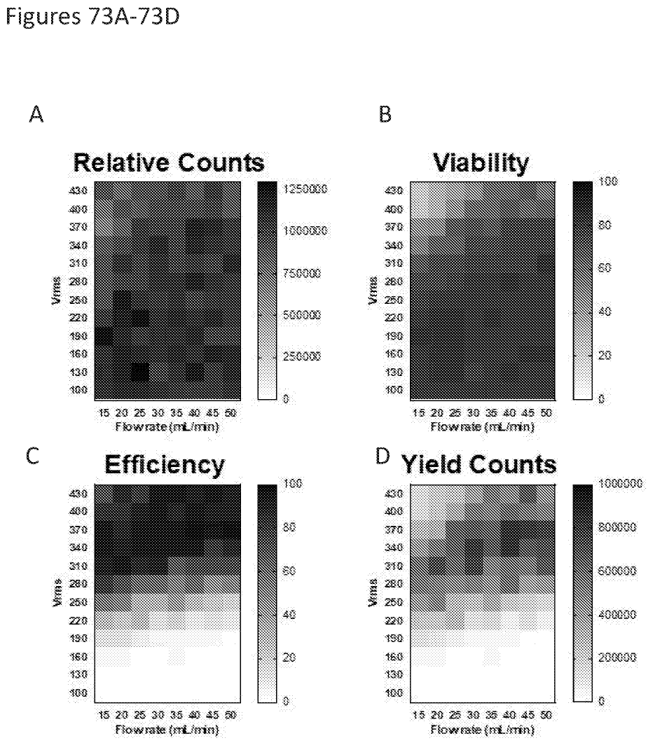

Human primary T cells were transfected with GFP reporter mRNA using multiple flowrate and applied energy combinations. Shown in FIGS. 72A-72D and 73A-73D are heatmaps for relative cell count, viability (percent 7AAD− cells), efficiency (percent live GFP+ cells), and yield cell counts (live GFP+ cell counts) per reaction at 24 hours after transfection, shown as a function of varying Vrms (y axis) and flow rate (x axis).

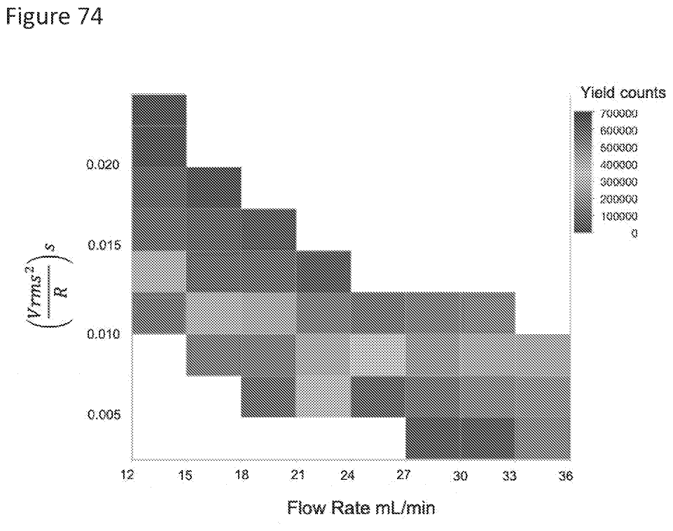

FIG. 74 shows live cell yield across varying applied energy (Vrms²/R)s and flow rates. Human primary T cells were transfected with FITC Dextran using multiple flowrate (FR) and applied energy combinations (VV). Shown is a density plot which shows that the same applied energy at different flow rates results in different yield counts (live FITC+ cell counts).

### Numerated Embodiments

Some embodiments of the technology described herein can be defined according to any of the following numbered paragraphs:
1. A device for electro-mechanical delivery of a composition into a plurality of cells suspended in a liquid, the device comprising:
   (a) a first electrode comprising a first inlet, a first outlet, and a first lumen comprising a minimum cross-sectional dimension;
   (b) a second electrode comprising a second inlet, a second outlet, and a second lumen comprising a minimum cross-sectional dimension; and
   (c) an electroporation zone disposed between the first outlet and the second inlet, wherein the electroporation zone comprises a minimum cross-sectional dimension greater than about 100 μm, wherein the electroporation zone has a substantially uniform cross-sectional area;
wherein the first outlet, the electroporation zone, and the second inlet are in fluidic communication,
wherein the composition is delivered to the plurality of cells suspended in the fluid upon entering the electroporation zone.
2. A device for electroporating a composition into a plurality of cells suspended in a fluid, comprising:
   (a) a first electrode comprising a first inlet and a first outlet, wherein a lumen of the first electrode comprises an entry zone;
   (b) a second electrode comprising a second inlet and a second outlet, wherein a lumen of the second electrode comprises a recovery zone; and
   (c) an electroporation zone, wherein the electroporation zone is fluidically connected to the first outlet of the first electrode and the second inlet of the second electrode, wherein the electroporation zone has a substantially uniform cross-section dimension, and wherein application of an electrical potential difference to the first and second electrodes produces an electric field in the electroporation zone,
wherein the plurality of cells suspended in the fluid are electroporated upon entering the electroporation zone.
3. The device of paragraph 1 or paragraph 2, further comprising a first reservoir fluidically connected to the entry zone.
4. The device of paragraph 1 or paragraph 2, further comprising a second reservoir fluidically connected to the recovery zone.
5. The device of paragraph 1 or paragraph 2, wherein the cross-section of the electroporation zone is selected from the group consisting of circular, cylindrical, ellipsoidal, polygonal, star, parallelogram, trapezoidal, and irregular.
6. The device of paragraph 1 or paragraph 2, wherein the cross-sectional dimension of the entry zone is between 0.01% and 100,000% of the cross-sectional dimension of the electroporation zone.
7. The device of paragraph 1 or paragraph 2, wherein the cross-sectional dimension of the recovery zone is between 0.01% and 100,000% of the largest cross-sectional dimension of the electroporation zone.
8. The device of paragraph 1 or paragraph 2, wherein the cross-sectional dimension of the electroporation zone is between 0.005 mm and 50 mm.
9. The device of paragraph 1 or paragraph 2, wherein the length of the electroporation zone is between 0.005 mm and 50 mm.
10. The device of paragraph 1 or paragraph 2, wherein the cross-sectional dimension of any of the first electrode or the second electrode is between 0.01 mm and 500 mm.
11. The device of paragraph 1 or paragraph 2, wherein none of the entry zone, recovery zone, or electroporation zone reduce a cross-section dimension of any of the plurality of cells suspended in the fluid.
12. The device of paragraph 1 or paragraph 2, wherein the plurality of cells has from 0% to about 25% phenotypic change relative to a baseline measurement of cell phenotype upon exiting the electroporation zone.
13. The device of paragraph 1 or paragraph 2, the plurality of cells has no phenotypic change upon exiting the electroporation zone

14. The device of paragraph 1 or paragraph 2, further comprising an outer structure comprising a housing configured to encase the first electrode, second electrode, and the electroporation zone of the device.
15. The device of paragraph 14, wherein the outer structure comprises a first electrical input operatively coupled to the first electrode and a second electrical input operatively coupled to the second electrode.
16. The device of paragraph 14 or paragraph 15, wherein the outer structure is integral to the device.
17. The device of paragraph 14 or paragraph 15, wherein the outer structure is releasably connected to the device.
18. A device for electro-mechanical delivery of a composition into a plurality of cells suspended in a fluid, comprising:
   (a) a first electrode comprising a first inlet and a first outlet, wherein a lumen of the first electrode comprises an entry zone;
   (b) a second electrode comprising a second inlet and a second outlet, wherein a lumen of the second electrode comprises a recovery zone;
   (c) a third inlet and a third outlet, wherein the third inlet and third outlet intersect the first electrode between the first inlet and the first outlet;
   (d) a fourth inlet and a fourth outlet, wherein the fourth inlet and fourth outlet intersect the second electrode between the second inlet and the second outlet;
   (e) an electroporation zone, wherein the electroporation zone is fluidically connected to the first outlet of the entry zone and the second inlet of the recovery zone, wherein the electroporation zone has a substantially uniform cross-section dimension, and wherein application of an electrical potential difference between the first and second electrodes produces an electric field in the electroporation zone,
wherein the composition is delivered to the plurality of cells suspended in the fluid upon entering the electroporation zone.
19. A device for electroporating a composition into a plurality of cells suspended in a fluid, comprising:
   (a) a first electrode comprising a first inlet and a first outlet, wherein a lumen of the first electrode comprises an entry zone;
   (b) a second electrode comprising a second inlet and a second outlet, wherein a lumen of the second electrode comprises a recovery zone,
   (c) a third inlet and a third outlet, wherein the third inlet and third outlet intersect the first electrode between the first inlet and the first outlet;
   (d) a fourth inlet and a fourth outlet, wherein the fourth inlet and fourth outlet intersect the second electrode between the second inlet and the second outlet;
   (e) an electroporation zone, wherein the electroporation zone is fluidically connected to the first outlet of the entry zone and the second inlet of the recovery zone, wherein the electroporation zone has a substantially uniform cross-section dimension, and wherein application of an electrical potential difference between the first and second electrodes produces an electric field in the electroporation zone,
   wherein the plurality of cells suspended in the fluid are electroporated upon entering the electroporation zone.
20. The device of paragraph 18 or paragraph 19, further comprising a first reservoir fluidically connected to the entry zone.
21. The device of paragraph 18 or paragraph 19, further comprising a second reservoir fluidically connected to the recovery zone.
22. The device of paragraph 18 or paragraph 19, further comprising a third reservoir fluidically connected to the third inlet and the third outlet.
23. The device of paragraph 18 or paragraph 19, further comprising a fourth reservoir fluidically connected to the fourth inlet and the fourth outlet.
24. The device of paragraph 18 or paragraph 19, wherein the cross-section of the electroporation zone is selected from the group consisting of circular, ellipsoidal, polygonal (e.g., regular polygon, irregular polygon), star, parallelogram, trapezoidal, and irregular.
25. The device of paragraph 18 or paragraph 19, wherein the cross-sectional dimension of the entry zone is between 0.01% and 100,000% of the cross-sectional dimension of the electroporation zone.
26. The device of paragraph 18 or paragraph 19, wherein the cross-sectional dimension of the recovery zone is between 0.01% and 100,000% of the cross-sectional dimension of the electroporation zone.
27. The device of paragraph 18 or paragraph 19, wherein the cross-sectional dimension of the electroporation zone is between 0.005 mm and 50 mm.
28. The device of paragraph 18 or paragraph 19, wherein the length of the electroporation zone is between 0.005 mm and 50 mm.
29. The device of paragraph 18 or paragraph 19, wherein the cross-sectional dimension of any of the first electrode or the second electrode is between 0.1 mm and 5 mm.
30. The device of paragraph 18 or paragraph 19, wherein any of the first electrode or the second electrode are porous.
31. The device of paragraph 18 or paragraph 19, wherein none of the entry zone, recovery zone, or electroporation zone reduce a cross-section dimension of any of the plurality of cells suspended in the fluid.
32. The device of paragraph 18 or paragraph 19, wherein the plurality of cells has from 0% to about 25% phenotypic change relative to a baseline measurement of cell phenotype upon exiting the electroporation zone.
33. The device of paragraph 18 or paragraph 19, wherein the plurality of cells has no phenotypic change upon exiting the electroporation zone.
34. The device of paragraph 18 or paragraph 19, further comprising an outer structure comprising a housing configured to encase the first electrode, second electrode, and the electroporation zone of the device.
35. The device of paragraph 34, wherein the outer structure comprises a first electrical input operatively coupled to the first electrode and a second electrical input operatively coupled to the second electrode.
36. The device of paragraph 34 or paragraph 35, wherein the outer structure is integral to the device.
37. The device of paragraph 34 or paragraph 35, wherein the outer structure is releasably connected to the device.
38. A system for electro-mechanical delivery of a composition into a plurality of cells suspended in a fluid, comprising:
   (a) a device, comprising:
      (i) a first electrode comprising a first inlet and a first outlet, wherein a lumen of the first electrode comprises an entry zone;
      (ii) a second electrode comprising a second inlet and a second outlet, wherein a lumen of the second electrode comprises a recovery zone; and
      (iii) an electroporation zone, wherein the electroporation zone is fluidically connected to the first outlet of the first electrode and the second inlet of the second electrode, wherein the electroporation zone has a substantially uniform cross-section dimension, and wherein application of an electrical potential difference to the first and second electrodes produces an electric field in the electroporation zone;
   (b) a source of electrical potential, wherein the first and second electrodes of the device are releasably connected to the source of electrical potential,
wherein the composition is delivered into the plurality of cells suspended in the fluid upon entering the electroporation zone.
39. A system for electroporating a composition into a plurality of cells suspended in a fluid, comprising:
   (a) a device comprising:
      (i) a first electrode comprising a first inlet and a first outlet, wherein a lumen of the first electrode comprises an entry zone;
      (ii) a second electrode comprising a second inlet and a second outlet, wherein a lumen of the second electrode comprises a recovery zone; and
      (iii) an electroporation zone, wherein the electroporation zone is fluidically connected to the first outlet of the first electrode and the second inlet of the second electrode, wherein the electroporation zone has a substantially uniform cross-section dimension, and wherein application of an electrical potential difference to the first and second electrodes produces an electric field in the electroporation zone;
   (b) a source of electrical potential, wherein the first and second electrodes of the device are releasably connected to the source of electrical potential,
wherein the plurality of cells suspended in the fluid are electroporated upon entering the electroporation zone.
40. The system of paragraph 38 or paragraph 39, wherein the plurality of cells has from 0% to about 25% phenotypic change relative to a baseline measurement of cell phenotype upon exiting the electroporation zone of the device.
41. The system of paragraph 38 or paragraph 39, wherein the plurality of cells has no phenotypic change upon exiting the electroporation zone.
42. The system of paragraph 38 or paragraph 39, wherein the device further comprises an outer structure comprising a housing configured to encase the first electrode, second electrode, and the electroporation zone of the device.
43. The system of paragraph 38 or paragraph 39, wherein the outer structure comprises a first electrical input operatively coupled to the first electrode and a second electrical input operatively coupled to the second electrode.
44. The system of paragraph 43, wherein the source of electrical potential is releasably connected to the first and second electrical inputs of the outer structure.
45. The system of paragraph 44, wherein the releasable connection between the first or second electrical inputs and the source of electrical potential is selected from the group consisting of a clamp, a clip, a spring, a sheath, a wire brush, or a combination thereof.
46. The system of paragraph 38 or paragraph 39, wherein the outer structure is integral to the device.
47. The system of paragraph 38 or paragraph 39, wherein the outer structure is releasably connected to the device.
48. The system of paragraph 38 or paragraph 39, wherein the electroporation is substantially non-thermal reversible electroporation.
49. The system of paragraph 38 or paragraph 39, wherein the electroporation is substantially non-thermal irreversible electroporation.
50. The system of paragraph 38 or paragraph 39, wherein the electroporation is substantially thermal irreversible electroporation.
51. The system of paragraph 38 or paragraph 39, wherein the releasable connection between the device and the source of electrical potential is selected from the group consisting of a clamp, a clip, a spring, a sheath, a wire brush, or a combination thereof.
52. The system of paragraph 51, wherein the releasable connection between the device and the source of electrical potential is a spring.
53. The system of paragraph 38 or paragraph 39, further comprising a first reservoir fluidically connected to the entry zone.
54. The system of paragraph 38 or paragraph 39, further comprising a second reservoir fluidically connected to the recovery zone.
55. The system of paragraph 38 or paragraph 39, wherein the cross-section of the electroporation zone is selected from the group consisting of circular, ellipsoidal, polygonal, star, parallelogram, trapezoidal, and irregular.
56. The system of paragraph 38 or paragraph 39, wherein the cross-sectional dimension of the entry zone is between 0.01% and 100,000% of the cross-sectional dimension of the electroporation zone.
57. The system of paragraph 38 or paragraph 39, wherein the cross-sectional dimension of the recovery zone is between 0.01% and 100,000% of the cross-sectional dimension of the electroporation zone.
58. The system of paragraph 38 or paragraph 39, wherein none of the entry zone, recovery zone, or electroporation zone reduce a cross-section dimension of any of the plurality of cells suspended in a fluid.
59. The system of paragraph 38 or paragraph 39, wherein the duty cycle of the electroporation is between 0.001% and 100%.
60. The system of paragraph 38 or paragraph 39, wherein the cross-sectional dimension of the electroporation zone is between is between 0.005 mm and 50 mm.
61. The system of paragraph 38 or paragraph 39, wherein the length of the electroporation zone is between is between 0.005 mm and 50 mm.
62. The system of paragraph 38 or paragraph 39, wherein the cross-sectional dimension of any of the first electrode or the second electrode is between 0.1 mm and 5 mm.
63. The system of paragraph 38 or paragraph 39, further comprising a fluid delivery source fluidically connected to the entry zone, wherein the fluid delivery source is configured to deliver the plurality of cells suspended in the fluid through the entry zone to the recovery zone.
64. The system of paragraph 63, wherein the delivery rate from the fluid delivery source is between 0.001 mL/min and 1,000 mL/min.
65. The system of paragraph 38 or paragraph 39, wherein the residence time of any of the plurality of cells suspended in the fluid is between 0.5 ms and 50 ms.
66. The system of paragraph 38 or paragraph 39, further comprising a controller operatively coupled to the source of electrical potential to deliver voltage pulses to the first electrode and second electrodes to generate an electrical potential difference between the first and second electrodes.
67. The system of paragraph 66, wherein the voltage pulses have an amplitude between 0.01 kV and 3 kV.
68. The system of paragraph 66, wherein the voltage pulses have a duration of between 0.01 ms and 1,000 ms.
69. The system of paragraph 66, wherein the voltage pulses are applied the first and second electrodes at a frequency between 1 Hz and 50,000 Hz.
70. The system of paragraph 66, wherein the waveform of the voltage pulse is selected from the group consisting of DC, square, pulse, bipolar, sine, ramp, asymmetric bipolar, arbitrary, and any superposition or combinations thereof.
71. The system of paragraph 66, wherein the electric field generated from the voltage pulses has a magnitude of between 1 V/cm and 50,000 V/cm.
72. The system of any one of paragraphs 38-71, wherein the fluid has a conductivity of between 0.001 mS/cm and 500 mS/cm.
73. The system of any one of paragraphs 36-68, further comprising a housing configured to house the device.
74. The system of paragraph 73, wherein the housing further comprises a thermal controller configured to increase or decrease the temperature of the housing.
75. The system of paragraph 74, wherein the thermal controller is a heating element selected from the group consisting of a heating block, liquid flow, battery powered heater, and a thin-film heater.
76. The system of paragraph 74, wherein the thermal controller is a cooling element selected from the group consisting of a liquid flow, evaporative cooler, and a Peltier device.
77. The system of any one of paragraphs 38-76, further comprising a plurality of cell porating devices.
78. The system of paragraph 77, further comprising a plurality of outer structures.
79. A system for electro-mechanical delivery of a composition into a plurality of cells suspended in a fluid, comprising:
   (a) a device comprising:
      (i) a first electrode comprising a first inlet and a first outlet, wherein a lumen of the first electrode comprises an entry zone;
      (ii) a second electrode comprising a second inlet and a second outlet, wherein a lumen of the second electrode comprises a recovery zone,
      (iii) a third inlet and a third outlet, wherein the third inlet and third outlet intersect the first electrode between the first inlet and the first outlet;
      (iv) a fourth inlet and a fourth outlet, wherein the fourth inlet and fourth outlet intersect the second electrode between the second inlet and the second outlet;
      (v) an electroporation zone, wherein the electroporation zone is fluidically connected to the first outlet of the entry zone and the second inlet of the recovery zone, wherein the electroporation zone has a substantially uniform cross-section dimension, and wherein application of an electrical potential to the first and second electrodes produces an electric field in the electroporation zone; and
   (b) a source of electrical potential, wherein the first and second electrodes of the device are releasably connected to the source of electrical potential,
wherein the composition is delivered into the plurality of cells suspended in the fluid upon entering the electroporation zone. 80. A system for electroporating a composition into a plurality of cells suspended in a fluid, comprising:
   (a) a device comprising:
      (i) a first electrode comprising a first inlet and a first outlet, wherein a lumen of the first electrode comprises an entry zone;
      (ii) a second electrode comprising a second inlet and a second outlet, wherein a lumen of the second electrode comprises a recovery zone,
      (iii) a third inlet and a third outlet, wherein the third inlet and third outlet intersect the first electrode between the first inlet and the first outlet;
      (iv) a fourth inlet and a fourth outlet, wherein the fourth inlet and fourth outlet intersect the second electrode between the second inlet and the second outlet;
      (v) an electroporation zone, wherein the electroporation zone is fluidically connected to the first outlet of the entry zone and the second inlet of the recovery zone, wherein the electroporation zone has a substantially uniform cross-section dimension, and wherein application of an electrical potential to the first and second electrodes produces an electric field in the electroporation zone; and
   (b) a source of electrical potential, wherein the first and second electrodes of the device are releasably connected to the source of electrical potential,
wherein the plurality of cells suspended in the fluid are electroporated upon entering the electroporation zone.
81. The system of paragraph 79 or paragraph 80, wherein the plurality of cells has from 0% to about 25% phenotypic change relative to a baseline measurement of cell phenotype upon exiting the electroporation zone of the device.
82. The system of paragraph 79 or paragraph 80, wherein the plurality of cells has no phenotypic change upon exiting the electroporation zone.
83. The system of paragraph 79 or paragraph 80, wherein the device further comprises an outer structure comprising a housing configured to encase the first electrode, second electrode, and the electroporation zone of the device.
84. The system of paragraph 79 or paragraph 80, wherein the outer structure comprises a first electrical input operatively coupled to the first electrode and a second electrical input operatively coupled to the second electrode.
85. The system of paragraph 79 or paragraph 80, wherein the source of electrical potential is releasably connected to the first and second electrical inputs of the outer structure.
86. The system of paragraph 85, wherein the releasable connection between the first or second electrical inputs and the source of electrical potential is selected from the group consisting of a clamp, a clip, a spring, a sheath, a wire brush, or a combination thereof.
87. The system of paragraph 83, wherein the outer structure is integral to the device.
88. The system of paragraph 83, wherein the outer structure is releasably connected to the device.
89. The system of paragraph 79 or paragraph 80, wherein the electroporation is substantially non-thermal reversible electroporation.
90. The system of paragraph 79 or paragraph 80, wherein the electroporation is substantially non-thermal irreversible electroporation.
91. The system of paragraph 79 or paragraph 80, wherein the electroporation is substantially thermal irreversible electroporation.
92. The system of paragraph 79 or paragraph 80, further comprising a first reservoir fluidically connected to the entry zone.
93. The system of paragraph 79 or paragraph 80, further comprising a second reservoir fluidically connected to the recovery zone.
94. The system of paragraph 79 or paragraph 80, further comprising a third reservoir fluidically connected to the third inlet and the third outlet.
95. The system of paragraph 79 or paragraph 80, further comprising a fourth reservoir fluidically connected to the fourth inlet and the fourth outlet.
96. The device of paragraph 79 or paragraph 80, wherein the cross-section of the electroporation zone is selected from the group consisting of circular, ellipsoidal, polygonal, star, parallelogram, trapezoidal, and irregular.
97. The system of paragraph 79 or paragraph 80, wherein the cross-sectional dimension of the entry zone is between 0.01% and 100,000% of the cross-sectional dimension of the electroporation zone.
98. The system of paragraph 79 or paragraph 80, wherein cross-sectional dimension of the recovery zone is between 0.01% and 100,000% of the cross-sectional dimension of the electroporation zone.
99. The system of paragraph 79 or paragraph 80, wherein none of the entry zone, recovery zone, or electroporation zone reduce a cross-section dimension of any of the plurality of cells suspended in a fluid.
100. The system of paragraph 79 or paragraph 80, wherein the duty cycle of the electroporation is between 0.001% and 100%.
101. The system of paragraph 79 or paragraph 80, wherein the cross-section dimension of the electroporation zone is between 0.005 mm and 50 mm.
102. The system of paragraph 79 or paragraph 80, wherein the length of the electroporation zone is between 0.005 mm and 50 mm.
103. The system of paragraph 79 or paragraph 80, wherein the cross-sectional dimension of any of the first electrode or the second electrode is between 0.1 mm and 5 mm.
104. The system of paragraph 79 or paragraph 80, further comprising a fluid delivery source fluidically connected to the entry zone, wherein the fluid delivery source is configured to deliver the plurality of cells suspended in the fluid through the entry zone to the recovery zone.
105. The system of paragraph 104, wherein the delivery rate from the fluid delivery source is between 0.001 mL/min and 1,000 mL/min.
106. The system of paragraph 79 or paragraph 80, wherein the residence time of any of the plurality of cells suspended in the fluid is between 0.5 ms and 50 ms.
107. The system of paragraph 79 or paragraph 80, further comprising a controller operatively coupled to the source of electrical potential to deliver voltage pulses to the first electrode and second electrodes to generate an electrical potential difference between the first and second electrodes.
108. The system of paragraph 107, wherein the voltage pulses have an amplitude between 0.01 kV and 3 kV.
109. The system of paragraph 107, wherein the voltage pulses have a duration of between 0.01 ms and 1,000 ms.
110. The system of paragraph 107, wherein the voltage pulses are applied to the first and second electrodes at a frequency between 1 Hz and 50,000 Hz.
111. The system of paragraph 107, wherein the waveform of the voltage pulse is selected from the group consisting of DC, square, pulse, bipolar, sine, ramp, asymmetric bipolar, arbitrary, and any superposition or combinations thereof.
112. The system of paragraph 107, wherein the electric field generated from the voltage pulses has a magnitude of between 1 V/cm and 50,000 V/cm.
113. The system of paragraph 79 or paragraph 80, wherein the fluid has a conductivity of between 0.001 mS/cm and 500 mS/cm.
114. The system of any one of paragraphs 79-113, further comprising a plurality of cell transfection devices.
115. The system of paragraph 114, further comprising a plurality of outer structures.
116. A method of electro-mechanical delivery of a composition into at least a portion of a plurality of cells suspended in a fluid, the method comprising:
   (a) providing a device comprising:
      (i) a first electrode comprising a first inlet and a first outlet, wherein a lumen of the first electrode comprises an entry zone;
      (ii) a second electrode comprising a second inlet and a second outlet, wherein a lumen of the second electrode comprises a recovery zone; and
      (iii) an electroporation zone, wherein the electroporation zone is fluidically connected to the first outlet of the first electrode and the second inlet of the second electrode, and wherein application of an electrical potential difference to the first and second electrodes produces an electric field in the electroporation zone,
   (b) energizing the first and second electrodes to produce an electrical potential difference between the first and second electrodes, thereby producing an electric field in the electroporation zone; and
   (c) passing the plurality of cells suspended in the fluid with the composition through the electric field in the electroporation zone of the device;
wherein flow of the plurality of cells suspended in fluid with the composition through the electric field in the electroporation zone enhances temporary permeability of the plurality of cells, thereby introducing the composition into at least a portion of the plurality of cells.
117. A method of electroporating a composition into at least a portion of a plurality of cells suspended in a fluid, the method comprising:
   (a) providing a device comprising:
      (i) a first electrode comprising a first inlet and a first outlet, wherein a lumen of the first electrode comprises an entry zone;
      (ii) a second electrode comprising a second inlet and a second outlet, wherein a lumen of the second electrode comprises a recovery zone; and
      (iii) an electroporation zone, wherein the electroporation zone is fluidically connected to the first outlet of the first electrode and the second inlet of the second electrode, and wherein application of an electrical potential difference to the first and second electrodes produces an electric field in the electroporation zone,
   (b) energizing the first and second electrodes to produce an electrical potential difference between the first and second electrodes, thereby producing an electric field in the electroporation zone; and
   (c) passing the plurality of cells suspended in the fluid with the composition through the electric field in the electroporation zone of the device;
wherein flow of the plurality of cells suspended in fluid with the composition through the electric field in the electroporation zone enhances temporary permeability of the plurality of cells, thereby electroporating the composition into at least a portion of the plurality of cells.
118. The method of paragraph 116 or paragraph 117, further comprising assessing the health of a portion of the plurality of cells suspended in the fluid.
119. The method of paragraph 118, wherein the assessing comprises measuring the viability of the portion of the plurality of cells suspended in the fluid.
120. The method of paragraph 118, wherein the assessing comprises measuring the transfection efficiency of the portion of the plurality of cells suspended in the fluid.
121. The method of paragraph 118, wherein the assessing comprises measuring the cell recovery rate of the portion of the plurality of cells suspended in the fluid.
122. The method of paragraph 118, wherein the assessing comprises flow cytometry analysis of cell surface marker expression.
123. The method of paragraph 116 or paragraph 117, wherein the plurality of cells has from 0% to about 25% phenotypic change relative to a baseline measurement of cell phenotype upon exiting the electroporation zone of the device.
124. The method of paragraph 116 or paragraph 117, wherein the plurality of cells has no phenotypic change upon exiting the electroporation zone of the device.
125. The method of paragraph 116 or paragraph 117, wherein the electroporation is substantially non-thermal reversible electroporation.
126. The method of paragraph 116 or paragraph 117, wherein the electroporation is substantially non-thermal irreversible electroporation.
127. The method of paragraph 116 or paragraph 117, wherein the electroporation is substantially thermal irreversible electroporation.
128. The method of paragraph 116 or paragraph 117, wherein the electroporation zone of the device has a uniform cross-sectional dimension.
129. The method of paragraph 116 or paragraph 117, wherein the electroporation zone of the device has a non-uniform cross-sectional dimension.
130. The method of paragraph 116 or paragraph 117, wherein the device further comprises a plurality of electroporation zones.
131. The method of paragraph 130, wherein each of the plurality of electroporating zones has a uniform cross section.
132. The method of paragraph 130, wherein each of the plurality of electroporating zones has a non-uniform cross section.
133. The method of paragraph 116 or paragraph 117, wherein part c) occurs by the application of a positive pressure.
134. The method of paragraph 116 or paragraph 117, wherein the cells in the plurality of cells in the sample are selected from the group consisting of mammalian cells, eukaryotes, synthetic cells, human cells, animal cells, plant cells, primary cells, cell lines, suspension cells, adherent cells, immune cells, stem cells, blood cells, red blood cells, T cells, B cells, neutrophils, dendritic cells, antigen presenting cells (APCs), natural killer (NK) cells, monocytes, macrophages, peripheral blood mononuclear cells (PBMCs), human embryonic kidney (HEK-293) cells, or Chinese hamster ovary (CHO) cells.
135. The method of paragraph 134, wherein the cells comprise Jurkat cells.
136. The method of paragraph 134, wherein the cells comprise primary human T-cells.
137. The method of paragraph 134, wherein the cells comprise THP-1 cells.
138. The method of paragraph 134, wherein the cells comprise primary human macrophages.
139. The method of paragraph 134, wherein the cells comprise primary human monocytes.
140. The method of paragraph 134, wherein the cells comprise natural killer cells.
141. The method of paragraph 134, wherein the cells comprise human embryonic kidney cells.
142. The method of paragraph 134, wherein the cells comprise B-cells.
143. The method of paragraph 116 or paragraph 117, wherein the composition comprises at least one compound selected from the group consisting of therapeutic agents, vitamins, nanoparticles, charged molecules, uncharged molecules, DNA, RNA, CRISPR-Cas complex, proteins, viruses, polymers, a ribonucleoprotein (RNP), and polysaccharides.
144. The method of paragraph 116 or paragraph 117, wherein the composition has a concentration in the fluid of between 0.0001 μg/mL and 1000 μg/mL.
145. The method of paragraph 116 or paragraph 117, further comprising a first reservoir fluidically connected to the entry zone.
146. The method of paragraph 116 or paragraph 117, further comprising a second reservoir fluidically connected to the recovery zone.
147. The method of paragraph 116 or paragraph 117, wherein the cross-section of the electroporation zone is selected from the group consisting of circular, ellipsoidal, polygonal, star, parallelogram, trapezoidal, and irregular.
148. The method of paragraph 116 or paragraph 117, wherein the cross-sectional dimension of the entry zone is between 0.01% and 100,000% of the cross-sectional dimension of the electroporation zone.
149. The method of paragraph 116 or paragraph 117, wherein the cross-sectional dimension of the recovery zone is between 0.01% and 100,000% of the cross-sectional dimension of the electroporation zone.
150. The method of paragraph 116 or paragraph 117, wherein none of the entry zone, recovery zone, or electroporation zone reduces a cross-section dimension of any of the plurality of cells suspended in the fluid.
151. The method of paragraph 116 or paragraph 117, wherein the duty cycle of the electroporation is between 0.001% and 100%.
152. The method of paragraph 116 or paragraph 117, wherein the largest cross-section dimension of the electroporation zone is between 0.005 mm and 50 mm.
153. The method of paragraph 116 or paragraph 117, wherein the length of the electroporation zone is between 0.005 mm and 50 mm.
154. The method of paragraph 116 or paragraph 117, wherein the cross-sectional dimension of any of the first electrode or the second electrode is between 0.1 mm and 5 mm.
155. The method of paragraph 116 or paragraph 117, wherein the device further comprises an outer structure comprising a housing configured to encase the first electrode, second electrode, and the electroporation zone of the device.
156. The method of paragraph 155, wherein the outer structure comprises a first electrical input operatively coupled to the first electrode and a second electrical input operatively coupled to the second electrode.
157. The method of paragraph 155, wherein the outer structure is integral to the device.
158. The method of paragraph 155, wherein the outer structure is releasably connected to the device.
159. The method of paragraph 116 or paragraph 117, wherein the delivery rate of step c) is between 0.001 mL/min and 1,000 mL/min.
160. The method of paragraph 116 or paragraph 117, wherein the residence time of any of the plurality of cells suspended in the fluid is between 0.5 ms to 50 ms.
161. The method of paragraph 116 or paragraph 117, further comprising a controller operatively coupled to the source of electrical potential to deliver voltage pulses to the first electrode and second electrodes to generate an electrical potential difference between the first and second electrodes.
162. The method of paragraph 161, wherein the voltage pulses have an amplitude between 0.01 kV and 3 kV.
163. The method of paragraph 161, wherein the voltage pulses have a duration of between 0.01 ms and 1,000 ms.
164. The method of paragraph 161, wherein the voltage pulses are applied to the first and second electrodes at a frequency between 1 Hz and 50,000 Hz.
165. The method of paragraph 161, wherein the waveform of the voltage pulse is selected from the group consisting of DC, square, pulse, bipolar, sine, ramp, asymmetric bipolar, arbitrary, and any superposition and combination thereof.
166. The method of paragraph 161, wherein the electric field generated from the voltage pulses has a magnitude of between 1 V/cm and 50,000 V/cm.
167. The method of paragraph 116 or paragraph 117, wherein the fluid has a conductivity of between 0.001 mS/cm and 500 mS/cm.
168. The method of paragraph 116 or paragraph 117, further comprising a housing configured to house the device.
169. The method of paragraph 168, wherein the housing further comprises a thermal controller configured to increase or decrease the temperature of the housing.
170. The method of paragraph 169, wherein the thermal controller is a heating element selected from the group consisting of a heating block, liquid flow, battery powered heater, and a thin-film heater.
171. The method of paragraph 169, wherein the thermal controller is a cooling element selected from the group consisting of a liquid flow, evaporative cooler, and a Peltier device.
172. The method of any one of paragraphs 118-171, wherein the temperature of the plurality of cells suspended in the fluid is between 0° C. to 50° C.
173. The method of any one of paragraphs 116-172, wherein the device comprises a plurality of cell porating devices.
174. The method of paragraph 173, wherein the device comprises a plurality of outer structures.
175. The method of any one of paragraphs 116-174, further comprising storing the plurality of cells suspended in the fluid in a recovery buffer after poration.
176. The method of any one of paragraphs 116-175, wherein the electroporated cells have a viability after introduction of the composition between 0.1 and 99.9%.
177. The method of any one of paragraphs 116-176, wherein the efficiency of the introduction of the composition into the cells is between 0.1 and 99.9%.
178. The method of any one of paragraphs 116-177, wherein the number of recovered cells is between 10⁴ cells and 10¹² cells.
179. The method of any one of paragraphs 116-178, wherein the live engineered cell yield is between 0.1 and 500%.
180. A kit for electro-mechanical delivery of a composition into a plurality of cells suspended in a fluid, comprising:
   (a) a plurality of devices, each of the plurality of devices comprising:
      (i) a first electrode comprising a first inlet and a first outlet, wherein a lumen of the first electrode comprises an entry zone;
      (ii) a second electrode comprising a second inlet and a second outlet, wherein a lumen of the second electrode comprises a recovery zone; and
      (iii) an electroporation zone, wherein the electroporation zone is fluidically connected to the first outlet of the first electrode and the second inlet of the second electrode, wherein the electroporation zone has a substantially uniform cross-section dimension, and wherein application of an electrical potential difference to the first and second electrodes produces an electric field in the electroporation zone;
   (b) a plurality of outer structures configured to encase the plurality of devices, wherein each of the plurality of outer structure comprises:
      (i) a housing configured to electro-mechanically engage the first electrode, the second electrode, and the electroporation zone of the at least one device;
      (ii) a first electrical input operatively coupled to the first electrode; and
      (iii) a second electrical input operatively coupled to the second electrode; and
   (c) a transfection buffer for electro-mechanical delivery of the composition into the plurality of cells suspended in the fluid.
181. A kit for electroporating a composition into a plurality of cells suspended in a fluid, comprising:
   (a) a plurality of devices, each of the plurality of devices comprising:
      (i) a first electrode comprising a first inlet and a first outlet, wherein a lumen of the first electrode comprises an entry zone;
      (ii) a second electrode comprising a second inlet and a second outlet, wherein a lumen of the second electrode comprises a recovery zone; and
      (iii) an electroporation zone, wherein the electroporation zone is fluidically connected to the first outlet of the first electrode and the second inlet of the second electrode, wherein the electroporation zone has a substantially uniform cross-section dimension, and wherein application of an electrical potential difference to the first and second electrodes produces an electric field in the electroporation zone;
   (b) a plurality of outer structures configured to encase the plurality of devices, wherein each of the plurality of outer structure comprises:
      (i) a housing configured to electromechanically engage the first electrode, the second electrode, and the electroporation zone of the at least one device;
      (ii) a first electrical input operatively coupled to the first electrode; and
      (iii) a second electrical input operatively coupled to the second electrode; and
   (c) a transfection buffer for electroporating the plurality of cells suspended in the fluid.
182. The kit of paragraph 180 or paragraph 181, wherein the outer structures are integral to the plurality of devices.
183. The kit of paragraph 180 or paragraph 181, wherein the outer structures are releasably connected to the plurality of devices.

Other embodiments of the technology described herein can be defined according to any of the following numbered paragraphs:
1. A device for electroporation of a composition into a plurality of cells suspended in a liquid, the device comprising:
   (a) a first electrode comprising a first inlet, a first outlet, and a first lumen comprising a minimum cross-sectional dimension;
   (b) a second electrode comprising a second inlet, a second outlet, and a second lumen comprising a minimum cross-sectional dimension; and
   (c) an electroporation zone disposed between the first outlet and the second inlet, wherein the electroporation zone comprises a minimum cross-sectional dimension greater than about 100 μm, wherein the electroporation zone has a substantially uniform cross-sectional area;
wherein the first outlet, the electroporation zone, and the second inlet are in fluidic communication.
2. A device for electro-mechanical delivery of a composition into a plurality of cells suspended in a liquid, the device comprising:
   (a) a first electrode comprising a first inlet, a first outlet, and a first lumen comprising a minimum cross-sectional dimension;
   (b) a second electrode comprising a second inlet, a second outlet, and a second lumen comprising a minimum cross-sectional dimension; and
   (c) an electroporation zone disposed between the first outlet and the second inlet, wherein the electroporation zone comprises a minimum cross-sectional dimension greater than about 100 μm, wherein the electroporation zone has a substantially uniform cross-sectional area;
wherein the first outlet, the electroporation zone, and the second inlet are in fluidic communication.
3. The device of paragraph 1 or paragraph 2, wherein a transverse cross-section of the electroporation zone is a shape selected from a group consisting of circular, disk, elliptical, regular polygon, irregular polygon, curvilinear shape, star, parallelogram, trapezoidal, and irregular.
4. The device of any one of paragraphs 1-3, wherein the electroporation zone has a substantially circular transverse cross-section.
5. The device of any one of paragraphs 1-4, wherein the electroporation zone has a minimum cross-sectional dimension of between 0.1 mm and 50 mm.
6. The device of any of paragraphs 1-5, wherein the electroporation zone has a transverse cross-sectional area of between about 7850 μm² and about 2000 mm².
7. The device of any one of paragraphs 1-6, wherein the electroporation zone has a length of between 0.1 mm and 50 mm.
8. The device of any one of paragraphs 1-7, wherein a lumen of any of the first electrode and/or the second electrode has a minimum cross-sectional dimension of between 0.01 mm and 500 mm.
9. The device of any one of paragraphs 1-8, wherein a ratio of the minimum cross-sectional dimension of a lumen of either of the first or second electrode to the minimum cross-sectional dimension of the electroporation zone is between 1:10 and 10:1.
10. The device of any one of paragraphs 1-9, wherein a ratio of the minimum cross-sectional dimension of the electroporation zone to the length of the electroporation zone is between 1:100 and 100:1.
11. The device of any one of paragraphs 1-10, wherein a ratio of a transverse cross-sectional area of a lumen of any of the first electrode and/or the second electrode to the transverse cross-sectional area of the electroporation zone is between 1:10 and 10:1.
12. The device of any one of paragraphs 1-11, further comprising a first reservoir in fluidic communication with the first inlet and/or a second reservoir in fluid communication with the second outlet.
13. The device of any one of paragraphs 1-12, further comprising a third reservoir in fluidic communication with the first lumen or the second lumen.
14. The device of paragraph 13, wherein either of the first electrode or the second electrode has an additional inlet or outlet for fluidic communication with the third reservoir.
15. The device of any one of paragraphs 1-14, wherein the device further comprises one or more additional electroporation zones.
16. A system for electroporation of a composition into a plurality of cells suspended in a liquid, comprising:
   (a) a device comprising:
      (i) a first electrode comprising a first inlet, a first outlet, and a first lumen comprising a minimum cross-sectional dimension;
      (ii) a second electrode comprising a second inlet, a second outlet, and a second lumen comprising a minimum cross-sectional dimension; and
      (iii) an electroporation zone disposed between the first outlet and the second inlet, wherein the electroporation zone comprises a minimum cross-sectional dimension greater than about 100 μm, wherein the electroporation zone has a substantially uniform cross-sectional area;
wherein the first outlet, the electroporation zone, and the second inlet are in fluidic communication; and
   (b) a source of electrical potential, wherein the first electrode and the second electrode of the device are releasably in operative contact with the source of electrical potential.
17. A system for electro-mechanical delivery of a composition into a plurality of cells suspended in a liquid, comprising:
   (a) a device comprising:
      (i) a first electrode comprising a first inlet, a first outlet, and a first lumen comprising a minimum cross-sectional dimension;
      (ii) a second electrode comprising a second inlet, a second outlet, and a second lumen comprising a minimum cross-sectional dimension; and
      (iii) an electroporation zone disposed between the first outlet and the second inlet, wherein the electroporation zone comprises a minimum cross-sectional dimension greater than about 100 μm, wherein the electroporation zone has a substantially uniform cross-sectional area;
wherein the first outlet, the electroporation zone, and the second inlet are in fluidic communication; and
   (b) a source of electrical potential, wherein the first electrode and the second electrode of the device are releasably in operative contact with the source of electrical potential.
18. The system of paragraph 16 or paragraph 17, further comprising a first reservoir in fluidic communication with the first inlet.
19. The system of any one of paragraphs 16-18, further comprising a second reservoir in fluidic communication with the second outlet.
20. The system of any one of paragraphs 16-19, further comprising a third reservoir in fluidic communication with a lumen of any of the first electrode or the second electrode, wherein any of the first electrode or the second electrode has an additional inlet for fluidic communication with the third reservoir.
21. The system of any one of paragraphs 16-20, further comprising a fluid delivery source in fluidic communication with the first inlet, wherein the fluid delivery source is configured to deliver the liquid and/or the plurality of cells in suspension through the first lumen to the second outlet.
22. The system of any one of paragraphs 16-21, further comprising a controller operatively coupled to the source of electrical potential to deliver voltage pulses to the first electrode and the second electrode, wherein the voltage pulses generate an electrical potential difference between the first electrode and the second electrode, thus producing an electric field in the electroporation zone.
23. The system of any one of paragraphs 16-22, wherein the device further comprises one or more additional electroporation zones.
24. The system of paragraph 23, further comprising a housing configured to energize the electroporation zones parallel, in series, or offset in time, wherein the housing further comprises a tray that accommodates a plurality of electroporation devices, wherein the tray is modified with two grid electrodes, wherein a first grid electrode is electrically isolated from a second grid electrode, wherein an exterior of the first electrode of each of the plurality of devices is releasably in operative contact with any of a first spring-loaded electrode, a first mechanically connected electrode, or a first inductively connected electrode, wherein an exterior of the second electrode of each of the plurality of devices is releasably in operative contact with any of a second spring-loaded electrode, a second mechanically connected electrode, or a second inductively coupled electrode, wherein each of the plurality of devices releasably enters the housing through an opening in the grid electrodes, wherein any of the first spring-loaded electrode, first mechanically connected electrode, or first inductively connected electrode of each device is in operative contact with the first grid electrode and any of the second spring-loaded electrode, second mechanically connected electrode, or second inductively connected electrode of each device is in operative contact with the second grid electrode, wherein the grid electrodes are connected to the source of electrical potential.
25. The system of paragraph 24, wherein the source of electrical potential delivers voltage pulses to the grid electrodes, wherein the first grid electrode is energized at a particular applied voltage while the second grid electrode is energized at a particular applied voltage, wherein each of the plurality of devices is energized by the grid electrodes with an identical applied voltage pulse such that a magnitude of an electric field generated within each of the at least one electroporation zones of each device is substantially identical.
26. The system of paragraph 25, wherein the source of electrical potential includes additional circuitry or programming configured to modulate the delivery of voltage pulses to the grid electrodes, wherein each of the plurality of devices receives a different voltage from the grid electrodes, wherein a magnitude of an electric field generated within each of the at least one electroporation zones of each device is different.
27. A system for electroporation of a composition into a plurality of cells suspended in a liquid, comprising:
   (a) a device, comprising:
      (i) a first electrode comprising a first inlet, a first outlet, and a first lumen;
      (ii) a second electrode comprising a second inlet, a second outlet, and a second lumen;
      (iii) a third inlet and a third outlet, wherein the third inlet and the third outlet are in fluidic communication with the first lumen, wherein the third inlet and third outlet intersect the first electrode between the first inlet and the first outlet;
      (iv) a fourth inlet and a fourth outlet, wherein the fourth inlet and the fourth outlet are in fluidic communication with the second lumen, wherein the fourth inlet and fourth outlet intersect the second electrode between the second inlet and the second outlet; and
      (v) an electroporation zone disposed between the first outlet and the second inlet, wherein the electroporation zone has a length of between 0.1 mm and 50 mm and comprises a minimum cross-sectional dimension greater than about 100 μm, wherein a transverse cross-sectional area of the electroporation zone is substantially uniform;
      wherein a ratio of a minimum cross-sectional dimension of the first lumen to the minimum cross-sectional dimension of the electroporation zone is between 1:10 and 10:1, wherein a ratio of a minimum cross-sectional dimension of the second lumen to the minimum cross-sectional dimension of the electroporation zone is between 1:10 and 10:1, and wherein the first outlet, the electroporation zone, and the second inlet are in fluidic communication; and
   (b) a source of electrical potential, wherein the first and second electrodes of the device are releasably in operative contact with the source of electrical potential.
28. A system for electro-mechanical delivery of a composition into a plurality of cells suspended in a liquid, comprising:
   (a) a device, comprising:
      (i) a first electrode comprising a first inlet, a first outlet, and a first lumen;
      (ii) a second electrode comprising a second inlet, a second outlet, and a second lumen;
      (iii) a third inlet and a third outlet, wherein the third inlet and the third outlet are in fluidic communication with the first lumen, wherein the third inlet and third outlet intersect the first electrode between the first inlet and the first outlet;
      (iv) a fourth inlet and a fourth outlet, wherein the fourth inlet and the fourth outlet are in fluidic communication with the second lumen, wherein the fourth inlet and fourth outlet intersect the second electrode between the second inlet and the second outlet; and
      (v) an electroporation zone disposed between the first outlet and the second inlet, wherein the electroporation zone has a length of between 0.1 mm and 50 mm and comprises a minimum cross-sectional dimension greater than about 100 μm, wherein a transverse cross-sectional area of the electroporation zone is substantially uniform;
      wherein a ratio of a minimum cross-sectional dimension of the first lumen to the minimum cross-sectional dimension of the electroporation zone is between 1:10 and 10:1, wherein a ratio of a minimum cross-sectional dimension of the second lumen to the minimum cross-sectional dimension of the electroporation zone is between 1:10 and 10:1, and wherein the first outlet, the electroporation zone, and the second inlet are in fluidic communication; and
   (b) a source of electrical potential, wherein the first and second electrodes of the device are releasably in operative contact with the source of electrical potential.
29. A method of electroporation of a composition into a plurality of cells suspended in a flowing liquid, the method comprising:
   (a) providing a device comprising:
      (i) a first electrode comprising a first outlet, a first inlet, and a first lumen comprising a minimum cross-sectional dimension;
      (ii) a second electrode comprising a second outlet, a second inlet, and a second lumen comprising a minimum cross-sectional dimension; and
      (iii) an electroporation zone disposed between the first outlet and the second inlet, wherein the electroporation zone comprises a minimum cross-sectional dimension greater than about 100 μm, wherein the electroporation zone has a substantially uniform cross sectional area;
      and wherein the first outlet, the electroporation zone, and the second inlet are in fluidic communication;
   (b) applying an electrical potential difference between the first and second electrodes, thereby producing an electric field in the electroporation zone; and
   (c) passing the plurality of cells and the composition through the electroporation zone, thereby enhancing permeability of the plurality of cells and introducing the composition into the plurality of cells.
30. A method of electro-mechanical delivery of a composition into a plurality of cells suspended in a flowing liquid, the method comprising:
   (a) providing a device comprising:
      (i) a first electrode comprising a first outlet, a first inlet, and a first lumen comprising a minimum cross-sectional dimension;
      (ii) a second electrode comprising a second outlet, a second inlet, and a second lumen comprising a minimum cross-sectional dimension; and
      (iii) an electroporation zone disposed between the first outlet and the second outlet, wherein the electroporation zone comprises a minimum cross-sectional dimension greater than about 100 μm, wherein the electroporation zone has a substantially uniform cross sectional area;
      and wherein the first outlet, the electroporation zone, and the second inlet are in fluidic communication;
   (b) applying an electrical potential difference between the first and second electrodes, thereby producing an electric field in the electroporation zone; and
   (c) passing the plurality of cells and the composition through the electroporation zone, thereby enhancing permeability of the plurality of cells and introducing the composition into the plurality of cells.
31. The method of paragraph 29 or paragraph 30, wherein the plurality of the cells is in a separate liquid than the composition before step (b).
32. The method of paragraph 30 or 31, wherein step (b) comprises applying a fluid-driven positive pressure.
33. The method of any one of paragraphs 30-32, wherein none of the first lumen, second lumen, or electroporation zone has a minimum cross-sectional dimension that causes a cross-sectional dimension of any of the plurality of cells suspended in the liquid to be compressed temporarily.
34. The method of any one of paragraphs 30-33, wherein a flow rate of a liquid and/or the plurality of cells in suspension delivered from a fluid delivery source from the first lumen to the electroporation zone is between 0.001 mL/min and 1,000 mL/min, wherein the fluid delivery source is configured to deliver the liquid and/or the plurality of cells in suspension through the first lumen to the second outlet.
35. The method of any one of paragraphs 30-34, wherein a Reynolds number of a liquid and/or the plurality of cells in suspension delivered from a fluid delivery source from the first lumen to the electroporation zone is between 0.04 and 2.43×10⁴, wherein the fluid delivery source is configured to deliver the liquid and/or the plurality of cells in suspension through the first lumen to the second outlet.
36. The method of any one of paragraphs 30-35, wherein a maximum velocity of a liquid and/or the plurality of cells in suspension delivered from a fluid delivery source from the first lumen to the electroporation zone is between 5×10⁵ m/s and 32.7 m/s, wherein the fluid delivery source is configured to deliver the liquid and/or the plurality of cells in suspension through the first lumen to the second outlet.
37. The method of any one of paragraphs 30-36, wherein shear rates of a liquid and/or the plurality of cells in suspension delivered from a fluid delivery source from the first lumen to the electroporation zone are between 0.1 1/s and 2×10⁶ 1/s, wherein the fluid delivery source is configured to deliver the liquid and/or the plurality of cells in suspension through the first lumen to the second outlet.
38. The method of any one of paragraphs 30-37, wherein a peak pressure of a liquid and/or the plurality of cells in suspension delivered from a fluid delivery source from the first lumen to the electroporation zone is between 1×10⁻³ Pa and 9.5×10⁴ Pa, wherein the fluid delivery source is configured to deliver the liquid and/or the plurality of cells in suspension through the first lumen to the second outlet.
39. The method of any one of paragraphs 30-38, wherein an average velocity of a liquid and/or the plurality of cells in suspension delivered from a fluid delivery source from the first lumen to the electroporation zone is between 1.5×10⁻⁵ m/s and 15.9 m/s, wherein the fluid delivery source is configured to deliver the liquid and/or the plurality of cells in suspension through the first lumen to the second outlet.
40. The method of any one of paragraphs 30-39, wherein a kinematic viscosity of a liquid and/or the plurality of cells in suspension delivered from a fluid delivery source from the first lumen to the electroporation zone is between 1×10⁻⁶ m²/s and 15×10⁻⁴ m²/s, wherein the fluid delivery source is configured to deliver the liquid and/or the plurality of cells in suspension through the first lumen to the second outlet.
41. The method of any one of paragraphs 30-40, wherein a residence time in the electroporation zone of the plurality of cells suspended in the liquid is between 0.5 ms and 50 ms.
42. The method of any one of paragraphs 30-41, wherein passing the plurality of cells and the composition through the electroporation zone induces a mechanical stress on the flowing liquid, thereby further enhancing permeability of the plurality of cells and introducing the composition into the plurality of cells.
43. The method of any one of paragraphs 30-41, wherein the electric field is produced by voltage pulses.
44. The method of paragraph 43, wherein the voltage pulses energize the first electrode at a first applied voltage and the second electrode is energized at a second applied voltage, thereby applying an electrical potential difference between the first and second electrodes.
45. The method of paragraph 43 or 44, wherein the voltage pulses each have an amplitude between −3 kV and 3 kV.
46. The method of any one of paragraphs 43-45, wherein the voltage pulses have a duration of between 0.01 ms and 1,000 ms.
47. The method of any one of paragraphs 43-46, wherein the voltage pulses are applied to the first and second electrodes at a frequency of between 1 Hz and 50,000 Hz.
48. The method of any one of paragraphs 43-47, wherein the voltage pulse comprises a waveform selected from a group consisting of DC, square, pulse, bipolar, sine, ramp, asymmetric bipolar, arbitrary, and any superposition or combinations thereof.
49. The method of any one of paragraphs 43-48, wherein the electric field generated from the voltage pulses has a magnitude of between 1 V/cm and 50,000 V/cm.
50. The method of any one of paragraphs 43-49, wherein a duty cycle of the voltage pulses is between 0.001% and 100%.
51. The method of any one of paragraphs 30-50, wherein the liquid has a conductivity of between 0.001 mS/cm and 500 mS/cm.
52. The method of any one of paragraphs 30-51, wherein a temperature of the liquid is between 0° C. and 50° C.
53. The method of any one of paragraphs 30-52, further comprising storing the plurality of cells in a recovery buffer after electro-mechanical delivery of the composition.
54. The method of any one of paragraphs 30-53, wherein the composition comprises at least one compound selected from the group consisting of therapeutic agents, vitamins, nanoparticles, charged molecules, uncharged molecules, DNA, RNA, CRISPR-Cas complex, proteins, enzymes, peptides, viruses, polymers, a ribonucleoprotein, polysaccharides, engineered nucleases, transcription activator-like effector nucleases (TALENs), zinc-finger nucleases (ZFNs), homing nucleases, meganucleases (MNs), megaTALs, and transposons.
55. The method of any one of paragraphs 30-54, wherein the composition has a concentration in the liquid of between 0.0001 μg/mL and 1000 μg/mL.
56. The method of any one of paragraphs 30-55, wherein the plurality of cells comprises eukaryotic cells, plant cells, prokaryotic cells, or synthetic cells.
57. The method of paragraph 56, wherein the plurality of cells comprises human cells or animal cells.
58. The method of any one of paragraphs 30-57, wherein the plurality of cells comprises primary cells, cells from a cell line, adherent cells, unstimulated cells, stimulated cells, activated cells, stem cells, blood cells, Chinese hamster ovary (CHO) cells, immune cells, red blood cells, or peripheral blood mononuclear cells (PBMCs).
59. The method of paragraph 58, wherein the plurality of cells comprises adaptive immune cells and/or innate immune cells.
60. The method of any one of paragraphs 30-58, wherein the plurality of cells comprises antigen presenting cells (APCs), monocytes, T-cells, B-cells, dendritic cells, macrophages, neutrophils, natural killer (NK) cells, Jurkat cells, THP-1 cells, human embryonic kidney (HEK-293) cells, or embryonic stem cells (ESCs), mesenchymal stem cells (MSCs), or hematopoietic stem cells (HSCs).
61. The method of any one of paragraphs 30-58, wherein the plurality of cells comprises primary human NK cells, primary human induced pluripotent stem cells (iPSCs), primary human macrophages, or primary human monocytes.
62. A kit for electroporation of a composition into a plurality of cells suspended in a liquid, comprising:
   (a) a plurality of devices, each of the plurality of devices comprising:
      (i) a first electrode comprising a first outlet, a first inlet, and a first lumen comprising a minimum cross-sectional dimension;
      (ii) a second electrode comprising a second outlet, a second inlet, and a second lumen comprising a minimum cross-sectional dimension; and
      (iii) an electroporation zone disposed between the first outlet and the second inlet, wherein the electroporation zone comprises a minimum cross-sectional dimension greater than about 100 μm, wherein the electroporation zone has a substantially uniform cross-sectional area; and
      wherein application of an electrical potential difference to the first and second electrodes produces an electric field in the electroporation zone; and
   (b) a plurality of outer structures configured to encase the plurality of devices, wherein each of the plurality of outer structures comprises:
      (i) a housing configured to encase the first electrode, second electrode, and the electroporation zone of the at least one device;
      (ii) a first electrical input operatively coupled to the first electrode; and
      (iii) a second electrical input operatively coupled to the second electrode.
63. A kit for electro-mechanical delivery of a composition into a plurality of cells suspended in a liquid, comprising:
   (a) a plurality of devices, each of the plurality of devices comprising:
      (i) a first electrode comprising a first outlet, a first inlet, and a first lumen comprising a minimum cross-sectional dimension;
      (ii) a second electrode comprising a second outlet, a second inlet, and a second lumen comprising a minimum cross-sectional dimension; and
      (iii) an electroporation zone disposed between the first outlet and the second inlet, wherein the electroporation zone comprises a minimum cross-sectional dimension greater than about 100 μm, wherein the electroporation zone has a substantially uniform cross-sectional area; and
      wherein application of an electrical potential difference to the first and second electrodes produces an electric field in the electroporation zone; and
   (b) a plurality of outer structures configured to encase the plurality of devices, wherein each of the plurality of outer structures comprises:
      (i) a housing configured to encase the first electrode, second electrode, and the electroporation zone of the at least one device;
      (ii) a first electrical input operatively coupled to the first electrode; and
      (iii) a second electrical input operatively coupled to the second electrode.
64. A kit for electroporation of a composition into a plurality of cells suspended in a liquid, comprising:
   (a) a plurality of devices, each of the plurality of devices of any one of paragraphs 1-15; and
   (b) a plurality of outer structures configured to encase the plurality of devices, wherein each of the plurality of outer structures comprises:
      (i) a housing configured to encase the first electrode, second electrode, and the electroporation zone of the at least one device;
      (ii) a first electrical input operatively coupled to the first electrode; and
      (vi) a second electrical input operatively coupled to the second electrode.
65. A kit for electro-mechanical delivery of a composition into a plurality of cells suspended in a liquid, comprising:
   (a) a plurality of devices, each of the plurality of devices of any one of paragraphs 1-15; and
   (b) a plurality of outer structures configured to encase the plurality of devices, wherein each of the plurality of outer structures comprises:
      (i) a housing configured to encase the first electrode, second electrode, and the electroporation zone of the at least one device;
      (ii) a first electrical input operatively coupled to the first electrode; and
      (vi) a second electrical input operatively coupled to the second electrode.

### Other Embodiments

All publications, patents, and patent applications mentioned in this specification are herein incorporated by reference to the same extent as if each independent publication or patent application was specifically and individually indicated to be incorporated by reference. In the event of a conflicting definition between this and any reference incorporated herein, the definition provided herein applies.

While the disclosure has been described in connection with specific embodiments thereof, it will be understood that it is capable of further modifications and this application is intended to cover any variations, uses, or adaptations of the disclosure following, in general, the principles of the disclosure and including such departures from the present disclosure that come within known or customary practice within the art to which the disclosure pertains and may be applied to the essential features hereinbefore set forth, and follows in the scope of the claims.

Other embodiments are within the claims.

What is claimed is:

1. A device for electro-mechanical delivery of a composition into a plurality of cells suspended in a liquid, the device comprising:
(a) a first electrode comprising a first inlet, a first outlet, and a first lumen comprising a minimum cross-sectional dimension;
(b) a second electrode comprising a second inlet, a second outlet, and a second lumen comprising a minimum cross-sectional dimension; and
(c) an electroporation zone disposed between the first outlet and the second inlet, wherein the electroporation zone comprises a minimum cross-sectional dimension greater than about 100 μm, wherein the electroporation zone has a substantially uniform cross-sectional area;
wherein the first outlet, the electroporation zone, and the second inlet are in fluidic communication.

2. The device of claim 1, wherein a transverse cross-section of the electroporation zone is a shape selected from a group consisting of circular, disk, elliptical, regular polygon, irregular polygon, curvilinear shape, star, parallelogram, trapezoidal, and irregular.

3. The device of claim 1 or 2, wherein the electroporation zone has a substantially circular transverse cross-section.

4. The device of any one of claims 1-3, wherein the electroporation zone has a minimum cross-sectional dimension of between 0.1 mm and 50 mm.

5. The device of any of claims 1-4, wherein the electroporation zone has a transverse cross-sectional area of between about 7850 μm² and about 2000 mm².

6. The device of any one of claims 1-5, wherein the electroporation zone has a length of between 0.1 mm and 50 mm.

7. The device of any one of claims 1-6, wherein a lumen of any of the first electrode and/or the second electrode has a minimum cross-sectional dimension of between 0.01 mm and 500 mm.

8. The device of any one of claims 1-7, wherein a ratio of the minimum cross-sectional dimension of a lumen of either of the first or second electrode to the minimum cross-sectional dimension of the electroporation zone is between 1:10 and 10:1.

9. The device of any one of claims 1-8, wherein a ratio of the minimum cross-sectional dimension of the electroporation zone to the length of the electroporation zone is between 1:100 and 100:1.

10. The device of any one of claims 1-9, wherein a ratio of a transverse cross-sectional area of a lumen of any of the first electrode and/or the second electrode to the transverse cross-sectional area of the electroporation zone is between 1:10 and 10:1.

11. The device of any one of claims 1-10, further comprising a first reservoir in fluidic communication with the first inlet and/or a second reservoir in fluid communication with the second outlet.

12. The device of any one of claims 1-11, further comprising a third reservoir in fluidic communication with the first lumen or the second lumen.

13. The device of claim 12, wherein either of the first electrode or the second electrode has an additional inlet or outlet for fluidic communication with the third reservoir.

14. The device of any one of claims 1-13, wherein the device further comprises one or more additional electroporation zones.

15. A system for electro-mechanical delivery of a composition into a plurality of cells suspended in a liquid, comprising:
(a) a device comprising:
(i) a first electrode comprising a first inlet, a first outlet, and a first lumen comprising a minimum cross-sectional dimension;
(ii) a second electrode comprising a second inlet, a second outlet, and a second lumen comprising a minimum cross-sectional dimension; and
(iii) an electroporation zone disposed between the first outlet and the second inlet, wherein the electroporation zone comprises a minimum cross-sectional dimension greater than about 100 μm, wherein the electroporation zone has a substantially uniform cross-sectional area;
wherein the first outlet, the electroporation zone, and the second inlet are in fluidic communication; and
(b) a source of electrical potential, wherein the first electrode and the second electrode of the device are releasably in operative contact with the source of electrical potential.

16. The system of claim 15, further comprising a first reservoir in fluidic communication with the first inlet.

17. The system of claim 15 or 16, further comprising a second reservoir in fluidic communication with the second outlet.

18. The system of any one of claims 15-17, further comprising a third reservoir in fluidic communication with a lumen of any of the first electrode or the second electrode, wherein any of the first electrode or the second electrode has an additional inlet for fluidic communication with the third reservoir.

19. The system of any one of claims 15-18, further comprising a fluid delivery source in fluidic communication with the first inlet, wherein the fluid delivery source is configured to deliver the liquid and/or the plurality of cells in suspension through the first lumen to the second outlet.

20. The system of any one of claims 15-19, further comprising a controller operatively coupled to the source of electrical potential to deliver voltage pulses to the first electrode and the second electrode, wherein the voltage pulses generate an electrical potential difference between the first electrode and the second electrode, thus producing an electric field in the electroporation zone.

21. The system of any one of claims 15-20, wherein the device further comprises one or more additional electroporation zones.

22. The system of claim 21, further comprising a housing configured to energize the electroporation zones parallel, in series, or offset in time, wherein the housing further comprises a tray that accommodates a plurality of electroporation devices, wherein the tray is modified with two grid electrodes, wherein a first grid electrode is electrically isolated from a second grid electrode, wherein an exterior of the first electrode of each of the plurality of devices is releasably in operative contact with any of a first spring-loaded electrode, a first mechanically connected electrode, or a first inductively connected electrode, wherein an exterior of the second electrode of each of the plurality of devices is releasably in operative contact with any of a second spring-loaded electrode, a second mechanically connected electrode, or a second inductively coupled electrode, wherein each of the plurality of devices releasably enters the housing through an opening in the grid electrodes, wherein any of the first spring-loaded electrode, first mechanically connected electrode, or first inductively connected electrode of each device is in operative contact with the first grid electrode and any of the second spring-loaded electrode, second mechanically connected electrode, or second inductively connected electrode of each device is in operative contact with the second grid electrode, wherein the grid electrodes are connected to the source of electrical potential.

23. The system of claim 22, wherein the source of electrical potential delivers voltage pulses to the grid electrodes, wherein the first grid electrode is energized at a particular applied voltage while the second grid electrode is energized at a particular applied voltage, wherein each of the plurality of devices is energized by the grid electrodes with an identical applied voltage pulse such that a magnitude of an electric field generated within each of the at least one electroporation zones of each device is substantially identical.

24. The system of claim 23, wherein the source of electrical potential includes additional circuitry or programming configured to modulate the delivery of voltage pulses to the grid electrodes, wherein each of the plurality of devices receives a different voltage from the grid electrodes, wherein a magnitude of an electric field generated within each of the at least one electroporation zones of each device is different.

25. A system for electro-mechanical delivery of a composition into a plurality of cells suspended in a liquid, comprising:
(a) a device comprising:
(i) a first electrode comprising a first inlet, a first outlet, and a first lumen;
(ii) a second electrode comprising a second inlet, a second outlet, and a second lumen;
(iii) a third inlet and a third outlet, wherein the third inlet and the third outlet are in fluidic communication with the first lumen, wherein the third inlet and third outlet intersect the first electrode between the first inlet and the first outlet;
(iv) a fourth inlet and a fourth outlet, wherein the fourth inlet and the fourth outlet are in fluidic communication with the second lumen, wherein the fourth inlet and fourth outlet intersect the second electrode between the second inlet and the second outlet; and
(v) an electroporation zone disposed between the first outlet and the second inlet, wherein the electroporation zone has a length of between 0.1 mm and 50 mm and comprises a minimum cross-sectional dimension greater than about 100 μm, wherein a transverse cross-sectional area of the electroporation zone is substantially uniform;
wherein a ratio of a minimum cross-sectional dimension of the first lumen to the minimum cross-sectional dimension of the electroporation zone is between 1:10 and 10:1, wherein a ratio of a minimum cross-sectional dimension of the second lumen to the minimum cross-sectional dimension of the electroporation zone is between 1:10 and 10:1, and wherein the first outlet, the electroporation zone, and the second inlet are in fluidic communication; and
(b) a source of electrical potential, wherein the first and second electrodes of the device are releasably in operative contact with the source of electrical potential.

26. A method of electro-mechanical delivery of a composition into a plurality of cells suspended in a flowing liquid, the method comprising:
(a) providing a device comprising:
(i) a first electrode comprising a first outlet, a first inlet, and a first lumen comprising a minimum cross-sectional dimension;
(ii) a second electrode comprising a second outlet, a second inlet, and a second lumen comprising a minimum cross-sectional dimension; and
(iii) an electroporation zone disposed between the first outlet and the second inlet, wherein the electroporation zone comprises a minimum cross-sectional dimension greater than about 100 μm, wherein the electroporation zone has a substantially uniform cross sectional area;
and wherein the first outlet, the electroporation zone, and the second inlet are in fluidic communication;
(b) applying an electrical potential difference between the first and second electrodes, thereby producing an electric field in the electroporation zone; and
(c) passing the plurality of cells and the composition through the electroporation zone, thereby enhancing permeability of the plurality of cells and introducing the composition into the plurality of cells.

27. The method of claim 26, wherein the plurality of the cells is in a separate liquid than the composition before step (b).

28. The method of claim 26 or 27, wherein step (b) comprises applying a fluid-driven positive pressure.

29. The method of any one of claims 26-28, wherein none of the first lumen, second lumen, or electroporation zone has a minimum cross-sectional dimension that causes a cross-sectional dimension of any of the plurality of cells suspended in the liquid to be compressed temporarily.

30. The method of any one of claims 26-29, wherein a flow rate of a liquid and/or the plurality of cells in suspension delivered from a fluid delivery source from the first lumen to the electroporation zone is between 0.001 mL/min and 1,000 mL/min, wherein the fluid delivery source is configured to deliver the liquid and/or the plurality of cells in suspension through the first lumen to the second outlet.

31. The method of any one of claims 26-30, wherein a Reynolds number of a liquid and/or the plurality of cells in suspension delivered from a fluid delivery source from the first lumen to the electroporation zone is between 0.04 and 2.43×10⁴, wherein the fluid delivery source is configured to deliver the liquid and/or the plurality of cells in suspension through the first lumen to the second outlet.

32. The method of any one of claims 26-31, wherein a maximum velocity of a liquid and/or the plurality of cells in suspension delivered from a fluid delivery source from the first lumen to the electroporation zone is between 5×10⁻⁵ m/s and 32.7 m/s, wherein the fluid delivery source is configured to deliver the liquid and/or the plurality of cells in suspension through the first lumen to the second outlet.

33. The method of any one of claims 26-32, wherein shear rates of a liquid and/or the plurality of cells in suspension delivered from a fluid delivery source from the first lumen to the electroporation zone are between 0.1 s⁻¹ and 2×10⁶ s⁻¹, wherein the fluid delivery source is configured to deliver the liquid and/or the plurality of cells in suspension through the first lumen to the second outlet.

34. The method of any one of claims 26-33, wherein a peak pressure of a liquid and/or the plurality of cells in suspension delivered from a fluid delivery source from the first lumen to the electroporation zone is between 1×10⁻³ Pa and 9.5×10⁴ Pa, wherein the fluid delivery source is configured to deliver the liquid and/or the plurality of cells in suspension through the first lumen to the second outlet.

35. The method of any one of claims 26-34, wherein an average velocity of a liquid and/or the plurality of cells in suspension delivered from a fluid delivery source from the first lumen to the electroporation zone is between 1.5×10⁻⁵ m/s and 15.9 m/s, wherein the fluid delivery source is configured to deliver the liquid and/or the plurality of cells in suspension through the first lumen to the second outlet.

36. The method of any one of claims 26-35, wherein a kinematic viscosity of a liquid and/or the plurality of cells in suspension delivered from a fluid delivery source from the first lumen to the electroporation zone is between 1×10⁻⁶ m²/s and 15×10⁻⁴ m²/s, wherein the fluid delivery source is configured to deliver the liquid and/or the plurality of cells in suspension through the first lumen to the second outlet.

37. The method of any one of claims 26-36, wherein a residence time in the electroporation zone of the plurality of cells suspended in the liquid is between 0.5 ms and 50 ms.

38. The method of claim 26, wherein passing the plurality of cells and the composition through the electroporation zone induces a mechanical stress on the flowing liquid, thereby further enhancing permeability of the plurality of cells and introducing the composition into the plurality of cells.

39. The method of any one of claims 26-37, wherein the electric field is produced by voltage pulses.

40. The method of claim 39, wherein the voltage pulses energize the first electrode at a first applied voltage and the second electrode is energized at a second applied voltage, thereby applying an electrical potential difference between the first and second electrodes.

41. The method of claim 39 or 40, wherein the voltage pulses each have an amplitude between −3 kV and 3 kV.

42. The method of any one of claims 39-41, wherein the voltage pulses have a duration of between 0.01 ms and 1,000 ms.

43. The method of any one of claims 39-42, wherein the voltage pulses are applied to the first and second electrodes at a frequency of between 1 Hz and 50,000 Hz.

44. The method of any one of claims 39-43, wherein the voltage pulse comprises a waveform selected from a group consisting of DC, square, pulse, bipolar, sine, ramp, asymmetric bipolar, arbitrary, and any superposition or combinations thereof.

45. The method of any one of claims 39-44, wherein the electric field generated from the voltage pulses has a magnitude of between 1 V/cm and 50,000 V/cm.

46. The method of any one of claims 39-45, wherein a duty cycle of the voltage pulses is between 0.001% and 100%.

47. The method of any one of claims 26-46, wherein the liquid has a conductivity of between 0.001 mS/cm and 500 mS/cm.

48. The method of any one of claims 26-47, wherein a temperature of the liquid is between 0° C. and 50° C.

49. The method of any one of claims 26-48, further comprising storing the plurality of cells in a recovery buffer after electro-mechanical delivery of the composition.

50. The method of any one of claims 26-49, wherein the composition comprises at least one compound selected from the group consisting of therapeutic agents, vitamins, nanoparticles, charged molecules, uncharged molecules, DNA, RNA, CRISPR-Cas complex, proteins, enzymes, peptides, viruses, polymers, a ribonucleoprotein, polysaccharides, engineered nucleases, transcription activator-like effector nucleases (TALENs), zinc-finger nucleases (ZFNs), homing nucleases, meganucleases (MNs), megaTALs, and transposons.

51. The method of any one of claims 26-50, wherein the composition has a concentration in the liquid of between 0.0001 μg/mL and 1000 μg/mL.

52. The method of any one of claims 26-51, wherein the plurality of cells comprises eukaryotic cells, plant cells, prokaryotic cells, or synthetic cells.

53. The method of claim 52, wherein the plurality of cells comprises human cells or animal cells.

54. The method of any one of claims 26-53, wherein the plurality of cells comprises primary cells, cells from a cell line, adherent cells, unstimulated cells, stimulated cells, activated cells, stem cells, blood cells, Chinese hamster ovary (CHO) cells, immune cells, red blood cells, or peripheral blood mononuclear cells (PBMCs).

55. The method of claim 54, wherein the plurality of cells comprises adaptive immune cells and/or innate immune cells.

56. The method of any one of claims 26-54, wherein the plurality of cells comprises antigen presenting cells (APCs), monocytes, T-cells, B-cells, dendritic cells, macrophages, neutrophils, natural killer (NK) cells, Jurkat cells, THP-1 cells, human embryonic kidney (HEK-293) cells, or embryonic stem cells (ESCs), mesenchymal stem cells (MSCs), or hematopoietic stem cells (HSCs).

57. The method of any one of claims 26-54, wherein the plurality of cells comprises primary human NK cells, primary human induced pluripotent stem cells (iPSCs), primary human macrophages, or primary human monocytes.

58. A kit for electro-mechanical delivery of a composition into a plurality of cells suspended in a liquid, comprising:
(a) a plurality of devices, each of the plurality of devices comprising:
(i) a first electrode comprising a first outlet, a first inlet, and a first lumen comprising a minimum cross-sectional dimension;
(ii) a second electrode comprising a second outlet, a second inlet, and a second lumen comprising a minimum cross-sectional dimension; and
(iii) an electroporation zone disposed between the first outlet and the second inlet, wherein the electroporation zone comprises a minimum cross-sectional dimension greater than about 100 μm, wherein the electroporation zone has a substantially uniform cross-sectional area; and
wherein application of an electrical potential difference to the first and second electrodes produces an electric field in the electroporation zone; and
(b) a plurality of outer structures configured to encase the plurality of devices, wherein each of the plurality of outer structures comprises:
(i) a housing configured to encase the first electrode, second electrode, and the electroporation zone of the at least one device;
(ii) a first electrical input operatively coupled to the first electrode; and
(iii) a second electrical input operatively coupled to the second electrode.

59. A kit for electro-mechanical delivery of a composition into a plurality of cells suspended in a liquid, comprising:
(a) a plurality of devices, each of the plurality of devices of any one of claims 1-14; and
(b) a plurality of outer structures configured to encase the plurality of devices, wherein each of the plurality of outer structures comprises:
(i) a housing configured to encase the first electrode, second electrode, and the electroporation zone of the at least one device;
(ii) a first electrical input operatively coupled to the first electrode; and
(vi) a second electrical input operatively coupled to the second electrode.
